(19) United States
(12) Patent Application Publication
(10) Pub. No.: US 2016/0108039 A1
(43) Pub. Date: Apr. 21, 2016

(54) TRIAZOLOPYRIDINES AS THROMBIN INHIBITORS FOR THE TREATMENT OF THROMBOEMBOLIC DISEASES

(71) Applicant: BAYER PHARMA AKTIENGESELLSCHAFT, Berlin (DE)

(72) Inventors: Swen ALLERHEILlGEN, Essen (DE); Anja BUCHMÜLLER, Essen (DE); Karen ENGEL, Rossdorf (DE); Christoph GERDES, Köln (DE); Kersten Matthias GERICKE, Wuppertal (DE); Michael GERISCH, Wuppertal (DE); Stefan HEITMEIER, Wülfrath (DE); Alexander HILLlSCH, Solingen (DE); Tom KINZEL, Düsseldorf (DE); Philip LIENAU, Berlin (DE); Bernd RIEDL, Wuppertal (DE); Susanne RÖHRIG, Hilden (DE); Martina Victoria SCHMIDT, Köln (DE); Julia STRASSBURGER, Wuppertal (DE); Adrian TERSTEEGEN, Wuppertal (DE)

(21) Appl. No.: 14/895,675

(22) PCT Filed: Jun. 2, 2014
(86) PCT No.: PCT/EP2014/061307

§ 371 Date: Dec. 3, 2015

(30) Foreign Application Priority Data
Jun. 3, 2013 (EP) 13170211.0

(51) Int. Cl. C07D 471/04; C07D 519/00

(52) U.S. Cl. CPC C07D 471/04; C07D 519/00

(57) ABSTRACT

The invention relates to substituted triazolopyridines and to processes for their preparation and to their use for preparing medicaments for the treatment and/or prophylaxis of diseases, in particular of cardiovascular disorders, preferably of thrombotic or thromboembolic disorders.

The invention relates to substituted triazolopyridines and to processes for their preparation and to their use for preparing medicaments for the treatment and/or prophylaxis of diseases, in particular of cardiovascular disorders, preferably of thrombotic or thromboembolic disorders.

Blood coagulation is a protective mechanism of the organism which helps to “seal” defects in the wall of the blood vessels quickly and reliably. Thus, loss of blood can be avoided or kept to a minimum. Haemostasis after injury of the blood vessels is effected mainly by the coagulation system in which an enzymatic cascade of complex reactions of plasma proteins is triggered. Numerous blood coagulation factors are involved in this process, each of which factors converts, on activation, the respectively next inactive precursor into its active form. At the end of the cascade comes the conversion of soluble fibrinogen into insoluble fibrin, resulting in the formation of a blood clot. In blood coagulation, traditionally the intrinsic and the extrinsic system, which end in a final joint reaction path, are distinguished. Here, factors Xa and IIa (thrombin) play key roles: Factor Xa bundles the signals of the two coagulation paths since it is formed both via factor VIIa/tissue factor (extrinsic path) and via the tenase complex (intrinsic path) by conversion of factor X. The activated serine protease Xa cleaves prothrombin to thrombin which, via a series of reactions, transduces the impulses from the cascade to the coagulation state of the blood: thrombin directly cleaves fibrinogen to fibrin. It activates factor XIII, required for stabilization of the fibrin clot, to factor XIIIa. In addition, thrombin is a potent trigger of platelet aggregation (via PAR-1 activation), which also contributes considerably to haemostasis. By activating TAFI (thrombin-activatable fibrinolysis inhibitor) to TAFIa, thrombin in the complex with thrombomodulin inhibits the dissolution of the clot. Activation of factors V and VIII potentiates the production of thrombin and thus in turn amplifies the coagulation reaction.

In addition to unbound thrombin in the blood, bound forms are also known: During the formation of a fibrin clot, thrombin and prothrombinase (factor Xa in a complex) are bound to the fibrin skeleton. These enzyme molecules are still active and cannot be inhibited by endogenous antithrombin III. Thus, in this manner, clots have a general procoagulative potential.

In addition, thrombin, in particular via activation of PAR-1 receptors on endothelial cells, is also involved in inflammatory processes which, in interaction with the coagulation system, accelerates both processes.

Uncontrolled activation of the coagulation system or defect inhibition of the activation processes may lead to the formation of local thromboses or embolisms in vessels (arteries, veins, lymph vessels) or cardiac cavities. In addition, systemic hypercoagulability may lead to system-wide formation of thrombi and finally to consumption coagulopathy in the context of a disseminated intravasal coagulation. Thromboembolic complications are furthermore encountered in microangiopathic haemolytic anaemias, extracorporeal circulatory systems, such as haemodialysis, and also prosthetic heart valves and stents.

In the course of many cardiovascular and metabolic disorders, owing to systemic factors such as hyperlipidaemia, diabetes or smoking, owing to changes in blood flow with stasis, for example in atrial fibrillation, or owing to pathological changes in vessel walls, for example endothelial dysfunctions or atherosclerosis, there is an increased tendency for coagulation and platelet activation which, via formation of fibrin- and platelet-rich thrombi, may lead to thromboembolic disorders and thrombotic complications with life-threatening conditions. Accordingly, thromboembolic disorders are still the most frequent cause of morbidity and mortality in most industrialized countries [Heart Disease: A Textbook of Cardiovascular Medicine, Eugene Braunwald, 5th edition, 1997, W.B. Saunders Company, Philadelphia].

The anticoagulants known from the prior art, for example substances for inhibiting or preventing blood coagulation, have various disadvantages. In the therapy and prophylaxis of thromboembolic disorders, use is made, firstly, of heparin which is administered parenterally or subcutaneously. Because of more favourable pharmacokinetic properties, preference is these days increasingly given to low-molecular-weight heparin; however, the known disadvantages described hereinbelow encountered in heparin therapy cannot be avoided either in this manner. Thus, heparin is orally ineffective and has only a comparatively short half-life. In addition, there is a high risk of bleeding, there may in particular be cerebral haemorrhages and bleeding in the gastrointestinal tract, and there may be thrombopaenia, alopecia medicomentosa or osteoporosis [Pschyrembel, Klinisches Wörterbuch [clinical dictionary], 257th edition, 1994, Walter de Gruyter Verlag, page 610, keyword “Heparin”; Römpp Lexikon Chemie, version 1.5, 1998, Georg Thieme Verlag Stuttgart, keyword “Heparin”]. Low-molecular-weight heparins do have a lower probability of leading to the development of heparin-induced thrombocytopaenia; however, they can likewise only be administered subcutaneously. This also applies to fondaparinux, a synthetically produced selective factor Xa inhibitor having a long half-life.

A second class of anticoagulants are the vitamin K antagonists. These include, for example, 1,3-indanediones and in particular compounds such as warfarin, phenprocoumon, dicumarol and other coumarin derivatives which non-selectively inhibit the synthesis of various products of certain vitamin K-dependent coagulation factors in the liver. Owing to the mechanism of action, the onset of action is only very slow (latency to the onset of action 36 to 48 hours). The compounds can be administered orally; however, owing to the high risk of bleeding and the narrow therapeutic index complicated individual adjustment and monitoring of the patient are required [J. Hirsh, J. Dalen, D. R. Anderson et al., “Oral anticoagulants: Mechanism of action, clinical effectiveness, and optimal therapeutic range” Chest 2001, 119, 8S-21S; J. Ansell, J. Hirsh, J. Dalen et al., “Managing oral anticoagulant therapy” Chest 2001, 119, 22S-38S; P. S. Wells, A. M. Holbrook, N. R. Crowther et al., “Interactions of warfarin with drugs and food” Ann. Intern. Med. 1994, 121, 676-683]. In addition, other side-effects such as gastrointestinal problems, hair loss and skin necroses have been described.

More recent approaches for oral anticoagulants are in various phases of clinical evaluation or in clinical use; however, they have also displayed disadvantages such as, for example, highly variable bioavailability, liver damage and bleeding complications, in particular in patients with damaged kidneys.

For antithrombotic medicaments, the therapeutic width is of importance: The distance between the therapeutically active dose for coagulation inhibition and the dose where bleeding may occur should be as big as possible so that maximum therapeutic activity is achieved at a minimum risk profile.

In particular under therapeutic conditions with thrombi already present, it may be advantageous to inhibit also the factor IIa present in the thrombus, and thereby promote a rapid degradation of the thrombus. Using, for example, argatroban or hirudin as FIIa inhibitors, the advantageous effect of FIIa inhibition on an existing thrombus alone or in the presence of tissue plaminogen activator (tPA) has been demonstrated in various in-vitro and in-vivo models.

Accordingly, it is an object of the present invention to provide novel compounds as thrombin inhibitors for the treatment of cardiovascular disorders, in particular of thrombotic or thromboembolic disorders, in humans and animals, which compounds have a broad therapeutic width and good pharmacokinetic properties.

WO2009/023179 describes inter alia the use of triazolopyridines for treating the hepatitis C virus.

The invention provides compounds of the formula

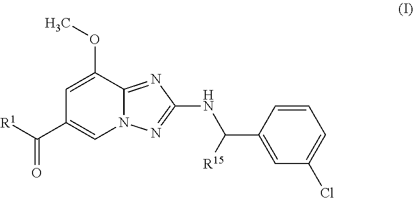

in which
R¹ represents a group of the formula

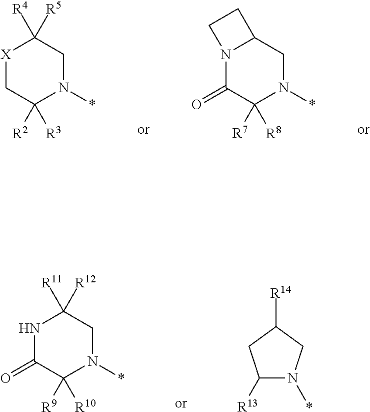

where * is the point of attachment to the carbonyl group,
X represents an oxygen atom, a sulphur atom or CH—R⁶,
   where
   R⁶ represents hydrogen or hydroxy,
R² represents hydrogen, aminocarbonyl, C₁-C₆-alkyl, C₃-C₆-cycloalkyl or phenyl,
   where alkyl and cycloalkyl may be substituted by a substituent selected from the group consisting of hydroxy, methoxy, cyano, hydroxycarbonyl, aminocarbonyl, methylsulphonyl, difluoromethoxy and trifluoromethoxy,
   or
   where alkyl and cycloalkyl may be substituted by 1 to 3 fluorine substituents,
R³ represents hydrogen or C₁-C₄-alkyl,
or
R² and R³ together with the carbon atom to which they are attached form a cyclopropyl ring, cyclobutyl ring or cyclopentyl ring,
   where the cyclobutyl ring and the cyclopentyl ring may be substituted by a hydroxy substituent,
R⁴ represents hydrogen or C₁-C₆-alkyl,
   where alkyl may be substituted by a hydroxy substituent,
   or
   where alkyl may be substituted by 1 to 3 fluorine substituents,
R⁵ represents C₁-C₄-alkyl,
or
R⁴ and R⁵ together with the carbon atom to which they are attached form a cyclopropyl ring, cyclobutyl ring or cyclopentyl ring,
   where the cyclobutyl ring and the cyclopentyl ring may be substituted by a hydroxy substituent,
R⁷ represents hydrogen or C₁-C₆-alkyl,
   where alkyl may be substituted by one substituent selected from the group consisting of cyano, hydroxy and methoxy,
   or
   where alkyl may be substituted by 1 to 3 fluorine substituents,
R⁸ represents hydrogen,
R⁹ represents hydrogen or C₁-C₆-alkyl,
   where alkyl may be substituted by one substituent selected from the group consisting of hydroxy and cyano,
   or
   where alkyl may be substituted by 1 to 3 fluorine substituents,
R¹⁰ represents hydrogen,
R¹¹ represents C₁-C₄-alkyl,
   where alkyl may be substituted by a hydroxy substituent,
R¹² represents hydrogen or C₁-C₄-alkyl,
or
R¹¹ and R¹² together with the carbon atom to which they are attached form a cyclopropyl ring, cyclobutyl ring or cyclopentyl ring,
   where the cyclobutyl ring and the cyclopentyl ring may be substituted by a hydroxy substituent,
R¹³ represents hydroxymethyl or hydroxyethyl,
R¹⁴ represents methoxy or ethoxy,
   where methoxy and ethoxy may be substituted by 1 to 3 substituents selected from the group consisting of deuterium and fluorine,
      and
      R¹⁵ represents hydrogen, methyl or fluoromethyl,
      and the salts thereof, the solvates thereof and the solvates of the salts thereof.

Compounds according to the invention are the compounds of the formula (I) and the salts, solvates and solvates of the salts thereof, and also the compounds encompassed by formula (I) and specified hereinafter as working example(s), and the salts, solvates and solvates of the salts thereof, to the extent that the compounds encompassed by formula (I) and specified hereinafter are not already salts, solvates and solvates of the salts.

The compounds according to the invention may, depending on their structure, exist in different stereoisomeric forms, i.e. in the form of configurational isomers or else optionally as conformational isomers (enantiomers and/or diastereomers, including those in the case of atropisomers). The present invention therefore encompasses the enantiomers and diastereomers, and the respective mixtures thereof. The stereoisomerically uniform constituents can be isolated from such mixtures of enantiomers and/or diastereomers in a known manner; chromatography processes are preferably used for this, especially HPLC chromatography on an achiral or chiral phase.

If the compounds according to the invention can occur in tautomeric forms, the present invention encompasses all the tautomeric forms.

The present invention also encompasses all suitable isotopic variants of the inventive compounds. An isotopic variant of an inventive compound is understood here to mean a compound in which at least one atom within the inventive compound has been exchanged for another atom of the same atomic number but with a different atomic mass from the atomic mass which usually or predominantly occurs in nature. Examples of isotopes which can be incorporated into a compound according to the invention are those of hydrogen, carbon, nitrogen, oxygen, phosphorus, sulphur, fluorine, chlorine, bromine and iodine, such as ²H (deuterium), ³H (tritium), ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I. Particular isotopic variants of a compound according to the invention, especially those in which one or more radioactive isotopes have been incorporated, may be beneficial, for example, for the examination of the mechanism of action or of the active ingredient distribution in the body; due to comparatively easy preparability and detectability, especially compounds labelled with ³H or ¹⁴C isotopes are suitable for this purpose. In addition, the incorporation of isotopes, for example of deuterium, can lead to particular therapeutic benefits as a consequence of greater metabolic stability of the compound, for example an extension of the half-life in the body or a reduction in the active dose required; such modifications of the compounds according to the invention may therefore in some cases also constitute a preferred embodiment of the present invention. Isotopic variants of the inventive compounds can be prepared by the processes known to those skilled in the art, for example by the methods described below and the procedures described in the working examples, by using corresponding isotopic modifications of the respective reagents and/or starting compounds.

Preferred salts in the context of the present invention are physiologically acceptable salts of the inventive compounds. However, the invention also encompasses salts which themselves are unsuitable for pharmaceutical applications but which can be used, for example, for the isolation or purification of the inventive compounds.

Physiologically acceptable salts of the inventive compounds include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention also include salts of conventional bases, by way of example and with preference alkali metal salts (e.g. sodium and potassium salts), alkaline earth metal salts (e.g. calcium and magnesium salts) and ammonium salts derived from ammonia or organic amines having 1 to 16 carbon atoms, by way of example and with preference ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine, N-methylpiperidine and choline.

Solvates in the context of the invention are described as those forms of the inventive compounds which form a complex in the solid or liquid state by coordination with solvent molecules. Hydrates are a specific form of the solvates in which the coordination is with water.

In addition, the present invention also encompasses prodrugs of the inventive compounds. The term “prodrugs” encompasses compounds which for their part may be biologically active or inactive but are converted during their residence time in the body into compounds according to the invention (for example by metabolism or hydrolysis).

In the context of the present invention, the term “treatment” or “treating” includes inhibition, retardation, checking, alleviating, attenuating, restricting, reducing, suppressing, repelling or healing of a disease, a condition, a disorder, an injury or a health problem, or the development, the course or the progression of such states and/or the symptoms of such states. The term “therapy” is understood here to be synonymous with the term “treatment”.

The terms “prevention”, “prophylaxis” and “preclusion” are used synonymously in the context of the present invention and refer to the avoidance or reduction of the risk of contracting, experiencing, suffering from or having a disease, a condition, a disorder, an injury or a health problem, or a development or advancement of such states and/or the symptoms of such states.

The treatment or prevention of a disease, a condition, a disorder, an injury or a health problem may be partial or complete.

In the context of the present invention, unless specified otherwise, the substituents are defined as follows:

Alkyl represents a straight-chain or branched alkyl radical having 1 to 6 carbon atoms, preferably 1 to 4 carbon atoms, by way of example and with preference methyl, ethyl, n-propyl, isopropyl, n-butyl, isobutyl, 1-methylpropyl, tert-butyl, n-pentyl, isopentyl, 1-ethylpropyl, 1-methylbutyl, 2-methylbutyl, 3-methylbutyl, n-hexyl, 1-methylpentyl, 2-methylpentyl, 3-methylpentyl, 4-methylpentyl, 3,3-dimethylbutyl, 1-ethylbutyl and 2-ethylbutyl.

Cycloalkyl represents a monocyclic cycloalkyl group having 3 to 6 carbon atoms, preferred examples of cycloalkyl being cyclopropyl, cyclobutyl, cyclopentyl and cyclohexyl.

In the formulae of the group which may represent R¹, the end point of the line marked by * in each case does not represent a carbon atom or a CH₂ group, but is part of the bond to the atom to which R¹ is attached.

Preference is given to compounds of the formula (I) in which

R¹ represents a group of the formula

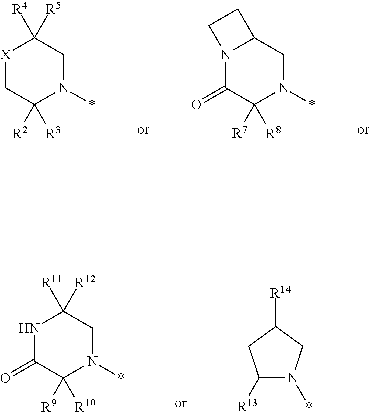

where * is the point of attachment to the carbonyl group,
X represents an oxygen atom or CH—R⁶,
   where
   R⁶ represents hydrogen,
R² represents aminocarbonyl, C₁-C₄-alkyl or C₃-C₆-cycloalkyl,
   where alkyl and cycloalkyl may be substituted by a substituent selected from the group consisting of hydroxy, methoxy and hydroxycarbonyl,
   or
   where alkyl may be substituted by 1 to 3 fluorine substituents,
R³ represents hydrogen or C₁-C₄-alkyl,
or
R² and R³ together with the carbon atom to which they are attached form a cyclobutyl ring,
   where the cyclobutyl ring may be substituted by a hydroxy substituent,
R⁴ represents hydrogen or C₁-C₄-alkyl,
   where alkyl may be substituted by a hydroxy substituent,
R⁵ represents C₁-C₄-alkyl,
R⁷ represents C₁-C₄-alkyl,
   where alkyl may be substituted by a methoxy substituent,
R⁸ represents hydrogen,
R⁹ represents C₁-C₄-alkyl,
R¹⁰ represents hydrogen,
R¹¹ represents C₁-C₄-alkyl,
R¹² represents hydrogen,
or
R¹¹ and R¹² together with the carbon atom to which they are bonded form a cyclopropyl ring,
R¹³ represents hydroxymethyl,
R¹⁴ represents ethoxy,
   where ethoxy may be substituted by 1 to 3 substituents selected from the group consisting of deuterium and fluorine,
      and
      R¹⁵ represents hydrogen, methyl or fluoromethyl,
      and the salts thereof, the solvates thereof and the solvates of the salts thereof.

Preference is also given to compounds of the formula (I) in which

R¹ represents a group of the formula

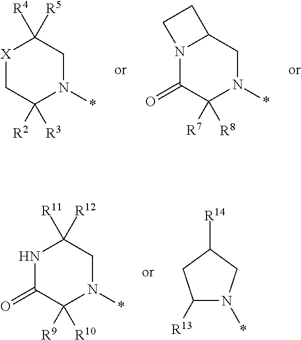

where * is the point of attachment to the carbonyl group,
X represents an oxygen atom,
R² represents C₁-C₄-alkyl or cyclobutyl,
   where alkyl may be substituted by one substituent selected from the group consisting of hydroxy and methoxy,
   or
   where alkyl may be substituted by 1 to 3 fluorine substituents,
   and
   where cyclobutyl is substituted by a hydroxy substituent,
R³ represents hydrogen or methyl,
R⁴ represents hydrogen or methyl,
and
R⁵ represents methyl,
or
R² represents methyl or ethyl,
   where methyl and ethyl may be substituted by 1 to 3 fluorine substituents,
R³ represents hydrogen or methyl,
R⁴ represents C₁-C₄-alkyl,
   where alkyl is substituted by a hydroxy substituent,
and
R⁵ represents methyl,
or
R² and R³ together with the carbon atom to which they are attached form a cyclobutyl ring,
   where the cyclobutyl ring is substituted by a hydroxy substituent,
R⁴ represents hydrogen or methyl,
and
R⁵ represents methyl,
R⁷ represents methyl or ethyl,
   where methyl and ethyl may be substituted by a methoxy substituent,
R⁸ represents hydrogen,
R⁹ represents methyl or ethyl,
R¹⁰ represents hydrogen,
R¹¹ represents methyl,
R¹² represents hydrogen,
or
R¹¹ and R¹² together with the carbon atom to which they are bonded form a cyclopropyl ring,
R¹³ represents hydroxymethyl,
R¹⁴ represents ethoxy,
   where ethoxy may be substituted by 1 to 3 substituents selected from the group consisting of deuterium and fluorine,
      and
      R¹⁵ represents hydrogen, methyl or fluoromethyl,
      and the salts thereof, the solvates thereof and the solvates of the salts thereof.

Preference is also given to compounds of the formula (I) in which

R¹ represents a group of the formula

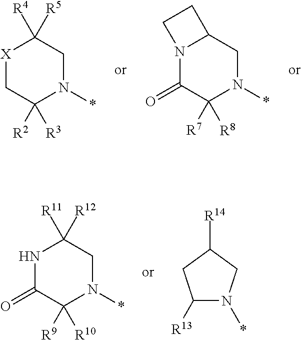

where * is the point of attachment to the carbonyl group,
X represents an oxygen atom,
R² represents C₁-C₄-alkyl or cyclobutyl,
   where alkyl is substituted by a hydroxy substituent,
   and
   where cyclobutyl is substituted by a hydroxy substituent,
R³ represents hydrogen,
R⁴ represents hydrogen or methyl,
and
R⁵ represents methyl,
or
R² represents methyl,
   where methyl may be substituted by 1 to 2 fluorine substituents,
R³ represents hydrogen or methyl,
R⁴ represents C₁-C₄-alkyl,
   where alkyl is substituted by a hydroxy substituent,
and
R⁵ represents methyl,
or
R² and R³ together with the carbon atom to which they are attached form a cyclobutyl ring, where the cyclobutyl ring is substituted by a hydroxy substituent,
R⁴ represents hydrogen,
and
R⁵ represents methyl,
R⁷ represents methyl,
R⁸ represents hydrogen,
R⁹ represents methyl or ethyl,
R¹⁰ represents hydrogen,
R¹¹ represents methyl,
R¹² represents hydrogen,
or
R¹¹ and R¹² together with the carbon atom to which they are bonded form a cyclopropyl ring,
R¹³ represents hydroxymethyl,
R¹⁴ represents ethoxy,
   where ethoxy may be substituted by 1 to 3 substituents selected from the group consisting of deuterium and fluorine,
      and
      R¹⁵ represents hydrogen, methyl or fluoromethyl,
      and the salts thereof, the solvates thereof and the solvates of the salts thereof.

Preference is also given to compounds of the formula (I) in which

R¹ represents a group of the formula

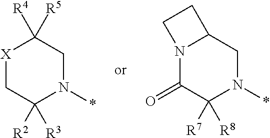

where * is the point of attachment to the carbonyl group,
X represents an oxygen atom, a sulphur atom or CH—R⁶,
   where
   R⁶ represents hydrogen or hydroxy,
R² represents hydrogen, aminocarbonyl, C₁-C₆-alkyl, C₃-C₆-cycloalkyl or phenyl,
   where alkyl and cycloalkyl may be substituted by a substituent selected from the group consisting of hydroxy, methoxy, cyano, hydroxycarbonyl, aminocarbonyl, methylsulphonyl, difluoromethoxy and trifluoromethoxy,
   or
   where alkyl and cycloalkyl may be substituted by 1 to 3 fluorine substituents,
R³ represents hydrogen or C₁-C₄-alkyl,
or
R² and R³ together with the carbon atom to which they are attached form a cyclopropyl ring, cyclobutyl ring or cyclopentyl ring,
   where the cyclobutyl ring and the cyclopentyl ring may be substituted by a hydroxy substituent,
R⁴ represents hydrogen or C₁-C₆-alkyl,
   where alkyl may be substituted by a hydroxy substituent,
   or
   where alkyl may be substituted by 1 to 3 fluorine substituents,
R⁵ represents C₁-C₄-alkyl,
or
R⁴ and R⁵ together with the carbon atom to which they are attached form a cyclopropyl ring, cyclobutyl ring or cyclopentyl ring,
   where the cyclobutyl ring and the cyclopentyl ring may be substituted by a hydroxy substituent,
R⁷ represents hydrogen or C₁-C₆-alkyl,
   where alkyl may be substituted by one substituent selected from the group consisting of cyano, hydroxy and methoxy,
   or
   where alkyl may be substituted by 1 to 3 fluorine substituents,
R⁸ represents hydrogen,
   and
   R¹⁵ represents hydrogen, methyl or fluoromethyl,
   and the salts thereof, the solvates thereof and the solvates of the salts thereof.

Preference is given to compounds of the formula (I) in which

R¹ represents a group of the formula

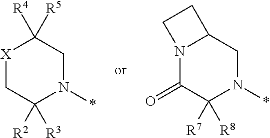

where * is the point of attachment to the carbonyl group,
X represents an oxygen atom or CH—R⁶,
   where
   R⁶ represents hydrogen,
R² represents aminocarbonyl, C₁-C₄-alkyl or C₃-C₆-cycloalkyl,
   where alkyl and cycloalkyl may be substituted by a substituent selected from the group consisting of hydroxy, methoxy and hydroxycarbonyl,
   or
   where alkyl may be substituted by 1 to 3 fluorine substituents,
R³ represents hydrogen or C₁-C₄-alkyl,
or
R² and R³ together with the carbon atom to which they are attached form a cyclobutyl ring,
   where the cyclobutyl ring may be substituted by a hydroxy substituent,
R⁴ represents hydrogen or C₁-C₄-alkyl,
   where alkyl may be substituted by a hydroxy substituent,
R⁵ represents C₁-C₄-alkyl,
R⁷ represents C₁-C₄-alkyl,
   where alkyl may be substituted by a methoxy substituent,
R⁸ represents hydrogen,
   and
   R¹⁵ represents hydrogen, methyl or fluoromethyl,
   and the salts thereof, the solvates thereof and the solvates of the salts thereof.

Preference is given to compounds of the formula (I) in which
R¹ represents a group of the formula

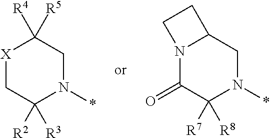

where * is the point of attachment to the carbonyl group,
X represents an oxygen atom,
R² represents C₁-C₄-alkyl or cyclobutyl,
   where alkyl may be substituted by one substituent selected from the group consisting of hydroxy and methoxy,
   or
   where alkyl may be substituted by 1 to 3 fluorine substituents,
   and
   where cyclobutyl is substituted by a hydroxy substituent,
R³ represents hydrogen or methyl,
R⁴ represents hydrogen or methyl,
and

R⁵ represents methyl,
or
R² represents methyl or ethyl,
   where methyl and ethyl may be substituted by 1 to 3 fluorine substituents,
R³ represents hydrogen or methyl,
R⁴ represents C₁-C₄-alkyl,
   where alkyl is substituted by a hydroxy substituent,
and
R⁵ represents methyl,
or
R² and R³ together with the carbon atom to which they are attached form a cyclobutyl ring,
   where the cyclobutyl ring is substituted by a hydroxy substituent,
R⁴ represents hydrogen or methyl,
and
R⁵ represents methyl,
R⁷ represents methyl or ethyl,
   where methyl and ethyl may be substituted by a methoxy substituent,
R⁸ represents hydrogen,
and
   R¹⁵ represents hydrogen, methyl or fluoromethyl,
   and the salts thereof, the solvates thereof and the solvates of the salts thereof.

Preference is given to compounds of the formula (I) in which

R¹ represents a group of the formula

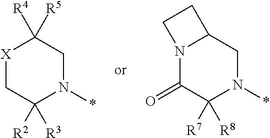

where * is the point of attachment to the carbonyl group,
X represents an oxygen atom,
R² represents C₁-C₄-alkyl or cyclobutyl,
   where alkyl is substituted by a hydroxy substituent,
   and
   where cyclobutyl is substituted by a hydroxy substituent,
R³ represents hydrogen,
R⁴ represents hydrogen or methyl,
and
R⁵ represents methyl,
or
R² represents methyl,
   where methyl may be substituted by 1 to 2 fluorine substituents,
R³ represents hydrogen or methyl,
R⁴ represents C₁-C₄-alkyl,
   where alkyl is substituted by a hydroxy substituent,
and
R⁵ represents methyl,
or
R² and R³ together with the carbon atom to which they are attached form a cyclobutyl ring,
   where the cyclobutyl ring is substituted by a hydroxy substituent,
R⁴ represents hydrogen,
and
R⁵ represents methyl,
R⁷ represents methyl,
R⁸ represents hydrogen,
   and
   R¹⁵ represents hydrogen, methyl or fluoromethyl,
   and the salts thereof, the solvates thereof and the solvates of the salts thereof.

Preference is given to compounds of the formula (I) in which

R¹ represents a group of the formula

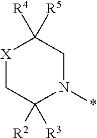

where * is the point of attachment to the carbonyl group,
X represents an oxygen atom, a sulphur atom or CH—R⁶,
   where
   R⁶ represents hydrogen or hydroxy,
R² represents hydrogen, aminocarbonyl, C₁-C₆-alkyl, C₃-C₆-cycloalkyl or phenyl,
   where alkyl and cycloalkyl may be substituted by a substituent selected from the group consisting of hydroxy, methoxy, cyano, hydroxycarbonyl, aminocarbonyl, methylsulphonyl, difluoromethoxy and trifluoromethoxy,
   or
   where alkyl and cycloalkyl may be substituted by 1 to 3 fluorine substituents,
R³ represents hydrogen or C₁-C₄-alkyl,
or
R² and R³ together with the carbon atom to which they are attached form a cyclopropyl ring, cyclobutyl ring or cyclopentyl ring,
   where the cyclobutyl ring and the cyclopentyl ring may be substituted by a hydroxy substituent,
R⁴ represents hydrogen or C₁-C₆-alkyl,
   where alkyl may be substituted by a hydroxy substituent,
   or
   where alkyl may be substituted by 1 to 3 fluorine substituents,
R⁵ represents C₁-C₄-alkyl,
or
R⁴ and R⁵ together with the carbon atom to which they are attached form a cyclopropyl ring, cyclobutyl ring or cyclopentyl ring,
   where the cyclobutyl ring and the cyclopentyl ring may be substituted by a hydroxy substituent,
      and
      R¹⁵ represents hydrogen, methyl or fluoromethyl,
      and the salts thereof, the solvates thereof and the solvates of the salts thereof.

Preference is given to compounds of the formula (I) in which

R¹ represents a group of the formula

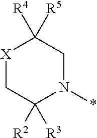

where * is the point of attachment to the carbonyl group,
X represents an oxygen atom or CH—R⁶,
where
   R⁶ represents hydrogen,
R² represents aminocarbonyl, C₁-C₄-alkyl or C₃-C₆-cycloalkyl,
   where alkyl and cycloalkyl may be substituted by a substituent selected from the group consisting of hydroxy, methoxy and hydroxycarbonyl,
   or
   where alkyl may be substituted by 1 to 3 fluorine substituents,
R³ represents hydrogen or C₁-C₄-alkyl,
or
R² and R³ together with the carbon atom to which they are attached form a cyclobutyl ring,
   where the cyclobutyl ring may be substituted by a hydroxy substituent,
R⁴ represents hydrogen or C₁-C₄-alkyl,
   where alkyl may be substituted by a hydroxy substituent,
R⁵ represents C₁-C₄-alkyl,
   and
   R¹⁵ represents hydrogen, methyl or fluoromethyl,
   and the salts thereof, the solvates thereof and the solvates of the salts thereof.

Preference is given to compounds of the formula (I) in which

R¹ represents a group of the formula

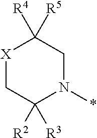

where * is the point of attachment to the carbonyl group,
X represents an oxygen atom,
R² represents C₁-C₄-alkyl or cyclobutyl,
   where alkyl may be substituted by one substituent selected from the group consisting of hydroxy and methoxy,
   or
   where alkyl may be substituted by 1 to 3 fluorine substituents,
   and
   where cyclobutyl is substituted by a hydroxy substituent,
R³ represents hydrogen or methyl,
R⁴ represents hydrogen or methyl,
   and
R⁵ represents methyl,
or
R² represents methyl or ethyl,
   where methyl and ethyl may be substituted by 1 to 3 fluorine substituents,
R³ represents hydrogen or methyl,
R⁴ represents C₁-C₄-alkyl,
   where alkyl is substituted by a hydroxy substituent,
and
R⁵ represents methyl,
or
R² and R³ together with the carbon atom to which they are attached form a cyclobutyl ring,
   where the cyclobutyl ring is substituted by a hydroxy substituent,
R⁴ represents hydrogen or methyl,
and
R⁵ represents methyl,
   and
   R¹⁵ represents hydrogen, methyl or fluoromethyl,
   and the salts thereof, the solvates thereof and the solvates of the salts thereof.

Preference is given to compounds of the formula (I) in which

R¹ represents a group of the formula

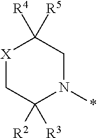

where * is the point of attachment to the carbonyl group,
X represents an oxygen atom,
R² represents C₁-C₄-alkyl or cyclobutyl,
   where alkyl is substituted by a hydroxy substituent,
   and
   where cyclobutyl is substituted by a hydroxy substituent,
R³ represents hydrogen,
R⁴ represents hydrogen or methyl,
and
R⁵ represents methyl,
or
R² represents methyl,
   where methyl may be substituted by 1 to 2 fluorine substituents,
R³ represents hydrogen or methyl,
R⁴ represents C₁-C₄-alkyl,
   where alkyl is substituted by a hydroxy substituent,
and
R⁵ represents methyl,
or
R² and R³ together with the carbon atom to which they are attached form a cyclobutyl ring,
   where the cyclobutyl ring is substituted by a hydroxy substituent,
R⁴ represents hydrogen,
and
R⁵ represents methyl,
   and
   R¹⁵ represents hydrogen, methyl or fluoromethyl,
   and the salts thereof, the solvates thereof and the solvates of the salts thereof.

Preference is given to compounds of the formula (I) in which

R¹ represents a group of the formula

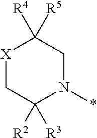

where * is the point of attachment to the carbonyl group,
X represents an oxygen atom,
R² represents C₁-C₄-alkyl or cyclobutyl,
   where alkyl is substituted by a hydroxy substituent,
   and
   where cyclobutyl is substituted by a hydroxy substituent,
R³ represents hydrogen,
R⁴ represents hydrogen or methyl,
and
R⁵ represents methyl,
and
   R¹⁵ represents hydrogen, methyl or fluoromethyl,
   and the salts thereof, the solvates thereof and the solvates of the salts thereof.

Preference is given to compounds of the formula (I) in which

R¹ represents a group of the formula

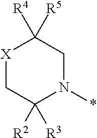

where * is the point of attachment to the carbonyl group,
X represents an oxygen atom,
R² represents methyl,
   where methyl may be substituted by 1 to 2 fluorine substituents,
R³ represents hydrogen or methyl,
R⁴ represents C₁-C₄-alkyl,
   where alkyl is substituted by a hydroxy substituent,
and
R⁵ represents methyl,
   and
   R¹⁵ represents hydrogen, methyl or fluoromethyl,
   and the salts thereof, the solvates thereof and the solvates of the salts thereof.

Preference is given to compounds of the formula (I) in which

R¹ represents a group of the formula

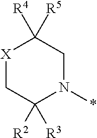

where * is the point of attachment to the carbonyl group,
X represents an oxygen atom,
R² and R³ together with the carbon atom to which they are attached form a cyclobutyl ring,
   where the cyclobutyl ring is substituted by a hydroxy substituent,
R⁴ represents hydrogen,
and
R⁵ represents methyl,
   and
   R¹⁵ represents hydrogen, methyl or fluoromethyl,
   and the salts thereof, the solvates thereof and the solvates of the salts thereof.

Preference is given to compounds of the formula (I) in which

R¹ represents a group of the formula

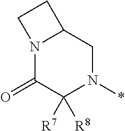

where * is the point of attachment to the carbonyl group,
R⁷ represents hydrogen or C₁-C₆-alkyl,
   where alkyl may be substituted by one substituent selected from the group consisting of cyano, hydroxy and methoxy,
   or
   where alkyl may be substituted by 1 to 3 fluorine substituents,
R⁸ represents hydrogen,
   and
   R¹⁵ represents hydrogen, methyl or fluoromethyl,
   and the salts thereof, the solvates thereof and the solvates of the salts thereof.

Preference is given to compounds of the formula (I) in which

R¹ represents a group of the formula

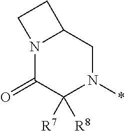

where * is the point of attachment to the carbonyl group,
R⁷ represents C₁-C₄-alkyl,
   where alkyl may be substituted by a methoxy substituent,
R⁸ represents hydrogen,
   and
   R¹⁵ represents hydrogen, methyl or fluoromethyl,
   and the salts thereof, the solvates thereof and the solvates of the salts thereof.

Preference is given to compounds of the formula (I) in which

R¹ represents a group of the formula

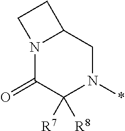

where * is the point of attachment to the carbonyl group,
X represents an oxygen atom,
R⁷ represents methyl or ethyl,
   where methyl and ethyl may be substituted by a methoxy substituent,
R⁸ represents hydrogen,
   and
   R¹⁵ represents hydrogen, methyl or fluoromethyl,
   and the salts thereof, the solvates thereof and the solvates of the salts thereof.

Preference is given to compounds of the formula (I) in which

R¹ represents a group of the formula

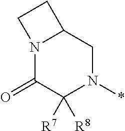

where * is the point of attachment to the carbonyl group,
R⁷ represents methyl,
R⁸ represents hydrogen,
   and
   R⁵ represents hydrogen, methyl or fluoromethyl,
   and the salts thereof, the solvates thereof and the solvates of the salts thereof.

Preference is given to compounds of the formula (I) in which

R¹ represents a group of the formula

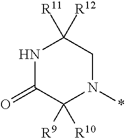

where * is the point of attachment to the carbonyl group,
R⁹ represents hydrogen or C₁-C₆-alkyl,
   where alkyl may be substituted by one substituent selected from the group consisting of hydroxy and cyano,
   or
   where alkyl may be substituted by 1 to 3 fluorine substituents,
R¹⁰ represents hydrogen,
R¹¹ represents C₁-C₄-alkyl,
   where alkyl may be substituted by a hydroxy substituent,
R¹² represents hydrogen or C₁-C₄-alkyl,
or
R¹¹ and R¹² together with the carbon atom to which they are attached form a cyclopropyl ring, cyclobutyl ring or cyclopentyl ring,
   where the cyclobutyl ring and the cyclopentyl ring may be substituted by a hydroxy substituent,
      and
      R¹⁵ represents hydrogen, methyl or fluoromethyl,
      and the salts thereof, the solvates thereof and the solvates of the salts thereof.

Preference is given to compounds of the formula (I) in which

R¹ represents a group of the formula

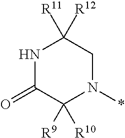

where * is the point of attachment to the carbonyl group,
R⁹ represents C₁-C₄-alkyl,
R¹⁰ represents hydrogen,
R¹¹ represents C₁-C₄-alkyl,
R¹² represents hydrogen,
or
R¹¹ and R¹² together with the carbon atom to which they are attached form a cyclopropyl ring,
   and
   R¹⁵ represents hydrogen, methyl or fluoromethyl,
   and the salts thereof, the solvates thereof and the solvates of the salts thereof.

Preference is given to compounds of the formula (I) in which
R¹ represents a group of the formula

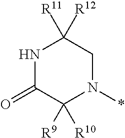

where * is the point of attachment to the carbonyl group,
X represents an oxygen atom,
R⁹ represents methyl or ethyl,
R¹⁰ represents hydrogen,
R¹¹ represents methyl,
R¹² represents hydrogen,
or
R¹¹ and R¹² together with the carbon atom to which they are attached form a cyclopropyl ring,
   and
   R¹⁵ represents hydrogen, methyl or fluoromethyl,
   and the salts thereof, the solvates thereof and the solvates of the salts thereof.

Preference is given to compounds of the formula (I) in which

R¹ represents a group of the formula

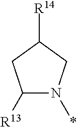

where * is the point of attachment to the carbonyl group,
R¹³ represents hydroxymethyl or hydroxyethyl,
R¹⁴ represents methoxy or ethoxy,
   where methoxy and ethoxy may be substituted by 1 to 3 substituents selected from the group consisting of deuterium and fluorine,
      and
      R¹⁵ represents hydrogen, methyl or fluoromethyl,
      and the salts thereof, the solvates thereof and the solvates of the salts thereof.

Preference is given to compounds of the formula (I) in which

R¹ represents a group of the formula

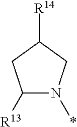

where * is the point of attachment to the carbonyl group,
R¹³ represents hydroxymethyl,
R¹⁴ represents ethoxy,
   where ethoxy may be substituted by 1 to 3 substituents selected from the group consisting of deuterium and fluorine,
      and
      R¹⁵ represents hydrogen, methyl or fluoromethyl,
      and the salts thereof, the solvates thereof and the solvates of the salts thereof.

Preference is also given to compounds of the formula (I) in which R¹⁵ represents hydrogen.

Preference is also given to compounds of the formula (I) in which R¹⁵ represents fluoromethyl.

Preference is also given to compounds of the formula (Ia)

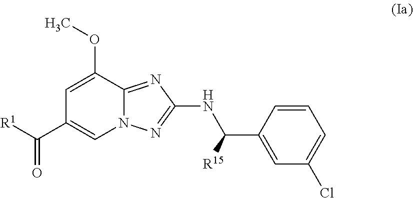

where R¹ and R¹⁵ are as defined above.

Preference is also given to
{2-[(3-chlorobenzyl)amino]-8-methoxy[1,2,4]triazolo[1,5-a]pyridin-6-yl}[5-(3-hydroxycyclobutyl)-2-methylmorpholin-4-yl]methanone [diastereomer 3+diastereomer 4]
or
{2-[(3-chlorobenzyl)amino]-8-methoxy[1,2,4]triazolo[1,5-a]pyridin-6-yl}[(5R)-2-(2-hydroxyethyl)-2,5-dimethylmorpholin-4-yl]methanone [enantiomerically pure isomer]
or
(2-{[1-(3-chlorophenyl)-2-fluoroethyl]amino}-8-methoxy[1,2,4]triazolo[1,5-a]pyridin-6-yl) [5-(2-hydroxyethyl)-2-methylmorpholin-4-yl]methanone [enantiomerically pure isomer 2]
or
(2-{[1-(3-chlorophenyl)-2-fluoroethyl]amino}-8-methoxy[1,2,4]triazolo[1,5-a]pyridin-6-yl)[(cis)-2-hydroxy-7-methyl-8-oxa-5-azaspiro[3.5]non-5-yl]methanone [enantiomerically pure isomer 1]
or
(2-{[1-(3-chlorophenyl)-2-fluoroethyl]amino}-8-methoxy[1,2,4]triazolo[1,5-a]pyridin-6-yl)[(cis)-2-hydroxy-7-methyl-8-oxa-5-azaspiro[3.5]non-5-yl]methanone [enantiomerically pure isomer 2]
or
{2-[(3-chlorobenzyl)amino]-8-methoxy[1,2,4]triazolo[1,5-a]pyridin-6-yl}[(5R)-2-(2-hydroxypropyl)-2,5-dimethylmorpholin-4-yl]methanone [enantiomerically pure isomer]
or
4-({2-[(3-chlorobenzyl)amino]-8-methoxy[1,2,4]triazolo[1,5-a]pyridin-6-yl}carbonyl)-3-methyl-1,4-diazabicyclo[4.2.0]octan-2-one [enantiomerically pure isomer]
   or one of the salts, the solvates or the solvates of the salts of these compounds.

The invention further provides a process for preparing the compounds of the formula (I), or the salts thereof, solvates thereof or the solvates of the salts thereof, wherein

[A] the compounds of the formula

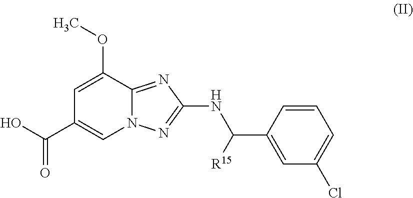

in which
R¹⁵ has the meaning given above,
are reacted with compounds of the formula

R¹—H (III)

in which
R¹ has the meaning given above,
in the presence of dehydrating reagents
or
[B] the compounds of the formula

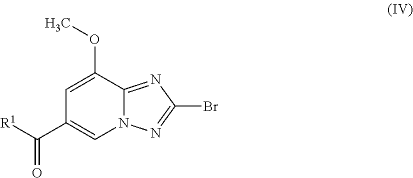

in which
R¹ has the meaning given above,
are reacted with compounds of the formula

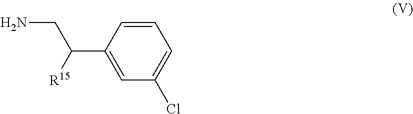

in which
R¹⁵ has the meaning given above,
in the presence of a palladium catalyst.

The reaction according to process [A] is generally carried out in inert solvents, if appropriate in the presence of a base, preferably in a temperature range from 0° C. to room temperature at atmospheric pressure.

Suitable dehydrating agents here are, for example, carbodiimides such as N,N′-diethyl-, N,N′-dipropyl-, N,N′-diisopropyl-, N,N′-dicyclohexylcarbodiimide, N-(3-dimethylaminoisopropyl)-N′-diethyl-, N,N′-dipropyl-, N,N′-diisopropyl-, N,N′-dicyclohexylcarbodiimide, N-(3-dimethylaminoisopropyl)-N′-ethylcarbodiimide hydrochloride (EDC) (optionally in the presence of pentafluorophenol (PFP)), N-cyclohexylcarbodiimid-N′-propyloxymethyl-polystyrene (PS-carbodiimide) or carbonyl compounds such as carbonyldiimidazole, or 1,2-oxazolium compounds such as 2-ethyl-5-phenyl-1,2-oxazolium 3-sulphate or 2-tert-butyl-5-methylisoxazolium perchlorate, or acylamino compounds such as 2-ethoxy-1-ethoxycarbonyl-1,2-dihydroquinoline, or propanephosphonic anhydride, or isobutyl chloroformate, or bis-(2-oxo-3-oxazolidinyl)phosphoryl chloride or benzotriazolyloxytri(dimethylamino)phosphonium hexafluorophosphate, or O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU), (benzotriazol-1-yloxy)bisdimethylaminomethylium fluoroborate (TBTU) or O-(7-azabenzotriazol-1-yl)-N,N,N′,N′-tetramethyluronium hexafluorophosphate (HATU), or 1-hydroxybenzotriazole (HOBt), or benzotriazol-1-yloxytris(dimethylamino)phosphonium hexafluorophosphate (BOP), or mixtures of these, with bases. The condensation is preferably carried out using HATU.

Bases are, for example, alkali metal carbonates such as sodium carbonate or potassium carbonate, or sodium bicarbonate or potassium bicarbonate, or organic bases such as trialkylamines, for example triethylamine, N-methylmorpholine, N-methylpiperidine, 4-dimethylaminopyridine or diisopropylethylamine; preference is given to diisopropylethylamine.

Inert solvents are, for example, halogenated hydrocarbons such as dichloromethane or trichloromethane, hydrocarbons such as benzene, or other solvents such as nitromethane, dioxane, dimethylformamide, dimethyl sulphoxide or acetonitrile, or mixtures of the solvents mentioned; preference is given to dimethylformamide.

The reaction according to process [B] is generally carried out under Buchwald-Hartwig conditions in the presence of a base, in inert solvents, preferably in a temperature range of from 0° C. to 200° C., preferably at from 10° C. to 150° C., at atmospheric pressure or at temperatures above the boiling point of the solvent at elevated pressure in sealed reaction vessels (microwave tubes) or optionally in a microwave oven at temperatures above the boiling point of the solvent and at elevated pressure.

Bases are, for example, alkali metal or alkaline earth metal carbonates such as lithium carbonate, sodium carbonate, potassium carbonate, calcium carbonate or caesium carbonate, alkali metal or alkaline earth metal hydroxides such as sodium hydroxide, potassium hydroxide or barium hydroxide, alkali metal or alkaline earth metal phosphates such as potassium phosphate, alkali metal alkoxides such as sodium tert-butoxide or potassium tert-butoxide and sodium methoxide, alkali metal phenoxides such as sodium phenoxide, amides such as sodium amide, lithium bis(trimethylsilyl)amide, sodium bis(trimethylsilyl)amide or potassium bis(trimethylsilyl)amide or lithium diisopropylamide or organic amines such as 1,5-diazabicyclo[4.3.0]non-5-ene (DBN), 1,8-diazabicyclo[5.4.0]undec-7-ene (DBU); preference is given to caesium carbonate, potassium carbonate, sodium tert-butoxide or potassium tert-butoxide or lithium bis(trimethylsilyl)amide.

Palladium catalysts are, for example, palladium on activated carbon, palladium(II) acetate, bis(dibenzylideneacetone)palladium(0), tetrakis(triphenylphosphine)palladium(0), bis(triphenylphosphine)palladium(II) chloride, bis(acetonitrile)palladium(II) chloride and [1,1′-bis(diphenylphosphino)ferrocene]dichloropalladium(II) and the corresponding dichloromethane complex, optionally in conjunction with additional phosphane ligands, for example 2,2′-bis(diphenylphosphino)-1,1′-binaphthyl (BINAP), (2-biphenyl)di-tert-butylphosphine, dicyclohexyl[2′,4′,6′-tris(1-methylethyl)biphenyl-2-yl]phosphane (XPhos), bis(2-phenylphosphinophenyl) ether (DPEphos) or 4,5-bis(diphenylphosphino)-9,9-dimethylxanthene (Xantphos) [cf., for example, Hassan J. et al., Chem. Rev. 2002, 102, 1359-1469], 2-(dicyclohexylphosphine)-3,6-dimethoxy-2′,4′,6′-triisopropyl-1,1′-biphenyl (BrettPhos), 2-dicyclohexylphosphino-2′,6′-dimethoxybiphenyl (SPhos), 2-dicyclohexylphosphino-2′,6′-diisopropoxybiphenyl (RuPhos), 2-(di-t-butylphosphino)-3-methoxy-6-methyl-2′,4′,6′-tri-i-propyl-1,1′-biphenyl (RockPhos) and 2-di-tert-butylphosphino-2′,4′,6′-triisopropylbiphenyl (tert-ButylXPhos). It is furthermore possible to use appropriate precatalysts such as chloro-[2-(dicyclohexylphosphine)-3,6-dimethoxy-2′,4′,6′-triisopropyl-1,1′-biphenyl][2-(2-aminoethyl)-phenyl]palladium(II) (BrettPhos precatalyst) [cf., for example, S. L. Buchwald et al., Chem. Sci. 2013, 4, 916], optionally in combination with additional phosphane ligands such as 2-(dicyclohexylphosphine)-3,6-dimethoxy-2′,4′,6′-triisopropyl-1,1′-biphenyl (BrettPhos); preference is given to bis(dibenzylideneacetone)palladium(0) in combination with 4,5-bis(diphenylphosphino)-9,9-dimethylxanthene (Xantphos) and chloro-[2-(dicyclohexylphosphine)-3,6-dimethoxy-2′,4′,6′-triisopropyl-1,1′-biphenyl][2-(2-aminoethyl)phenyl]palladium(II) (BrettPhos precatalyst) or a mixture of chloro-[2-(dicyclohexylphosphine)-3,6-dimethoxy-2′,4′,6′-triisopropyl-1,1′-biphenyl][2-(2-aminoethyl)phenyl]palladium(II) (BrettPhos precatalyst) and 2-(dicyclohexylphosphine)-3,6-dimethoxy-2′,4′,6′-triisopropyl-1,1′-biphenyl (BrettPhos).

Inert solvents are, for example, ethers such as 1,4-dioxane, tetrahydrofuran, 2-methyltetrahydrofuran, diethyl ether, di-n-butylether, glycol dimethyl ether or diethylene glycol dimethyl ether, alcohols such as tert-butanol or amyl alcohols or other solvents such as dimethylformamide (DMF), dimethyl sulphoxide (DMSO), dimethylacetamide (DMA), toluene or acetonitrile, or mixtures of the solvents mentioned; preference is given to tert-butanol, 1,4-dioxane or toluene.

The compounds of the formulae (III) and (V) are known, can be synthesized from the corresponding starting compounds by known processes or can be prepared analogously to the processes described in the Examples section.

The compounds of the formula (II) are known or can be prepared by reacting the compounds of the formula

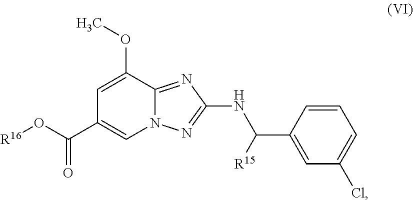

in which
R¹⁵ has the meaning given above and
R¹⁶ represents methyl, ethyl or tert-butyl,
if R¹⁶ represents methyl or ethyl, with a base or, if R¹⁶ represents tert-butyl, with an acid.

The reaction is generally carried out in inert solvents, preferably in a temperature range of from 0° C. to room temperature at atmospheric pressure.

Bases are, for example, alkali metal hydroxides such as sodium hydroxide, lithium hydroxide or potassium hydroxide, or alkali metal carbonates such as caesium carbonate, sodium carbonate or potassium carbonate; preference is given to sodium hydroxide.

Acids are, for example, trifluoroacetic acid or hydrogen chloride in dioxane. Optionally, triethylsilane may be added to the reaction mixture; preference is given to the mixture of trifluoroacetic acid and triethylsilane.

Inert solvents are, for example, halogenated hydrocarbons such as dichloromethane, trichloromethane, carbon tetrachloride, trichloroethane, tetrachloroethane, 1,2-dichloroethane or trichloroethylene, ethers such as diethyl ether, methyl tert-butyl ether, 1,2-dimethoxyethane, dioxane, tetrahydrofuran, glycol dimethyl ether or diethylene glycol dimethyl ether, alcohols such as methanol, ethanol, n-propanol, isopropanol, n-butanol or tert-butanol, hydrocarbons such as benzene, xylene, toluene, hexane, cyclohexane or mineral oil fractions, or other solvents such as dimethylformamide, dimethylacetamide, dimethyl sulphoxide, acetonitrile or pyridine, or mixtures of solvents; preference is given to dioxane.

The compounds of the formula (VI) are known or can be prepared by reacting

[C] compounds of the formula

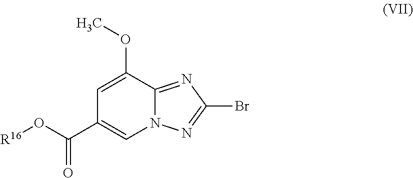

in which
R¹⁶ represents tert-butyl,
with compounds of the formula

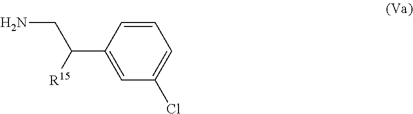

in which
R¹⁵ represents methyl or fluoromethyl,
or
[D] compounds of the formula

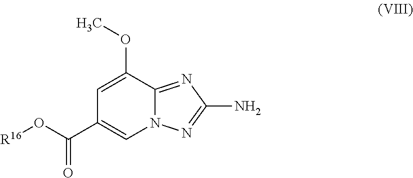

in which
R¹⁶ represents methyl, ethyl or tert-butyl,
with the compound of the formula

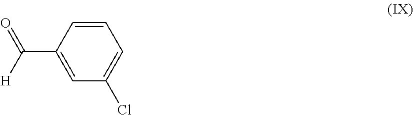

in the presence of a reducing agent.

The reaction in process [C] is effected as described for process [B].

The reaction in process [D] is generally effected in inert solvents, preferably within a temperature range from 0° C. up to the reflux of the solvents at standard pressure.

Reducing agents are, for example, hydrides such as complex borohydrides or aluminium hydrides and also boranes such as sodium borohydride, lithium borohydride, sodium cyanoborohydride, lithium aluminium hydride, sodium bis-(2-methoxyethoxy)aluminium hydride or borane/tetrahydrofuran; preference is given to sodium borohydride.

Inert solvents are, for example, alcohols such as methanol, ethanol, n-propanol or isopropanol, or ethers such as diethyl ether, dioxane, tetrahydrofuran, or other solvents such as dimethylformamide; preference is given to ethanol.

The compounds of the formula (Va) are a subset of the compounds of the formula (V).

The compounds of the formulae (VII), (VIII) and (IX) are known, can be synthesized from the corresponding starting compounds by known processes or can be prepared analogously to the processes described in the Examples section.

The compounds of the formula (IV) are known or can be prepared by reacting compounds of the formula

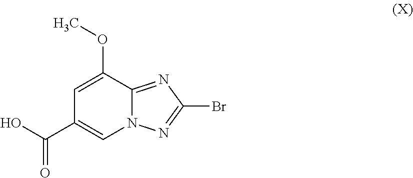

with compounds of the formula (III) in the presence of dehydrating reagents.

The reaction is effected as described for process [A].

The compounds of the formula (X) are known, can be synthesized from the corresponding starting compounds by known processes or can be prepared analogously to the processes described in the Examples section.

The preparation of the starting compounds and of the compounds of the formula (I) can be illustrated by the synthesis scheme below.

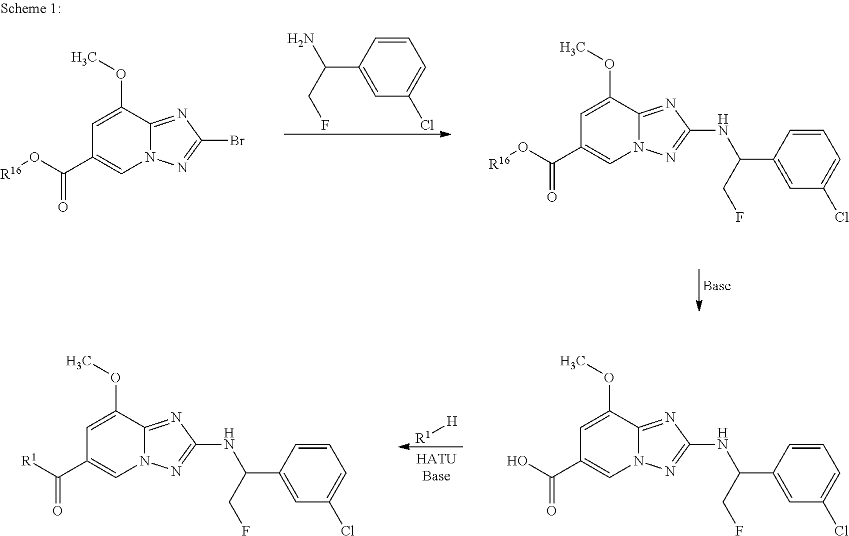

The compounds according to the invention have an unforeseeable useful pharmacological activity spectrum and good pharmacokinetic behaviour. They are compounds modulating the proteolytic activity of the serine protease thrombin. The compounds according to the invention inhibit the thrombin-catalysed enzymatic cleavage of substrates which play an essential role in the activation of blood coagulation, platelet aggregation (via PAR-1 activation of the platelets) and thrombin-induced inflammation, fibrosis and angiogenesis processes.

They are therefore suitable for use as medicaments for the treatment and/or prophylaxis of diseases in humans and animals.

The present invention further provides for the use of the compounds according to the invention for treatment and/or prophylaxis of disorders, in particular cardiovascular disorders, preferably thrombotic or thromboembolic disorders and/or thrombotic or thromboembolic complications.

As a key enzyme at the end of the coagulation cascade, thrombin translates, via a series of conversions, the impulses of the cascade into the coagulation state of the blood. By conversion of fibrinogen into insoluble fibrin, fibrin clots are formed, which are likewise stabilized by thrombin-activated factor XIIIa. By activating TAFI (thrombin-activatable fibrinolysis inhibitor) to TAFIa, thrombin in a complex with thrombomodulin inhibits the dissolution of the clot. Activation of factors V and VIII potentiates the production of thrombin and thus in turn amplifies the coagulation reaction. In addition, thrombin is a potent trigger of platelet aggregation (via PAR-1 activation), which also contributes considerably to haemostasis.

Accordingly, the compounds according to the invention are suitable for the treatment and/or prophylaxis of disorders or complications which arise or may arise from the formation of clots.

For the purpose of the present invention, the “thrombotic or thromboembolic disorders” include disorders which occur both in the arterial and in the venous vasculature and which can be treated with the compounds according to the invention, in particular disorders in the coronary arteries of the heart, such as acute coronary syndrome (ACS), myocardial infarction with ST segment elevation (STEMI) and without ST segment elevation (non-STEMI), stable angina pectoris, unstable angina pectoris, reocclusions and restenoses after coronary interventions such as angioplasty, stent implantation or aortocoronary bypass, but also thrombotic or thromboembolic disorders in further vessels leading to peripheral arterial occlusive disorders, pulmonary embolisms, venous thromboembolisms, venous thromboses, in particular in deep leg veins and kidney veins, transitory ischaemic attacks and also thrombotic stroke and thromboembolic stroke.

Stimulation of the coagulation system may occur by various causes or associated disorders. In the context of surgical interventions, immobility, confinement to bed, infection or cancer or cancer therapy, inter alia, the coagulation system can be highly activated, and there may be thrombotic complications, in particular venous thromboses. The compounds according to the invention are therefore suitable for the prophylaxis of thromboses in the context of surgical interventions in patients suffering from cancer. The compounds according to the invention are therefore also suitable for the prophylaxis of thromboses in patients having an activated coagulation system, for example in the stimulation situations described.

The inventive compounds are therefore also suitable for the prevention and treatment of cardiogenic thromboembolisms, for example brain ischaemias, stroke and systemic thromboembolisms and ischaemias, in patients with acute, intermittent or persistent cardial arrhythmias, for example atrial fibrillation, and those undergoing cardioversion, and also in patients with heart valve disorders or with artificial heart valves.

Thromboembolic complications are also encountered in microangiopathic haemolytic anaemias, extracorporeal circulatory systems, such as haemodialysis, and also prosthetic heart valves.

Moreover, the compounds according to the invention are particularly suitable for the treatment of disorders where a clot is already present, since thrombin incorporated in particular in the clot stabilizes the clot. Since the inhibition of these thrombin molecules accelerates the degradation of the clot, the compounds according to the invention can be used for the treatment of existing clots. These clots may be formed in the entire vascular system and may cause grave complications in various organs, in particular via ischaemia, inflammatory reactions or formation of embolisms, for example myocardial infarction or stroke, but also pulmonary embolism or post-thrombotic syndrome in particular after deep vein thromboses in the leg. Accordingly, the compounds according to the invention are also suitable for the treatment of venous and arterial occlusions of the occular blood vessels caused by clots, for example age-related macular degeneration.

By virtue of the synergistic effects observed with lytic therapeutic principles such as the tissue plasminogen activator (tPA), the compounds are suitable for adjunctive use in the context of thrombolysis therapy.

Moreover, the compounds according to the invention are suitable for the treatment and/or prophylaxis of disorders involving microclot formation, for example fibrin deposits in cerebral blood vessels which may lead to dementia disorders such as vascular dementia or Alzheimer's disease. Here, the clot may contribute to the disorder both via occlusions and by binding further disease-relevant factors.

Moreover, the compounds according to the invention are suitable in particular for the treatment and/or prophylaxis of disorders where, in addition to the pro-coagulant component, the pro-inflammotory component of thrombin action plays an essential role. Mutual enhancement of coagulation and inflammation in particular can be prevented by the compounds according to the invention, thus decisively lowering the probability of thrombotic complications. Here, the treatment and/or prophylaxis in the context of atherosclerotic vascular disorders, inflammations in the context of rheumatic disorders of the locomotor system, inflammatory disorders of the lung, such as pulmonary fibroses, inflammatory disorders of the kidney, such as glomerulonephritides, inflammatory disorders of the intestine, such as Crohn's disease or ulcerative colitis, or disorders which may be present in the context of a diabetic underlying disease, such as diabetic retinopathy or nephropathy, may be considered, inter alia.

Moreover, the compounds according to the invention can be used for inhibiting tumour growth and the formation of metastases, and also for the prophylaxis and/or treatment of thromboembolic complications, such as, for example, venous thromboembolisms, for tumour patients, in particular those undergoing major surgical interventions or chemo- or radiotherapy.

In addition, the inventive compounds are also suitable for the prophylaxis and/or treatment of pulmonary hypertension.

In the context of the present invention, the term “pulmonary hypertension” includes pulmonary arterial hypertension, pulmonary hypertension associated with disorders of the left heart, pulmonary hypertension associated with pulmonary disorders and/or hypoxia and pulmonary hypertension owing to chronic thromboembolisms (CTEPH).

“Pulmonary arterial hypertension” includes idiopathic pulmonary arterial hypertension (IPAH, formerly also referred to as primary pulmonary hypertension), familial pulmonary arterial hypertension (FPAH) and associated pulmonary-arterial hypertension (APAH), which is associated with collagenoses, congenital systemic-pulmonary shunt vitia, portal hypertension, HIV infections, the ingestion of certain drugs and medicaments, with other disorders (thyroid disorders, glycogen storage disorders, Morbus Gaucher, hereditary teleangiectasia, haemoglobinopathies, myeloproliferative disorders, splenectomy), with disorders having a significant venous/capillary contribution, such as pulmonary-venoocclusive disorder and pulmonary-capillary haemangiomatosis, and also persisting pulmonary hypertension of neonatants

Pulmonary hypertension associated with disorders of the left heart includes a diseased left atrium or ventricle and mitral or aorta valve defects.

Pulmonary hypertension associated with pulmonary disorders and/or hypoxia includes chronic obstructive pulmonary disorders, interstitial pulmonary disorder, sleep apnoea syndrome, alveolar hypoventilation, chronic high-altitude sickness and inherent defects.

Pulmonary hypertension owing to chronic thromboembolisms (CTEPH) comprises the thromboembolic occlusion of proximal pulmonary arteries, the thromboembolic occlusion of distal pulmonary arteries and non-thrombotic pulmonary embolisms (tumour, parasites, foreign bodies).

The present invention further provides for the use of the inventive compounds for production of medicaments for treatment and/or prophylaxis of pulmonary hypertension associated with sarcoidosis, histiocytosis X and lymphangiomatosis.

In addition, the inventive substances may also be useful for treatment of pulmonary and hepatic fibroses.

In addition, the inventive compounds may also be suitable for treatment and/or prophylaxis of disseminated intravascular coagulation in the context of an infectious disease, and/or of systemic inflammatory syndrome (SIRS), septic organ dysfunction, septic organ failure and multiorgan failure, acute respiratory distress syndrome (ARDS), acute lung injury (ALI), septic shock and/or septic organ failure.

In the course of an infection, there may be a generalized activation of the coagulation system (disseminated intravascular coagulation or consumption coagulopathy, hereinbelow referred to as “DIC”) with microthrombosis in various organs and secondary haemorrhagic complications. Moreover, there may be endothelial damage with increased permeability of the vessels and seeping of fluids and proteins into the extravasal lumen. As the infection progresses, there may be failure of an organ (for example kidney failure, liver failure, respiratory failure, central-nervous deficits and cardiovascular failure) or multiorgan failure.

In the case of DIC, there is a massive activation of the coagulation system at the surface of damaged endothelial cells, the surfaces of foreign bodies or injured extravascular tissue. As a consequence, there is coagulation in small vessels of various organs with hypoxia and subsequent organ dysfunction. A secondary effect is the consumption of coagulation factors (for example factor X, prothrombin and fibrinogen) and platelets, which reduces the coagulability of the blood and may result in heavy bleeding.

The compounds according to the invention are very particularly suitable for the treatment and/or prophylaxis of acute coronary syndrome (ACS), venous thromboembolisms, venous thromboses, in particular in deep leg veins and kidney veins, pulmonary embolisms, stroke and/or thrombosis prophylaxis in the context of surgical interventions, in particular in the context of surgical interventions in patients suffering from cancer.

The present invention further provides for the use of the compounds according to the invention for treatment and/or prophylaxis of disorders, especially the disorders mentioned above.

The present invention further provides for the use of the compounds according to the invention for production of a medicament for treatment and/or prophylaxis of disorders, especially the disorders mentioned above.

The present invention further provides a method for treatment and/or prophylaxis of disorders, especially the disorders mentioned above, using a therapeutically effective amount of a compound according to the invention.

The present invention further provides the compounds according to the invention for use in a method for the treatment and/or prophylaxis of disorders, especially the disorders mentioned above, using a therapeutically effective amount of a compound according to the invention.

The present invention further provides medicaments comprising a compound according to the invention and one or more further active compounds.

In addition, the compounds according to the invention can also be used for preventing coagulation ex vivo, for example for the protection of organs to be transplanted against organ damage caused by formation of clots and for protecting the organ recipient against thromboemboli from the transplanted organ, for preserving blood and plasma products, for cleaning/pretreating catheters and other medical auxiliaries and instruments, for coating synthetic surfaces of medical auxiliaries and instruments used in vivo or ex vivo or for biological samples which may comprise factor IIa.

The present invention further provides a method for preventing the coagulation of blood in vitro, in particular in banked blood or biological samples which may contain factor IIa, which method is characterized in that an anticoagulatory effective amount of the compound according to the invention is added.

The present invention further provides medicaments comprising a compound according to the invention and one or more further active compounds, in particular for treatment and/or prophylaxis of the disorders mentioned above. Preferred examples of active compounds suitable for combinations include:
lipid-lowering substances, especially HMG-CoA (3-hydroxy-3-methylglutaryl-coenzyme A) reductase inhibitors, for example lovastatin (Mevacor), simvastatin (Zocor), pravastatin (Pravachol), fluvastatin (Lescol) and atorvastatin (Lipitor);
coronary therapeutics/vasodilatators, especially ACE (angiotensin converting enzyme) inhibitors, for example captopril, lisinopril, enalapril, ramipril, cilazapril, benazepril, fosinopril, quinapril and perindopril, or AII (angiotensin II) receptor antagonists, for example embusartan, losartan, valsartan, irbesartan, candesartan, eprosartan and temisartan, or β-adrenoceptor antagonists, for example carvedilol, alprenolol, bisoprolol, acebutolol, atenolol, betaxolol, carteolol, metoprolol, nadolol, penbutolol, pindolol, propanolol and timolol, or alpha-1-adrenoceptor antagonists, for example prazosine, bunazosine, doxazosine and terazosine, or diuretics, for example hydrochlorothiazide, furosemide, bumetanide, piretanide, torasemide, amiloride and dihydralazine, or calcium channel blockers, for example verapamil and diltiazem, or dihydropyridine derivatives, for example nifedipin (Adalat) and nitrendipine (Bayotensin), or nitro preparations, for example isosorbide 5-mononitrate, isosorbide dinitrate and glycerol trinitrate, or substances causing an increase in cyclic guanosine monophosphate (cGMP), for example stimulators of soluble guanylate cyclase, for example riociguat;
plasminogen activators (thrombolytics/fibrinolytics) and compounds which promote thrombolysis/fibrinolysis such as inhibitors of the plasminogen activator inhibitor (PAI inhibitors) or inhibitors of the thrombin-activated fibrinolysis inhibitor (TAFI inhibitors) such as, for example, tissue plasminogen activator (t-PA, for example Actilyse®), streptokinase, reteplase and urokinase;
anticoagulatory substances (anticoagulants), for example heparin (UFH), low-molecular-weight heparins (LMW), for example tinzaparin, certoparin, parnaparin, nadroparin, ardeparin, enoxaparin, reviparin, dalteparin, danaparoid, semuloparin (AVE 5026), adomiparin (M118) and EP-42675/ORG42675;
direct thrombin inhibitors (DTI) such as, for example, Pradaxa (dabigatran), atecegatran (AZD-0837), DP-4088, SSR-182289A, argatroban, bivalirudin and tanogitran (BIBT-986 and prodrug BIBT-1011), hirudin;
direct factor Xa inhibitors such as, for example, rivaroxaban, apixaban, edoxaban (DU-176b), betrixaban (PRT-54021), R-1663, darexaban (YM-150), otamixaban (FXV-673/RPR-130673), letaxaban (TAK-442), razaxaban (DPC-906), DX-9065a, LY-517717, idraparinux and fondaparinux;
platelet aggregation-inhibiting substances (platelet aggregation inhibitors, thrombocyte aggregation inhibitors), for example acetylsalicylic acid (for example Aspirin), ticlopidine (Ticlid), clopidogrel (Plavix), prasugrel, ticagrelor, cangrelor, elinogrel, vorapaxar;
fibrinogen receptor antagonists (glycoprotein-IIb/IIIa antagonists), for example abciximab, eptifibatide, tirofiban, lamifiban, lefradafiban and fradafiban;
recombinant human activated protein C such as, for example, Xigris;
and also antiarrhythmics

The present invention furthermore provides the combination of a compound according to the invention and 5-chloro-N-({(5S)-2-oxo-3-[4-(3-oxo-4-morpholinyl)phenyl]-1,3-oxazolidin-5-yl}methyl)-2-thiophenecarboxamide (rivaroxaban) [WO 01/47919] having the structural formula

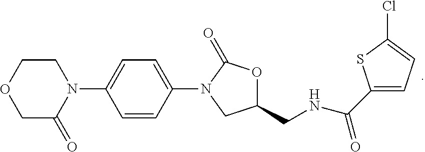 [SMILES: O=C(NC[C@H]1CN(c2ccc(N3CCOCC3=O)cc2)C(=O)O1)c1ccc(Cl)s1]

The present invention furthermore provides the combination of
{2-[(3-chlorobenzyl)amino]-8-methoxy[1,2,4]triazolo[1,5-a]pyridin-6-yl}[(5R)-2-(2-hydroxyethyl)-2,5-dimethylmorpholin-4-yl]methanone [enantiomerically pure isomer]
or
(2-{[1-(3-chlorophenyl)-2-fluoroethyl]amino}-8-methoxy[1,2,4]triazolo[1,5-a]pyridin-6-yl)[(cis)-2-hydroxy-7-methyl-8-oxa-5-azaspiro[3.5]non-5-yl]methanone [enantiomerically pure isomer 1]
or
{2-[(3-chlorobenzyl)amino]-8-methoxy[1,2,4]triazolo[1,5-a]pyridin-6-yl}[(5R)-2-(2-hydroxypropyl)-2,5-dimethylmorpholin-4-yl]methanone [enantiomerically pure isomer]
or
4-({2-[(3-chlorobenzyl)amino]-8-methoxy[1,2,4]triazolo[1,5-a]pyridin-6-yl}carbonyl)-3-methyl-1,4-diazabicyclo[4.2.0]octan-2-one [enantiomerically pure isomer]
   or one of the salts thereof, solvates thereof or solvates of the salts thereof
   with 5-chloro-N-({(5S)-2-oxo-3-[4-(3-oxo-4-morpholinyl)phenyl]-1,3-oxazolidin-5-yl}methyl)-2-thiophenecarboxamide (rivaroxaban) of the structural formula

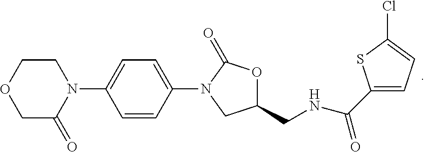 [SMILES: O=C(NC[C@H]1CN(c2ccc(N3CCOCC3=O)cc2)C(=O)O1)c1ccc(Cl)s1]

The compounds according to the invention can act systemically and/or locally. For this purpose, they can be administered in a suitable manner, for example by the oral, parenteral, pulmonal, nasal, sublingual, lingual, buccal, rectal, dermal, transdermal, conjunctival or otic route, or as an implant or stent.

The compounds according to the invention can be administered in suitable administration forms for these administration routes.

Suitable administration forms for oral administration are those which function according to the prior art and deliver the compounds according to the invention rapidly and/or in modified fashion, and which contain the compounds according to the invention in crystalline and/or amorphized and/or dissolved form, for example tablets (uncoated or coated tablets, for example having enteric coatings or coatings which are insoluble or dissolve with a delay and control the release of the compound according to the invention), tablets which disintegrate rapidly in the mouth, or films/wafers, films/lyophilizates, capsules (for example hard or soft gelatin capsules), sugar-coated tablets, granules, pellets, powders, emulsions, suspensions, aerosols or solutions.

Parenteral administration can be accomplished with avoidance of a resorption step (for example by an intravenous, intraarterial, intracardiac, intraspinal or intralumbar route) or with inclusion of a resorption (for example by an intramuscular, subcutaneous, intracutaneous, percutaneous or intraperitoneal route). Administration forms suitable for parenteral administration include preparations for injection and infusion in the form of solutions, suspensions, emulsions, lyophilizates or sterile powders.

Oral administration is preferred.

Suitable administration forms for the other administration routes are, for example, pharmaceutical forms for inhalation (including powder inhalers, nebulizers), nasal drops, solutions or sprays; tablets for lingual, sublingual or buccal administration, films/wafers or capsules, suppositories, preparations for the ears or eyes, vaginal capsules, aqueous suspensions (lotions, shaking mixtures), lipophilic suspensions, ointments, creams, transdermal therapeutic systems (for example patches), milk, pastes, foams, dusting powders, implants or stents.

The inventive compounds can be converted to the administration forms mentioned. This can be accomplished in a manner known per se by mixing with inert, nontoxic, pharmaceutically suitable excipients. These excipients include carriers (for example microcrystalline cellulose, lactose, mannitol), solvents (e.g. liquid polyethylene glycols), emulsifiers and dispersing or wetting agents (for example sodium dodecylsulphate, polyoxysorbitan oleate), binders (for example polyvinylpyrrolidone), synthetic and natural polymers (for example albumin), stabilizers (e.g. antioxidants, for example ascorbic acid), colourants (e.g. inorganic pigments, for example iron oxides) and flavour and/or odour correctants.

The present invention further provides medicaments comprising at least one inventive compound, preferably together with one or more inert nontoxic pharmaceutically suitable excipients, and the use thereof for the purposes mentioned above.

In the case of parenteral administration, it has generally been found to be advantageous to administer amounts of about 5 to 250 mg every 24 hours to achieve effective results. In the case of oral administration, the amount is about 5 to 500 mg every 24 hours.

It may nevertheless be necessary in some cases to deviate from the stated amounts, specifically as a function of the body weight, route of administration, individual response to the active ingredient, nature of the preparation and time or interval over which administration takes place.

Unless stated otherwise, the percentages in the tests and examples which follow are percentages by weight; parts are parts by weight. Solvent ratios, dilution ratios and concentration data for the liquid/liquid solutions are based in each case on volume. “w/v” means “weight/volume”. For example, “10% w/v” means: 100 ml of solution or suspension comprise 10 g of substance.

### A) EXAMPLES

### Abbreviations

d day(s), doublet (in NMR)
TLC thin-layer chromatography
DCI direct chemical ionization (in MS)
dd doublet of doublets (in NMR)
DMAP 4-dimethylaminopyridine
DMF N,N-dimethylformamide
DMSO dimethyl sulphoxide
of th. of theory (in yield)
ESI electrospray ionization (in MS)
h hour(s)
HATU O-(7-azabenzotriazol-1-yl)-N,N,N′,N′-tetramethyluronium-hexafluorophosphate
HPLC high-pressure, high-performance liquid chromatography
LC-MS liquid chromatography-coupled mass spectroscopy
m multiplet (in NMR)
M molar
min minute(s)
MS mass spectroscopy
prep. preparative (in HPLC)
N normal
NMR nuclear magnetic resonance spectroscopy
q quartet (in NMR)
quant. quantitative
RP reversed phase (in HPLC)
RT room temperature
Rₜ retention time (in HPLC)
s singlet (in NMR)
t triplet (in NMR)
TFA trifluoroacetic acid
THF tetrahydrofuran

### LC-MS Methods:

Method 1A:

Instrument: Waters ACQUITY SQD UPLC system; column: Waters Acquity UPLC HSS T3 1.8μ 50×1 mm; mobile phase A: 1 l of water+0.25 ml of 99% strength formic acid; mobile phase B: 1 l of acetonitrile+0.25 ml of 99% strength formic acid; gradient: 0.0 min 90% A→1.2 min 5% A→2.0 min 5% A; oven: 50° C.; flow rate: 0.40 ml/min; UV detection: 208-400 nm.

Method 2A:

Instrument: Waters ACQUITY SQD UPLC system; column: Waters Acquity UPLC HSS T3 1.8μ 30×2 mm; mobile phase A: 1 l of water+0.25 ml of 99% strength formic acid; mobile phase B: 1 l of acetonitrile+0.25 ml of 99% strength formic acid; gradient: 0.0 min 90% A→1.2 min 5% A→2.0 min 5% A; oven: 50° C.; flow rate: 0.60 ml/min; UV detection: 208-400 nm.

Method 3A:

Instrument: Micromass Quattro Premier with Waters UPLC Acquity; column: Thermo Hypersil GOLD 1.9μ 50×1 mm; mobile phase A: 1 l of water+0.5 ml of 50% strength formic acid; mobile phase B: 1 l of acetonitrile+0.5 ml of 50% strength formic acid; gradient: 0.0 min 97% A→0.5 min 97% A→3.2 min 5% A→4.0 min 5% A; oven: 50° C.; flow rate: 0.3 ml/min; UV detection: 210 nm.

Method 4A:

MS instrument: Waters (Micromass) Quattro Micro; HPLC instrument: Agilent 1100 series; column: YMC-Triart C18 3μ 50×3 mm; mobile phase A: 1 l of water+0.01 mol of ammonium carbonate, mobile phase B: 1 l of acetonitrile; gradient: 0.0 min 100% A→2.75 min 5% A→4.5 min 5% A; oven: 40° C.; flow rate: 1.25 ml/min; UV detection: 210 nm.

Method 5A:

MS instrument: Waters (Micromass) QM; HPLC instrument: Agilent 1100 series; column: Agient ZORBAX Extend-C18 3.0×50 mm 3.5-Micron; mobile phase A: 1 l of water+0.01 mol of ammonium carbonate, mobile phase B: 1 l of acetonitrile; gradient: 0.0 min 98% A→0.2 min 98% A→3.0 min 5% A→4.5 min 5% A; oven: 40° C.; flow rate: 1.75 ml/min; UV detection: 210 nm.

Method 6A:

MS instrument: Waters (Micromass) ZQ; HPLC instrument: Agilent 1100 series; column: Agient ZORBAX Extend-C18 3.0×50 mm 3.5-Micron; mobile phase A: 1 l of water+0.01 mol of ammonium carbonate, mobile phase B: 1 l of acetonitrile; gradient: 0.0 min 98% A→0.2 min 98% A→3.0 min 5% A→4.5 min 5% A; oven: 40° C.; flow rate: 1.75 ml/min; UV detection: 210 nm.

Method 7A:

Instrument: Waters ACQUITY SQD UPLC system; column: Waters Acquity UPLC HSS T3 1.8μ 50×1 mm; mobile phase A: 1 l of water+0.25 ml of 99% strength formic acid; mobile phase B: 1 l of acetonitrile+0.25 ml of 99% strength formic acid; gradient: 0.0 min 95% A→6.0 min 5% A→7.5 min 5% A; oven: 50° C.; flow rate: 0.35 ml/min; UV detection: 210-400 nm.

Method 8A:

Instrument: Waters ACQUITY SQD UPLC system; column: Waters Acquity UPLC HSS T3 1.8μ 50×1 mm; mobile phase A: 1 l of water+0.25 ml of 99% strength formic acid; mobile phase B: 1 l of acetonitrile+0.25 ml of 99% strength formic acid; gradient: 0.0 min 90% A→1.2 min 5% A→2.0 min 5% A; oven: 50° C.; flow rate: 0.40 ml/min; UV detection: 210-400 nm.

### GC-MS Methods:

Method 1B:

Instrument: Thermo DFS, Trace GC Ultra; column: Restek RTX-35, 15 m×200 μm×0.33 μm; constant helium flow rate: 1.20 ml/min; oven: 60° C.; inlet: 220° C.; gradient: 60° C., 30° C./min→300° C. (maintained for 3.33 min)

Method 2B:

Instrument: Micromass GCT, GC6890; column: Restek RTX-35, 15 m×200 μm×0.33 μm; constant helium flow rate: 0.88 ml/min; oven: 70° C.; inlet: 250° C.; gradient: 70° C., 30° C./min 310° C. (maintained for 3 min).

### MS Methods:

Method 1C:

Instrument: Thermo Fisher-Scientific DSQ; chemical ionization; reactant gas NH₃; source temperature: 200° C.; ionization energy 70 eV.

Method 2C:

Instrument: Waters ZQ 2000; electrospray ionization; mobile phase A: 1 l of water+0.25 ml of 99% strength formic acid; mobile phase B: 1 l of acetonitrile+0.25 ml of 99% strength formic acid; 25% A, 75% B; flow rate: 0.25 ml/min

### Preparative Enantiomer/Diastereomer Separation on a Chiral Phase:

Method 1D:

phase: Daicel Chiralpak AZ-H, 5 μm 250 mm×30 mm, mobile phase: isohexane/ethanol 50:50; flow rate: 40 ml/min; temperature: 20° C.; UV detection: 220 nm.

Method 2D:

phase: Daicel Chiralpak AZ-H, 5 μm 250 mm×30 mm, mobile phase: isohexane/ethanol 50:50; flow rate: 40 ml/min; temperature: 25° C.; UV detection: 220 nm.

Method 3D:

phase: Daicel Chiralpak AD-H SFC, 10 μm 250 mm×20 mm, mobile phase: carbon dioxide/ethanol 70:30; flow rate: 100 ml/min, makeup flow rate: 30 ml/min, back pressure: 80 bar; temperature: 40° C.; UV detection: 220 nm.

Method 4D:

phase: Daicel Chiralpak AD-H, 5 μm 250 mm×20 mm, mobile phase: isohexane/isopropanol 70:30; flow rate: 20 ml/min; temperature: 25° C.; UV detection: 230 nm.

Method 5D:

phase: Daicel Chiralpak AZ-H, 5 μm 250 mm×30 mm, mobile phase: isohexane/ethanol 90:10; flow rate: 40 ml/min; temperature: 25° C.; UV detection: 220 nm.

Method 6D:

phase: Daicel Chiralpak AY-H, 5 μm 250 mm×20 mm, mobile phase: isohexane/ethanol 90:10; flow rate: 40 ml/min; temperature: 40° C.; UV detection: 220 nm.

Method 7D:

phase: Daicel Chiralpak AS-H, 5 μm 250 mm×20 mm, mobile phase: isohexane/ethanol 70:30; flow rate: 20 ml/min; temperature: 25° C.; UV detection: 230 nm.

Method 8D:

phase: Daicel Chiralpak AZ-H, 5 μm 250 mm×30 mm, mobile phase: isohexane/ethanol 50:50; flow rate: 20 ml/min; temperature: 25° C.; UV detection: 220 nm.

Method 9D:

phase: Daicel Chiralpak OZ-H, 5 μm 250 mm×20 mm, mobile phase: isohexane/ethanol 50:50; flow rate: 15 ml/min; temperature: 30° C.; UV detection: 220 nm.

Method 10D:

phase: Daicel Chiralpak OD-H, 5 μm 250 mm×20 mm, mobile phase: isohexane/ethanol 60:40; flow rate: 20 ml/min; temperature: 22° C.; UV detection: 230 nm.

Method 11D:

phase: Daicel Chiralpak AD-H SFC, 10 μm 250 mm×20 mm, mobile phase: carbon dioxide/methanol 70:30; flow rate: 100 ml/min, makeup flow rate: 30 ml/min, back pressure: 80 bar; temperature: 40° C.; UV detection: 210 nm.

Method 12D:

phase: Daicel Chiralcel AD-H, 5 μm 250 mm×20 mm; mobile phase: isohexane/ethanol 50:50+0.2% diethylamine; flow rate: 20 ml/min; temperature: 20° C.; UV detection: 220 nm.

Method 13D:

phase: Daicel Chiralcel AD-H, 5 μm 250 mm×20 mm; mobile phase: isohexane/isopropanol 50:50+0.2% diethylamine; flow rate: 20 ml/min; temperature: 20° C.; UV detection: 230 nm.

Method 14D:

phase: Daicel Chiralpak OD-H, 5 μm 250 mm×20 mm, mobile phase: isohexane/isopropanol 50:50; flow rate: 20 ml/min; temperature: 25° C.; UV detection: 230 nm.

Method 15D:

phase: Daicel Chiralpak IC, 5 μm 250 mm×20 mm; mobile phase: tert-butyl methyl ether/methanol 50:50; flow rate: 20 ml/min; temperature: 25° C.; UV detection: 220 nm.

Method 16D:

phase: Daicel Chiralpak AY-H, 5 μm 250 mm×20 mm, mobile phase: isohexane/ethanol 50:50; flow rate: 20 ml/min; temperature: 20° C.; UV detection: 230 nm.

Method 17D:

phase: Daicel Chiralpak AS-H, 5 μm 250 mm×20 mm, mobile phase: isohexane/ethanol 90:10; flow rate: 20 ml/min; temperature: 25° C.; UV detection: 220 nm.

Method 18D:

phase: Daicel Chiralcel AZ-H, 5 μm 250 mm×40 mm; mobile phase: isohexane/ethanol 90:10+0.2% diethylamine; flow rate: 35 ml/min; temperature: 25° C.; UV detection: 230 nm.

Method 19D:

phase: Daicel IA, 5 μm 250 mm×40 mm; mobile phase: tert-butyl methyl ether/methanol 50:50; flow rate: 20 ml/min; temperature: 25° C.; UV detection: 230 nm.

Method 20D:

phase: Daicel Chiralcel AD-H, 5 μm 250 mm×20 mm; mobile phase: isohexane/isopropanol 60:40+0.2% diethylamine; flow rate: 20 ml/min; temperature: 20° C.; UV detection: 220 nm.

Method 21D:

phase: Daicel Chiralpak IC, 5 μm 250 mm×20 mm; mobile phase: tert-butyl methyl ether/methanol/acetonitrile 50:25:25; flow rate: 15 ml/min; temperature: 35° C.; UV detection: 220 nm.

Method 22D:

phase: Daicel Chiralcel AZ-H, 5 μm 250 mm×40 mm; mobile phase: isohexane/ethanol 90:10+0.2% diethylamine; flow rate: 15 ml/min; temperature: 30° C.; UV detection: 220 nm.

Method 23D:

phase: Daicel Chiralcel AD-H, 5 μm 250 mm×20 mm; mobile phase: isohexane/isopropanol 50:50; flow rate: 20 ml/min; temperature: 40° C.; UV detection: 210 nm.

Method 24D:

phase: Daicel Chiralpak IC, 5 μm 250 mm×20 mm; mobile phase: acetonitrile/methanol 30:70; flow rate: 30 ml/min; temperature: 25° C.; UV detection: 220 nm.

Method 25D:

phase: Daicel Chiralpak OD-H, 5 μm 250 mm×20 mm, mobile phase: isohexane/ethanol 50:50; flow rate: 20 ml/min; temperature: 20° C.; UV detection: 220 nm.

Method 26D:

phase: Daicel Chiralpak AS-H, 5 μm 250 mm×20 mm; mobile phase: isohexane/ethanol 70:30+0.2% diethylamine; flow rate: 20 ml/min; temperature: 20° C.; UV detection: 220 nm.

Method 27D:

phase: Daicel Chiralpak AD-H SFC, 5 μm 250 mm×30 mm, mobile phase: carbon dioxide/methanol 80:20; flow rate: 100 ml/min, stepped gradient after 3 min for 1.5 min carbon dioxide/methanol 70:30; makeup flow rate: 30 ml/min, back pressure: 120 bar; temperature: 40° C.; UV detection: 210 nm.

Method 28D:

phase: Daicel Chiralpak AS-H, 5 μm, 250 mm×20 mm, mobile phase: 50% isohexane, 50% ethanol; flow rate: 20 ml/min; temperature: 25° C.; detection: 220 nm.

Method 29D:

phase: Daicel Chiralpak OD-H, 5 μm 250 mm×4 mm, mobile phase: 95% isohexane, 5% ethanol+1% diethylamine; flow rate: 20 ml/min; temperature: 40° C.; detection: 220 nm.

Method 30D:

phase: Daicel Chiralpak AZ-H 5 μm 250 mm×30 mm, mobile phase: 10% isohexane, 90% ethanol+0.2% diethylamine; flow rate: 40 ml/min; temperature: 20° C.; detection: 220 nm.

Method 31D:

phase: Daicel Chiralpak OD-H, 5 μm 250 mm×20 mm, mobile phase: 95% isohexane, 5% ethanol; flow rate: 20 ml/min; temperature: 40° C.; detection: 220 nm.

Method 32D:

phase: Daicel Chiralpak AD-H, 5 μm, 250 mm×4 mm; mobile phase: 50% isohexane, 50% ethanol/isopropanol, 0.2% diethylamine; flow rate: 1 ml/min; temperature: 40° C.; detection: 230 nm.

Method 33D:

phase: Daicel Chiralpak AS-H, 5 μm, 250 mm×20 mm, mobile phase: 70% isohexane, 30% ethanol/isopropanol, 0.2% diethylamine; flow rate: 20 ml/min; temperature: 40° C.; detection: 230 nm.

Method 34D:

phase: Daicel Chiralpak OZ-H, 5 μm 250 mm×20 mm, mobile phase: 30% isohexane, 70% ethanol with 0.2% diethylamine; flow rate: 15 ml/min; temperature: 40° C.; detection: 220 nm.

Method 35D:

phase: Daicel Chiralpak AS-H, 5 μm, 250 mm×20 mm, mobile phase: 65% isohexane, 35% ethanol with 0.2% diethylamine; flow rate: 20 ml/min; temperature: 25° C.; detection: 220 nm.

Method 36D:

phase: Daicel Chiralpak AS-H, 5 μm, 250 mm×20 mm, mobile phase: 50% isohexane, 50% isopropanol; flow rate: 20 ml/min; temperature: 25° C.; detection: 220 nm.

Method 37D:

phase: Daicel Chiralpak AS-H, 5 μm, 250 mm×20 mm, mobile phase: 50% isohexane, 50% ethanol; flow rate: 20 ml/min; temperature: 25° C.; detection: 230 nm.

### Analytical Enantiomer/Diastereomer Separation on a Chiral Phase:

Method 1E:

phase: Daicel Chiralcel OZ-H, 5 μm 250 mm×4.6 mm; mobile phase: isohexane/ethanol 50:50; flow rate: 1 ml/min; temperature: 30° C.; UV detection: 220 nm.

Method 2E:

phase: Daicel Chiralcel AZ-H, 5 μm 250 mm×4.6 mm; mobile phase: isohexane/ethanol 50:50; flow rate: 1 ml/min; temperature: 30° C.; UV detection: 220 nm.

Method 3E:

phase: Daicel Chiralpak AD-H SFC, 5 μm 250 mm×4.6 mm, mobile phase: carbon dioxide/ethanol 70:30; flow rate: 3 ml/min; temperature: 30° C.; UV detection: 220 nm.

Method 4E:

phase: Daicel Chiralpak AD-H, 5 μm 250 mm×4.6 mm, mobile phase: isohexane/isopropanol 50:50; flow rate: 1 ml/min; temperature: 30° C.; UV detection: 220 nm.

Method 5E:

phase: LUX Amylose-2, 5 μm 250 mm×4.6 mm; mobile phase: isohexane/ethanol 90:10; flow rate: 1 ml/min; temperature: 30° C.; UV detection: 220 nm.

Method 6E:

phase: Daicel Chiralpak AS-H, 5 μm 250 mm×4.6 mm, mobile phase: isohexane/isopropanol 50:50; flow rate: 1 ml/min; temperature: 30° C.; UV detection: 220 nm.

Method 7E:

phase: Daicel Chiralcel OD-H, 5 μm 250 mm×4.6 mm; mobile phase: isohexane/ethanol 80:20+0.2% diethylamine; flow rate: 1 ml/min; temperature: 40° C.; UV detection: 220 nm.

Method 8E:

phase: Daicel Chiralpak AD-H, 5 μm 250 mm×4.6 mm, mobile phase: isohexane/ethanol 50:50; flow rate: 1 ml/min; temperature: 30° C.; UV detection: 220 nm.

Method 9E:

phase: Daicel Chiralcel OZ-H, 5 μm 250 mm×4.6 mm; mobile phase: isohexane/ethanol 50:50; flow rate: 1 ml/min; temperature: 40° C.; UV detection: 220 nm.

Method 10E:

phase: Daicel Chiralcel OD-H, 5 μm 250 mm×4.6 mm; mobile phase: isohexane/ethanol 50:50; flow rate: 1 ml/min; temperature: 30° C.; UV detection: 220 nm.

Method 11E:

phase: Daicel Chiralpak AD-H SFC, 5 μm 250 mm×4.6 mm, mobile phase: carbon dioxide/ethanol 70:30; flow rate: 4 ml/min; temperature: 30° C.; UV detection: 220 nm.

Method 12E:

phase: Daicel Chiralcel AD-H, 5 μm 250 mm×4.6 mm; mobile phase: isohexane/ethanol 50:50+0.2% diethylamine; flow rate: 1 ml/min; temperature: 40° C.; UV detection: 220 nm.

Method 13E:

phase: Daicel Chiralpak OD-H, 5 μm 250 mm×4.6 mm, mobile phase: isohexane/isopropanol 50:50; flow rate: 1 ml/min; temperature: 25° C.; UV detection: 220 nm.

Method 14E:

phase: Daicel Chiralpak IC, 5 μm 250 mm×4.6 mm; mobile phase: tert-butyl methyl ether/methanol 50:50; flow rate: 1 ml/min; temperature: 30° C.; UV detection: 220 nm.

Method 15E:

phase: Daicel Chiralpak AY-H, 5 μm 250 mm×4.6 mm, mobile phase: isohexane/ethanol 50:50; flow rate: 1 ml/min; temperature: 45° C.; UV detection: 220 nm.

Method 16E:

phase: Daicel Chiralcel AZ-H, 5 μm 250 mm×4.6 mm; mobile phase: isohexane/ethanol 90:10; flow rate: 1 ml/min; temperature: 30° C.; UV detection: 220 nm.

Method 17E:

phase: Daicel Chiralpak AS-H, 5 μm 250 mm×4.6 mm, mobile phase: isohexane/ethanol 90:10; flow rate: 1 ml/min; temperature: 30° C.; UV detection: 220 nm.

Method 18E:

phase: Daicel Chiralcel AZ-H, 5 μm 250 mm×4.6 mm; mobile phase: isohexane/ethanol 90:10+0.2% diethylamine; flow rate: 1 ml/min; temperature: 40° C.; UV detection: 230 nm.

Method 19E:

phase: Daicel IA, 5 μm 250 mm×4.6 mm; mobile phase: tert-butyl methyl ether/methanol 50:50; flow rate: 1 ml/min; temperature: 30° C.; UV detection: 220 nm.

Method 20E:

phase: Daicel Chiralcel AD-H, 5 μm 250 mm×4.6 mm; mobile phase: isohexane/isopropanol 50:50+0.2% diethylamine; flow rate: 1 ml/min; temperature: 40° C.; UV detection: 220 nm.

Method 21E:

phase: Daicel Chiralpak IC, 5 μm 250 mm×4.6 mm; mobile phase: tert-butyl methyl ether/methanol 50:50; flow rate: 1 ml/min; temperature: 40° C.; UV detection: 220 nm.

Method 22E:

phase: Daicel Chiralpak IC, 5 μm 250 mm×4.6 mm; mobile phase: acetonitrile/methanol 30:70; flow rate: 1 ml/min; temperature: 30° C.; UV detection: 220 nm.

Method 23E:

phase: Daicel Chiralcel OD-H, 5 μm 250 mm×4.6 mm; mobile phase: isohexane/ethanol 80:20; flow rate: 1 ml/min; temperature: 40° C.; UV detection: 220 nm.

Method 24E:

phase: Daicel Chiralcel OZ-H, 5 μm, 250 mm×4.6 mm; mobile phase: isohexane/ethanol 50:50; flow rate: 1 ml/min; temperature: 30° C.; UV detection: 220 nm.

Method 25E:

phase: Daicel Chiralpak AD-H SFC, 5 μm 250 mm×4.6 mm; mobile phase: carbon dioxide/methanol 70:30; flow rate: 3 ml/min; temperature: 30° C.; UV detection: 220 nm.

Method 26E:

phase: Daicel Chiralpak AS-H, 5 μm, 250 mm×4.6 mm, mobile phase: 50% isohexane, 50% ethanol; flow rate: 1 ml/min; temperature: 25° C.; detection: 220 nm.

Method 27E:

phase: Daicel Chiralpak AS-H, 5 μm, 250 mm×4.6 mm, mobile phase: 30% isohexane, 70% ethanol; flow rate: 1 ml/min; temperature: 30° C.; detection: 220 nm.

Method 28E:

phase: Daicel Chiralpak OD-H, 5 μm 250 mm×4.6 mm, mobile phase: 95% isohexane, 5% ethanol; flow rate: 1 ml/min; temperature: 30° C.; detection: 220 nm.

Method 29E:

phase: Daicel Chiralpak AD-H, 5 μm, 250 mm×4 mm; mobile phase: 50% isohexane, 50% ethanol/isopropanol, 0.2% diethylamine; flow rate: 1 ml/min; temperature: 40° C.; detection: 230 nm.

Method 30E:

phase: Daicel Chiralpak AS-H, 5 μm, 250 mm×4 mm, mobile phase: 50% isohexane, 50% ethanol/isopropanol, 0.2% diethylamine; flow rate: 1 ml/min; temperature: 40° C.; detection: 230 nm.

Method 31E:

phase: Daicel Chiralpak OZ-H, 5 μm 250 mm×4.6 mm, mobile phase: 30% isohexane, 70% ethanol+0.2% diethylamine; flow rate: 1 ml/min; temperature: 40° C.; detection: 230 nm.

Method 32E:

phase: Daicel Chiralpak AS-H, 5 μm, 250 mm×4.6 mm, mobile phase: 50% isohexane, 50% ethanol with 0.2% diethylamine; flow rate: 1 ml/min; temperature: 40° C.; detection: 220 nm.

Method 33E:

phase: Daicel Chiralpak AS-H, 5 μm, 250 mm×4.6 mm, mobile phase: 50% isohexane, 50% isopropanol with 0.2% diethylamine; flow rate: 1 ml/min; temperature: 40° C.; detection: 220 nm.

Method 34E:

phase: Daicel Chiralpak AS-H, 5 μm, 250 mm×4.6 mm, mobile phase: 50% isohexane, 50% ethanol; flow rate: 1 ml/min; temperature: 30° C.; detection: 220 nm.

### Preparative Purification:

Method 1F:

phase: Sunfire C-18, 5 μm 250 mm×20 mm, mobile phase: water/acetonitrile gradient 80:20→5:95; flow rate: 23.75 ml/min+constant addition of 2% strength formic acid, flow rate: 1.25 ml/min; UV detection: 210 nm.

Method 2F:

phase: Sunfire C-18, 5 μm 250 mm×20 mm, mobile phase: water/acetonitrile 50:50+1% trifluoroacetic acid in water; flow rate: 25 ml/min; temperature: 40° C.; UV detection: 210 nm.

Method 3F:

phase: Agilent Prep 100, Xbridge C18, 5 μm 150 mm×19 mm, mobile phase: water/acetonitrile 40:60, flow rate: 23.75 ml/min+constant addition of 2% strength ammonia solution, flow rate: 1.25 ml/min; UV detection: 210 nm.

### Preparative Diastereomer Separation on an Achiral Phase:

Method 1G:

phase: Sunfire C-18, 5 μm 250 mm×20 mm, mobile phase: water/methanol 60:40, flow rate: 60 ml/min; temperature: 23° C., UV detection: 210 nm.

Method 2G:

phase: Sunfire C-18, 5 μm 250 mm×20 mm, mobile phase: acetonitrile/water/1% strength trifluoroacetic acid in water 65:30:5; flow rate: 56 ml/min; temperature: 23° C.; UV detection: 210 nm.

Method 3G:

phase: Kromasil 100 C-18, 5 μm 250 mm×20 mm, mobile phase: acetonitrile/water/1% strength trifluoroacetic acid in water 20:64:16; flow rate: 23.8 ml/min; temperature: 40° C.; UV detection: 210 nm.

### Microwave

The microwave reactor used was a single-mode instrument of the Biotage Initiator Microwave Synthesizer type.

When inventive compounds are purified by preparative HPLC by the above-described methods in which the eluents contain additives, for example trifluoroacetic acid, formic acid or ammonia, the inventive compounds may be obtained in salt form, for example as trifluoroacetate, formate or ammonium salt, if the inventive compounds contain a sufficiently basic or acidic functionality. Such a salt can be converted to the corresponding free base or acid by various methods known to the person skilled in the art.

In the case of the synthesis intermediates and working examples of the invention described hereinafter, any compound specified in the form of a salt of the corresponding base or acid is generally a salt of unknown exact stoichiometric composition, as obtained by the respective preparation and/or purification process. Unless specified in more detail, additions to names and structural formulae, such as “hydrochloride”, “trifluoroacetate”, “sodium salt” or “x HCl”, “x CF₃COOH”, “x Na⁺” should not be understood in a stoichiometric sense in the case of such salts, but have merely descriptive character with regard to the salt-forming components present therein.

This applies correspondingly if synthesis intermediates or working examples or salts thereof were obtained in the form of solvates, for example hydrates, of unknown stoichiometric composition (if they are of a defined type) by the preparation and/or purification processes described.

### Starting Materials

### Example 1A

### Methyl 6-{[(ethoxycarbonyl)carbamothioyl]amino}-5-methoxynicotinate

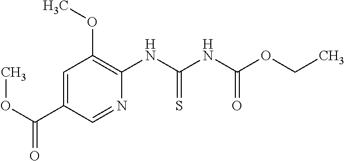 [SMILES: CCOC(=O)NC(=S)Nc1ncc(C(=O)OC)cc1OC]

Under argon, 7.72 g (42.3 mmol) of methyl 6-amino-5-methoxynicotinate [G. Brooks, E. Hunt, WO 01/74788 A1, 2001] were initially charged in 1,4-dioxane (200 ml), and 8.34 g (7.19 ml, 63.6 mmol) of ethoxycarbonyl isothiocyanate were then added dropwise. The mixture was stirred at RT overnight and most of the 1,4-dioxane was then removed under reduced pressure. The residue was taken up in dichloromethane and then washed with water and with saturated aqueous sodium chloride solution. The organic phase was dried over sodium sulphate, filtered and concentrated under reduced pressure. The target compound was used for the next step without further purification. Yield: 14.7 g (99% of theory, purity: 88%).

LC-MS (method 1A): Rₜ=0.88 min; MS (ESIpos): m/z=314 [M+H]⁺.

### Example 2A

### Methyl 2-amino-8-methoxy[1,2,4]triazolo[1,5-a]pyridine-6-carboxylate

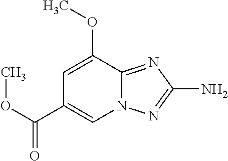 [SMILES: COC(=O)c1cc(OC)c2nc(N)nn2c1]

14.8 g (41.6 mmol, purity: 88%) of methyl 6-{[(ethoxycarbonyl)carbamothioyl]amino}-5-methoxynicotinate were initially charged in methanol/ethanol (1:1, 600 ml), 15.6 g (224 mmol) of hydroxylamine hydrochloride were added and the mixture was then stirred at 60° C. for 1 h. The reaction solution was cooled to RT and the precipitated solid was filtered off under reduced pressure. The solid was washed with methanol and dried under high vacuum. Yield: 6.35 g (69% of theory).

LC-MS (method 1A): Rₜ=0.49 min; MS (ESIpos): m/z=223 [M+H]⁺;

¹H-NMR (500 MHz, DMSO-d₆): δ [ppm]=8.68 (d, 1H), 9.26-8.36 (m, 1H), 7.21 (d, 1H), 6.26 (s, 2H), 3.97 (s, 3H), 3.87 (s, 3H).

### Example 3A

### Ethyl 2-[(3-chlorobenzyl)amino]-8-methoxy[1,2,4]triazolo[1,5-a]pyridine-6-carboxylate

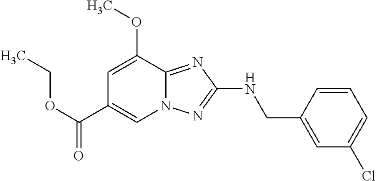 [SMILES: CCOC(=O)c1cc(OC)c2nc(NCc3cccc(Cl)c3)nn2c1]

4.00 g (18.0 mmol) of methyl 2-amino-8-methoxy[1,2,4]triazolo[1,5-a]pyridine-6-carboxylate were initially charged in N,N-dimethylformamide (80.0 ml), 3.54 g (2.86 ml, 25.2 mmol) of benzaldehyde were added and the mixture was then stirred under reflux for 2 h. The reaction solution was cooled to RT and concentrated under reduced pressure. The residue was taken up in ethanol (80.0 ml), and 1.23 g (32.4 mmol) of sodium borohydride were added carefully at 50° C. The mixture was then stirred under reflux for 1.5 h and cooled to RT, and water was then added. The precipitated solid was filtered off under reduced pressure and dried under high vacuum. Yield: 3.10 g (45% of theory).

LC-MS (method 1A): Rₜ=1.08 min; MS (ESIpos): m/z=361 [M+H]⁺;

¹H-NMR (500 MHz, DMSO-d₆): δ [ppm]=8.72 (d, 1H), 7.48 (t, 1H), 7.40 (s, 1H), 7.36-7.30 (m, 2H), 7.29-7.26 (m, 1H), 7.23 (d, 1H), 4.48 (d, 2H), 4.33 (q, 2H), 3.97 (s, 3H), 1.33 (t, 3H).

### Example 4A

### 2-[(3-Chlorobenzyl)amino]-8-methoxy[1,2,4]triazolo[1,5-a]pyridine-6-carboxylic acid

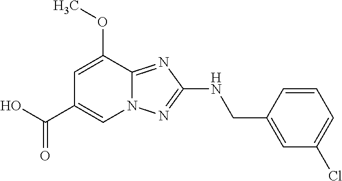 [SMILES: COc1cc(C(=O)O)cn2nc(NCc3cccc(Cl)c3)nc12]

3.05 g (8.03 mmol) of ethyl 2-[(3-chlorobenzyl)amino]-8-methoxy[1,2,4]triazolo[1,5-a]pyridine-6-carboxylate were initially charged in tetrahydrofuran/water (3:1, 100 ml), 962 mg (40.2 mmol) of lithium hydroxide were added and the mixture was then stirred at RT overnight. The reaction solution was substantially freed from the tetrahydrofuran and then acidified with 1 N aqueous hydrogen chloride solution. The precipitated solid was filtered off under reduced pressure, washed with water and dried under high vacuum. Yield: 2.45 g (91% of theory).

LC-MS (method 1A): Rₜ=0.86 min; MS (ESIpos): m/z=333 [M+H]⁺;

¹H-NMR (400 MHz, DMSO-d₆): δ [ppm]=13.25 (br. s., 1H), 8.66 (d, 1H), 7.46 (t, 1H), 7.40 (s, 1H), 7.37-7.26 (m, 3H), 7.24 (d, 1H), 4.48 (d, 2H), 3.96 (s, 3H).

### Example 5A

### Methyl 2-bromo-8-methoxy[1,2,4]triazolo[1,5-a]pyridine-6-carboxylate

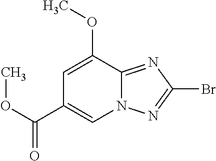 [SMILES: COC(=O)c1cc(OC)c2nc(Br)nn2c1]

510 mg (59 μl, 4.95 mmol) of tert-butyl nitrite and 1.11 g (4.95 mmol) of copper(II) bromide were initially charged in acetonitrile (50.0 ml), and the mixture was then stirred at 60° C. for 30 min. 1.00 g (4.50 mmol) of methyl 2-amino-8-methoxy[1,2,4]triazolo[1,5-a]pyridine-6-carboxylate was then added in five portions, and the mixture was stirred at 75° C. for 2 h. A further 556 mg (648 μl, 5.40 mmol) of tert-butyl nitrite and 1.11 g (4.95 mmol) of copper(II) bromide were added and the mixture was then stirred at 75° C. for 2 h. Water was then added, and the reaction solution was extracted with dichloromethane. The organic phase was dried over sodium sulphate, filtered and concentrated under reduced pressure. The residue was purified by trituration with acetonitrile/water. Yield: 898 mg (67% of theory).

LC-MS (method 1A): Rₜ=0.79 min; MS (ESIpos): m/z=286 [M+H]⁺;

¹H-NMR (500 MHz, DMSO-d₆): δ [ppm]=9.09 (s, 1H), 7.43 (s, 1H), 4.06 (s, 3H), 3.92 (s, 3H).

### Example 6A

### 2-Bromo-8-methoxy[1,2,4]triazolo[1,5-a]pyridine-6-carboxylic acid

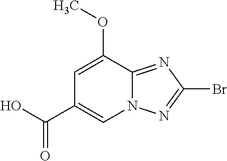

3.02 g (9.42 mmol) of methyl 2-bromo-8-methoxy[1,2,4]triazolo[1,5-a]pyridine-6-carboxylate were initially charged in tetrahydrofuran/water (3:1, 160 ml), 1.13 g (47.1 mmol) of lithium hydroxide were added and the mixture was stirred at RT overnight. The reaction solution was acidified with 1 N aqueous hydrogen chloride solution and the aqueous phase was saturated with sodium chloride and extracted with tetrahydrofuran. The organic phase was dried over sodium sulphate, filtered and concentrated under reduced pressure. Yield: 2.93 g (quant.).

LC-MS (method 1A): Rₜ=0.59 min; MS (ESIpos): m/z=272 [M+H]⁺;

¹H-NMR (400 MHz, DMSO-d₆): δ [ppm]=13.72 (br. s., 1H), 9.02 (s, 1H), 7.43 (s, 1H), 4.04 (s, 3H).

### Example 7A

### tert-Butyl 2-bromo-8-methoxy[1,2,4]triazolo[1,5-a]pyridine-6-carboxylate

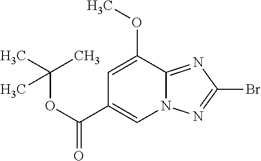 [SMILES: COc1cc(C(=O)OC(C)(C)C)cn2nc(Br)nc12]

4.14 g (15.2 mmol) of 2-bromo-8-methoxy[1,2,4]triazolo[1,5-a]pyridine-6-carboxylic acid, 1.29 g (1.66 ml, 17.3 mmol) of tert-butanol and 930 mg (7.61 mmol) of dimethylaminopyridine were initially charged in dichloromethane (69.3 ml), and 3.18 g (16.6 mmol) of 1-(3-dimethylaminopropyl)-3-ethylcarbodiimide hydrochloride were added at 0° C. The mixture was stirred at 0° C. for 2 h and then at RT for 5 h. A further 645 mg (830 μl, 8.65 mmol) of tert-butanol, 465 mg (3.81 mmol) of dimethylaminopyridine and 1.59 g (8.30 mmol) of 1-(3-dimethylaminopropyl)-3-ethylcarbodiimide hydrochloride were added and the mixture was stirred overnight. The reaction solution was washed first with water and then with saturated aqueous sodium bicarbonate solution. The organic phase was dried over magnesium sulphate, filtered and concentrated under reduced pressure. The residue was purified by trituration (acetonitrile/water). Yield: 3.97 g (79% of theory).

LC-MS (method 1A): Rₜ=1.12 min; MS (ESIpos): m/z=338 [M+H]⁺.

### Example 8A

### 1-(3-Chlorophenyl)-2-fluoroethanone

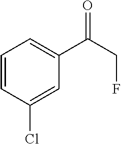 [SMILES: O=C(CF)c1cccc(Cl)c1]

### Method 1:

50.7 g (161 mmol) of tetra-n-butylammonium fluoride trihydrate, 22.1 g (214 mmol) of zinc fluoride and 6.22 g (107 mmol) of potassium fluoride were initially charged in acetonitrile (850 ml) and then stirred at 80° C. for 1 h (lit.: X. Zou et al., J. Fluorine Chem. 2010, 131, 340-344.). 50.0 g (214 mmol) of 2-bromo-1-(3-chlorophenyl)ethanone in acetonitrile (210 ml) were then added dropwise at this temperature over a period of 3 h, and the mixture was subsequently stirred at 80° C. for a further 3 h. The reaction solution was cooled to RT and the precipitated salts were filtered off over a glass frit. The filtrate was concentrated under reduced pressure, water was added to the residue and the mixture was then extracted repeatedly with tert-butyl methyl ether (further precipitated salts were removed by filtration). The organic phases were dried over magnesium sulphate, filtered and concentrated under reduced pressure. The crude product was then purified by flash chromatography on silica gel (petroleum ether and then petroleum ether/ethyl acetate 10:1). Yield: 27.0 g (58% of theory, purity: 80%).

GC-MS (method 1B): Rₜ=3.73 min; MS (EIpos): m/z=172 [M]⁺;

¹H-NMR (400 MHz, DMSO-d₆): δ [ppm]=7.91 (m_{c}, 1H), 7.85 (d_{br}, 1H), 7.77 (dd, 1H), 7.60 (m_{c}, 1H), 5.85 (d, 2H).

### Method 2:

10.0 g (64.7 mmol) of 1-(3-chlorophenyl)ethanone were initially charged in methanol (80.0 ml), 45.8 g (129 mmol) of 1-(chloromethyl)-4-fluoro-1,4-diazoniabicyclo[2.2.2]octane bistetrafluoroborate (Selectfluor) were added and the mixture was then, in ten portions, stirred in the microwave (Biotage Synthesizer) at 110° C. for 2.5 h (lit.: B. H. Hoff et al., Tetrahedron 2009, 65, 9550-9556.). Water (5.0 ml) was then added to each of the ten portions, and the mixtures were stirred in a microwave at 110° C. for 1 h. The portions were combined, most of the methanol was removed under reduced pressure and the residue was diluted with water and then extracted with ethyl acetate. The organic phase was dried over sodium sulphate, filtered and concentrated under reduced pressure. The crude product was used without further purification in the next step. Yield: 12.5 g (86% of theory, purity: 77%).

GC-MS (method 2B): Rₜ=3.56 min; MS (EIpos): m/z=172 [M]⁺.

### Example 9A

### 1-(3-Chlorophenyl)-2-fluoroethanamine [racemate]

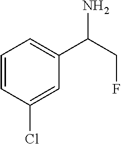 [SMILES: NC(CF)c1cccc(Cl)c1]

### Method 1:

82.0 g (86.3 ml, 289 mmol) of titanium tetraisopropoxide were added dropwise to 24.9 g (144.0 mmol) of 1-(3-chlorophenyl)-2-fluoroethanone in 2 M ethanolic ammonia solution (361 ml, 722 mmol), the temperature being kept at 20° C. by ice cooling, and the mixture was stirred at RT overnight. At 10° C., 8.19 g (216 mmol) of sodium borohydride were then added a little at a time, and the mixture was stirred at RT for 6 h. 1.64 g (43.2 mmol) of sodium borohydride were added and the mixture was stirred overnight. Semiconcentrated aqueous hydrogen chloride solution (300 ml) was added to the reaction solution, and the mixture was then diluted with water (1.0 l) (pH=2). The mixture was extracted with tert-butyl methyl ether (3×500 ml), and the organic phases were dried over sodium sulphate, filtered and concentrated under reduced pressure. Yield fraction 1: 10.5 g (purity: 58%).

The aqueous phase was adjusted to pH=10 with 45% strength aqueous sodium hydroxide solution, saturated with sodium chloride and extracted with tert-butyl methyl ether (3×500 ml). The combined organic phases were dried over sodium sulphate, filtered and concentrated under reduced pressure. Yield fraction 2: 12.9 g (51% of theory).

Fraction 1 and fraction 2 were then combined and used for the next step without further purification.

Fraction 2: LC-MS (method 5A): Rₜ=1.93 min; MS (ESIpos): m/z=174 [M+H]⁺;

Fraction 2: ¹H-NMR (400 MHz, DMSO-d₆): δ [ppm]=7.50 (s, 1H), 7.40-7.27 (m, 3H), 4.45 (m_{c}, 1H), 4.32 (m_{c}, 1H), 4.12 (dt, 1H), 2.10 (br. s., 2H).

### Method 2:

Under an atmosphere of argon, 21.6 g (22.8 ml, 76.0 mmol) of titanium tetraisopropoxide were added to 8.20 g (38.0 mmol, purity: 80%) of 1-(3-chlorophenyl)-2-fluoroethanone in 2 M ethanolic ammonia solution (95 ml, 190 mmol), and the mixture was stirred at RT for 16 h. 1.51 g (57.3 mmol) of sodium borohydride were then added, and the mixture was stirred at RT for 5 h. A further 700 mg (18.5 mmol) of sodium borohydride were added, and the mixture was stirred overnight. Using 6 M aqueous hydrogen chloride solution, the reaction solution was adjusted to pH=2 and then extracted three times with tert-butyl methyl ether. The aqueous phase was adjusted to pH=10 with solid sodium hydroxide, saturated with sodium chloride and extracted four times with tert-butyl methyl ether. The combined organic phases were dried over sodium sulphate, filtered and concentrated under reduced pressure. The residue was taken up in dichloromethane and washed with saturated aqueous sodium bicarbonate solution. The organic phase was dried over sodium sulphate, filtered and concentrated under reduced pressure. The target compound was used for the next step without further purification. Yield: 7.32 g (90% of theory, purity: 81%).

LC-MS (method 5A): Rₜ=1.92 min; MS (ESIpos): m/z=174 [M+H]⁺.

### Example 10A

### 1-(3-Chlorophenyl)-2-fluoroethanamine hydrochloride [racemate]

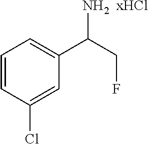 [SMILES: NC(CF)c1cccc(Cl)c1 ; "xHCl"]

### Method 1:

Under an atmosphere of argon, 6.52 g (6.87 ml, 23.0 mmol) of titanium tetraisopropoxide were added to 2.00 g (11.5 mmol) of 1-(3-chlorophenyl)-2-fluoroethanone in 2 M ethanolic ammonia solution (28.7 ml, 57.4 mmol), and the mixture was stirred at RT for 16 h. 654 mg (17.3 mmol) of sodium borohydride were then added, and the mixture was stirred at RT for 5 h. A further 350 mg (9.25 mmol) of sodium borohydride were added, and the mixture was stirred at RT overnight. The reaction solution was poured into 25% strength aqueous ammonia solution (100 ml) and then filtered through kieselguhr. tert-Butyl methyl ether (200 ml) was added to the filtrate, the mixture was extracted and, after phase separation, the aqueous phase was extracted once more with tert-butyl methyl ether (100 ml). The combined organic phases were dried over sodium sulphate, filtered and concentrated under reduced pressure. The crude product was dissolved in diethyl ether/tetrahydrofuran (5:1; 60 ml), and a 4 N solution of hydrogen chloride in 1,4-dioxane (10.0 ml) was then added. The precipitated white solid was filtered off under reduced pressure, washed with a little diethyl ether and then dried under high vacuum. Yield: 1.54 g (63% of theory).

LC-MS (method 5A): Rₜ=1.94 min; MS (ESIpos): m/z=174 [M+H-HCl]⁺;

¹H-NMR (400 MHz, DMSO-d₆): δ [ppm]=8.98 (br. s., 3H), 7.70 (s, 1H), 7.60-7.47 (m, 3H), 4.88-4.67 (m, 3H).

### Method 2:

1.10 kg (1.16 μl, 3.88 mol) of titanium tetraisopropoxide were added dropwise to 335 g (1.94 mol) of 1-(3-chlorophenyl)-2-fluoroethanone in 2 M ethanolic ammonia solution (4.85 l, 9.71 mol), the temperature being kept at 20° C. by ice cooling, and the mixture was stirred at RT overnight. At 10° C., 110 g (2.91 mol) of sodium borohydride were then added in four portions, and the mixture was stirred at RT for 36 h. A further 29.4 g (776 mmol) of sodium borohydride were then added, and the mixture was stirred for 1 h. The reaction solution was poured into 2 M aqueous ammonia solution (4.85 l) and the precipitated salts were filtered off over a frit under reduced pressure. tert-Butyl methyl ether (14 l) and water (50 l) were added to the filtrate, the mixture was extracted and 5% aqueous sodium chloride solution was then added to facilitate phase separation. After phase separation, the aqueous phase was re-extracted with tert-butyl methyl ether (5 l) and the combined organic phases were dried over sodium sulphate, filtered and concentrated under reduced pressure. The crude product was dissolved in diethyl ether/tetrahydrofuran (10:1; 1.1 l), and a 4 N solution of hydrogen chloride in 1,4-dioxane (385 ml) was added with stirring and ice cooling. The precipitated white solid was filtered off under reduced pressure, washed with a little diethyl ether and dried under high vacuum. Yield: 261 g (77% of theory).

LC-MS (method 5A): Rₜ=1.93 min; MS (ESIpos): m/z=174 [M+H-HCl]⁺;

### Example 11A

### tert-Butyl[1-(3-chlorophenyl)-2-fluoroethyl]carbamate [racemate]

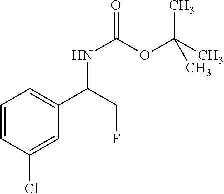 [SMILES: CC(C)(C)OC(=O)NC(CF)c1cccc(Cl)c1]

### Method 1:

7.47 g (43.3 mmol) of 1-(3-chlorophenyl)-2-fluoroethanamine [racemate] were suspended in dichloromethane (150 ml), subsequently first 9.14 g (12.6 ml, 90.4 mmol) of triethylamine and then 10.3 g (47.3 mmol) of di-tert-butyl dicarbonate were added and the mixture was stirred at RT overnight. The reaction solution was then washed with 0.5 N aqueous hydrogen chloride solution (100 ml), saturated aqueous sodium bicarbonate solution (100 ml) and water (100 ml), and the organic phase was dried over sodium sulphate, filtered and concentrated under reduced pressure. The crude product was purified by preparative RP-HPLC (water/acetonitrile). Yield: 7.23 g (61% of theory).

LC-MS (method 1A): Rₜ=1.10 min; MS (ESIpos): m/z=218 [M+H-C₄H₉]⁺;

¹H-NMR (400 MHz, DMSO-d₆): δ [ppm]=7.72 (d, 1H), 7.45 (s, 1H), 7.41-7.29 (m, 3H), 4.89 (m_{c}, 1H), 4.59-4.45 (m, 1H), 4.45-4.32 (m, 1H), 1.38 (s, 9H).

### Method 2:

Under an atmosphere of argon, 41.5 g (198 mmol) of 1-(3-chlorophenyl)-2-fluoroethanamine hydrochloride [racemate] were suspended in dichloromethane (200 ml), and subsequently first 80.0 g (110 ml, 790 mmol) of triethylamine and then more dichloromethane (200 ml) were added. 31.0 g (142 mmol) of di-tert-butyl dicarbonate in dichloromethane (100 ml) were then added, and the mixture was stirred at RT overnight. A further 9.91 g (45.4 mmol) of di-tert-butyl dicarbonate were added, the mixture was stirred to virtually complete conversion (checked by TLC) and the reaction solution was subsequently washed with 1 N aqueous hydrogen chloride solution (2×100 ml) and saturated aqueous sodium bicarbonate solution (100 ml). The organic phase was dried over sodium sulphate, filtered and concentrated under reduced pressure. Yield: 51.0 g (94% of theory).

LC-MS (method 7A): Rₜ=3.25 min; MS (ESIpos): m/z=218 [M+H-C₄H₉]⁺.

### Example 12A

### tert-Butyl[1-(3-chlorophenyl)-2-fluoroethyl]carbamate [enantiomerically pure isomer 1]

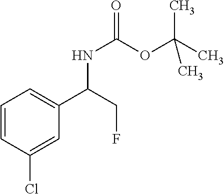 [SMILES: CC(C)(C)OC(=O)NC(CF)c1cccc(Cl)c1]

Enantiomer separation on a chiral phase of 7.23 g of the compound from Example 11A according to Method 6D gave 3.05 g of Example 12A (enantiomerically pure isomer 1) and 3.05 g of Example 13A (enantiomerically pure isomer 2).

HPLC (Method 6E): Rₜ=5.01 min, 99.0% ee;

LC-MS (method 7A): Rₜ=3.26 min; MS (ESIpos): m/z=218 [M+H-C₄H₉]⁺;

¹H-NMR (400 MHz, DMSO-d₆): δ [ppm]=7.72 (d, 1H), 7.45 (s, 1H), 7.42-7.27 (m, 3H), 4.89 (m_{c}, 1H), 4.59-4.45 (m, 1H), 4.44-4.31 (m, 1H), 1.38 (s, 9H).

### Example 13A

### tert-Butyl[1-(3-chlorophenyl)-2-fluoroethyl]carbamate [enantiomerically pure isomer 2]

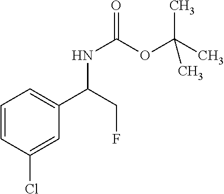 [SMILES: CC(C)(C)OC(=O)NC(CF)c1cccc(Cl)c1]

Enantiomer separation on a chiral phase of 7.23 g of the compound from Example 11A according to Method 6D gave 3.05 g of Example 12A (enantiomerically pure isomer 1) and 3.05 g of Example 13A (enantiomerically pure isomer 2).

HPLC (Method 6E): Rₜ=7.46 min, 99.0% ee;

LC-MS (method 7A): Rₜ=3.26 min; MS (ESIpos): m/z=218 [M+H-C₄H₉]⁺;

¹H-NMR (400 MHz, DMSO-d₆): δ [ppm]=7.72 (d, 1H), 7.45 (s, 1H), 7.42-7.28 (m, 3H), 4.89 (m_{c}, 1H), 4.58-4.45 (m, 1H), 4.44-4.30 (m, 1H), 1.38 (s, 9H).

### Example 14A

### 1-(3-Chlorophenyl)-2-fluoroethanamine [enantiomerically pure isomer 2]

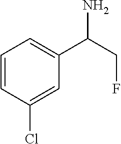 [SMILES: NC(CF)c1cccc(Cl)c1]

19.6 g (71.6 mmol) of tert-butyl [1-(3-chlorophenyl)-2-fluoroethyl]carbamate [Example 13A, enantiomerically pure isomer 2] were initially charged in 1,4-dioxane (199 ml), and 179 ml (716 mmol) of 4 N hydrogen chloride solution in 1,4-dioxane were then added at RT. The reaction solution was stirred at RT for 60 h and the resulting suspension was then concentrated completely under reduced pressure. The residue was stirred with tert-butyl methyl ether (100 ml) and filtered. The solid was dissolved in dichloromethane (250 ml) and washed with saturated aqueous sodium bicarbonate solution (100 ml). The organic phase was dried over magnesium sulphate, filtered and concentrated under reduced pressure. Yield: 11.0 g (86% of theory).

LC-MS (method 5A): Rₜ=1.93 min; MS (ESIpos): m/z=174 [M+H]⁺;

¹H-NMR (400 MHz, DMSO-d₆): δ [ppm]=7.50 (s, 1H), 7.40-7.25 (m, 3H), 4.44 (m_{c}, 1H), 4.32 (m_{c}, 1H), 4.12 (m_{c}, 1H), 2.06 (br. s., 2H).

### Example 15A

### 1-(3-Chlorophenyl)-2-fluoroethanamine hydrochloride [enantiomerically pure isomer 2]

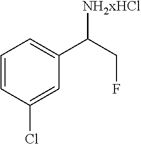

17.3 g (63.2 mmol) of tert-butyl [1-(3-chlorophenyl)-2-fluoroethyl]carbamate [Example 13A, enantiomerically pure isomer 2] were initially charged in 1,4-dioxane (50 ml), and 79 ml (316 mmol) of 4 N hydrogen chloride solution in 1,4-dioxane were added at RT. After a short while, a solid was formed which was diluted with 1,4-dioxane (250 ml), and a further 31.6 ml (126 mmol) of 4 N hydrogen chloride solution in 1,4-dioxane were added. The mixture was stirred at RT overnight and the resulting suspension was then concentrated completely under reduced pressure. The residue was stirred with tert-butyl methyl ether (200 ml) and filtered and the filter residue was washed with tert-butyl methyl ether (2×50 ml). The solid formed was dried under high vacuum. Yield: 13.2 g (99% of theory).

optical rotation: [α]_{D}²⁰=27.06° (c=0.51, methanol);

LC-MS (method 5A): Rₜ=1.94 min; MS (ESIpos): m/z=174 [M+H-HCl]⁺;

¹H-NMR (400 MHz, DMSO-d₆): δ [ppm]=8.91 (br. s., 3H), 7.68 (s, 1H), 7.55-7.47 (m, 3H), 4.89-4.66 (m, 3H).

### Example 16A

### tert-Butyl 2-{[(1R)-1-(3-chlorophenyl)ethyl]amino}-8-methoxy[1,2,4]triazolo[1,5-a]pyridine-6-carboxylate [enantiomerically pure isomer]

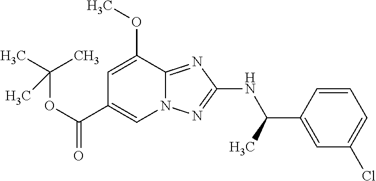 [SMILES: COc1cc(C(=O)OC(C)(C)C)cn2nc(N[C@H](C)c3cccc(Cl)c3)nc12]

144 mg (0.44 mmol) of tert-butyl 2-bromo-8-methoxy[1,2,4]triazolo[1,5-a]pyridine-6-carboxylate, 171 mg (1.10 mmol) of (1R)-1-(3-chlorophenyl)ethanamine [enantiomerically pure isomer], 146 mg (1.05 mmol) of potassium carbonate, 17.5 mg (0.02 mmol) of chloro-[2-(dicyclohexylphosphine)-3,6-dimethoxy-2′,4′,6′-triisopropyl-1,1′-biphenyl][2-(2-aminoethyl)phenyl]palladium(II) (BrettPhos precatalyst) and 11.8 mg (0.02 mmol) of 2-(dicyclohexylphosphine)-3,6-dimethoxy-2′,4′,6′-triisopropyl-1,1′-biphenyl (BrettPhos) were initially charged in degassed tert-butanol (1.90 ml) in a microwave tube. The tube was sealed and the reaction mixture was stirred at 110° C. (preheated oil bath) for 6 h. 17.5 mg (0.02 mmol) of chloro-[2-(dicyclohexylphosphine)-3,6-dimethoxy-2′,4′,6′-triisopropyl-1,1′-biphenyl][2-(2-aminoethyl)phenyl]palladium(II) (BrettPhos precatalyst) and 11.8 mg (0.02 mmol) of 2-(dicyclohexylphosphine)-3,6-dimethoxy-2′,4′,6′-triisopropyl-1,1′-biphenyl (BrettPhos) were then added, and the mixture was stirred at 110° C. for a further 6 h. The reaction solution was concentrated under reduced pressure and the residue was purified by preparative RP-HPLC (acetonitrile/water). Yield: 59.4 mg (31% of theory).

LC-MS (method 1A): Rₜ=1.28 min; MS (ESIpos): m/z=403 [M+H]⁺.

### Example 17A

### 2-{[(1R)-1-(3-Chlorophenyl)ethyl]amino}-8-methoxy[1,2,4]triazolo[1,5-a]pyridine-6-carboxylic acid [enantiomerically pure isomer]

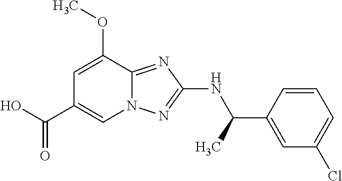 [SMILES: COc1cc(C(=O)O)cn2nc(N[C@H](C)c3cccc(Cl)c3)nc12]

59.4 mg (0.15 mmol) of tert-butyl 2-{[(1R)-1-(3-chlorophenyl)ethyl]amino}-8-methoxy[1,2,4]triazolo[1,5-a]pyridine-6-carboxylate [enantiomerically pure isomer] were initially charged in dichloromethane (3.00 ml), 437 mg (295 μl, 3.83 mmol) of trifluoroacetic acid and 85.7 mg (118 μl, 0.74 mmol) of triethylsilane were added at RT and the mixture was then stirred at RT for 60 h. The mixture was then stirred at 40° C. for 6 h, and 219 mg (148 μl, 1.92 mmol) of trifluoroacetic acid and 42.9 mg (59 μl, 0.37 mmol) of triethylsilane were added. The reaction solution was stirred at 40° C. for 4 h and then concentrated under reduced pressure, and the residue was purified by trituration with a little diethyl ether. Yield: 73.0 mg (95% of theory, purity: 67%).

LC-MS (method 1A): Rₜ=0.92 min; MS (ESIpos): m/z=347 [M+H]⁺;

### Example 18A

### tert-Butyl 2-{[1-(3-chlorophenyl)-2-fluoroethyl]amino}-8-methoxy[1,2,4]triazolo[1,5-a]pyridine-6-carboxylate [enantiomerically pure isomer]

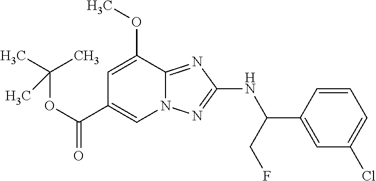 [SMILES: COc1cc(C(=O)OC(C)(C)C)cn2nc(NC(CF)c3cccc(Cl)c3)nc12]

100 mg (0.31 mmol) of tert-butyl 2-bromo-8-methoxy[1,2,4]triazolo[1,5-a]pyridine-6-carboxylate, 135 mg (0.76 mmol) of 1-(3-chlorophenyl)-2-fluoroethanamine [enantiomerically pure isomer 2], 33.0 mg (183 μl, 0.37 mmol, 2 M solution in tetrahydrofuran) of sodium tert-butoxide, 12.1 mg (0.02 mmol) of chloro-[2-(dicyclohexylphosphine)-3,6-dimethoxy-2′,4′,6′-triisopropyl-1,1′-biphenyl][2-(2-aminoethyl)phenyl]palladium(II) (BrettPhos precatalyst) and 8.1 mg (0.02 mmol) of 2-(dicyclohexylphosphine)-3,6-dimethoxy-2′,4′,6′-triisopropyl-1,1′-biphenyl (BrettPhos) were initially charged in degassed tert-butanol (2.00 ml) in a microwave tube. The tube was sealed and the reaction mixture was subsequently stirred at 90° C. in the microwave oven (Biotage Synthesizer) for 2 h. The reaction solution was diluted with dichloromethane (20 ml) and washed with 0.5 N aqueous hydrogen chloride solution (20 ml), and the organic phase was concentrated under reduced pressure. The residue was purified by preparative RP-HPLC (acetonitrile/water). Yield: 34.3 mg (27% of theory).

LC-MS (method 1A): Rₜ=1.22 min; MS (ESIpos): m/z=421 [M+H]⁺;

¹H-NMR (400 MHz, DMSO-d₆): δ [ppm]=8.64 (d, 1H), 7.78 (d, 1H), 7.57 (s, 1H), 7.49-7.31 (m, 3H), 7.19 (d, 1H), 5.18 (m_{c}, 1H), 4.75-4.49 (m, 2H), 3.96 (s, 3H), 1.55 (s, 9H).

### Example 19A

### 2-{[1-(3-Chlorophenyl)-2-fluoroethyl]amino}-8-methoxy[1,2,4]triazolo[1,5-a]pyridine-6-carboxylic acid [enantiomerically pure isomer]

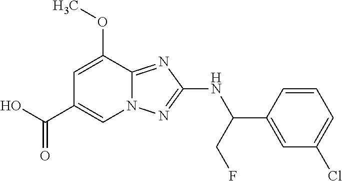 [SMILES: COc1cc(C(=O)O)cn2nc(NC(CF)c3cccc(Cl)c3)nc12]

378 mg (0.898 mmol) of tert-butyl 2-{[1-(3-chlorophenyl)-2-fluoroethyl]amino}-8-methoxy[1,2,4]triazolo[1,5-a]pyridine-6-carboxylate [enantiomerically pure isomer] were initially charged in dichloromethane (5.00 ml), 1.48 g (1.00 ml, 13.0 mmol) of trifluoroacetic acid and 437 mg (600 μl, 3.76 mmol) of triethylsilane were added at RT and the mixture was then stirred for 60 h. The reaction solution was concentrated under reduced pressure and the residue was purified by trituration with a little diethyl ether. Yield: 310 mg (94% of theory).

LC-MS (method 1A): Rₜ=0.89 min; MS (ESIpos): m/z=365 [M+H]⁺;

¹H-NMR (400 MHz, DMSO-d₆): δ [ppm]=13.30 (br. s., 1H), 8.66 (s, 1H), 7.77 (d, 1H), 7.58 (s, 1H), 7.50-7.43 (m, 1H), 7.41-7.29 (m, 2H), 7.24 (s, 1H), 5.20 (m_{c}, 1H), 4.61 (m_{c}, 2H), 3.95 (s, 3H).

### Example 20A

### tert-Butyl 2-[(benzylamino)methyl]azetidine-1-carboxylate [racemate]

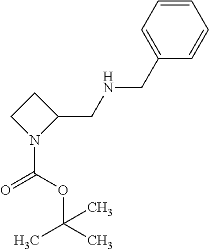 [SMILES: CC(C)(C)OC(=O)N1CCC1CNCc1ccccc1]

10.0 g (53.7 mmol) of tert-butyl 2-(aminomethyl)azetidine-1-carboxylate and 2.03 g (37.8 mmol) of benzaldehyde in 100 ml of methanol were heated under reflux for 2.5 h. The mixture was then cooled to 0° C., and 2.03 g (53.7 mmol) of sodium borohydride were added slowly at this temperature over a period of 15 min. The mixture was stirred at RT overnight. The mixture was then concentrated under reduced pressure, dichloromethane and water were added to the residue, the phases were separated and the aqueous phase was extracted twice with dichloromethane. The combined organic phases were washed with saturated aqueous sodium chloride solution, dried over sodium sulphate and filtered, and the filtrate was concentrated under reduced pressure. Dichloromethane was added to the residue obtained, and the product was purified by silica gel chromatography (dichloromethane, then dichloromethane:methanol=100:4). Yield: 7.43 g (50% of theory).

LC-MS (method 6A): Rₜ=2.41 min; MS (ESIpos): m/z=277 [M+H]⁺.

### Example 21A

### tert-Butyl 2-{[benzyl(1-methoxy-1-oxopropan-2-yl)amino]methyl}azetidine-1-carboxylate [diastereomer mixture, 4 isomers]

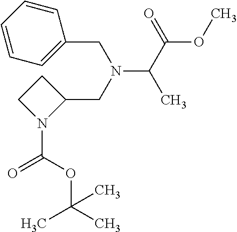 [SMILES: COC(=O)C(C)N(Cc1ccccc1)CC1CCN1C(=O)OC(C)(C)C]

2.50 g (9.05 mmol) of tert-butyl 2-[(benzylamino)methyl]azetidine-1-carboxylate [racemate] were dissolved in dichloromethane (150 ml), 5.55 ml (4.03 g, 39.8 mmol) of triethylamine and 3.04 ml (4.53 g, 27.1 mmol) of methyl 2-bromopropanoate [racemate] were added and the mixture was stirred at RT overnight. 5.55 ml (4.03 g, 39.8 mmol) of triethylamine and 3.04 ml (4.53 g, 27.1 mmol) of methyl 2-bromopropanoate [racemate] were added, and the mixture was stirred at 40° C. overnight. A further 5.55 ml (4.03 g, 39.8 mmol) of triethylamine and 3.04 ml (4.53 g, 27.1 mmol) of methyl 2-bromopropanoate [racemate] were then added, and the mixture was stirred at 40° C. overnight. After cooling to room temperature, the mixture was diluted with water and dichloromethane, and the phases were separated. The aqueous phase was extracted twice with dichloromethane and the combined organic phases were washed with saturated aqueous sodium chloride solution, dried over sodium sulphate, filtered and then freed of the solvent under reduced pressure. The crude product obtained was purified by silica gel chromatography (dichloromethane, then dichloromethane/methanol=100:1). Yield: 3.22 g (94% of theory).

LC-MS (Method 1A): Rₜ=1.00 min (diastereomer 1), Rₜ=1.13 min (diastereomer 2);

MS (ESIpos): m/z=363 [M+H]⁺;

¹H-NMR (400 MHz, DMSO-d₆): δ [ppm]=7.35-7.28 (m, 4H), 7.27-7.20 (m, 1H), 4.18-3.98 (m, 1H), 3.85-3.73 (m, 1H), 3.71-3.51 (m, 6H), 3.51-3.38 (m, 1H), 3.04-2.88 (m, 1H), 2.85-2.69 (m, 1H), 2.15-1.96 (m, 1H), 1.93-1.65 (m, 1H), 1.34 (d, 9H), 1.26-1.15 (m, 3H).

### Example 22A

### Methyl N-(azetidin-2-ylmethyl)-N-benzylalaninate [diastereomer mixture, 4 isomers]

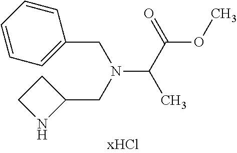 [SMILES: COC(=O)C(C)N(Cc1ccccc1)CC1CCN1]

14.9 ml (59.7 mmol) of a 4 N solution of hydrogen chloride in 1,4-dioxane were added to 3.2 g (8.5 mmol) of tert-butyl 2-{[benzyl(1-methoxy-1-oxopropan-2-yl)amino]methyl}-azetidine-1-carboxylate [diastereomer mixture, 4 isomers] in dioxane (74 ml), and the mixture was stirred at room temperature overnight. 14 ml (59.7 mmol) of a 4 N solution of hydrogen chloride in 1,4-dioxane were then added, and the mixture was stirred at RT overnight. The mixture was then concentrated under reduced pressure and the product was dried under high vacuum. Yield: 3.13 g (98% of theory, purity: 80%).

LC-MS (Method 1A): Rₜ=0.68 min (diastereomer 1), Rₜ=0.70 min (diastereomer 2); MS (ESIpos): m/z=263 [M+H-HCl]⁺.

### Example 23A

### 4-Benzyl-3-methyl-1,4-diazabicyclo[4.2.0]octan-2-one [enantiomerically pure isomer 3]

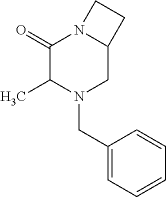 [SMILES: CC1C(=O)N2CCC2CN1Cc1ccccc1]

28.2 g (204 mmol) of potassium carbonate were added to 21.8 g (51.0 mmol, purity: 70%) of methyl N-(azetidin-2-ylmethyl)-N-benzylalaninate [diastereomer mixture, 4 isomers] in methanol (562 ml), and the mixture was stirred at RT for 2.5 d. The reaction solution was filtered and most of the solvent was removed at 20° C. under reduced pressure. The residue was taken up in water and extracted repeatedly with dichloromethane and then repeatedly with chloroform/isopropanol (7:3). The collected organic phases were dried over sodium sulphate, filtered and concentrated under reduced pressure. Using Method 7D, the crude product (12.1 g) was separated into the corresponding isomers. Here, the target compound eluted as third component. Yield: 2.47 g (21% of theory).

HPLC (Method 6E): Rₜ=7.49 min, 99.0% ee;

LC-MS (method 1A): Rₜ=0.50 min; MS (ESIpos): m/z=231 [M+H]⁺.

### Example 24A

### 3-Methyl-1,4-diazabicyclo[4.2.0]octan-2-one [enantiomerically pure isomer 3]

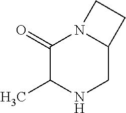

2.40 g (10.4 mmol) of 4-benzyl-3-methyl-1,4-diazabicyclo[4.2.0]octan-2-one [enantiomerically pure isomer 3, Example 23A] were initially charged in ethanol (85 ml), 250 mg of palladium on carbon (10%) and 130 mg of palladium hydroxide on carbon (20%) were added under argon and the mixture was then stirred under an atmosphere of hydrogen at standard pressure overnight. The reaction solution was filtered through kieselguhr and the filter residue was washed with hot ethanol (100 ml). The filtrate was concentrated under reduced pressure and the product was dried under high vacuum. Yield: 1.56 g (quant.).

GC-MS (method 2B): Rₜ=4.50 min; MS (EIpos): m/z=140 [M]⁺;

MS (method 1C): m/z=141 [M+H]⁺;

¹H-NMR (400 MHz, DMSO-d₆): δ [ppm]=4.59 (m_{c}, 1H), 4.09-3.89 (m, 2H), 3.27 (q, 1H), 2.95 (dd, 1H), 2.58-2.53 (m, 2H), 2.33-2.04 (m, 2H), 1.12 (d, 3H).

### Example 25A

### Methyl [3-(benzyloxy)cyclobutylidene][(tert-butoxycarbonyl)amino]acetate

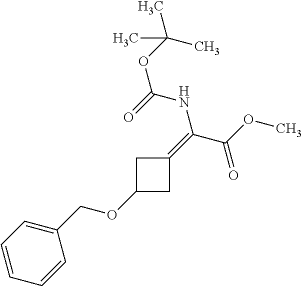 [SMILES: COC(=O)C(NC(=O)OC(C)(C)C)=C1CC(OCc2ccccc2)C1]

At RT, 605 mg (0.590 ml, 3.97) of 1,8-diazabicyclo[5.4.0]undec-7-ene were added to 928 mg (3.12 mmol) of methyl [(tert-butoxycarbonyl)amino](dimethoxyphosphoryl)acetate [racemate] and 500 mg (2.84 mmol) of 3-(benzyloxy)cyclobutanone [K. Ogura, G. Tsuchihashi et al., Bull. Chem. Soc. Jpn. 1984, 57, 1637-1642] in dichloromethane (50 ml), and the mixture was then stirred overnight. The reaction solution was concentrated under reduced pressure and the residue was taken up in ethyl acetate. The organic phase was washed with water, 0.5 N aqueous hydrogen chloride solution, saturated aqueous sodium bicarbonate solution and saturated aqueous sodium chloride solution, dried over sodium sulphate and filtered and concentrated under reduced pressure. The residue was purified by preparative RP-HPLC (acetonitrile/water). Yield: 651 mg (60% of theory).

LC-MS (method 1A): Rₜ=1.15 min; MS (ESIpos): m/z=348 [M+H]⁺;

¹H-NMR (400 MHz, DMSO-d₆): δ [ppm]=8.11 (br. s., 1H), 7.41-7.25 (m, 5H), 4.42 (s, 2H), 4.13 (quin, 1H), 3.63 (s, 3H), 3.25 (br. d., 1H), 2.99 (br. d., 1H), 2.85 (br. d., 1H), 2.65 (m, 1H), 1.37 (s, 9H).

### Example 26A

### Methyl [3-(benzyloxy)cyclobutyl][(tert-butoxycarbonyl)amino]acetate [cis and trans isomer mixture, 4 isomers]

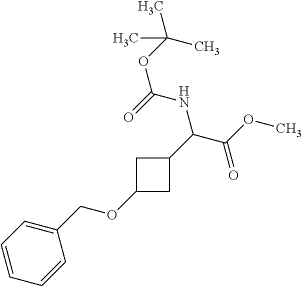 [SMILES: COC(=O)C(NC(=O)OC(C)(C)C)C1CC(OCc2ccccc2)C1]

650 mg (1.87 mmol) of methyl [3-(benzyloxy)cyclobutylidene][(tert-butoxycarbonyl)amino]acetate and 455 mg (18.7 mmol) of magnesium turnings in methanol (50 ml) were reacted at RT in an ultrasonic bath [Elma, Transsonic T 780] for 3 h. Semisaturated aqueous ammonium chloride solution was added, and the reaction solution was extracted repeatedly with dichloromethane. The organic phases were dried over sodium sulphate, filtered and concentrated under reduced pressure. The crude product was used without further purification in the next step. Yield: 630 mg (96% of theory).

LC-MS (method 1A): Rₜ=1.16 min; MS (ESIpos): m/z=350 [M+H]⁺, 250 [M+H-Boc];

¹H-NMR (400 MHz, DMSO-d₆): δ [ppm]=7.39-7.20 (m, 6H), 4.34 (s, 2H), 4.07 (quin, 0.3H), 3.99-3.73 (m, 1.7H), 3.60 (s, 3H), 2.34-1.94 (m, 3.5H), 1.74-1.59 (m, 1.5H), 1.45-1.27 (m, 9H).

### Example 27A

tert-Butyl {1-[3-(benzyloxy)cyclobutyl]-2-hydroxyethyl}carbamate [cis and trans isomer mixture, 4 isomers]

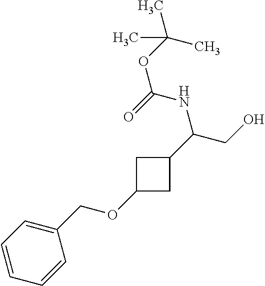 [SMILES: CC(C)(C)OC(=O)NC(CO)C1CC(OCc2ccccc2)C1]

At 0° C., 4.44 ml (8.87 mmol) of 2 M lithium borohydride solution in tetrahydrofuran were added to 620 mg (1.77 mmol) of methyl [3-(benzyloxy)cyclobutyl][(tert-butoxycarbonyl)amino]acetate [cis and trans isomer mixture, 4 isomers] in tetrahydrofuran (6.0 ml). The mixture was then stirred for 4 h and allowed to warm to RT during this time. The reaction was terminated by addition of ethyl acetate (50.0 ml) and the reaction solution was subsequently washed with 0.5 N aqueous hydrogen chloride solution. The organic phase was dried over sodium sulphate, filtered and concentrated under reduced pressure. The crude product was used without further purification in the next step. Yield: 560 mg (96% of theory).

LC-MS (method 1A): Rₜ=0.99 min; MS (ESIpos): m/z=322 [M+H]⁺, 222 [M+H-COOC(CH₃)_{3]};

¹H-NMR (400 MHz, DMSO-d₆): δ [ppm]=7.47-7.15 (m, 5H), 6.65-6.41 (m, 1H), 4.46 (br. s., 0.5H), 4.33 (s, 2H), 3.88-3.70 (m, 0.7H), 3.67-3.09 (m, 3.8H), 2.36-1.78 (m, 3.5H), 1.74-1.48 (m, 1.5H), 1.38 (s, 9H).

### Example 28A

### 2-Amino-2-[3-(benzyloxy)cyclobutyl]ethanol trifluoroacetate [cis and trans isomer mixture, 4 isomers]

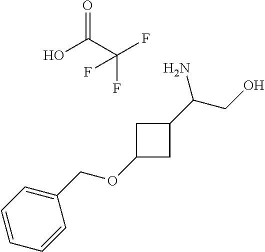

560 mg (1.74 mmol) of tert-butyl {1-[3-(benzyloxy)cyclobutyl]-2-hydroxyethyl}carbamate [cis and trans isomer mixture, 4 isomers] were initially charged in dichloromethane (8.0 ml) and 1.0 ml (12.9 mmol) of trifluoroacetic acid was added at RT. The reaction solution was then stirred for 2 h and concentrated completely under reduced pressure. Excess trifluoroacetic acid was removed by repeated co-evaporation with dichloromethane. The crude product was used without further purification in the next step. Yield: 580 mg (95% of theory).

LC-MS (method 4A): Rₜ=2.10 min; MS (ESIpos): m/z=222 [M+H-TFA]+.

### Example 29A

### N-{1-[3-(Benzyloxy)cyclobutyl]-2-hydroxyethyl}-2-chloropropanamide [diastereomer mixture, 8 isomers]

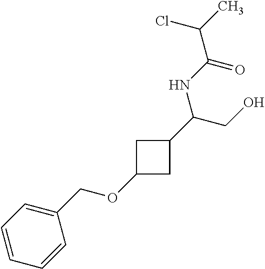 [SMILES: CC(Cl)C(=O)NC(CO)C1CC(OCc2ccccc2)C1]

580 mg (1.73 mmol) of 2-amino-2-[3-(benzyloxy)cyclobutyl]ethanol trifluoroacetate [cis and trans isomer mixture, 4 isomers] in isopropanol (15 ml) were cooled to 0° C., and 700 mg (960 μl, 6.92 mmol) of triethylamine were added. 242 mg (190 μl, 1.90 mmol) of 2-chloropropionyl chloride [racemate] were then added dropwise, and the mixture was stirred at 0° C. for 1 h and then concentrated completely under reduced pressure. 0.5 N aqueous hydrogen chloride solution (50 ml) was added to the residue, and the mixture was extracted repeatedly with dichloromethane. The organic phases were dried over sodium sulphate, filtered and concentrated under reduced pressure. The crude product was used without further purification in the next step. Yield: 638 mg (91% of theory, purity: 77%).

LC-MS (method 4A): Rₜ=2.36 min; MS (ESIpos): m/z=312 [M+H]⁺.

### Example 30A

### 5-[3-(Benzyloxy)cyclobutyl]-2-methylmorpholin-3-one [diastereomer mixture, 8 isomers]

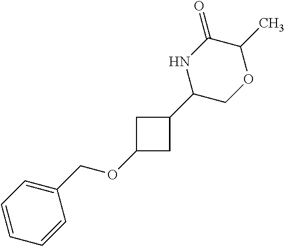 [SMILES: CC1OCC(C2CC(OCc3ccccc3)C2)NC1=O]

1.15 g (3.69 mmol) of N-{1-[3-(benzyloxy)cyclobutyl]-2-hydroxyethyl}-2-chloropropanamide [diastereomer mixture, 8 isomers] were initially charged in isopropanol (30.0 ml), the mixture was cooled to 0° C. and 1.66 g (14.8 mmol) of potassium tert-butoxide were then added in one portion. The mixture was allowed to warm to RT and stirred at 50° C. for 1 h. Most of the isopropanol was then removed under reduced pressure and the residue was taken up in ethyl acetate. The organic phase was washed with 1 N aqueous hydrogen chloride solution, dried over sodium sulphate, filtered and concentrated under reduced pressure. The residue was purified by preparative RP-HPLC (acetonitrile/water). Yield: 953 mg (93% of theory).

LC-MS (method 1A): Rₜ=0.88 min; MS (ESIpos): m/z=276 [M+H]⁺;

¹H-NMR (400 MHz, CDCl₃): δ [ppm]=7.43-7.27 (m, 5H), 6.40 (br. s., 0.16H), 6.24 (br. s., 0.38H), 6.12-5.94 (m, 0.46H), 4.41 (s, 2H), 4.24-4.05 (m, 1.25H), 4.03-3.86 (m, 1.25H), 3.82-3.51 (m, 1.5H), 3.31-3.21 (m, 1H), 2.54-1.57 (m, 5H), 1.48-1.41 (m, 3H).

### Example 31A

### 5-[3-(Benzyloxy)cyclobutyl]-2-methylmorpholine [diastereomer mixture, 8 isomers]

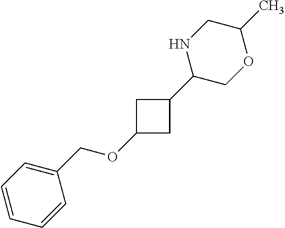 [SMILES: CC1CNC(C2CC(OCc3ccccc3)C2)CO1]

Under argon, 6.92 ml (13.8 mmol) of 2 M borane/dimethyl sulphide complex solution in tetrahydrofuran were added to 953 mg (3.46 mmol) of 5-[3-(benzyloxy)cyclobutyl]-2-methylmorpholin-3-one [diastereomer mixture, 8 isomers] in tetrahydrofuran (10 ml), and the mixture was then stirred under reflux for 3 h. The reaction solution was then carefully added dropwise to ethanol (50.0 ml) and stirred under reflux for 8 h. The mixture was then concentrated completely under reduced pressure, and the residue was taken up in acetonitrile and purified by preparative RP-HPLC (acetonitrile/water). Yield: 780 mg (84% of theory).

LC-MS (method 1A): Rₜ=0.57, 0.60 min; MS (ESIpos): m/z=262 [M+H]⁺;

¹H-NMR (400 MHz, DMSO-d₆): δ [ppm]=7.39-7.24 (m, 5H), 4.37-4.31 (m, 2H), 4.11-3.98 (m, 0.3H), 3.92-3.78 (m, 0.7H), 3.72-3.54 (m, 0.5H), 3.50-3.40 (m, 1.5H), 2.94-2.70 (m, 1H), 2.61 (td, 0.3H), 2.48-1.82 (m, 5.7H), 1.73-1.40 (m, 2H), 1.06-0.94 (m, 3H), one proton obscured

### Example 32A

### Benzyl 5-[3-(benzyloxy)cyclobutyl]-2-methylmorpholine-4-carboxylate [diastereomer mixture, 4 isomers]

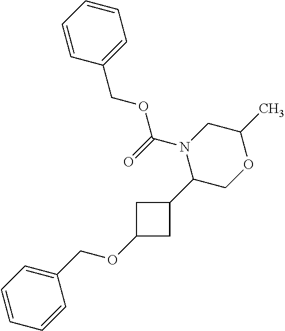 [SMILES: CC1CN(C(=O)OCc2ccccc2)C(C2CC(OCc3ccccc3)C2)CO1]

At 0° C., 881 mg (0.74 ml, 5.17 mmol) of benzyl chloroformate were added dropwise to 900 mg (3.44 mmol) of 5-[3-(benzyloxy)cyclobutyl]-2-methylmorpholine [diastereomer mixture, 8 isomers] and 890 mg (1.20 ml, 6.89 mmol) of N,N-diisopropylethylamine in dichloromethane (45.0 ml). The reaction was stirred overnight and allowed to warm to RT during this time. The reaction solution was concentrated under reduced pressure and the residue was taken up in acetonitrile. Purification and diastereomer separation by RP-HPLC on an achiral phase (acetonitrile/water) gave 537 mg (36% of theory) of the target compound of Example 32A (diastereomer mixture, 4 isomers) and 588 mg (43% of theory) of the target compound of Example 33A (diastereomer mixture, 4 isomers).

LC-MS (method 1A): Rₜ=1.26 min; MS (ESIpos): m/z=396 [M+H]⁺;

¹H-NMR (400 MHz, DMSO-d₆): δ [ppm]=7.41-7.24 (m, 10H), 5.22-5.01 (m, 2H), 4.33-4.26 (m, 2H), 4.09-3.66 (m, 4H), 3.51 (d, 1H), 3.29-3.10 (m, 2H), 2.82 (br. s., 0.3H), 2.48-1.79 (m, 3.3H), 1.69-1.52 (m, 1.4H), 1.14-1.07 (m, 3H).

### Example 33A

### Benzyl 5-[3-(benzyloxy)cyclobutyl]-2-methylmorpholine-4-carboxylate [diastereomer mixture, 4 isomers]

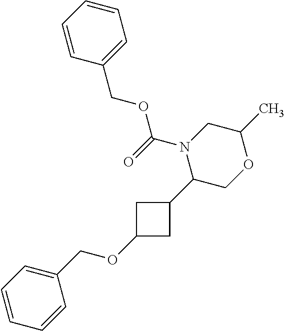 [SMILES: CC1CN(C(=O)OCc2ccccc2)C(C2CC(OCc3ccccc3)C2)CO1]

At 0° C., 881 mg (0.74 ml, 5.17 mmol) of benzyl chloroformate were added dropwise to 900 mg (3.44 mmol) of 5-[3-(benzyloxy)cyclobutyl]-2-methylmorpholine [diastereomer mixture, 8 isomers] and 890 mg (1.20 ml, 6.89 mmol) of N,N-diisopropylethylamine in dichloromethane (45.0 ml). The reaction was stirred overnight and allowed to warm to RT during this time. The reaction solution was concentrated under reduced pressure and the residue was taken up in acetonitrile. Purification and diastereomer separation by RP-HPLC on an achiral phase (acetonitrile/water) gave 537 mg (36% of theory) of the target compound of Example 32A (diastereomer mixture, 4 isomers) and 588 mg (43% of theory) of the target compound of Example 33A (diastereomer mixture, 4 isomers).

LC-MS (method 1A): Rₜ=1.29 min; MS (ESIpos): m/z=396 [M+H]⁺;

¹H-NMR (400 MHz, DMSO-d₆): δ [ppm]=7.44-7.22 (m, 10H), 5.20-4.98 (m, 2H), 4.36-4.20 (m, 2H), 4.14-3.34 (m, 6H), 2.88-2.57 (m, 1.5H), 2.44-1.53 (m, 4.5H), 1.10-1.03 (m, 3H).

### Example 34A

### 3-(6-Methylmorpholin-3-yl)cyclobutanol [diastereomer mixture, 4 isomers]

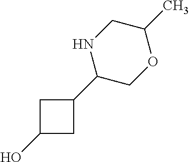

Under argon, 58 mg of palladium on carbon (10%) and 58 mg of palladium hydroxide on carbon (20%) were added to 580 mg (1.47 mmol) of benzyl 5-[3-(benzyloxy)cyclobutyl]-2-methylmorpholine-4-carboxylate [Example 51A, diastereomer mixture, 4 isomers] in ethanol (100 ml), and the mixture was stirred under an atmosphere of hydrogen at standard pressure overnight. The reaction solution was filtered through kieselguhr and the filter residue was washed with ethanol. The filtrate was concentrated under reduced pressure and the product was dried under high vacuum. Yield: 245 mg (97% of theory).

GC-MS (method 1B): Rₜ=4.60, 4.67 min; MS (EIpos): m/z=171 [M]⁺;

¹H-NMR (400 MHz, DMSO-d₆): δ [ppm]=4.94-4.84 (m, 1H), 4.16-4.05 (d, 0.6H), 3.93-3.82 (m, 0.7H), 3.55-3.40 (m, 3.3H), 3.19-3.14 (m, 0.7H), 3.17 (d, 1H), 2.47-1.76 (m, 6H), 1.58-1.28 (m, 1.5H), 1.08-0.94 (m, 3.5H).

### Example 35A

### N-Benzyl-2-chloro-N-[(2R)-1-hydroxypropan-2-yl]propanamide [diastereomer mixture, 2 isomers]

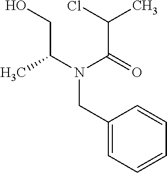 [SMILES: CC(Cl)C(=O)N(Cc1ccccc1)[C@H](C)CO]

16.4 g (99.3 mmol) of (2R)-2-(benzylamino)propan-1-ol [lit.: T. J. Tewson et al., Synthesis 2002, 6, 766-770] in isopropanol (500 ml) were cooled to 0° C., and 20.1 g (27.7 ml, 199 mmol) of triethylamine were added. 13.9 g (10.8 ml, 109 mmol) of 2-chloropropionyl chloride [racemate] were then added dropwise, and the mixture was stirred overnight and allowed to warm to RT during this time. The reaction solution was concentrated under reduced pressure, 0.5 N aqueous hydrogen chloride solution was added to the residue and the mixture was extracted with ethyl acetate. The organic phases were dried over sodium sulphate, filtered and concentrated under reduced pressure. The crude product was used without further purification in the next step. Yield: 24.3 g (88% of theory, purity: 92%, diastereomer ratio about 1:1).

LC-MS (Method 1A): Rₜ=0.80 min (diastereomer 1), Rₜ=0.84 min (diastereomer 2);

MS (ESIpos): m/z=256 [M+H]⁺.

### Example 36A

### (5R)-4-Benzyl-2,5-dimethylmorpholin-3-one [diastereomer mixture, 2 isomers]

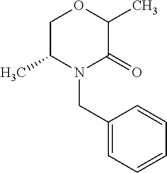 [SMILES: CC1OC[C@@H](C)N(Cc2ccccc2)C1=O]

30.0 g (109 mmol, purity: 93%) of N-benzyl-2-chloro-N-[(2R)-1-dihydroxypropan-2-yl]propanamide [diastereomer mixture, 2 isomers] in isopropanol (588 ml) were cooled to 0° C., and 49.0 g (436 mmol) of potassium tert-butoxide were then added in one portion. The mixture was allowed to slowly warm to RT and stirred overnight. Most of the isopropanol was removed under reduced pressure and the residue was taken up in water. The mixture was extracted with ethyl acetate, and the organic phases were then dried over magnesium sulphate, filtered and concentrated under reduced pressure. The crude product was used without further purification in the next step. Yield: 22.8 g (93% of theory).

LC-MS (method 1A): Rₜ=0.85 min; MS (ESIpos): m/z=220 [M+H]⁺.

### Example 37A

### (5R)-4-Benzyl-2,5-dimethylmorpholine [enantiomerically pure isomers 1+2]

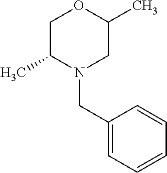

Under argon, 184 ml (369 mmol) of 2 M borane/dimethyl sulphide complex solution in tetrahydrofuran were added to 27.0 g (123 mmol) of (5R)-4-benzyl-2,5-dimethylmorpholin-3-one [diastereomer mixture, 2 isomers] in tetrahydrofuran (400 ml), and the mixture was stirred under reflux for 2 h. The mixture was subsequently cooled to 0° C. and methanol (200 ml) was added carefully. The mixture was stirred under reflux for 2 h and then concentrated completely under reduced pressure. The residue was taken up in acetonitrile and subjected directly to purification and diastereomer separation by preparative RP-HPLC (acetonitrile/water, isocratic). Here, the enantiomerically pure diastereomer 1 (minor isomer) was the compound which eluted as the first component. Yield: 2.60 g (10% of theory, enantiomerically pure isomer 1). Here, the enantiomerically pure diastereomer 2 (main isomer) was the compound which eluted as the second component. Yield: 9.00 g (35% of theory, enantiomerically pure isomer 2).

Enantiomerically pure isomer 1:

LC-MS (method 6A): Rₜ=2.30 min; MS (ESIpos): m/z=206 [M+H]⁺;

Enantiomerically pure isomer 2:

LC-MS (method 6A): Rₜ=2.46 min; MS (ESIpos): m/z=206 [M+H]⁺.

### Example 38A

### (5R)-2,5-Dimethylmorpholine hydrochloride [enantiomerically pure isomer 2]

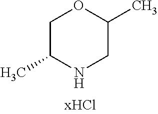

9.00 g (43.8 mmol) of (5R)-4-benzyl-2,5-dimethylmorpholine (Example 37A, main isomer, enantiomerically pure isomer 2) were initially charged in ethanol (441 ml), and 2 N aqueous hydrogen chloride solution (40.0 ml) was added. Under argon, 1.26 g of palladium on carbon (10%) and 628 mg of palladium hydroxide on carbon (20%) were added, and the mixture was then stirred under an atmosphere of hydrogen at standard pressure overnight. The reaction solution was filtered through kieselguhr and the filter residue was washed with ethanol. The filtrate was concentrated under reduced pressure and the product was dried under high vacuum. Yield: 7.83 g (quant.).

MS (method 1C): m/z=116 [M+H-HCl]⁺;

¹H-NMR (400 MHz, DMSO-d₆): δ [ppm]=9.97 (br. s., 1H), 9.43 (br. s., 1H), 3.90-3.71 (m, 2H), 3.62 (d, 1H), 3.40 (d, 1H), 3.06-2.91 (m, 1H), 2.89-2.71 (m, 1H), 1.32 (d, 3H), 1.14 (d, 3H).

### Example 39A

### (2-Bromo-8-methoxy[1,2,4]triazolo[1,5-a]pyridin-6-yl)[(5R)-2,5-dimethylmorpholin-4-yl]methanone [enantiomerically pure isomer]

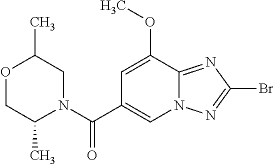 [SMILES: COc1cc(C(=O)N2CC(C)OC[C@H]2C)cn2nc(Br)nc12]

500 mg (1.84 mmol) of 2-bromo-8-methoxy[1,2,4]triazolo[1,5-a]pyridine-6-carboxylic acid and 334 mg (2.21 mmol) of (5R)-2,5-dimethylmorpholine hydrochloride [enantiomerically pure isomer 2] were initially charged in N,N-dimethylformamide (12.1 ml), and 950 mg (1.28 ml, 7.35 mmol) of N,N-diisopropylethylamine were added. Subsequently, 839 mg (2.21 mmol) of HATU were added at RT and the mixture was stirred overnight. Without further work-up, the reaction solution was then purified directly by preparative RP-HPLC (acetonitrile/water). Yield: 662 mg (97% of theory).

LC-MS (method 1A): Rₜ=0.78 min; MS (ESIpos): m/z=369 [M+H]⁺;

¹H-NMR (400 MHz, DMSO-d₆): δ [ppm]=8.70 (br. s., 1H), 7.23-7.04 (m, 1H), 4.52 (br. s., 0.5H), 4.20 (br. d., 0.5H), 4.01 (s, 3H), 3.82-3.58 (m, 2H), 3.56-3.41 (br. s., 2H), 3.04 (m_{c}, 0.5H), 2.76 (m_{c}, 0.5H), 1.45-0.91 (m, 6H).

### Example 40A

### (5R)-2-Allyl-4-benzyl-2,5-dimethylmorpholin-3-one [diastereomer mixture, 2 isomers]

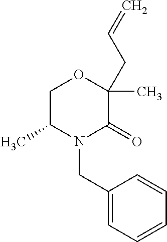 [SMILES: C=CCC1(C)OC[C@@H](C)N(Cc2ccccc2)C1=O]

22.8 g (104 mmol) of (5R)-4-benzyl-2,5-dimethylmorpholin-3-one [diastereomer mixture, 2 isomers] were initially charged in tetrahydrofuran (1.34 l), 146 ml (146 mmol) of 1 M lithium hexamethyldisilazide solution in tetrahydrofuran were added under argon at −78° C. and the mixture was stirred for 15 min. Subsequently, at −78° C., 21.0 g (11.4 ml, 125 mmol) of allyl iodide were added, and the reaction mixture was allowed to warm to RT and stirred for 3 h. The reaction was terminated by addition of saturated aqueous ammonium chloride solution and the mixture was then extracted with ethyl acetate. The organic phase was washed with saturated aqueous sodium chloride solution, dried over sodium sulphate, filtered and concentrated under reduced pressure. The crude product was used without further purification in the next step. Yield: 27.5 g (77% of theory, purity: 75%).

LC-MS (method 1A): Rₜ=0.99 min; MS (ESIpos): m/z=260 [M+H]⁺.

### Example 41A

### [(5R)-4-Benzyl-2,5-dimethyl-3-oxomorpholin-2-yl]acetaldehyde [diastereomer mixture, 2 isomers]

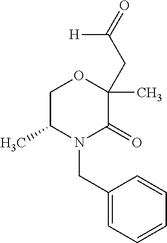 [SMILES: C[C@@H]1COC(C)(CC=O)C(=O)N1Cc1ccccc1]

27.4 g (79.9 mmol, purity: 75%) of (5R)-2-allyl-4-benzyl-2,5-dimethylmorpholin-3-one [diastereomer mixture, 2 isomers] were initially charged in tetrahydrofuran (620 ml) and water (370 ml), and 4.35 ml (1.60 mmol) of a 2.5% solution of osmium tetroxide in tert-butanol and 51.2 g (240 mmol) of sodium periodate were added at 0° C. The mixture was allowed to warm to RT and stirred overnight. The reaction solution was filtered through kieselguhr and the filter residue was washed with tetrahydrofuran. The filtrate was concentrated under reduced pressure and the residue was taken up in ethyl acetate and water. After separation of the phases, the organic phase was washed with 1 N aqueous sodium sulphite solution (2×400 ml), dried over sodium sulphate, filtered and concentrated under reduced pressure. The crude product was used without further purification in the next step. Yield: 23.6 g of crude product.

LC-MS (Method 1A): Rₜ=0.81 min (diastereomer 1), Rₜ=0.84 min (diastereomer 2);

MS (ESIpos): m/z=262 [M+H]⁺.

### Example 42A

### (5R)-4-Benzyl-2-(2-hydroxyethyl)-2,5-dimethylmorpholin-3-one [diastereomer mixture, 2 isomers]

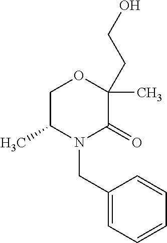 [SMILES: C[C@@H]1COC(C)(CCO)C(=O)N1Cc1ccccc1]

7.00 g (about 26.8 mmol, crude product) of [(5R)-4-benzyl-2,5-dimethyl-3-oxomorpholin-2-yl]acetaldehyde [diastereomer mixture, 2 isomers] were initially charged in methanol (200 ml), and 3.04 g (80.4 mmol) of sodium borohydride were added at 0° C. The mixture was allowed to warm to RT and stirred for 30 min. Water was then added to the reaction solution, methanol was removed under reduced pressure and the residue was extracted with ethyl acetate. The organic phase was dried over sodium sulphate, filtered and concentrated under reduced pressure. The crude product was purified by preparative RP-HPLC (acetonitrile/water). Yield: 6.82 g (70% of theory, purity: 73%).

LC-MS (method 1A): Rₜ=0.71 min; MS (ESIpos): m/z=264 [M+H]⁺.

### Example 43A

### 2-[(5R)-4-Benzyl-2,5-dimethylmorpholin-2-yl]ethanol [enantiomerically pure isomers 1+2]

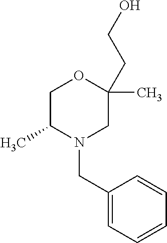 [SMILES: C[C@@H]1COC(C)(CCO)CN1Cc1ccccc1]

6.80 g (18.9 mmol, purity: 73%) of (5R)-4-benzyl-2-(2-hydroxyethyl)-2,5-dimethylmorpholin-3-one [diastereomer mixture, 2 isomers] were initially charged in tetrahydrofuran (191 ml), 37.7 ml (75.4 mmol) of 2 M borane/dimethyl sulphide complex solution in tetrahydrofuran were added under argon and the mixture was stirred under reflux for 2 h. The mixture was subsequently cooled to 0° C., methanol (37 ml) was added carefully and the mixture was stirred under reflux for 30 min. The mixture was subsequently concentrated completely under reduced pressure, and the residue was taken up in acetonitrile and subjected to purification and diastereomer separation by preparative RP-HPLC (acetonitrile/water, isocratic). Here, the enantiomerically pure diastereomer 1 (minor isomer) was the compound which eluted as the first component. Yield: 1.34 g (28% of theory, enantiomerically pure isomer 1). Here, the enantiomerically pure diastereomer 2 (main isomer) was the compound which eluted as the second component. Yield: 2.28 g (47% of theory, enantiomerically pure isomer 2).

Enantiomerically pure isomer 1:

LC-MS (method 4A): Rₜ=2.55 min; MS (ESIpos): m/z=250 [M+H]⁺;

Enantiomerically pure isomer 2:

LC-MS (method 4A): Rₜ=2.64 min; MS (ESIpos): m/z=250 [M+H]⁺.

### Example 44A

### 2-[(5R)-2,5-Dimethylmorpholin-2-yl]ethanol [enantiomerically pure isomer]

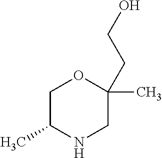

Under argon, 227 mg of palladium on carbon (10%) and 113 mg of palladium hydroxide on carbon (20%) were added to 2.25 g (9.02 mmol) of 2-[(5R)-4-benzyl-2,5-dimethylmorpholin-2-yl]ethanol [enantiomerically pure isomer 2, Example 43A] in ethanol (90.7 ml), and the mixture was then stirred under an atmosphere of hydrogen at standard pressure overnight. The reaction solution was filtered through kieselguhr and the filter residue was washed with ethanol. The filtrate was concentrated under reduced pressure and the product was dried under high vacuum. Yield: 1.46 g (quant.).

MS (method 1C): m/z=160 [M+H]⁺;

¹H-NMR (400 MHz, DMSO-d₆): δ [ppm]=4.21 (t, 1H), 3.53-3.44 (d, 2H), 3.34 (dd, 1H), 3.14 (t, 1H), 2.65-2.52 (m, 3H), 2.07 (br. s., 1H), 1.52 (td, 2H), 1.18 (s, 3H), 0.85 (d, 3H).

### Example 45A

### N-Benzyl-2-chloro-N-[(2R)-1,4-dihydroxybutan-2-yl]propanamide [diastereomer mixture, 2 isomers]

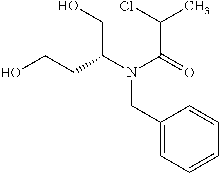 [SMILES: CC(Cl)C(=O)N(Cc1ccccc1)[C@@H](CO)CCO]

45.1 g (55.3 mmol, purity: 72%) of (2R)-2-(benzylamino)butane-1,4-diol [B. L. Feringa, Tetrahedron 1989, 45, 6799-6818] in isopropanol (239 ml) were cooled to 0° C., and 11.2 g (15.4 ml, 111 mmol) of triethylamine were added. 10.5 g (8.23 ml, 83.0 mmol) of 2-chloropropionyl chloride [racemate] were then added dropwise. After 10 min of stirring, the reaction solution was concentrated under reduced pressure and the residue was taken up in ethyl acetate and washed with water. The organic phase was dried over sodium sulphate, filtered and concentrated under reduced pressure. The crude product was used without further purification in the next step. Yield: 21.4 g (quant., purity: 82%, diastereomer ratio about 3:2).

LC-MS (Method 1A): Rₜ=0.65 min (diastereomer 1), Rₜ=0.67 min (diastereomer 2);

MS (ESIpos): m/z=286 [M+H]⁺.

### Example 46A

### (5R)-4-Benzyl-5-(2-hydroxyethyl)-2-methylmorpholin-3-one [diastereomer mixture, 2 isomers]

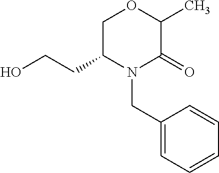 [SMILES: CC1OC[C@@H](CCO)N(Cc2ccccc2)C1=O]

21.4 g (62.1 mmol, purity: 82%) of N-benzyl-2-chloro-N-[(2R)-1,4-dihydroxybutan-2-yl]propanamide [diastereomer mixture, 2 isomers] in isopropanol (335 ml) were cooled to 0° C., and 27.9 g (249 mmol) of potassium tert-butoxide were added in one portion. The reaction was stirred overnight and allowed to warm to RT during this time. Isopropanol was removed under reduced pressure, and the residue was taken up in water (300 ml) and extracted with ethyl acetate. The organic phases were dried over sodium sulphate, filtered and concentrated under reduced pressure. The crude product was used without further purification in the next step. Yield: 13.3 g, 69% of theory, purity: 81%, diastereomer ratio about 3:2).

LC-MS (Method 7A): Rₜ=3.23 min (diastereomer 1), Rₜ=3.34 min (diastereomer 2);

MS (ESIpos): m/z=250 [M+H]⁺.

### Example 47A

### (5R)-4-Benzyl-5-({[tert-butyl(dimethyl)silyl]oxy}ethyl)-2-methylmorpholin-3-one [diastereomer mixture, 2 isomers]

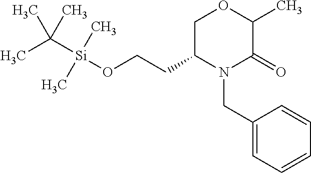 [SMILES: CC1OC[C@@H](CCO[Si](C)(C)C(C)(C)C)N(Cc2ccccc2)C1=O]

13.3 g (43.3 mmol) of (5R)-4-benzyl-5-(2-hydroxyethyl)-2-methylmorpholin-3-one [diastereomer mixture, 2 isomers] were initially charged in N,N-dimethylformamide (60.0 ml), and 8.85 g (130 mmol) of imidazole were added at RT. At 0° C., 9.80 g (65.0 mmol) of tert-butyldimethylsilyl chloride were then added and the mixture was stirred overnight and allowed to warm to RT during this time. The reaction solution was subsequently concentrated under reduced pressure, and the residue was taken up in ethyl acetate and washed repeatedly with water and once with saturated aqueous sodium chloride solution. The organic phase was dried over magnesium sulphate, filtered and concentrated under reduced pressure. The crude product was then purified by chromatography on silica gel (cyclohexane/ethyl acetate 6:1, then cyclohexane/ethyl acetate 5:1). Yield: 8.03 g (49% of theory, diastereomer ratio about 2.3:1).

LC-MS (method 1A): Rₜ=1.41 min; MS (ESIpos): m/z=364 [M+H]⁺;

¹H-NMR (400 MHz, DMSO-d₆): δ [ppm]=7.40-7.18 (m, 5H), 5.12-5.03 (m, 1H), 4.33-4.21 (m, 1H), 4.14 (d, 0.3H), 4.05 (m, 0.7H), 3.95-3.84 (m, 1H), 3.74-3.56 (m, 3H), 3.39 (dd, 0.3H), 3.28 (d, 0.7H), 1.98-1.70 (m, 2H), 1.39 (d, 0.9H), 1.35 (d, 2.1H), 0.82 (s, 9H), 0.02 (s, 1.8H), 0.00 (s, 4.2H).

### Example 48A

### (5R)-4-Benzyl-5-(2-{[tert-butyl(dimethyl)silyl]oxy}ethyl)-2,2-dimethylmorpholin-3-one [enantiomerically pure isomer]

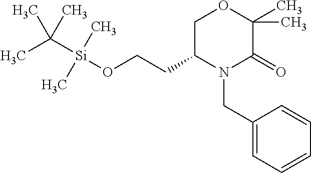 [SMILES: CC1(C)OC[C@@H](CCO[Si](C)(C)C(C)(C)C)N(Cc2ccccc2)C1=O]

7.00 g (18.6 mmol) of (5R)-4-benzyl-5-(2-{[tert-butyl(dimethyl)silyl]oxy}ethyl)-2-methylmorpholin-3-one [diastereomer mixture, 2 isomers] were initially charged in tetrahydrofuran (233 ml), and 13.0 ml (26.1 mmol) of lithium diisopropylamide solution (2.0 M in tetrahydrofuran/n-heptane/ethylbenzene) were added dropwise at −78° C. The mixture was stirred for 15 min, and 3.17 g (1.39 ml, 22.4 mmol) of iodomethane were added. The mixture was allowed to warm to RT and stirred for 2 h. Saturated aqueous ammonium chloride solution was added and the mixture was extracted with ethyl acetate. The organic phase was washed with saturated aqueous sodium chloride solution, dried over magnesium sulphate, filtered and concentrated under reduced pressure. The crude product was used without further purification in the next step. Yield: 8.36 g (70% of theory, purity: 59%).

LC-MS (method 1A): Rₜ=1.47 min; MS (ESIpos): m/z=378 [M+H]⁺.

### Example 49A

### (5R)-4-Benzyl-5-(2-{[tert-butyl(dimethyl)silyl]oxy}ethyl)-2,2-dimethylmorpholine [enantiomerically pure isomer]

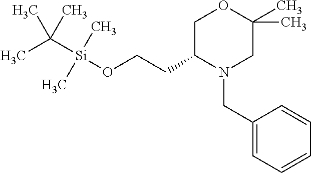 [SMILES: CC1(C)CN(Cc2ccccc2)[C@H](CCO[Si](C)(C)C(C)(C)C)CO1]

8.36 g (13.1 mmol, purity: 59%) of (5R)-4-benzyl-5-(2-{[tert-butyl(dimethyl)silyl]oxy}ethyl)-2,2-dimethylmorpholin-3-one [enantiomerically pure isomer] were initially charged in tetrahydrofuran (133 ml), 26.2 ml (52.3 mmol) of 2 M borane/dimethyl sulphide complex solution in tetrahydrofuran were added under argon and the mixture was stirred under reflux for 4 h. The mixture was subsequently cooled to 0° C., methanol (30 ml) was added carefully and the mixture was stirred under reflux for 30 min and then concentrated completely under reduced pressure. The crude product was used without further purification in the next step. Yield: 8.39 g (96% of theory, purity: 55%).

LC-MS (method 1A): Rₜ=1.15 min; MS (ESIpos): m/z=364 [M+H]⁺.

### Example 50A

### 2-[(3R)-4-Benzyl-6,6-dimethylmorpholin-3-yl]ethanol [enantiomer mixture, 2 isomers]

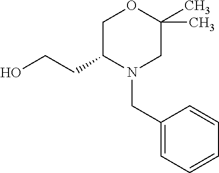 [SMILES: CC1(C)CN(Cc2ccccc2)[C@H](CCO)CO1]

7.39 g (11.2 mmol, purity: 55%) of (5R)-4-benzyl-5-(2-{[tert-butyl(dimethyl)silyl]oxy}ethyl)-2,2-dimethylmorpholine [enantiomerically pure isomer] were initially charged in tetrahydrofuran (148 ml), and 30.5 ml (30.5 mmol) of tetra-n-butylammonium fluoride solution (1.0 M in tetrahydrofuran) were added at RT. The reaction solution was stirred at RT for 1 h and the reaction solution was then concentrated under reduced pressure. The residue was purified by preparative RP-HPLC (acetonitrile/water, isocratic). Yield: 1.97 g (38% of theory, enantiomer ratio: about 85:15); at this stage, a proportional isomerization of the stereocentre to one of the earlier precursors was noticed.

HPLC (Method 7E): Rₜ=4.41 min, 85:15 R:S enantiomer ratio;

LC-MS (method 1A): Rₜ=0.35 min; MS (ESIpos): m/z=250 [M+H]⁺;

¹H-NMR (400 MHz, DMSO-d₆): δ [ppm]=7.31 (d, 4H), 7.22 (m_{c}, 1H), 4.45 (t, 1H), 3.93 (d, 1H), 3.60 (dd, 1H), 3.54-3.40 (m, 3H), 3.10 (d, 1H), 2.40-2.29 (m, 2H), 1.85 (d, 1H), 1.79-1.69 (m, 1H), 1.59 (m_{c}, 1H), 1.14 (s, 3H), 1.04 (s, 3H).

### Example 51A

### 2-[(3R)-6,6-Dimethylmorpholin-3-yl]ethanol [enantiomer mixture, 2 isomers]

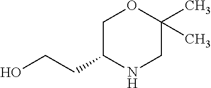

Under argon, 150 mg of palladium on carbon (10%) and 150 mg of palladium hydroxide on carbon (20%) were added to 1.00 g (4.01 mmol) of 2-[(3R)-4-benzyl-6,6-dimethylmorpholin-3-yl]ethanol [enantiomer mixture, 2 isomers] in ethanol (40.0 ml), and the mixture was stirred under an atmosphere of hydrogen at standard pressure for 4 h. The reaction solution was filtered through kieselguhr and concentrated under reduced pressure. Yield: 680 mg (quant.).

GC-MS (method 2B): Rₜ=3.71 min; MS (EIpos): m/z=159 [M]⁺;

¹H-NMR (500 MHz, DMSO-d₆): δ [ppm]=4.32 (br. s., 1H), 3.46 (t, 2H), 3.38 (dd, 1H), 3.21 (t, 1H), 2.64-2.54 (m, 2H), 2.47-2.42 (m, 1H), 1.36 (m_{c}, 2H), 1.18 (s, 3H), 1.02 (s, 3H), one proton obscured

### Example 52A

### 2-Methyl-2-{[(E)-phenylmethylene]amino}propane-1,3-diol

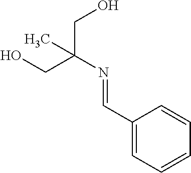

183 g (947 mmol) of 2-amino-2-methylpropane-1,3-diol were suspended in ethyl acetate (200 ml), and 103 g (98.6 ml, 970 mmol) of benzaldehyde were added dropwise with ice cooling. The mixture was allowed to warm to RT and stirred for 2 h. The mixture was concentrated at 70° C. under reduced pressure and the residue was used for the next step without further purification. Yield: 183 g (99% of theory).

### Example 53A

### 2-(Benzylamino)-2-methylpropane-1,3-diol

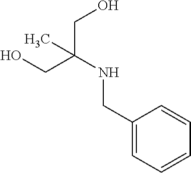

100 g (951 mmol) of 2-methyl-2-{[(E)-phenylmethylene]amino}propane-1,3-diol [lit.: J. Cossy et al., J. Org. Chem. 2012, 77, 6087-6099] were initially charged in ethanol (1.00 l), and 71.6 g (1.89 mol) of sodium borohydride were added a little at a time at 0° C. (strong evolution of gas). The mixture was allowed to warm to RT and stirred overnight. The mixture was concentrated under reduced pressure and the residue was taken up in water (700 ml). The pH was adjusted to about pH=1 with concentrated aqueous hydrogen chloride solution and the mixture was extracted with dichloromethane. The aqueous phase was adjusted to about pH=10 with 50% strength aqueous sodium hydroxide solution and then extracted repeatedly with dichloromethane. The combined organic phases were dried over sodium sulphate, filtered and concentrated under reduced pressure. Yield: 151 g (79% of theory).

LC-MS (method 4A): Rₜ=1.86 min; MS (ESIpos): m/z=196 [M+H]⁺.

### Example 54A

### N-Benzyl-2-chloro-N-(1,3-dihydroxy-2-methylpropan-2-yl)propanamide [racemate]

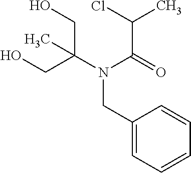 [SMILES: CC(Cl)C(=O)N(Cc1ccccc1)C(C)(CO)CO]

10.7 g (54.8 mmol) of 2-(benzylamino)-2-methylpropane-1,3-diol were initially charged in dichloromethane (400 ml), the mixture was cooled to 0° C. and 8.32 g (11.5 ml, 82.2 mmol) of triethylamine were added. First 8.35 g (6.52 ml, 65.8 mmol) of 2-chloropropionyl chloride [racemate] and then a further 5.57 g (4.35 ml, 49.3 mmol) of 2-chloropropionyl chloride [racemate] were subsequently added dropwise. After 10 min of stirring, the reaction solution was concentrated under reduced pressure, and the residue was taken up in 1 N aqueous hydrogen chloride solution and extracted repeatedly with dichloromethane. The collected organic phases were dried over sodium sulphate, filtered and concentrated under reduced pressure. The crude product was used without further purification in the next step. Yield: 15.6 g (99% of theory).

### Example 55A

### 4-Benzyl-5-(hydroxymethyl)-2,5-dimethylmorpholin-3-one [diastereomer mixture, 4 isomers]

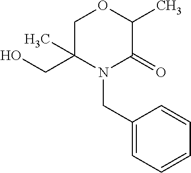 [SMILES: CC1OCC(C)(CO)N(Cc2ccccc2)C1=O]

15.6 g (54.8 mmol) of N-benzyl-2-chloro-N-(1,3-dihydroxy-2-methylpropan-2-yl)propanamide [racemate] were initially charged in isopropanol (300 ml), the mixture was cooled to 0° C. and 24.6 g (219 mmol) of potassium tert-butoxide were added in one portion. The reaction was stirred overnight and allowed to warm to RT during this time. Most of the isopropanol was removed under reduced pressure, and the residue was taken up in 2 N aqueous hydrogen chloride solution (300 ml) and extracted repeatedly with dichloromethane. The organic phases were dried over sodium sulphate, filtered and concentrated under reduced pressure. The crude product was used without further purification in the next step. Yield: 15.2 g (96% of theory, purity: 86%, diastereomer ratio about 1:1).

LC-MS (Method 1A): Rₜ=0.72 min (diastereomer 1, 2 isomers), Rₜ=0.74 min (diastereomer 2, 2 isomers); MS (ESIpos): m/z=250 [M+H]⁺.

### Example 56A

### 4-Benzyl-5-({[tert-butyl(dimethyl)silyl]oxy}methyl)-2,5-dimethylmorpholin-3-one [diastereomer mixture, 4 isomers]

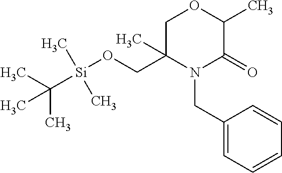 [SMILES: CC1OCC(C)(CO[Si](C)(C)C(C)(C)C)N(Cc2ccccc2)C1=O]

20.0 g (80.2 mmol) of 4-benzyl-5-(hydroxymethyl)-2,5-dimethylmorpholin-3-one [diastereomer mixture, 4 isomers] were initially charged in N,N-dimethylformamide (132 ml), and 10.9 g (160 mmol) of imidazole were added at RT. 12.7 g (84.2 mmol) of tert-butyldimethylsilyl chloride were then added, and the mixture was stirred overnight. The reaction solution was concentrated under reduced pressure, taken up in ethyl acetate and washed repeatedly with water, once with 0.4 N aqueous hydrogen chloride solution, once with saturated aqueous sodium bicarbonate solution and again with water. The organic phase was dried over magnesium sulphate, filtered and concentrated under reduced pressure. Yield: 27.9 g (93% of theory, diastereomer ratio: about 1:1).

LC-MS (Method 1A): Rₜ=1.45 min (diastereomer 1, 2 isomers), Rₜ=1.47 min (diastereomer 2, 2 isomers). MS (ESIpos): m/z=364 [M+H]⁺.

### Example 57A

### 4-Benzyl-5-({[tert-butyl(dimethyl)silyl]oxy}methyl)-2,2,5-trimethylmorpholin-3-one [racemate]

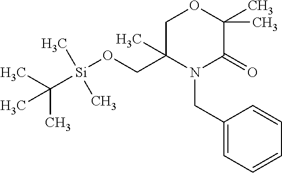 [SMILES: CC1(C)OCC(C)(CO[Si](C)(C)C(C)(C)C)N(Cc2ccccc2)C1=O]

27.9 g (76.7 mmol) of 4-benzyl-5-({[tert-butyl(dimethyl)silyl]oxy}methyl)-2,5-dimethylmorpholin-3-one [diastereomer mixture, 4 isomers] were initially charged in tetrahydrofuran (959 ml), and 59.7 ml (107 mmol) of lithium diisopropylamide solution (2.0 M in tetrahydrofuran/n-heptane/ethylbenzene) were added dropwise at −78° C. The mixture was stirred for 15 min, and 13.1 g (5.73 ml, 92.1 mmol) of iodomethane were then added. The mixture was allowed to warm to RT and stirred for 2 h. Saturated aqueous ammonium chloride solution was added and the mixture was extracted with ethyl acetate. The organic phase was washed with saturated aqueous sodium chloride solution, dried over magnesium sulphate, filtered and concentrated under reduced pressure. The crude product was used without further purification in the next step. Yield: 31.9 g (59% of theory, purity: 54%).

LC-MS (method 1A): Rₜ=1.49 min; MS (ESIpos): m/z=378 [M+H]⁺.

### Example 58A

### 4-Benzyl-5-(hydroxymethyl)-2,2,5-trimethylmorpholin-3-one [racemate]

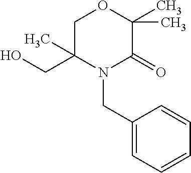 [SMILES: CC1(C)OCC(C)(CO)N(Cc2ccccc2)C1=O]

31.8 g (45.5 mmol, purity: 54%) of 4-benzyl-5-({[tert-butyl(dimethyl)silyl]oxy}methyl)-2,2,5-trimethylmorpholin-3-one [racemate] were initially charged in tetrahydrofuran (991 ml) and 158 ml (158 mmol) of tetra-n-butylammonium fluoride solution (1.0 M in tetrahydrofuran) were added at RT. The reaction solution was stirred at RT overnight and then concentrated under reduced pressure. The residue was taken up in dichloromethane and washed with water. The organic phase was dried over sodium sulphate, filtered and concentrated under reduced pressure. The crude product was then purified by preparative RP-HPLC (acetonitrile/water). Yield: 15.2 g (91% of theory, purity: 72%).

LC-MS (method 1A): Rₜ=0.79 min; MS (ESIpos): m/z=264 [M+H]⁺.

### Example 59A

### (4-Benzyl-3,6,6-trimethylmorpholin-3-yl)methanol [racemate]

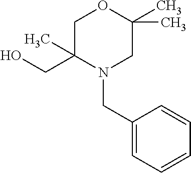 [SMILES: CC1(C)CN(Cc2ccccc2)C(C)(CO)CO1]

3.00 g (11.4 mmol) of 4-benzyl-5-(hydroxymethyl)-2,2,5-trimethylmorpholin-3-one [racemate] were initially charged in tetrahydrofuran (112 ml), 22.8 ml (45.6 mmol) of 2 M borane/dimethyl sulphide complex solution in tetrahydrofuran were added under argon and the mixture was stirred under reflux for 2 h. The mixture was subsequently cooled to 0° C. and methanol (26 ml) was added carefully. The mixture was stirred under reflux for 30 min and then concentrated completely under reduced pressure. The crude product was purified by preparative RP-HPLC (acetonitrile/water). Yield: 1.74 g (58% of theory).

LC-MS (method 1A): Rₜ=0.46 min; MS (ESIpos): m/z=250 [M+H]⁺.

### Example 60A

### (3,6,6-Trimethylmorpholin-3-yl)methanol [racemate]

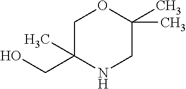

1.74 g (6.98 mmol) of 2-(4-benzyl-3,6,6-trimethylmorpholin-3-yl)methanol [racemate] were initially charged in ethanol (70.1 ml), 200 mg of palladium on carbon (10%) and 100 mg of palladium hydroxide on carbon (20%) were added under argon and the mixture was then stirred under an atmosphere of hydrogen at standard pressure overnight. The reaction solution was filtered through kieselguhr and concentrated under reduced pressure. Yield: 1.16 g (quant.).

MS (method 1C): m/z=160 [M+H]⁺.

### Example 61A

### N-Benzyl-2-chloro-N-(1,3-dihydroxypropan-2-yl)propanamide [racemate]

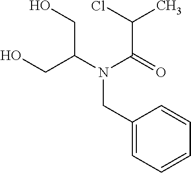 [SMILES: CC(Cl)C(=O)N(Cc1ccccc1)C(CO)CO]

60.5 g (334 mmol) of 2-(benzylamino)propane-1,3-diol [lit.: W. Lacôte et al., Org. Lett. 2011, 13, 5990-5993] were initially charged in isopropanol (0.931), the mixture was cooled to 0° C. and 50.7 g (69.8 ml, 501 mmol) of triethylamine were added. 50.9 g (38.9 ml, 401 mmol) of 2-chloropropionyl chloride [racemate] were then added dropwise. The reaction solution was allowed to warm to RT and concentrated under reduced pressure. 0.5 N aqueous hydrogen chloride solution was added to the residue, and the mixture was extracted with dichloromethane. The organic phases were dried over sodium sulphate, filtered and concentrated under reduced pressure. The crude product was used without further purification in the next step. Yield: 91.7 g (94% of theory).

LC-MS (method 1A): Rₜ=0.71 min; MS (ESIpos): m/z=272 [M+H]⁺.

### Example 62A

### 4-Benzyl-5-(hydroxymethyl)-2-methylmorpholin-3-one [diastereomer mixture, 4 isomers]

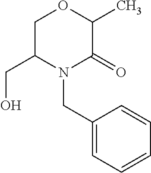 [SMILES: CC1OCC(CO)N(Cc2ccccc2)C1=O]

81.3 g (272 mmol, purity: 91%) of N-benzyl-2-chloro-N-(1,3-dihydroxypropan-2-yl)propanamide [racemate] in isopropanol (600 ml) were cooled to 0° C., and 91.6 g (817 mmol) of potassium tert-butoxide were then added in one portion. The mixture was allowed to slowly warm to RT and stirred overnight. Most of the isopropanol was removed under reduced pressure and the residue was taken up in dichloromethane. The mixture was washed with water and the organic phase was then dried over magnesium sulphate, filtered and concentrated under reduced pressure. The crude product was used without further purification in the next step. Yield: 61.7 g (96% of theory, diastereomer ratio about 7:3).

LC-MS (Method 2A): Rₜ=0.61 min (diastereomer 1, 2 isomers), Rₜ=0.62 min (diastereomer 2, 2 isomers); MS (ESIpos): m/z=236 [M+H]⁺.

### Example 63A

### 4-Benzyl-5-(methoxymethyl)-2-methylmorpholin-3-one [diastereomer mixture, 4 isomers]

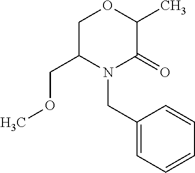 [SMILES: COCC1COC(C)C(=O)N1Cc1ccccc1]

15.0 g (63.8 mmol) of 4-benzyl-5-(hydroxymethyl)-2-methylmorpholin-3-one [diastereomer mixture, 4 isomers] were initially charged in N,N-dimethylformamide (600 ml), and 5.10 g (128 mmol, 60% suspension in paraffin oil) of sodium hydride and 22.6 g (9.92 ml, 159 mmol) of iodomethane were added at RT. The reaction mixture was stirred at RT for 2 h, and water (30 ml) was then added carefully. The solvent was removed under reduced pressure, and the residue was taken up in water and repeatedly extracted with ethyl acetate. The organic phases were dried over sodium sulphate, filtered and concentrated under reduced pressure. The residue was taken up in toluene and washed with water and saturated aqueous sodium chloride solution. The organic phase was dried over sodium sulphate, filtered and concentrated under reduced pressure. The crude product was purified by preparative RP-HPLC (acetonitrile/water). Yield: 16.7 g (96% of theory, purity: 91%).

LC-MS (method 1A): Rₜ=0.82 min; MS (ESIpos): m/z=250 [M+H]⁺.

### Example 64A

### 5-(Methoxymethyl)-2,2-dimethyl-4-(1-phenylethyl)morpholin-3-one [diastereomer mixture, 4 isomers]

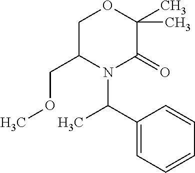 [SMILES: COCC1COC(C)(C)C(=O)N1C(C)c1ccccc1]

8.30 g (30.5 mmol) of 4-benzyl-5-(methoxymethyl)-2-methylmorpholin-3-one [diastereomer mixture, 4 isomers] were initially charged in tetrahydrofuran (381 ml), and 21.3 ml (42.7 mmol) of lithium diisopropylamide solution (1.8 M in tetrahydrofuran/n-heptane/ethylbenzene) were added at −78° C. After 15 min, 5.19 g (2.28 ml, 36.6 mmol) of iodomethane were added at −78° C. The mixture was stirred for 2 h and allowed to warm to RT during this time. Saturated aqueous ammonium chloride solution was then added and the reaction mixture was extracted with ethyl acetate. The combined organic phases were washed with saturated aqueous sodium chloride solution, dried over magnesium sulphate, filtered and concentrated under reduced pressure. The crude product was purified by preparative RP-HPLC (acetonitrile/water). Yield: 3.68 g (43% of theory).

LC-MS (method 1A): Rₜ=0.97 min; MS (ESIpos): m/z=278 [M+H]⁺.

### Example 65A

### 5-(Methoxymethyl)-2,2-dimethyl-4-(1-phenylethyl)morpholine [diastereomer mixture, 4 isomers]

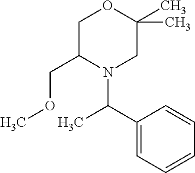 [SMILES: COCC1COC(C)(C)CN1C(C)c1ccccc1]

3.60 g (13.0 mmol) of 5-(methoxymethyl)-2,2-dimethyl-4-(1-phenylethyl)morpholin-3-one [diastereomer mixture, 4 isomers] were initially charged in tetrahydrofuran (128 ml), 26.0 ml (51.9 mmol) of 2 M borane/dimethyl sulphide complex solution in tetrahydrofuran were added under argon and the mixture was then stirred under reflux for 2 h. The mixture was subsequently cooled to 0° C., methanol (70 ml) was added carefully and the mixture was stirred under reflux for 30 min. The mixture was then concentrated completely under reduced pressure, and the residue was taken up in acetonitrile and purified directly by preparative RP-HPLC (acetonitrile/water, isocratic). Yield: 2.65 g (73% of theory).

LC-MS (method 5A): Rₜ=0.74 min; MS (ESIpos): m/z=264 [M+H]⁺.

### Example 66A

### 5-(Methoxymethyl)-2,2-dimethylmorpholine [racemate]

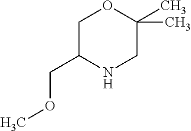

2.65 g (10.1 mmol) of 5-(methoxymethyl)-2,2-dimethyl-4-(1-phenylethyl)morpholine [diastereomer mixture, 4 isomers] were initially charged in ethanol (80.7 ml), 283 mg of palladium on carbon (10%) and 139 mg of palladium hydroxide on carbon (20%) were added under argon, and the mixture was then stirred under an atmosphere of hydrogen at standard pressure overnight. The reaction solution was filtered through kieselguhr and the filter residue was washed with ethanol. The filtrate was concentrated under reduced pressure and the product was dried under high vacuum. Yield: 1.43 g (89% of theory).

GC-MS (method 2B): Rₜ=2.62 min; MS (EIpos): m/z=160 [M]⁺.

### Example 67A

### 4-Benzyl-5-({[tert-butyl(dimethyl)silyl]oxy}methyl)-2-methylmorpholin-3-one [diastereomer mixture, 4 isomers]

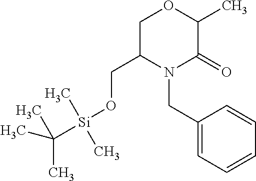 [SMILES: CC1OCC(CO[Si](C)(C)C(C)(C)C)N(Cc2ccccc2)C1=O]

21.5 g (91.4 mmol) of 4-benzyl-5-(hydroxymethyl)-2-methylmorpholin-3-one [diastereomer mixture, 4 isomers] were initially charged in N,N-dimethylformamide (126 ml), and 12.4 g (183 mmol) of imidazole and then 14.5 g (96.0 mmol) of tert-butyldimethylsilyl chloride were added at RT. The mixture was stirred for 2 h, and most of the solvent was then removed under reduced pressure. The residue was taken up in ethyl acetate/water and the organic phase was washed with water, 0.4 N aqueous hydrogen chloride solution, saturated aqueous sodium bicarbonate solution and water. The organic phase was dried over sodium sulphate, filtered and concentrated under reduced pressure. The crude product was used without further purification in the next step. Yield: 31.2 g (97% of theory, diastereomer ratio about 7:3).

LC-MS (method 1A): Rₜ=1.41 min; MS (ESIpos): m/z=350 [M+H]⁺;

¹H-NMR (400 MHz, DMSO-d₆): δ [ppm]=7.38-7.18 (m, 5H), 5.00 (d, 0.3H), 4.95 (d, 0.7H), 4.32-4.19 (m, 2H), 3.92-3.85 (m, 1H), 3.75-3.62 (m, 3H), 3.32-3.26 (m, 0.3H), 3.19-3.13 (m., 0.7H), 1.35 (d, 0.9H), 1.32 (d, 2.1H), 0.84-0.80 (m, 9H), 0.04-0.03 (m, 6H).

### Example 68A

### 4-Benzyl-5-({[tert-butyl(dimethyl)silyl]oxy}methyl)-2,2-dimethylmorpholin-3-one [racemate]

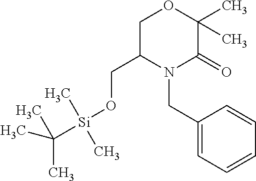 [SMILES: CC1(C)OCC(CO[Si](C)(C)C(C)(C)C)N(Cc2ccccc2)C1=O]

17.0 g (70.7 mmol) of 4-benzyl-5-({[tert-butyl(dimethyl)silyl]oxy}methyl)-2-methylmorpholin-3-one [diastereomer mixture, 4 isomers] were initially charged in tetrahydrofuran (340 ml), and 32.4 ml (58.4 mmol) of lithium diisopropylamide solution (1.8 M in tetrahydrofuran/n-heptane/ethylbenzene) were added at −78° C. The mixture was warmed slowly to 0° C., and 8.97 g (3.94 ml, 63.2 mmol) of iodomethane were then added. After 1.5 h, the mixture was again cooled to −78° C., and a further 5.40 ml (9.73 mmol) of lithium diisopropylamide solution (1.8 M in THF/n-heptane/ethylbenzene) were added. The mixture was warmed to 0° C., and 2.07 g (0.91 ml, 14.6 mmol) of iodomethane were then added. After 1 h, with cooling, water was added to the reaction solution, and the tetrahydrofuran was removed under reduced pressure. The residue was taken up in ethyl acetate and washed with water and saturated aqueous sodium chloride solution. The organic phase was dried over sodium sulphate, filtered and concentrated under reduced pressure. The crude product was used without further purification in the next step. Yield: 19.8 g (98% of theory, purity: 88%).

LC-MS (method 1A): Rₜ=1.45 min; MS (ESIpos): m/z=364 [M+H]⁺.

### Example 69A

### 4-Benzyl-5-(hydroxymethyl)-2,2-dimethylmorpholin-3-one [racemate]

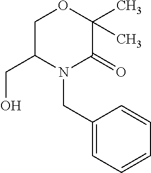 [SMILES: CC1(C)OCC(CO)N(Cc2ccccc2)C1=O]

18.1 g (43.8 mmol, purity: 88%) of 4-benzyl-5-({[tert-butyl(dimethyl)silyl]oxy}methyl)-2,2-dimethylmorpholin-3-one [racemate] were initially charged in tetrahydrofuran (329 ml) and 110 ml (110 mmol) of tetra-n-butylammonium fluoride solution (1.0 M in tetrahydrofuran) were added at RT. The reaction solution was stirred overnight and then concentrated under reduced pressure. The residue was taken up in ethyl acetate and washed with water and the organic phase was dried over sodium sulphate, filtered and concentrated under reduced pressure. The crude product was purified by column chromatography on silica gel (dichloromethane, dichloromethane/methanol 100:3). Yield: 9.99 g (89% of theory).

LC-MS (method 1A): Rₜ=0.73 min; MS (ESIpos): m/z=250 [M+H]⁺;

¹H-NMR (400 MHz, DMSO-d₆): δ [ppm]=7.43-7.35 (m, 2H), 7.34-7.21 (m, 3H), 5.09-4.98 (m, 2H), 4.23 (d, 1H), 3.90-3.75 (m, 2H), 3.65-3.55 (m, 2H), 3.15 (br. t., 1H), 1.42 (s, 3H), 1.39 (s, 3H).

### Example 70A

### 4-Benzyl-5-(fluoromethyl)-2,2-dimethylmorpholin-3-one [racemate]

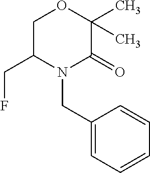 [SMILES: CC1(C)OCC(CF)N(Cc2ccccc2)C1=O]

2.00 g (8.02 mmol) of 4-benzyl-5-(hydroxymethyl)-2,2-dimethylmorpholin-3-one [racemate] were initially charged in tetrahydrofuran (40.1 ml), and 8.09 ml (18.8 mmol) of bis(2-methoxyethyl)aminosulphur trifluoride (Deoxofluor, 50% strength solution in tetrahydrofuran) were added slowly at RT. 2 drops of ethanol were then added and the mixture was subsequently stirred under reflux for 5 h. The reaction solution was carefully added dropwise to saturated aqueous sodium bicarbonate solution and the aqueous phase was extracted with dichloromethane. The combined organic phases were dried over sodium sulphate, filtered and concentrated under reduced pressure. The crude product was used without further purification in the next step. Yield: 2.15 g (87% of theory, purity: 81%).

LC-MS (method 1A): Rₜ=0.91 min; MS (ESIpos): m/z=252 [M+H]⁺.

### Example 71A

### 4-Benzyl-5-(fluoromethyl)-2,2-dimethylmorpholine [racemate]

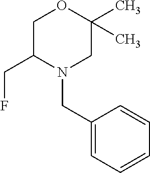 [SMILES: CC1(C)CN(Cc2ccccc2)C(CF)CO1]

2.15 g (8.56 mmol) of 4-benzyl-5-(fluoromethyl)-2,2-dimethylmorpholin-3-one [racemate] was initially charged in tetrahydrofuran (84.2 ml), 17.1 ml (34.2 mmol) of 2 M borane/dimethyl sulphide complex solution in tetrahydrofuran were added under argon and the mixture was stirred under reflux for 2 h. The mixture was subsequently cooled to 0° C., methanol (10 ml) was added carefully and the mixture was stirred under reflux for 30 min. The mixture was then concentrated completely under reduced pressure, and the residue was taken up in acetonitrile and purified by preparative RP-HPLC (acetonitrile/water, isocratic). Yield: 1.03 g (43% of theory, purity: 86%).

LC-MS (method 1A): Rₜ=0.85 min; MS (ESIpos): m/z=238 [M+H]⁺.

### Example 72A

### 5-(Fluoromethyl)-2,2-dimethylmorpholine [racemate]

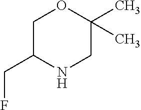

1.00 g (4.21 mmol) of 4-benzyl-5-(fluoromethyl)-2,2-dimethylmorpholine [racemate] was initially charged in ethanol (33.8 ml), 99 mg of palladium on carbon (10%) and 50 mg of palladium hydroxide on carbon (20%) were added under argon and the mixture was then stirred under an atmosphere of hydrogen at standard pressure overnight. The reaction solution was filtered through kieselguhr and the filter residue was washed with ethanol. The filtrate was concentrated under reduced pressure and the product was dried under high vacuum. Yield: 192 g (31% of theory).

GC-MS (method 2B): Rₜ=1.98 min

### Example 73A

### N-Benzyl-2-chloro-N-(1,4-dihydroxybutan-2-yl)propanamide [diastereomer mixture, 4 isomers]

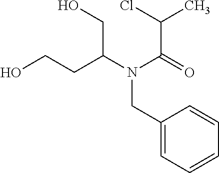 [SMILES: CC(Cl)C(=O)N(Cc1ccccc1)C(CO)CCO]

20.6 g (106 mmol) of 2-(benzylamino)butane-1,4-diol [racemate] [lit.: B. L. Feringa, B. de Lange, Heterocycles 1988, 27, 1197-1205] were initially charged in isopropanol (500 ml), the mixture was cooled to 0° C. and 21.4 g (29.4 ml, 211 mmol) of triethylamine were added. 16.1 g (12.6 ml, 127 mmol) of 2-chloropropionyl chloride [racemate] were then added dropwise. After 30 min of stirring, a further 10.4 g (8.37 ml, 84.4 mmol) of 2-chloropropionyl chloride [racemate] were added dropwise, and the reaction solution was allowed to warm to RT. The solution was then concentrated under reduced pressure and the residue was taken up in ethyl acetate (500 ml) and washed with 0.5 N aqueous hydrogen chloride solution (400 ml). The aqueous phase was extracted repeatedly with ethyl acetate. The organic phases were dried over sodium sulphate, filtered and concentrated under reduced pressure. The crude product was used without further purification in the next step. Yield: 37.5 g, 78% of theory, purity: 63%, diastereomer ratio about 2:1).

LC-MS (Method 1A): Rₜ=0.71 min (diastereomer 1, 2 isomers), Rₜ=0.72 min (diastereomer 2, 2 isomers); MS (ESIpos): m/z=286 [M+H]⁺.

### Example 74A

### 4-Benzyl-5-(2-hydroxyethyl)-2-methylmorpholin-3-one [diastereomer mixture, 4 isomers]

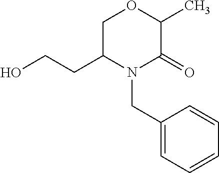 [SMILES: CC1OCC(CCO)N(Cc2ccccc2)C1=O]

37.5 g (82.5 mmol, purity: 63%) of N-benzyl-2-chloro-N-(-1,4-dihydroxybutan-2-yl)propanamide [diastereomer mixture, 4 isomers] in isopropanol (500 ml) were cooled to 0° C., and 73.5 g (655 mmol) of potassium tert-butoxide were then added in one portion. The mixture was stirred at 0° C. for 1 h and most of the isopropanol was then removed under reduced pressure. The residue was taken up in ethyl acetate and washed with a 1 N aqueous hydrogen chloride solution (400 ml). The organic phase was dried over sodium sulphate, filtered and concentrated under reduced pressure. The crude product was used without further purification in the next step. Yield: 28.8 g (quant., purity: 82%, diastereomer ratio about 2.5:1).

LC-MS (Method 7A): Rₜ=1.42 min (diastereomer 1, 2 isomers), Rₜ=1.46 min (diastereomer 2, 2 isomers); MS (ESIpos): m/z=250 [M+H]⁺.

### Example 75A

### 2-(4-Benzyl-6-methylmorpholin-3-yl)ethanol [racemate]

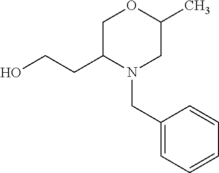 [SMILES: CC1CN(Cc2ccccc2)C(CCO)CO1]

28.8 g (94.7 mmol, purity: 82%) of 4-benzyl-5-(2-hydroxyethyl)-2-methylmorpholin-3-one [diastereomer mixture, 4 isomers] were initially charged in tetrahydrofuran (800 ml), 231 ml (462 mmol) of 2 M borane/dimethyl sulphide complex solution in tetrahydrofuran were added under argon and the mixture was stirred under reflux for 2 h. The mixture was subsequently cooled to 0° C., methanol (220 ml) was added carefully and the mixture was stirred under reflux for 30 min. The mixture was subsequently concentrated completely under reduced pressure, and 6.0 g of the residue were taken up in acetonitrile and subjected to purification and diastereomer separation by preparative RP-HPLC (acetonitrile/water, isocratic). Here, the target compound eluted as second component and was the main isomer. Yield: target compound (diastereomer 2, racemate, main isomer): 1.95 g; minor isomer: 698 mg.

target compound (diastereomer 2, racemate, main isomer): LC-MS (method 4A): Rₜ=2.33 min; MS (ESIpos): m/z=236 [M+H]⁺.

minor isomer: LC-MS (method 4A): Rₜ=2.23 min; MS (ESIpos): m/z=236 [M+H]⁺.

### Example 76A

### 2-(6-Methylmorpholin-3-yl)ethanol [racemate]

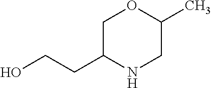

1.95 g (8.29 mmol) of 2-(4-benzyl-6-methylmorpholin-3-yl)ethanol [racemate, diastereomer 2 from Example 75A] were initially charged in ethanol (83 ml), 208 mg of palladium on carbon (10%) and 104 mg of palladium hydroxide on carbon (20%) were added under argon, and the mixture was then stirred under an atmosphere of hydrogen at standard pressure overnight. The reaction solution was filtered through kieselguhr and the filter residue was washed with ethanol. The filtrate was concentrated under reduced pressure and the product was dried under high vacuum. Yield: 1.37 g (quant.).

MS (method 1C): m/z=146 [M+H]⁺;

¹H-NMR (400 MHz, DMSO-d₆): δ [ppm]=3.53-3.41 (m, 5H), 2.69 (m_{c}, 1H), 2.60-2.43 (m, 2H), 1.82-1.69 (m, 1H), 1.58-1.44 (m, 1H), 1.03 (d, 3H), two protons not visible

### Example 77A

### (2-Bromo-8-methoxy[1,2,4]triazolo[1,5-a]pyridin-6-yl)[5-(2-hydroxyethyl)-2-methylmorpholin-4-yl]methanone [racemate]

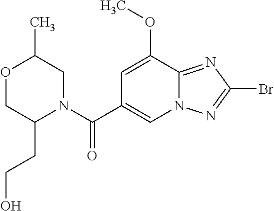 [SMILES: COc1cc(C(=O)N2CC(C)OCC2CCO)cn2nc(Br)nc12]

500 mg (1.84 mmol) of 2-bromo-8-methoxy[1,2,4]triazolo[1,5-a]pyridine-6-carboxylic acid and 320 mg (2.21 mmol) of 2-(6-methylmorpholin-3-yl)ethanol [racemate] were initially charged in N,N-dimethylformamide (12.1 ml), and 950 mg (1.28 ml, 7.35 mmol) of N,N-diisopropylethylamine were added. Subsequently, 839 mg (2.21 mmol) of HATU were added at RT and the mixture was stirred overnight. Without further work-up, the reaction solution was then purified by preparative RP-HPLC (acetonitrile/water). Yield: 666 mg (67% of theory, purity: 73%).

LC-MS (method 1A): Rₜ=0.68 min; MS (ESIpos): m/z=399 [M+H]⁺.

### Example 78A

### (2-Bromo-8-methoxy[1,2,4]triazolo[1,5-a]pyridin-6-yl)(2-methyl-5-{2-[(triisopropylsilyl)oxy]-ethyl}morpholin-4-yl)methanone [racemate]

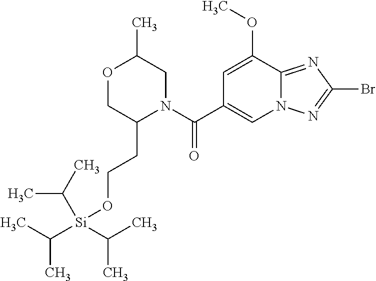 [SMILES: COc1cc(C(=O)N2CC(C)OCC2CCO[Si](C(C)C)(C(C)C)C(C)C)cn2nc(Br)nc12]

200 mg (0.501 mmol) of (2-bromo-8-methoxy[1,2,4]triazolo[1,5-a]pyridin-6-yl)[5-(2-hydroxyethyl)-2-methylmorpholin-4-yl]methanone [racemate] were initially charged in N,N-dimethylformamide (6.29 ml), 106 mg (0.551 mmol) of chlorotriisopropylsilane, 68.2 mg (1.00 mmol) of imidazole and a catalytic amount of N,N′-dimethylaminopyridine (about 5 mg) were added at RT and the mixture was stirred for 24 h. A further 106 mg (0.551 mmol) of chlorotriisopropylsilane, 68.2 mg (1.00 mmol) of imidazole and N,N′-dimethylaminopyridine (about 5 mg) were then added and the mixture was stirred at 40° C. for 4 h. The reaction solution was concentrated under reduced pressure, the residue was taken up in ethyl acetate and the organic phase was washed first with 0.5 N aqueous hydrogen chloride solution and then with saturated aqueous sodium bicarbonate solution. The organic phase was dried over sodium sulphate, filtered and concentrated under reduced pressure. The residue was purified by flash chromatography on silica gel (dichloromethane/methanol 100:1). Yield: 203 mg (62% of theory, purity: 85%).

LC-MS (method 1A): Rₜ=1.48 min; MS (ESIpos): m/z=555 [M+H]⁺.

### Example 79A

### (2-{[1-(3-Chlorophenyl)-2-fluoroethyl]amino}-8-methoxy[1,2,4]triazolo[1,5-a]pyridin-6-yl)(2-methyl-5-{2-[(triisopropylsilyl)oxy]ethyl}morpholin-4-yl)methanone [diastereomer mixture, 2 isomers]

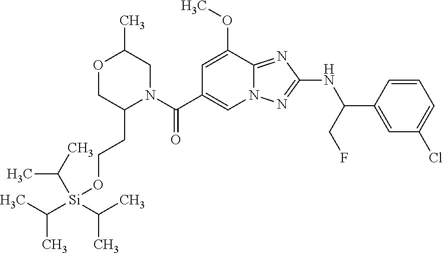 [SMILES: COc1cc(C(=O)N2CC(C)OCC2CCO[Si](C(C)C)(C(C)C)C(C)C)cn2nc(NC(CF)c3cccc(Cl)c3)nc12]

150 mg (0.270 mmol) of (2-bromo-8-methoxy[1,2,4]triazolo[1,5-a]pyridin-6-yl)(2-methyl-5-{2-[(triisopropylsilyl)oxy]ethyl}morpholin-4-yl)methanone [racemate], 68.1 mg (0.324 mmol) of 1-(3-chlorophenyl)-2-fluoroethanamine hydrochloride [enantiomerically pure isomer], 82.7 mg (459 μl, 1.75 mmol, 2 M solution in tetrahydrofuran) of sodium tert-butoxide, 23.3 mg (0.040 mmol) of bis(dibenzylideneacetone)palladium(0) and 23.4 mg (0.040 mmol) of 4,5-bis(diphenylphosphino)-9,9-dimethylxanthene (XantPhos) were initially charged in degassed 1,4-dioxane (4.00 ml) in a microwave tube. The tube was sealed and the reaction mixture was subsequently stirred at 160° C. in the microwave oven (Biotage Synthesizer) for 1 h. The mixture was then diluted with ethyl acetate and washed with water. The organic phase was dried over sodium sulphate, filtered and concentrated under reduced pressure. The residue was purified by preparative RP-HPLC (acetonitrile/water). Yield: 50.1 mg (28% of theory).

LC-MS (method 1A): Rₜ=1.48 min; MS (ESIpos): m/z=648 [M+H]⁺.

### Example 81A

### [1-Amino-3-(benzyloxy)cyclobutyl]methanol [diastereomer mixture, 2 isomers, cis/trans about 4:1]

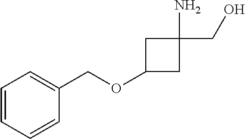 [SMILES: NC1(CO)CC(OCc2ccccc2)C1]

I) 5.00 g (20.3 mmol) of 2-(benzyloxy)-5,7-diazaspiro[3.4]octane-6,8-dione [diastereomer mixture, 2 isomers, cis/trans about 4:1; T. M. Shoup, M. M. Goodman, J. Labelled. Cpd. Radiopharm. 1999, 42, 215-225; US2006/292073 A1] were initially charged in water (100 ml), and 32.0 g (102 mmol) of barium hydroxide octahydrate were added. In seven portions, the suspension was stirred in the microwave (Biotage Synthesizer), in each case for 1.5 h at 140° C. The suspensions were combined and adjusted to a pH of about 4 using a 6 N aqueous sulphuric acid solution. The precipitated solid was filtered off under reduced pressure, the filtrate was then concentrated under reduced pressure and the solid obtained was dried under high vacuum. This gave 6.2 g of crude product.

II) 21.3 g (24.9 ml, 196 mmol) of chlorotrimethylsilane were added dropwise to 49.1 ml of a 2 M solution of lithium borohydride in tetrahydrofuran (98.2 mmol). The suspension obtained was cooled to 0° C., and 5.43 g of the crude product from I) were then added a little at a time. The mixture was then warmed to RT and stirred at RT overnight. The reaction was terminated by dropwise addition of methanol (15 ml) and the reaction solution was then concentrated under reduced pressure. The residue was taken up in ethyl acetate and washed with an aqueous 2 N sodium hydroxide solution. The aqueous phase was extracted with ethyl acetate, and the combined organic phases were dried over sodium sulphate, filtered and concentrated under reduced pressure. The crude product was used without further purification in the next step. Yield: 3.76 g (crude product).

LC-MS (method 4A): Rₜ=2.10 min; MS (ESIpos): m/z=208 [M+H]⁺;

¹H-NMR (400 MHz, DMSO-d₆): δ [ppm]=7.39-7.22 (m, 5H), 4.66 (br. s., 1H), 4.32 (s, 2H), 4.15 (quin, 0.2H), 3.70 (quin, 0.8H), 3.22-3.14 (m, 2H), 2.34-2.26 (m, 2H), 1.91-1.74 (m, 2H), 1.72-1.61 (m, 2H).

### Example 82A

### tert-Butyl[3-(benzyloxy)-1-(hydroxymethyl)cyclobutyl]carbamate [enantiomerically pure cis and trans isomer]

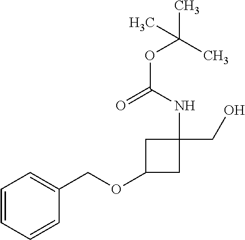 [SMILES: CC(C)(C)OC(=O)NC1(CO)CC(OCc2ccccc2)C1]

3.76 g (18.1 mmol) of [1-amino-3-(benzyloxy)cyclobutyl]methanol [diastereomer mixture, 2 isomers cis/trans about 4:1] were initially charged in dichloromethane (150 ml), and 4.36 g (20.0 mmol) of di-tert-butyl dicarbonate and 3.86 g (5.31 ml, 38.1 mmol) of triethylamine were added at RT. The reaction solution was stirred at RT overnight and then washed with an aqueous 0.5 N hydrogen chloride solution, saturated aqueous sodium bicarbonate solution and water. The organic phase was dried over sodium sulphate, filtered and concentrated under reduced pressure. The crude product (6.4 g) was purified by preparative RP-HPLC (Method 1G) and separated into the diastereomers. Here, the more rapidly eluting major diastereomer was the cis isomer, and the slower eluting minor diastereomer was the trans isomer. Yield: 3.45 g (61% of theory, enantiomerically pure cis isomer); 690 mg (12% of theory, enantiomerically pure trans isomer).

enantiomerically pure cis diastereomer:

LC-MS (method 1A): Rₜ=2.00 min; MS (ESIpos): m/z=308 [M+H]⁺;

enantiomerically pure trans diastereomer:

LC-MS (method 1A): Rₜ=2.02 min; MS (ESIpos): m/z=308 [M+H]⁺.

### Example 83A

### [cis-1-Amino-3-(benzyloxy)cyclobutyl]methanol hydrochloride [enantiomerically pure cis diastereomer]

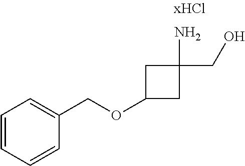 [SMILES: NC1(CO)CC(OCc2ccccc2)C1]

3.45 g (11.2 mmol) of tert-butyl [cis-3-(benzyloxy)-1-(hydroxymethyl)cyclobutyl]carbamate [enantiomerically pure cis diastereomer from Example 82A] were initially charged in 1,4-dioxane (30 ml) and 11.2 ml of a 4 N solution of hydrogen chloride in 1,4-dioxane/water were added at RT. The mixture was stirred at RT for 20 h and then concentrated under reduced pressure, and the product was dried under high vacuum. The crude product was used without further purification in the next step. Yield: 2.81 g (quant.).

LC-MS (method 1A): Rₜ=0.40 min; MS (ESIpos): m/z=208 [M+H-HCl]⁺;

¹H-NMR (400 MHz, DMSO-d₆): δ [ppm]=8.24 (br. s., 3H), 7.43-7.25 (m, 5H), 5.54 (br. s., 1H), 4.39 (s, 2H), 3.90 (quin, 1H), 3.46 (br. d., 2H), 2.42 (m_{c}, 2H), 2.12 (m_{c}, 2H).

### Example 84A

### [trans-1-Amino-3-(benzyloxy)cyclobutyl]methanol hydrochloride [enantiomerically pure trans diastereomer]

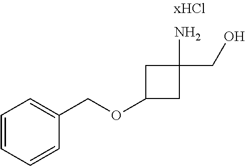 [SMILES: NC1(CO)CC(OCc2ccccc2)C1]

683 g (2.22 mmol) of tert-butyl [trans-3-(benzyloxy)-1-(hydroxymethyl)cyclobutyl]carbamate [enantiomerically pure trans diastereomer from Example 82A] were initially charged in 1,4-dioxane (30 ml) and 2.22 ml of a 4 N solution of hydrogen chloride in 1,4-dioxane/water were added at RT. The mixture was stirred at RT for 18 h and then concentrated under reduced pressure, and the product was dried under high vacuum. The crude product was used without further purification in the next step. Yield: 594 mg (quant.).

LC-MS (method 1A): Rₜ=0.39 min; MS (ESIpos): m/z=208 [M+H-HCl]⁺;

¹H-NMR (400 MHz, DMSO-d₆): δ [ppm]=8.26 (br. s., 3H), 7.40-7.24 (m, 5H), 5.54 (br. s., 1H), 4.36 (s, 2H), 4.26 (m_{c}, 1H), 3.55 (s, 2H), 2.37 (m_{c}, 2H), 2.11 (m_{c}, 2H).

### Example 85A

### N-[cis-3-(Benzyloxy)-1-(hydroxymethyl)cyclobutyl]-2-chloropropanamide [racemate]

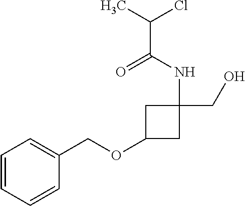 [SMILES: CC(Cl)C(=O)NC1(CO)CC(OCc2ccccc2)C1]

2.81 g (11.5 mmol) of [cis-1-amino-3-(benzyloxy)cyclobutyl]methanol hydrochloride [enantiomerically pure cis diastereomer] in isopropanol (70.0 ml) were cooled to 0° C., and 4.67 g (6.43 ml, 46.1 mmol) of triethylamine were added. 1.61 g (1.26 ml, 12.7 mmol) of 2-chloropropionyl chloride [racemate] were then added dropwise. The reaction solution was allowed to warm to RT, stirred for 1 h and concentrated under reduced pressure. The residue was taken up in dichloromethane and washed with an aqueous 1 N hydrogen chloride solution. The organic phases were dried over sodium sulphate, filtered and concentrated under reduced pressure. The crude product was used without further purification in the next step. Yield: 3.38 g (97% of theory).

LC-MS (method 1A): Rₜ=0.85 min; MS (ESIpos): m/z=298 [M+H]⁺.

### Example 86A

### N-[trans-3-(Benzyloxy)-1-(hydroxymethyl)cyclobutyl]-2-chloropropanamide [racemate]

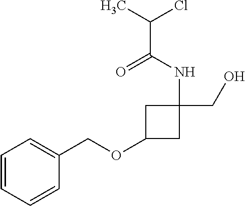 [SMILES: CC(Cl)C(=O)NC1(CO)CC(OCc2ccccc2)C1]

594 mg (2.44 mmol) of [trans-1-amino-3-(benzyloxy)cyclobutyl]methanol hydrochloride [enantiomerically pure trans diastereomer] in isopropanol (30.0 ml) were cooled to 0° C., and 986 g (1.36 ml, 9.75 mmol) of triethylamine were added. 340 mg (266 μl, 2.68 mmol) of 2-chloropropionyl chloride [racemate] were then added dropwise. The reaction solution was allowed to warm to RT, stirred for 1 h and concentrated under reduced pressure. The residue was taken up in dichloromethane and washed with an aqueous 1 N hydrogen chloride solution. The organic phases were dried over sodium sulphate, filtered and concentrated under reduced pressure. The crude product was used without further purification in the next step. Yield: 688 mg (89% of theory).

LC-MS (method 1A): Rₜ=0.87 min; MS (ESIpos): m/z=298 [M+H]⁺.

### Example 87A

### cis-2-(Benzyloxy)-7-methyl-8-oxa-5-azaspiro[3.5]nonan-6-one [racemate]

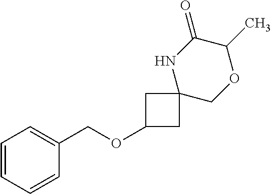 [SMILES: CC1OCC2(CC(OCc3ccccc3)C2)NC1=O]

3.38 g (11.4 mmol) of N-[cis-3-(benzyloxy)-1-(hydroxymethyl)cyclobutyl]-2-chloropropanamide [racemate] were initially charged in isopropanol (250 ml), the mixture was cooled to 0° C. and 3.82 g (34.1 mmol) of potassium tert-butoxide were then added in one portion. The mixture was allowed to warm to RT and stirred at 50° C. for 1 h. The isopropanol was then removed under reduced pressure and the residue was taken up in dichloromethane. The organic phase was washed with 1 N aqueous hydrogen chloride solution, dried over sodium sulphate, filtered and concentrated under reduced pressure. The residue was purified by preparative RP-HPLC (acetonitrile/water). Yield: 2.96 g (99% of theory).

LC-MS (method 1A): Rₜ=0.86 min; MS (ESIpos): m/z=262 [M+H]⁺.

### Example 88A

### trans-2-(Benzyloxy)-7-methyl-8-oxa-5-azaspiro[3.5]nonan-6-one [racemate]

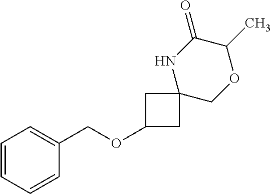 [SMILES: CC1OCC2(CC(OCc3ccccc3)C2)NC1=O]

688 mg (2.31 mmol) of N-[trans-3-(benzyloxy)-1-(hydroxymethyl)cyclobutyl]-2-chloropropanamide [racemate] were initially charged in isopropanol (30 ml), the mixture was cooled to 0° C. and 778 mg (6.93 mmol) of potassium tert-butoxide were then added in one portion. The mixture was allowed to warm to RT and stirred at 50° C. for 1 h. Most of the isopropanol was then removed under reduced pressure and the residue was taken up in dichloromethane. The organic phase was washed with 1 N aqueous hydrogen chloride solution, dried over sodium sulphate, filtered and concentrated under reduced pressure. The residue was purified by preparative RP-HPLC (acetonitrile/water). Yield: 600 mg (99% of theory).

LC-MS (method 1A): Rₜ=0.86 min; MS (ESIpos): m/z=262 [M+H]⁺.

### Example 89A

### cis-2-(Benzyloxy)-7-methyl-8-oxa-5-azaspiro[3.5]nonane [racemate]

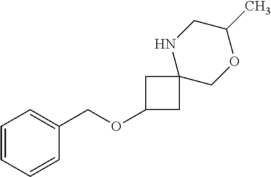 [SMILES: CC1CNC2(CO1)CC(OCc1ccccc1)C2]

2.96 g (11.3 mmol) of cis-2-(benzyloxy)-7-methyl-8-oxa-5-azaspiro[3.5]nonan-6-one [racemate] were initially charged in tetrahydrofuran (200 ml), 22.7 ml (45.3 mmol) of 2 M borane/dimethyl sulphide complex solution in tetrahydrofuran were added under argon and the mixture was stirred under reflux for 2 h. The reaction solution was subsequently cooled to 0° C., methanol (100 ml) was added carefully dropwise and the mixture was then stirred under reflux for 12 h. The mixture was then concentrated completely under reduced pressure, and the residue was taken up in acetonitrile and purified by preparative RP-HPLC (acetonitrile/water). Yield: 2.80 g (91% of theory).

LC-MS (method 1A): Rₜ=0.61 min; MS (ESIpos): m/z=248 [M+H]⁺.

### Example 90A

### trans-2-(Benzyloxy)-7-methyl-8-oxa-5-azaspiro[3.5]nonane [racemate]

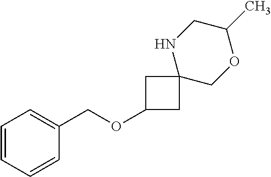 [SMILES: CC1CNC2(CO1)CC(OCc1ccccc1)C2]

600 mg (11.3 mmol) of trans-2-(benzyloxy)-7-methyl-8-oxa-5-azaspiro[3.5]nonan-6-one [racemate] were initially charged in tetrahydrofuran (100 ml), 4.59 ml (9.18 mmol) of 2 M borane/dimethyl sulphide complex solution in tetrahydrofuran were added under argon and the reaction mixture was stirred under reflux for 2 h. The mixture was cooled to 0° C., methanol (50 ml) was added carefully dropwise and the mixture was stirred under reflux for 12 h. The mixture was then concentrated completely under reduced pressure, and the residue was taken up in acetonitrile and purified by preparative RP-HPLC (acetonitrile/water). Yield: 583 g (82% of theory, purity: 80%).

LC-MS (method 1A): Rₜ=0.59 min; MS (ESIpos): m/z=248 [M+H]⁺.

### Example 91A

### tert-Butyl cis-2-(benzyloxy)-7-methyl-8-oxa-5-azaspiro[3.5]nonane-5-carboxylate [racemate]

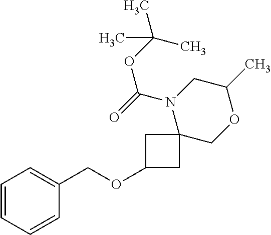 [SMILES: CC1CN(C(=O)OC(C)(C)C)C2(CO1)CC(OCc1ccccc1)C2]

2.80 g (11.3 mmol) of cis-2-(benzyloxy)-7-methyl-8-oxa-5-azaspiro[3.5]nonane [racemate] were initially charged in dichloromethane (150 ml), and 3.71 g (17.0 mmol) of di-tert-butyl dicarbonate and 5.73 g (7.89 ml, 56.6 mmol) of triethylamine were added at RT. The reaction solution was subsequently stirred at RT overnight and then washed with an aqueous 0.5 N hydrogen chloride solution, saturated aqueous sodium bicarbonate solution and water. The organic phase was dried over sodium sulphate, filtered and concentrated under reduced pressure. Yield: 3.33 g (84% of theory).

LC-MS (method 1A): Rₜ=1.28 min; MS (ESIpos): m/z=348 [M+H]⁺.

### Example 92A

### tert-Butyl cis-2-(benzyloxy)-7-methyl-8-oxa-5-azaspiro[3.5]nonane-5-carboxylate [enantiomerically pure isomer 1]

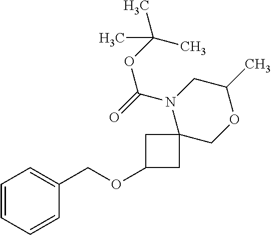 [SMILES: CC1CN(C(=O)OC(C)(C)C)C2(CO1)CC(OCc1ccccc1)C2]

The enantiomer separation of 3.33 g of the compound from Example 91A (Method 5D) gave 1.06 g of the compound from Example 92A (enantiomerically pure isomer 1) and 928 mg of the compound from Example 93A (enantiomerically pure isomer 2).

HPLC (Method 16E): Rₜ=5.06 min, 99.9% ee;

LC-MS (method 1A): Rₜ=1.30 min; MS (ESIpos): m/z=348 [M+H]⁺;

¹H-NMR (400 MHz, DMSO-d₆): δ [ppm]=7.40-7.23 (m, 5H), 4.37 (m_{c}, 2H), 3.79 (quin, 1H), 3.62 (dd, 1H), 3.49-3.33 (m, 3H), 2.69-2.56 (m, 2H), 2.43 (dd, 1H), 2.32-2.23 (m, 1H), 1.78 (m_{c}, 1H), 1.38 (s, 9H), 1.01 (d, 3H).

### Example 93A

### tert-Butyl cis-2-(benzyloxy)-7-methyl-8-oxa-5-azaspiro[3.5]nonane-5-carboxylate [enantiomerically pure isomer 2]

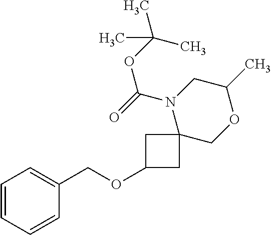 [SMILES: CC1CN(C(=O)OC(C)(C)C)C2(CO1)CC(OCc1ccccc1)C2]

The enantiomer separation of 3.33 g of the compound from Example 91A (Method 5D) gave 1.06 g of the compound from Example 92A (enantiomerically pure isomer 1) and 928 mg of the compound from Example 93A (enantiomerically pure isomer 2).

HPLC (Method 16E): Rₜ=13.5 min, 99.9% ee;

LC-MS (method 1A): Rₜ=1.30 min; MS (ESIpos): m/z=348 [M+H]⁺;

¹H-NMR (400 MHz, DMSO-d₆): δ [ppm]=7.38-7.25 (m, 5H), 4.37 (m_{c}, 1H), 3.79 (quin, 1H), 3.62 (dd, 1H), 3.47-3.34 (m, 2H), 2.68-2.56 (m, 2H), 2.43 (dd, 1H), 2.32-2.22 (m, 1H), 1.78 (m_{c}, 1H), 1.38 (s, 9H), 1.01 (d, 3H).

### Example 94A

### tert-Butyl trans-2-(benzyloxy)-7-methyl-8-oxa-5-azaspiro[3.5]nonane-5-carboxylate [racemate]

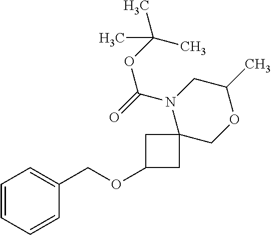 [SMILES: CC1CN(C(=O)OC(C)(C)C)C2(CO1)CC(OCc1ccccc1)C2]

583 mg (2.36 mmol) of trans-2-(benzyloxy)-7-methyl-8-oxa-5-azaspiro[3.5]nonane [racemate] were initially charged in dichloromethane (50 ml), and 772 mg (3.54 mmol) of di-tert-butyl dicarbonate and 1.19 g (1.64 ml, 11.8 mmol) of triethylamine were added at RT. The reaction solution was subsequently stirred at RT overnight and then washed with an aqueous 0.5 N hydrogen chloride solution, saturated aqueous sodium bicarbonate solution and water. The organic phase was dried over sodium sulphate, filtered and concentrated under reduced pressure. Yield: 523 mg (62% of theory).

LC-MS (method 1A): Rₜ=1.33 min; MS (ESIpos): m/z=248 [M+H-C₅H₈O₂]⁺.

### Example 95A

### tert-Butyl trans-2-(benzyloxy)-7-methyl-8-oxa-5-azaspiro[3.5]nonane-5-carboxylate [enantiomerically pure isomer 1]

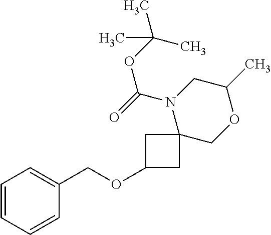 [SMILES: CC1CN(C(=O)OC(C)(C)C)C2(CO1)CC(OCc1ccccc1)C2]

The enantiomer separation of 523 mg of the compound from Example 94A (Method 5D) gave 125 mg of the compound from Example 95A (enantiomerically pure isomer 1) and 250 mg of the compound from Example 96A (enantiomerically pure isomer 2).

HPLC (Method 16E): Rₜ=4.64 min, 99.9% ee;

LC-MS (method 1A): Rₜ=1.34 min; MS (ESIpos): m/z=348 [M+H]⁺;

¹H-NMR (400 MHz, DMSO-d₆): δ [ppm]=7.40-7.24 (m, 5H), 4.35 (m_{c}, 2H), 4.14-4.04 (m, 1H), 3.83 (d, 1H), 3.62 (dd, 1H), 3.46 (d, 1H), 3.42-3.34 (m, 1H), 2.89 (dd, 1H), 2.64-2.56 (m, 1H), 2.29-2.17 (m, 2H), 1.95-1.86 (m, 1H), 1.39 (s, 9H), 1.02 (d, 3H).

### Example 96A

### tert-Butyl trans-2-(benzyloxy)-7-methyl-8-oxa-5-azaspiro[3.5]nonane-5-carboxylate [enantiomerically pure isomer 2]

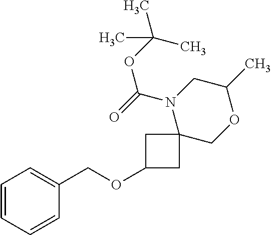 [SMILES: CC1CN(C(=O)OC(C)(C)C)C2(CO1)CC(OCc1ccccc1)C2]

The enantiomer separation of 523 mg of the compound from Example 94A (Method 5D) gave 125 mg of the compound from Example 95A (enantiomerically pure isomer 1) and 250 mg of the compound from Example 96A (enantiomerically pure isomer 2).

HPLC (Method 16E): Rₜ=6.38 min, 99.8% ee;

LC-MS (method 1A): Rₜ=1.35 min; MS (ESIpos): m/z=348 [M+H]⁺;

¹H-NMR (400 MHz, DMSO-d₆): δ [ppm]=7.42-7.24 (m, 5H), 4.35 (m_{c}, 2H), 4.14-4.05 (m, 1H), 3.83 (d, 1H), 3.62 (dd, 1H), 3.46 (d, 1H), 3.41-3.35 (m, 1H), 2.89 (dd, 1H), 2.59 (dd, 1H), 2.28-2.16 (m, 2H), 1.95-1.86 (m, 1H), 1.39 (s, 9H), 1.02 (d, 3H).

### Example 97A

### cis-2-(Benzyloxy)-7-methyl-8-oxa-5-azaspiro[3.5]nonane hydrochloride [enantiomerically pure isomer 1]

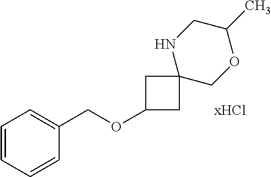 [SMILES: CC1CNC2(CO1)CC(OCc1ccccc1)C2]

1.06 g (3.06 mmol) of tert-butyl cis-2-(benzyloxy)-7-methyl-8-oxa-5-azaspiro[3.5]nonane-5-carboxylate [enantiomerically pure isomer 1 from Example 92A] were initially charged in 1,4-dioxane (30 ml), and 10.0 ml of a 4 N solution of hydrogen chloride in 1,4-dioxane were added at RT. The mixture was stirred at RT overnight and then concentrated under reduced pressure, and the product was dried under high vacuum. Yield: 1.04 g (quant.).

LC-MS (method 1A): Rₜ=0.48 min; MS (ESIpos): m/z=248 [M+H-HCl]⁺.

### Example 98A

### cis-2-(Benzyloxy)-7-methyl-8-oxa-5-azaspiro[3.5]nonane hydrochloride [enantiomerically pure isomer 2]

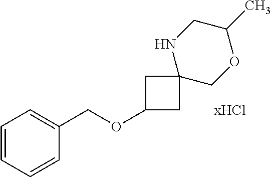 [SMILES: CC1CNC2(CO1)CC(OCc1ccccc1)C2]

928 mg (2.67 mmol) of tert-butyl cis-2-(benzyloxy)-7-methyl-8-oxa-5-azaspiro[3.5]nonane-5-carboxylate [enantiomerically pure isomer 2 from Example 93A] were initially charged in 1,4-dioxane (30 ml), and 10.0 ml of a 4 N solution of hydrogen chloride in 1,4-dioxane were added at RT. The mixture was stirred at RT overnight and then concentrated under reduced pressure, and the product was dried under high vacuum. Yield: 1.16 g (quant.).

LC-MS (method 1A): Rₜ=0.51 min; MS (ESIpos): m/z=248 [M+H-HCl]⁺.

### Example 99A

### trans-2-(Benzyloxy)-7-methyl-8-oxa-5-azaspiro[3.5]nonane hydrochloride [enantiomerically pure isomer 1]

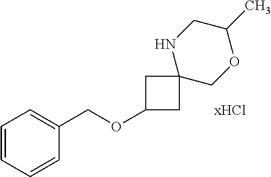 [SMILES: CC1CNC2(CO1)CC(OCc1ccccc1)C2]

125 mg (0.360 mmol) of tert-butyl trans-2-(benzyloxy)-7-methyl-8-oxa-5-azaspiro[3.5]nonane-5-carboxylate [enantiomerically pure isomer 1 from Example 95A] were initially charged in 1,4-dioxane (12 ml), and 4.00 ml of a 4 N solution of hydrogen chloride in 1,4-dioxane were added at RT. The mixture was stirred at RT overnight and then concentrated under reduced pressure, and the product was dried under high vacuum. Yield: 102 mg (92% of theory).

LC-MS (method 1A): Rₜ=0.64 min; MS (ESIpos): m/z=248 [M+H-HCl]⁺.

### Example 100A

### trans-2-(Benzyloxy)-7-methyl-8-oxa-5-azaspiro[3.5]nonane hydrochloride [enantiomerically pure isomer 2]

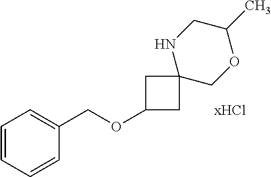 [SMILES: CC1CNC2(CO1)CC(OCc1ccccc1)C2]

250 mg (0.720 mmol) of tert-butyl trans-2-(benzyloxy)-7-methyl-8-oxa-5-azaspiro[3.5]nonane-5-carboxylate [enantiomerically pure isomer 2 from Example 96A] were initially charged in 1,4-dioxane (15 ml), and 5.00 ml of a 4 N solution of hydrogen chloride in 1,4-dioxane were added at RT. The mixture was stirred at RT overnight and then concentrated under reduced pressure, and the product was dried under high vacuum. Yield: 204 mg (86% of theory).

LC-MS (method 1A): Rₜ=0.64 min; MS (ESIpos): m/z=248 [M+H-HCl]⁺.

### Example 101A

### cis-7-Methyl-8-oxa-5-azaspiro[3.5]nonan-2-ol hydrochloride [enantiomerically pure isomer 1]

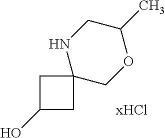

1.03 g (3.66 mmol) of cis-2-(benzyloxy)-7-methyl-8-oxa-5-azaspiro[3.5]nonane hydrochloride [enantiomerically pure isomer 1 from Example 97A] in methanol (36.7 ml) and 3.34 ml of an aqueous 2 N hydrogen chloride solution were initially charged, 119 mg of palladium on carbon (10%) and 59.7 mg of palladium hydroxide on carbon (20%) were added under argon and the mixture was then stirred under an atmosphere of hydrogen at standard pressure overnight. The reaction solution was filtered through kieselguhr and the filter residue was washed with methanol. The filtrate was concentrated under reduced pressure and the product was dried under high vacuum. Yield: 785 mg (99% of theory).

MS (method 1C): m/z=158 [M+H-HCl]⁺;

¹H-NMR (400 MHz, DMSO-d₆): δ [ppm]=9.84 (br. s., 1H), 9.57 (br. s., 1H), 3.78-3.60 (m, 4H), 3.11 (d, 1H), 2.27-2.18 (m, 1H), 2.13-2.00 (m, 2H), 1.09 (d, 3H), three protons obscured

### Example 102A

### cis-7-Methyl-8-oxa-5-azaspiro[3.5]nonan-2-ol hydrochloride [enantiomerically pure isomer 2]

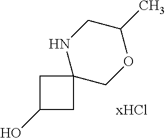

1.16 g (4.11 mmol) of cis-2-(benzyloxy)-7-methyl-8-oxa-5-azaspiro[3.5]nonane hydrochloride [enantiomerically pure isomer 2 from Example 98A] in methanol (41.3 ml) and 3.75 ml of an aqueous 2 N hydrogen chloride solution were initially charged, 134 mg of palladium on carbon (10%) and 67.1 mg of palladium hydroxide on carbon (20%) were added under argon and the mixture was then stirred under an atmosphere of hydrogen at standard pressure overnight. The reaction solution was filtered through kieselguhr and the filter residue was washed with methanol. The filtrate was concentrated under reduced pressure and the product was dried under high vacuum. Yield: 870 mg (98% of theory).

MS (method 1C): m/z=158 [M+H-HCl]⁺;

¹H-NMR (400 MHz, DMSO-d₆): δ [ppm]=9.96 (br. s., 1H), 9.67 (br. s., 1H), 3.84-3.59 (m, 4H), 3.10 (d, 1H), 2.29-2.17 (m, 1H), 2.15-1.99 (m, 2H), 1.09 (d, 3H), three protons obscured.

### Example 103A

### trans-7-Methyl-8-oxa-5-azaspiro[3.5]nonan-2-ol hydrochloride [enantiomerically pure isomer 1]

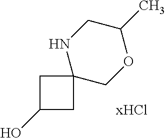

292 mg (1.03 mmol) of trans-2-(benzyloxy)-7-methyl-8-oxa-5-azaspiro[3.5]nonane hydrochloride [enantiomerically pure isomer 1 from Example 99A] in methanol (28.6 ml) and 5.73 ml of an aqueous 2 N hydrogen chloride solution were initially charged, 85.9 mg of palladium on carbon (10%) and 85.9 mg of palladium hydroxide on carbon (20%) were added under argon and the mixture was then stirred under an atmosphere of hydrogen at standard pressure overnight. The reaction solution was filtered through kieselguhr and the filter residue was washed with methanol. The filtrate was concentrated under reduced pressure and the product was dried under high vacuum. Yield: 132 mg (94% of theory).

LC-MS (method 1A): MS (ESIpos): m/z=158 [M+H-HCl]⁺.

¹H-NMR (400 MHz, DMSO-d₆): δ [ppm]=9.71-9.32 (m, 2H), 4.37 (m_{c}, 1H), 3.89 (d, 2H), 3.63 (d, 2H), 3.15 (d, 1H), 2.75-2.56 (m, 2H), 2.42-2.29 (m, 1H), 2.00 (d, 1H), 1.89-1.73 (m, 1H), 1.10 (d, 3H).

### Example 104A

### trans-7-Methyl-8-oxa-5-azaspiro[3.5]nonan-2-ol hydrochloride [enantiomerically pure isomer 2]

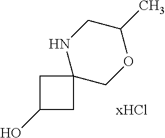

292 mg (1.03 mmol) of trans-2-(benzyloxy)-7-methyl-8-oxa-5-azaspiro[3.5]nonane hydrochloride [enantiomerically pure isomer 2 from Example 100A] in methanol (41.3 ml) and 3.75 ml of an aqueous 2 N hydrogen chloride solution were initially charged, 85.9 mg of palladium on carbon (10%) and 85.9 mg of palladium hydroxide on carbon (20%) were added under argon and the mixture was then stirred under an atmosphere of hydrogen at standard pressure overnight. The reaction solution was filtered through kieselguhr and the filter residue was washed with methanol. The filtrate was concentrated under reduced pressure and the product was dried under high vacuum. Yield: 231 mg (quant.).

MS (method 1C): m/z=158 [M+H-HCl]⁺;

¹H-NMR (400 MHz, DMSO-d₆): δ [ppm]=9.81-9.26 (m, 2H), 5.35 (br. s., 1H), 4.38 (m_{c}, 1H), 3.89 (d, 1H), 3.74-3.57 (m, 2H), 3.15 (d, 1H), 2.82-2.56 (m, 2H), 2.38 (ddd, 1H), 1.99 (m_{c}, 1H), 1.81 (m_{c}, 1H), 1.10 (d, 3H).

### Example 105A

### N-Benzyl-2-chloro-N-(1-hydroxy-2-methylpropan-2-yl)propanamide [racemate]

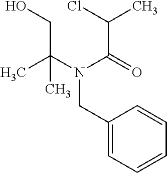 [SMILES: CC(Cl)C(=O)N(Cc1ccccc1)C(C)(C)CO]

20.0 g (106 mmol) of 2-(benzylamino)-2-methylpropan-1-ol [lit.: M. Le Hyaric et al., Chem. Biol. Drug Des. 2011, 78, 876-880] were initially charged in isopropanol (350 ml), the mixture was cooled to 0° C. and 22.6 g (31.1 ml, 223 mmol) of triethylamine were added. 15.6 g (12.2 ml, 123 mmol) of 2-chloropropionyl chloride [racemate] were then added dropwise. After 30 min of stirring, a further 7.09 g (5.55 ml, 55.9 mmol) of 2-chloropropionyl chloride [racemate] were added dropwise, and the reaction solution was allowed to warm to RT. The reaction solution was concentrated under reduced pressure and the residue was taken up in ethyl acetate (700 ml) and washed with water (400 ml). The organic phases were dried over sodium sulphate, filtered and concentrated under reduced pressure. The crude product was used without further purification in the next step. Yield: 37.1 g (quant.).

LC-MS (method 1A): Rₜ=0.95 min; MS (ESIpos): m/z=270 [M+H]⁺.

### Example 106A

### 4-Benzyl-2,5,5-trimethylmorpholin-3-one [racemate]

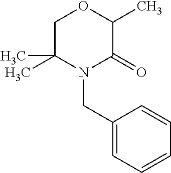 [SMILES: CC1OCC(C)(C)N(Cc2ccccc2)C1=O]

37.1 g (72.9 mmol, purity: 53%) of N-benzyl-2-chloro-N-(1-hydroxy-2-methylpropan-2-yl)propanamide [racemate] in isopropanol (500 ml) were cooled to 0° C., and 24.5 g (219 mmol) of potassium tert-butoxide were added in one portion. The mixture was stirred at 0° C. for 1 h and most of the isopropanol was then removed under reduced pressure. The residue was taken up in dichloromethane and washed with an 1 N aqueous hydrogen chloride solution (400 ml). The organic phase was dried over sodium sulphate, filtered and concentrated under reduced pressure. The crude product was used without further purification in the next step. Yield: 23.2 g (quant.).

LC-MS (method 1A): Rₜ=0.91 min; MS (ESIpos): m/z=234 [M+H]⁺.

### Example 107A

### 4-Benzyl-2,5,5-trimethylmorpholine [racemate]

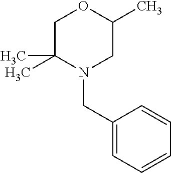 [SMILES: CC1CN(Cc2ccccc2)C(C)(C)CO1]

5.10 g (20.3 mmol) of 4-benzyl-2,5,5-trimethylmorpholin-3-one [racemate] were initially charged in tetrahydrofuran (200 ml), 30.5 ml (61.0 mmol) of 2 M borane/dimethyl sulphide complex solution in tetrahydrofuran were added under argon and the mixture was stirred under reflux for 2 h. The mixture was subsequently cooled to 0° C., ethanol (150 ml) was added carefully and the mixture was stirred under reflux for 2 h. The mixture was then concentrated under reduced pressure and the residue was purified by preparative RP-HPLC (acetonitrile/water, isocratic). Yield: 2.42 g (53% of theory).

LC-MS (method 4A): Rₜ=3.04 min; MS (ESIpos): m/z=236 [M+H]⁺;

¹H-NMR (400 MHz, DMSO-d₆): δ [ppm]=7.34-7.17 (m, 5H), 3.96 (d, 1H), 3.50-3.36 (m, 2H), 3.25 (d, 1H), 2.92 (d, 1H), 2.26 (dd, 1H), 2.04 (m_{c}, 1H), 1.04 (d, 6H), 0.98 (d, 3H).

### Example 108A

### 2,5,5-Trimethylmorpholine [racemate]

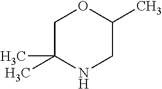

2.40 g (8.29 mmol) of 4-benzyl-2,5,5-trimethylmorpholine [racemate] were initially charged in methanol (80 ml), 240 mg of palladium on carbon (10%) and 120 mg of palladium hydroxide on carbon (20%) were added under argon and the mixture was then stirred under an atmosphere of hydrogen at standard pressure overnight. The reaction solution was filtered through kieselguhr and the filter residue was washed with methanol. The filtrate was concentrated under reduced pressure and the product was dried under high vacuum. Yield: 1.12 g (79% of theory).

¹H-NMR (400 MHz, DMSO-d₆): δ [ppm]=3.37 (d, 1H), 3.27 (m_{c}, 1H), 3.17 (s, 1H), 3.09 (dd, 1H), 1.78 (br. s., 1H), 1.08 (s, 3H), 1.01 (d, 3H), 0.87 (s, 3H), One proton not visible.

### Example 109A

### N-Benzyl-2-chloro-N-(1,3-dihydroxy-2-methylpropan-2-yl)propanamide [racemate]

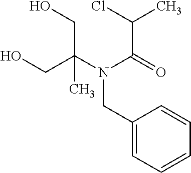 [SMILES: CC(Cl)C(=O)N(Cc1ccccc1)C(C)(CO)CO]

10.7 g (54.8 mmol) of 2-(benzylamino)-2-methylpropane-1,3-diol [lit.: J. Cossy et al., J. Org. Chem. 2012, 77, 6087-6099] were initially charged in dichloromethane (400 ml), the mixture was cooled to 0° C. and 8.32 g (11.5 ml, 82.2 mmol) of triethylamine were added. 8.35 g (6.52 ml, 65.8 mmol) of 2-chloropropionyl chloride [racemate] were then added dropwise. After 30 min of stirring, a further 5.57 g (3.70 ml, 37.3 mmol) of 2-chloropropionyl chloride [racemate] were added dropwise, and the reaction solution was allowed to warm to RT. The reaction solution was concentrated under reduced pressure, and the residue was taken up in 1 N aqueous hydrogen chloride solution and extracted repeatedly with dichloromethane. The organic phases were dried over sodium sulphate, filtered and concentrated under reduced pressure. The crude product was used without further purification in the next step. Yield: 15.6 g (99% of theory).

LC-MS (method 1A): Rₜ=0.58 min; MS (ESIpos): m/z=286 [M+H]⁺.

### Example 110A

### 4-Benzyl-5-(hydroxymethyl)-2,5-dimethylmorpholin-3-one [diastereomer mixture, 4 isomers]

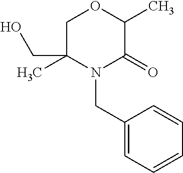 [SMILES: CC1OCC(C)(CO)N(Cc2ccccc2)C1=O]

15.7 g (54.8 mmol) of N-benzyl-2-chloro-N-(1,3-dihydroxy-2-methylpropan-2-yl)propanamide [racemate] in isopropanol (300 ml) were cooled to 0° C., and 24.6 g (219 mmol) of potassium tert-butoxide were added in one portion. The reaction was stirred overnight and allowed to warm to RT during this time. Most of the isopropanol was then removed under reduced pressure. The residue was taken up in 2 N aqueous hydrogen chloride solution and extracted repeatedly with dichloromethane. The organic phase was dried over sodium sulphate, filtered and concentrated under reduced pressure. The crude product was used without further purification in the next step. Yield: 15.2 g (96% of theory, diastereomer ratio: about 1:1).

LC-MS (Method 1A): Rₜ=0.72 min (racemic diastereomer 1), Rₜ=0.74 min (racemic diastereomer 2); MS (ESIpos): m/z=250 [M+H]⁺.

### Example 111A

### (4-Benzyl-3,6-dimethylmorpholin-3-yl)methanol [diastereomer mixture, 4 isomers]

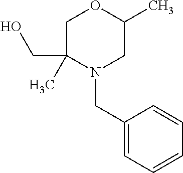 [SMILES: CC1CN(Cc2ccccc2)C(C)(CO)CO1]

15.6 g (62.6 mmol) of 4-benzyl-5-(2-hydroxymethyl)-2,5-dimethylmorpholin-3-one [diastereomer mixture, 4 isomers] were initially charged in tetrahydrofuran (300 ml), 93.9 ml (188 mmol) of 2 M borane/dimethyl sulphide complex solution in tetrahydrofuran were added under argon and the mixture was stirred under reflux for 3 h. The mixture was subsequently cooled to RT, methanol (150 ml) was added carefully and the mixture was stirred under reflux for 4 h. The mixture was then concentrated completely under reduced pressure and the residue was purified by preparative RP-HPLC (acetonitrile/water). Yield: 6.53 g (44% of theory).

LC-MS (method 4A): Rₜ=0.28 min; MS (ESIpos): m/z=236 [M+H]⁺.

### Example 112A

### (4-Benzyl-3,6-dimethylmorpholin-3-yl)methanol [enantiomerically pure isomer 1]

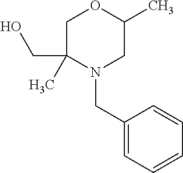 [SMILES: CC1CN(Cc2ccccc2)C(C)(CO)CO1]

Enantiomer separation on a chiral phase of 6.53 g of the compound from Example 111A according to Method 18D and re-purification by preparative RP-HPLC (acetonitrile/water) gave 1.12 g of Example 112A (enantiomerically pure isomer 1), 1.23 g of Example 113A (enantiomerically pure isomer 2), 441 mg of Example 114A (enantiomerically pure isomer 3) and 457 mg of Example 115A (enantiomerically pure isomer 4).

HPLC (Method 18E): Rₜ=5.13 min, >99.0% ee;

LC-MS (method 4A): Rₜ=2.56 min; MS (ESIpos): m/z=236 [M+H]⁺.

### Example 113A

### (4-Benzyl-3,6-dimethylmorpholin-3-yl)methanol [enantiomerically pure isomer 2]

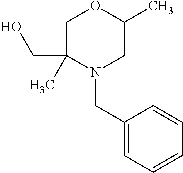 [SMILES: CC1CN(Cc2ccccc2)C(C)(CO)CO1]

Enantiomer separation on a chiral phase of 6.53 g of the compound from Example 111A according to Method 18D and re-purification by preparative RP-HPLC (acetonitrile/water) gave 1.12 g of Example 112A (enantiomerically pure isomer 1), 1.23 g of Example 113A (enantiomerically pure isomer 2), 441 mg of Example 114A (enantiomerically pure isomer 3) and 457 mg of Example 115A (enantiomerically pure isomer 4).

HPLC (Method 18E): Rₜ=5.73 min, >99.0% ee;

LC-MS (method 4A): Rₜ=2.52 min; MS (ESIpos): m/z=236 [M+H]⁺.

### Example 114A

### (4-Benzyl-3,6-dimethylmorpholin-3-yl)methanol [enantiomerically pure isomer 3]

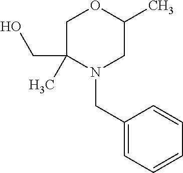 [SMILES: CC1CN(Cc2ccccc2)C(C)(CO)CO1]

Enantiomer separation on a chiral phase of 6.53 g of the compound from Example 111A according to Method 18D and re-purification by preparative RP-HPLC (acetonitrile/water) gave 1.12 g of Example 112A (enantiomerically pure isomer 1), 1.23 g of Example 113A (enantiomerically pure isomer 2), 441 mg of Example 114A (enantiomerically pure isomer 3) and 457 mg of Example 115A (enantiomerically pure isomer 4).

HPLC (Method 18E): Rₜ=6.57 min, >99.0% ee;

LC-MS (method 4A): Rₜ=2.60 min; MS (ESIpos): m/z=236 [M+H]⁺.

### Example 115A

### (4-Benzyl-3,6-dimethylmorpholin-3-yl)methanol [enantiomerically pure isomer 4]

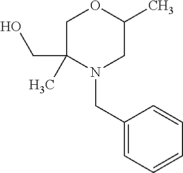 [SMILES: CC1CN(Cc2ccccc2)C(C)(CO)CO1]

Enantiomer separation on a chiral phase of 6.53 g of the compound from Example 111A according to Method 18D and re-purification by preparative RP-HPLC (acetonitrile/water) gave 1.12 g of Example 112A (enantiomerically pure isomer 1), 1.23 g of Example 113A (enantiomerically pure isomer 2), 441 mg of Example 114A (enantiomerically pure isomer 3) and 457 mg of Example 115A (enantiomerically pure isomer 4).

HPLC (Method 18E): Rₜ=6.92 min, >99.0% ee;

LC-MS (method 4A): Rₜ=2.61 min; MS (ESIpos): m/z=236 [M+H]⁺.

### Example 116A

### (3,6-Dimethylmorpholin-3-yl)methanol [enantiomerically pure isomer 1]

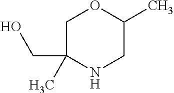

1.12 g (4.76 mmol) of (4-benzyl-3,6-dimethylmorpholin-3-yl)methanol [enantiomerically pure isomer 1, Example 112A] were initially charged in ethanol (120 ml), 112 mg of palladium on carbon (10%) and 112 mg of palladium hydroxide on carbon (20%) were added under argon and the mixture was then stirred under an atmosphere of hydrogen at standard pressure overnight. The reaction solution was filtered through kieselguhr and the filter residue was washed with methanol. The filtrate was concentrated under reduced pressure and the product was dried under high vacuum. Yield: 651 mg (94% of theory).

¹H-NMR (400 MHz, DMSO-d₆): δ [ppm]=4.44 (br. s., 1H), 3.56 (d, 1H), 3.40 (d, 2H), 3.36-3.22 (m, 2H), 3.06 (d, 1H), 1.98 (br. s., 1H), 0.99 (d, 3H), 0.77 (s, 3H), one proton not visible.

### Example 117A

### (3,6-Dimethylmorpholin-3-yl)methanol [enantiomerically pure isomer 4]

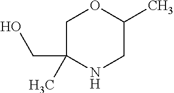

457 g (1.94 mmol) of (4-benzyl-3,6-dimethylmorpholin-3-yl)methanol [enantiomerically pure isomer 4, Example 115A] were initially charged in ethanol (50 ml), 46 mg of palladium on carbon (10%) and 46 mg of palladium hydroxide on carbon (20%) were added under argon and the mixture was then stirred under an atmosphere of hydrogen at standard pressure overnight. The reaction solution was filtered through kieselguhr and the filter residue was washed with methanol. The filtrate was concentrated under reduced pressure and the product was dried under high vacuum. Yield: 280 mg (99% of theory).

¹H-NMR (400 MHz, DMSO-d₆): δ [ppm]=4.62 (br. s., 1H), 3.40 (d, 1H), 3.30-3.18 (m, 2H), 3.11 (br. s., 2H), 2.58-2.55 (m, 1H), 1.81 (m_{c}, 1H), 1.05-0.96 (m, 6H), One proton not visible.

### Example 118A

### 2-Allyl-4-benzyl-2,5,5-trimethylmorpholin-3-one [racemate]

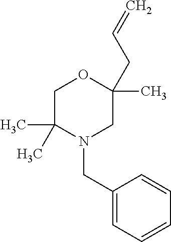 [SMILES: C=CCC1(C)CN(Cc2ccccc2)C(C)(C)CO1]

21.58 g (92.1 mmol) of 4-benzyl-2,5,5-trimethylmorpholin-3-one [racemate] were initially charged in tetrahydrofuran (1.19 l), 129 ml (129 mmol) of 1 M lithium hexamethyldisilazide solution in tetrahydrofuran were added under argon and at −78° C. and the mixture was stirred for 15 min. Subsequently, at −78° C., 23.2 g (12.6 ml, 138 mmol) of allyl iodide were added, and the mixture was warmed to RT and stirred overnight. The mixture was once more cooled to −78° C., 92.1 ml (92.1 mmol) of a 1 M solution of lithiumhexamethyldisilazide in tetrahydrofuran were added and the reaction mixture was then stirred for 30 min. A further 15.5 g (8.42 ml, 92.1 mmol) of allyl iodide were then added and the mixture was warmed to RT. The reaction was terminated by addition of saturated aqueous ammonium chloride solution and the mixture was then extracted with ethyl acetate. The organic phase was washed with saturated aqueous sodium chloride solution, dried over sodium sulphate, filtered and concentrated under reduced pressure. The crude product was used without further purification in the next step. Yield: 23.4 g (93% of theory).

LC-MS (method 1A): Rₜ=1.09 min; MS (ESIpos): m/z=274 [M+H]⁺.

### Example 119A

### (4-Benzyl-2,5,5-trimethyl-3-oxomorpholin-2-yl)acetaldehyde [racemate]

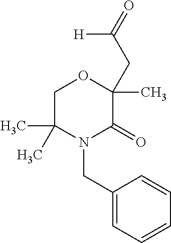 [SMILES: CC1(CC=O)OCC(C)(C)N(Cc2ccccc2)C1=O]

At 0° C., 4.00 ml (1.47 mmol) of a 2.5% strength solution of osmium tetroxide in tert-butanol and 47.1 g (220 mmol) of sodium periodate were added to 23.6 g (73.4 mmol) of (2-allyl-4-benzyl-2,5,5-trimethylmorpholin-3-one [racemate] in tetrahydrofuran (570 ml) and water (340 ml). The mixture was then allowed to warm to RT and stirred overnight. The reaction solution was filtered through kieselguhr and the filter residue was washed with tetrahydrofuran. The mixture was taken up in ethyl acetate and diluted with water. After separation of the phases, the organic phase was washed with 1 N aqueous sodium sulphite solution (2×800 ml), dried over magnesium sulphate, filtered and concentrated under reduced pressure. The crude product was used without further purification in the next step. Yield: 19.8 g of crude product.

LC-MS (method 1A): Rₜ=0.93 min; MS (ESIpos): m/z=276 [M+H]⁺.

### Example 120A

### 4-Benzyl-2-(2-hydroxyethyl)-2,5,5-trimethylmorpholin-3-one [racemate]

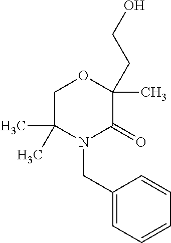 [SMILES: CC1(CCO)OCC(C)(C)N(Cc2ccccc2)C1=O]

At 0° C., 2.68 g (70.8 mmol) of sodium borohydride were added to 6.50 g (about 23.6 mmol, crude product) of (4-benzyl-2,5,5-trimethyl-3-oxomorpholin-2-yl)acetaldehyde [racemate] in methanol (176 ml). The mixture was then allowed to warm to RT and stirred for 30 min. Water was then added to the reaction solution, most of the methanol was removed under reduced pressure and the residue was extracted with ethyl acetate. The organic phase was dried over sodium sulphate, filtered and concentrated under reduced pressure. The crude product was purified by preparative RP-HPLC (acetonitrile/water). Yield: 5.89 g (62% of theory, purity: 69%).

LC-MS (method 4A): Rₜ=2.31 min; MS (ESIpos): m/z=278 [M+H]⁺.

### Example 121A

### 2-(4-Benzyl-2,5,5-trimethylmorpholin-2-yl)ethanol [racemate]

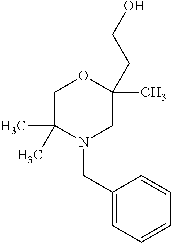 [SMILES: CC1(CCO)CN(Cc2ccccc2)C(C)(C)CO1]

5.85 g (21.1 mmol) of 4-benzyl-2-(2-hydroxyethyl)-2,5,5-trimethylmorpholin-3-one [racemate] were initially charged in tetrahydrofuran (210 ml), 42.2 ml (84.4 mmol) of 2 M borane/dimethyl sulphide complex solution in tetrahydrofuran were added under argon and the mixture was stirred under reflux for 2 h. The mixture was subsequently cooled to 0° C., methanol (45 ml) was added carefully and the mixture was stirred under reflux for 30 min to destroy any boron complexes. The mixture was then concentrated completely under reduced pressure, and the residue was taken up in acetonitrile and purified directly by preparative RP-HPLC (acetonitrile/water). Yield: 3.10 g (55% of theory).

LC-MS (method 4A): Rₜ=2.82 min; MS (ESIpos): m/z=264 [M+H]⁺.

### Example 122A

### 2-(2,5,5-Trimethylmorpholin-2-yl)ethanol [racemate]

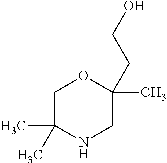

3.00 g (11.4 mmol) of 2-(4-benzyl-2,5,5-trimethylmorpholin-2-yl)ethanol [racemate] were initially charged in ethanol (115 ml), 286 mg of palladium on carbon (10%) and 143 mg of palladium hydroxide on carbon (20%) were added under argon and the mixture was stirred under an atmosphere of hydrogen at standard pressure overnight. Subsequently, a further 286 mg of palladium on carbon (10%) and 143 mg of palladium hydroxide on carbon (20%) were added, and the mixture was once more stirred under an atmosphere of hydrogen at standard pressure overnight. The reaction solution was filtered through kieselguhr and the filter residue was washed with ethanol. The filtrate was concentrated under reduced pressure and the product was dried under high vacuum. Yield: 2.06 g (quant.).

MS (method 1C): m/z=174 [M+H]⁺.

### Example 123A

### 4-Benzyl-2-(2-hydroxypropyl)-2,5,5-trimethylmorpholin-3-one [diastereomer mixture, 4 isomers]

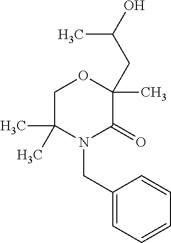 [SMILES: CC(O)CC1(C)OCC(C)(C)N(Cc2ccccc2)C1=O]

6.50 g (about 23.6 mmol, crude product) of (4-benzyl-2,5,5-trimethyl-3-oxomorpholin-2-yl)acetaldehyde [racemate] were initially charged in tetrahydrofuran (101 ml), and 28.3 ml (28.3 mmol) of methylmagnesium bromide (2 M solution in tetrahydrofuran) were added slowly at −78° C. The mixture was stirred at −78° C. for 15 min and then allowed to warm to RT. The reaction was terminated by addition of saturated aqueous ammonium chloride solution (about 70 ml) and the tetrahydrofuran was removed under reduced pressure. The residue was taken up in dichloromethane and washed with water. The organic phase was dried over sodium sulphate, filtered and concentrated under reduced pressure. The crude product was used without further purification in the next step. Yield: 6.17 g (54% of theory).

LC-MS (method 4A): Rₜ=2.46 min; MS (ESIpos): m/z=292 [M+H]⁺.

### Example 124A

### 2-(4-Benzyl-2,5,5-trimethylmorpholin-2-yl)propan-2-ol [diastereomer 1, 2 isomers+diastereomer 2, 2 isomers]

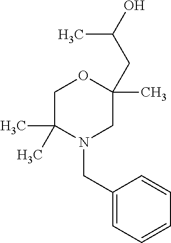 [SMILES: CC(O)CC1(C)CN(Cc2ccccc2)C(C)(C)CO1]

6.15 g (21.1 mmol) of 4-benzyl-2-(2-hydroxypropyl)-2,5,5-trimethylmorpholin-3-one [diastereomer mixture, 4 isomers] were initially charged in tetrahydrofuran (210 ml), 42.2 ml (84.4 mmol) of 2 M borane/dimethyl sulphide complex solution in tetrahydrofuran were added under argon and the mixture was stirred under reflux for 2 h. The mixture was subsequently cooled to 0° C., methanol (45 ml) was added carefully and the mixture was stirred under reflux for 30 min. The mixture was subsequently concentrated completely under reduced pressure, and the residue was taken up in acetonitrile and subjected directly to purification and diastereomer separation by preparative RP-HPLC (acetonitrile/water, isocratic). A mixed fraction (1.10 g) was re-purified on an achiral phase according to Method 3F. Yield: 1.74 g (29% of theory, diastereomer 1, 2 isomers) and 434 mg (26% of theory, diastereomer 2, 2 isomers).

Diastereomer 1 (2 isomers):

LC-MS (method 4A): Rₜ=3.06 min; MS (ESIpos): m/z=279 [M+H]⁺.

Diastereomer 2 (2 isomers):

LC-MS (method 4A): Rₜ=3.18 min; MS (ESIpos): m/z=279 [M+H]⁺.

### Example 125A

### 1-(2,5,5-Trimethylmorpholin-2-yl)propan-2-ol [diastereomer 1, 2 isomers]

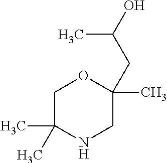

3.00 g (11.4 mmol) of 2-(4-benzyl-2,5,5-trimethylmorpholin-2-yl)propan-2-ol [diastereomer 1, 2 isomers, Example 124A] were initially charged in ethanol (54.0 ml), 135 mg of palladium on carbon (10%) and 67.0 mg of palladium hydroxide on carbon (20%) were added under argon, and the mixture was then stirred under an atmosphere of hydrogen at standard pressure overnight. The reaction solution was filtered through kieselguhr and the filter residue was washed with ethanol. The filtrate was concentrated under reduced pressure and the product was dried under high vacuum. Yield: 1.09 g (quant.).

MS (method 1C): m/z=188 [M+H]⁺.

### Example 126A

### 1-(2,5,5-Trimethylmorpholin-2-yl)propan-2-ol [diastereomer 2, 2 isomers]

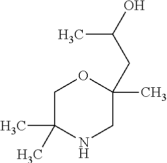

464 g (1.67 mmol) of 2-(4-benzyl-2,5,5-trimethylmorpholin-2-yl)propan-2-ol [diastereomer 2, 2 isomers, Example 124A] were initially charged in ethanol (16.8 ml), 42.0 mg of palladium on carbon (10%) and 21.0 mg of palladium hydroxide on carbon (20%) were added under argon, and the mixture was stirred under an atmosphere of hydrogen at standard pressure overnight. The reaction solution was filtered through kieselguhr and the filter residue was washed with ethanol. The filtrate was concentrated under reduced pressure and the product was dried under high vacuum. Yield: 341 mg (quant.).

GC-MS (method 1B): Rₜ=3.89 min; MS (ESIpos): m/z=287 [M]⁺.

### Example 127A

### N-Benzyl-2-chloro-N-(1,3-dihydroxypropan-2-yl)propanamide [racemate]

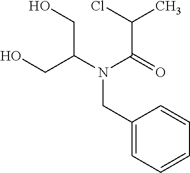 [SMILES: CC(Cl)C(=O)N(Cc1ccccc1)C(CO)CO]

60.5 g (334 mmol) of 2-(benzylamino)propane-1,3-diol [lit.: W. Lacôte et al., Org. Lett. 2011, 13, 5990-5993] in isopropanol (0.93 l) were cooled to 0° C. and 50.7 g (69.8 ml, 501 mmol) of triethylamine were added. 50.9 g (38.9 ml, 401 mmol) of 2-chloropropionyl chloride [racemate] were then added dropwise. The reaction solution was allowed to warm to RT and the reaction solution was concentrated under reduced pressure. 0.5 N aqueous hydrogen chloride solution was added to the residue, and the mixture was extracted with dichloromethane. The organic phases were dried over sodium sulphate, filtered and concentrated under reduced pressure. The crude product was used without further purification in the next step. Yield: 91.7 g (94% of theory).

LC-MS (method 1A): Rₜ=0.71 min; MS (ESIpos): m/z=272 [M+H]⁺.

### Example 128A

### 4-Benzyl-5-(hydroxymethyl)-2-methylmorpholin-3-one [diastereomer mixture, 4 isomers]

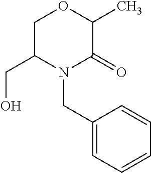 [SMILES: CC1OCC(CO)N(Cc2ccccc2)C1=O]

81.3 g (272 mmol, purity: 91%) of N-benzyl-2-chloro-N-(1,3-dihydroxypropan-2-yl)propanamide [racemate] in isopropanol (600 ml) were cooled to 0° C., and 91.6 g (817 mmol) of potassium tert-butoxide were added in one portion. The mixture was allowed to slowly warm to RT and stirred overnight. The isopropanol was removed under reduced pressure and the residue was taken up in dichloromethane. The mixture was washed with water and the organic phase was dried over magnesium sulphate, filtered and concentrated under reduced pressure. The crude product was used without further purification in the next step. Yield: 61.7 g (96% of theory, diastereomer ratio about 7:3).

LC-MS (Method 2A): Rₜ=0.61 min (diastereomer 1, 2 isomers), Rₜ=0.62 min (diastereomer 2, 2 isomers);

MS (ESIpos): m/z=236 [M+H]⁺.

### Example 129A

### 4-Benzyl-6-methyl-5-oxomorpholine-3-carboxylic acid [diastereomer mixture, 4 isomers]

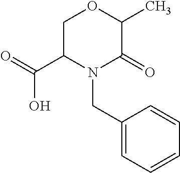 [SMILES: CC1OCC(C(=O)O)N(Cc2ccccc2)C1=O]

20.0 g (85.0 mmol) of 4-benzyl-5-(hydroxymethyl)-2-methylmorpholin-3-one [diastereomer mixture, 4 isomers] were initially charged in acetonitrile (1.50 l), 42.6 g (187 mmol) of periodic acid were added at RT and the mixture was stirred for 15 min. The mixture was then cooled to 0° C., and 733 mg (3.40 mmol) of pyridinium chlorochromate in acetonitrile (30 ml) were added. The mixture was stirred at 0° C. for 2 h and the reaction solution was then concentrated under reduced pressure to about 50 ml. Water (1.00 l) was added and the mixture was extracted with ethyl acetate. The organic phase was dried over sodium sulphate, filtered and concentrated under reduced pressure. The crude product was used without further purification in the next step. Yield: 21.4 g (60% of theory, purity: 60%).

LC-MS (method 1A): Rₜ=0.65 min; MS (ESIpos): m/z=250 [M+H]⁺.

### Example 130A

### Methyl 4-benzyl-6-methyl-5-oxomorpholin-3-carboxylate [diastereomer mixture, 4 isomers]

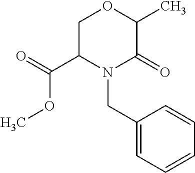 [SMILES: COC(=O)C1COC(C)C(=O)N1Cc1ccccc1]

21.3 g (crude product) of 4-benzyl-6-methyl-5-oxomorpholine-3-carboxylic acid [diastereomer mixture, 4 isomers] in methanol (500 ml) were cooled to 0° C., and 12.5 ml (171 mmol) of thionyl chloride were added slowly. The reaction mixture was stirred under reflux for 2 h and then concentrated completely under reduced pressure. The crude product was used without further purification in the next step. Yield: 19.8 g (88% of theory, crude product).

LC-MS (method 1A): Rₜ=0.80 min; MS (ESIpos): m/z=264 [M+H]⁺.

### Example 131A

### 4-Benzyl-5-(2-hydroxypropan-2-yl)-2-methylmorpholin-3-one [racemate]

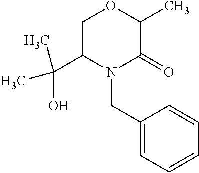 [SMILES: CC1OCC(C(C)(C)O)N(Cc2ccccc2)C1=O]

4.00 g (about 15.2 mmol, crude product) of methyl 4-benzyl-6-methyl-5-oxomorpholin-3-carboxylate [diastereomer mixture, 4 isomers] were initially charged in tetrahydrofuran (55.8 ml), and 53.2 ml (53.2 mmol) of a 1 M solution of methylmagnesium bromide in tetrahydrofuran were added at −78° C. The mixture was stirred at −78° C. for 15 min and then allowed to warm to RT. Saturated aqueous ammonium chloride solution (70 ml) was then added carefully to the reaction solution, most of the tetrahydrofuran was removed under reduced pressure and the residue was taken up in dichloromethane. After separation of the phases, the organic phase was washed with water, dried over sodium sulphate, filtered and concentrated under reduced pressure. The crude product was purified by preparative RP-HPLC (acetonitrile/water). Yield: 968 mg (24% of theory); at this or any of the preceding stages, there was complete epimerization to one of the two possible diastereomers.

LC-MS (method 4A): Rₜ=0.79 min; MS (ESIpos): m/z=264 [M+H]⁺.

### Example 132A

### 2-(4-Benzyl-6-methylmorpholin-3-yl)propan-2-ol [racemate]

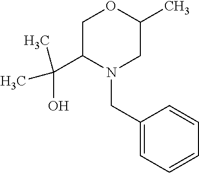 [SMILES: CC1CN(Cc2ccccc2)C(C(C)(C)O)CO1]

967 mg (3.67 mmol) of 4-benzyl-5-(2-hydroxypropan-2-yl)-2-methylmorpholin-3-one [racemate] were initially charged in tetrahydrofuran (36.1 ml), 7.34 ml (14.7 mmol) of 2 M borane/dimethyl sulphide complex solution in tetrahydrofuran were added under argon and the mixture was stirred under reflux for 2 h. The mixture was subsequently cooled to 0° C., methanol (10 ml) was added carefully and the mixture was stirred under reflux for 30 min. The mixture was then concentrated completely under reduced pressure, and the residue was taken up in acetonitrile and purified directly by preparative RP-HPLC (acetonitrile/water). Yield: 433 mg (47% of theory).

LC-MS (method 1A): Rₜ=0.44 min; MS (ESIpos): m/z=250 [M+H]⁺.

### Example 133A

### 2-(4-Benzyl-6-methylmorpholin-3-yl)propan-2-ol [enantiomerically pure isomer 1]

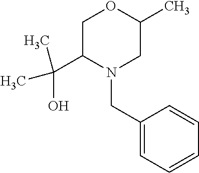 [SMILES: CC1CN(Cc2ccccc2)C(C(C)(C)O)CO1]

Enantiomer separation on a chiral phase of 433 mg of the compound from Example 132A according to Method 22D gave 179 mg of Example 133A (enantiomerically pure isomer 1) and 183 mg of Example 134A (enantiomerically pure isomer 2).

HPLC (Method 18E): Rₜ=5.50 min, 99.0% ee;

LC-MS (method 1A): Rₜ=0.43 min; MS (ESIpos): m/z=250 [M+H]⁺.

### Example 134A

### 2-(4-Benzyl-6-methylmorpholin-3-yl)propan-2-ol [enantiomerically pure isomer 2]

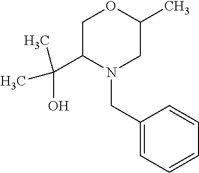 [SMILES: CC1CN(Cc2ccccc2)C(C(C)(C)O)CO1]

Enantiomer separation on a chiral phase of 433 mg of the compound from Example 132A according to Method 22D gave 179 mg of Example 133A (enantiomerically pure isomer 1) and 183 mg of Example 134A (enantiomerically pure isomer 2).

HPLC (Method 18E): Rₜ=6.88 min, 99.0% ee;

LC-MS (method 1A): Rₜ=0.44 min; MS (ESIpos): m/z=250 [M+H]⁺.

### Example 135A

### 2-(6-Methylmorpholin-3-yl)propan-2-ol [enantiomerically pure isomer 1]

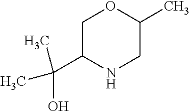

179 mg (0.718 mmol) of 2-(4-benzyl-6-methylmorpholin-3-yl)propan-2-ol [enantiomerically pure isomer 1, Example 133A] were initially charged in ethanol (7.22 ml), 20.9 mg of palladium on carbon (10%) and 10.5 mg of palladium hydroxide on carbon (20%) were added under argon, and the mixture was then stirred under an atmosphere of hydrogen at standard pressure overnight. The reaction solution was filtered through kieselguhr and the filter residue was washed with ethanol. The filtrate was concentrated under reduced pressure and the product was dried under high vacuum. Yield: 94.4 mg (82% of theory).

MS (method 1C): m/z=160 [M+H]⁺.

### Example 137A

### N-Benzyl-2-chloro-N-[(2R)-1-hydroxypropan-2-yl]propanamide [diastereomer mixture, 2 isomers]

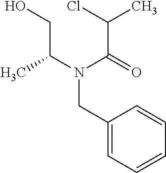 [SMILES: CC(Cl)C(=O)N(Cc1ccccc1)[C@H](C)CO]

16.4 g (99.3 mmol) of (2R)-2-(benzylamino)propan-1-ol [lit.: T. J. Tewson et al., Synthesis 2002, 6, 766-770] in isopropanol (500 ml) were cooled to 0° C., and 20.1 g (27.7 ml, 199 mmol) of triethylamine were added. 13.9 g (10.8 ml, 109 mmol) of 2-chloropropionyl chloride [racemate] were then added dropwise, and the reaction solution was allowed to warm to RT. The reaction solution was stirred overnight and then concentrated under reduced pressure. 0.5 N aqueous hydrogen chloride solution was added to the residue, and the mixture was extracted with ethyl acetate. The organic phases were dried over sodium sulphate, filtered and concentrated under reduced pressure. The crude product was used without further purification in the next step. Yield: 24.3 g (88% of theory, purity: 92%, diastereomer ratio about 1:1).

LC-MS (Method 1A): Rₜ=0.80 min (enantiomerically pure isomer 1), Rₜ=0.84 min (enantiomerically pure isomer 2);

MS (ESIpos): m/z=256 [M+H]⁺.

### Example 138A

### (5R)-4-Benzyl-2,5-dimethylmorpholin-3-one [diastereomer mixture, 2 isomers]

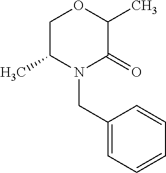 [SMILES: CC1OC[C@@H](C)N(Cc2ccccc2)C1=O]

30.0 g (109 mmol, purity: 93%) of N-benzyl-2-chloro-N-[(2R)-1-dihydroxypropan-2-yl]propanamide [diastereomer mixture, 2 isomers] were initially charged in isopropanol (588 ml), the mixture was cooled to 0° C. and 49.0 g (436 mmol) of potassium tert-butoxide were then added in one portion. The mixture was allowed to slowly warm to RT and stirred overnight. Most of the isopropanol was removed under reduced pressure and the residue was taken up in water. The mixture was extracted with ethyl acetate, and the organic phases were dried over magnesium sulphate, filtered and concentrated under reduced pressure. The crude product was used without further purification in the next step. Yield: 22.8 g (93% of theory).

LC-MS (method 1A): Rₜ=0.85 min; MS (ESIpos): m/z=220 [M+H]⁺.

### Example 139A

### (5R)-2-Allyl-4-benzyl-2,5-dimethylmorpholin-3-one [diastereomer mixture, 2 isomers]

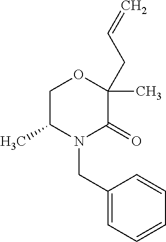 [SMILES: C=CCC1(C)OC[C@@H](C)N(Cc2ccccc2)C1=O]

22.8 g (104 mmol) of (5R)-4-benzyl-2,5-dimethylmorpholin-3-one [diastereomer mixture, 2 isomers] were initially charged in tetrahydrofuran (1.34 l), 146 ml (146 mmol) of 1 M lithium hexamethyldisilazide solution in tetrahydrofuran were added under argon and at −78° C. and the mixture was stirred for 15 min. Subsequently, at −78° C., 21.0 g (11.4 ml, 125 mmol) of allyl iodide were added, and the reaction mixture was warmed to RT and stirred for 3 h. The reaction was terminated by addition of saturated aqueous ammonium chloride solution and the mixture was then extracted with ethyl acetate. The organic phase was washed with saturated aqueous sodium chloride solution, dried over sodium sulphate, filtered and concentrated under reduced pressure. The crude product was used without further purification in the next step. Yield: 27.5 g (77% of theory, purity: 75%).

LC-MS (method 1A): Rₜ=0.99 min; MS (ESIpos): m/z=260 [M+H]⁺.

### Example 140A

[(5R)-4-Benzyl-2,5-dimethyl-3-oxomorpholin-2-yl]acetaldehyde [diastereomer mixture, 2 isomers]

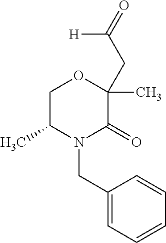 [SMILES: C[C@@H]1COC(C)(CC=O)C(=O)N1Cc1ccccc1]

At 0° C., 4.35 ml (1.60 mmol) of a 2.5% solution of osmium tetroxide in tert-butanol and 51.2 g (240 mmol) of sodium periodate were added to 27.4 g (79.9 mmol, purity: 75%) of (5R)-2-allyl-4-benzyl-2,5-dimethylmorpholin-3-one [diastereomer mixture, 2 isomers] in tetrahydrofuran (620 ml) and water (370 ml). The mixture was then allowed to warm to RT and stirred overnight. The reaction solution was filtered through kieselguhr and the filter residue was washed with tetrahydrofuran. The reaction solution was taken up in ethyl acetate and diluted with water. After separation of the phases, the organic phase was washed with 1 N aqueous sodium sulphite solution (2×400 ml), dried over sodium sulphate, filtered and concentrated under reduced pressure. The crude product was used without further purification in the next step. Yield: 23.6 g of crude product.

LC-MS (Method 1A): Rₜ=0.81 min (enantiomerically pure isomer 1), Rₜ=0.84 min (enantiomerically pure isomer 2);

MS (ESIpos): m/z=262 [M+H]⁺.

### Example 141A

### (5R)-4-Benzyl-2-(2-hydroxypropyl)-2,5-dimethylmorpholin-3-one [diastereomer mixture, 4 isomers]

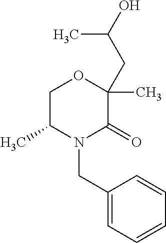 [SMILES: CC(O)CC1(C)OC[C@@H](C)N(Cc2ccccc2)C1=O]

16.8 g (about 64.3 mmol, crude product) of [(5R)-4-benzyl-2,5-dimethyl-3-oxomorpholin-2-yl]acetaldehyde [Example 140A, diastereomer mixture, 2 isomers] were initially charged in tetrahydrofuran (275 ml), and 77.2 ml (77.2 mmol) of a 1 M solution of methylmagnesium bromide in tetrahydrofuran were added at −78° C. The mixture was stirred at −78° C. for 15 min and then allowed to warm to RT. Saturated aqueous ammonium chloride solution (400 ml) was then added carefully to the reaction solution, most of the tetrahydrofuran was removed under reduced pressure and the residue was taken up in dichloromethane. After separation of the phases, the organic phase was washed with water, dried over sodium sulphate, filtered and concentrated under reduced pressure. The crude product was used without further purification in the next step. Yield: 16.2 g of crude product.

LC-MS (method 1A): Rₜ=0.78, 0.80 min; MS (ESIpos): m/z=278 [M+H]⁺.

### Example 142A

### 1-[(5R)-4-Benzyl-2,5-dimethylmorpholin-2-yl]propan-2-ol [enantiomerically pure diastereomers 1+2+3+4]

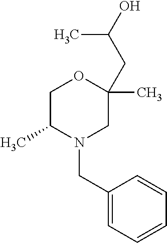 [SMILES: CC(O)CC1(C)CN(Cc2ccccc2)[C@H](C)CO1]

16.2 g (about 39.1 mmol, crude product) of (5R)-4-benzyl-2-(2-hydroxypropyl)-2,5-dimethylmorpholin-3-one [diastereomer mixture, 4 isomers] were initially charged in tetrahydrofuran (397 ml), 78.3 ml (157 mmol) of 2 M borane/dimethyl sulphide complex solution in tetrahydrofuran were added under argon and the mixture was stirred under reflux for 2 h. The mixture was subsequently cooled to 0° C., methanol (80 ml) was added carefully and the mixture was then stirred under reflux for 30 min. The mixture was subsequently concentrated completely under reduced pressure, and the residue was taken up in acetonitrile and subjected directly to purification and diastereomer separation by preparative RP-HPLC (acetonitrile/water, isocratic). Here, the target compound eluted as third component. Yield: target compound (enantiomerically pure isomer 3): 3.11 g (29% of theory); enantiomerically pure isomer 1: 2.12 g (20% of theory); enantiomerically pure isomer 2: 506 mg (5% of theory); enantiomerically pure isomer 4: 1.72 g (16% of theory).

Enantiomerically pure isomer 3 (target compound):

LC-MS (method 1A): Rₜ=0.39 min; MS (ESIpos): m/z=264 [M+H]⁺;

¹H-NMR (400 MHz, DMSO-d₆): δ [ppm]=7.34-7.18 (m, 5H), 4.10 (d, 1H), 3.96 (d, 1H), 3.79 (m_{c}, 1H), 3.48 (dd, 1H), 3.36 (m_{c}, 1H), 3.04 (d, 1H), 2.46 (d, 1H), 2.28 (m_{c}, 1H), 1.88 (d, 1H), 1.44 (dd, 1H), 1.36 (dd, 1H), 1.23 (s, 3H), 1.01 (d, 3H), 0.98 (d, 3H).

Enantiomerically pure isomer 1:

LC-MS (method 1A): Rₜ=0.43 min; MS (ESIpos): m/z=264 [M+H]⁺;

¹H-NMR (400 MHz, DMSO-d₆): δ [ppm]=7.33-7.18 (m, 5H), 4.16 (d, 1H), 3.90 (d, 1H), 3.76 (m_{c}, 1H), 3.50 (dd, 1H), 3.26 (dd, 1H), 3.10 (d, 1H), 2.43 (d, 1H), 2.32 (m_{c}, 1H), 2.10 (dd, 1H), 1.84 (d, 1H), 1.27 (dd, 1H), 1.09-1.06 (m, 6H), 0.98 (d, 3H).

Enantiomerically pure isomer 2:

LC-MS (method 1A): Rₜ=0.45 min; MS (ESIpos): m/z=264 [M+H]⁺;

¹H-NMR (400 MHz, DMSO-d₆): δ [ppm]=7.32-7.20 (m, 5H), 4.11 (d, 1H), 3.92 (d, 1H), 3.57 (m_{c}, 1H), 3.51 (dd, 1H), 3.41 (dd, 1H), 3.06 (d, 1H), 2.47 (d, 1H), 2.34 (m_{c}, 1H), 1.85-1.74 (m, 2H), 1.59 (dd, 1H), 1.06 (s, 3H), 1.03-0.97 (t, 6H).

Enantiomerically pure isomer 4:

LC-MS (method 1A): Rₜ=0.44 min; MS (ESIpos): m/z=264 [M+H]⁺;

¹H-NMR (400 MHz, DMSO-d₆): δ [ppm]=7.49 (s, 5H), 4.69 (d, 1H), 4.28-4.15 (m, 2H), 3.86-3.73 (m, 3H), 3.63 (t, 1H), 3.32 (t, 1H), 3.21 (br. s., 1H), 2.84 (d, 1H), 1.52-1.38 (m, 4H), 1.28 (s, 3H), 1.01 (d, 3H).

### Example 143A

### 1-[(5R)-2,5-Dimethylmorpholin-2-yl]propan-2-ol [enantiomerically pure isomer 3]

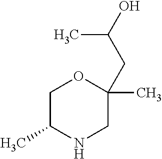

3.10 g (11.8 mmol) of 1-[(5R)-4-benzyl-2,5-dimethylmorpholin-2-yl]propan-2-ol [Example 142A, enantiomerically pure isomer 3] were initially charged in ethanol (118 ml), 296 mg of palladium on carbon (10%) and 148 mg of palladium hydroxide on carbon (20%) were added under argon and the mixture was stirred under an atmosphere of hydrogen at standard pressure overnight. The reaction solution was filtered through kieselguhr and the filter residue was washed with hot ethanol (100 ml). The filtrate was concentrated under reduced pressure and the product was dried under high vacuum. Yield: 2.06 g (quant.).

GC-MS (method 1B): Rₜ=3.86 min; MS (EIpos): m/z=173 [M]⁺;

¹H-NMR (400 MHz, DMSO-d₆): δ [ppm]=4.20 (d, 1H), 3.87 (br. s., 1H), 3.35 (dd, 1H), 3.16 (t, 1H), 2.67-2.53 (m, 3H), 2.05 (br. s., 1H), 1.44 (dd, 1H), 1.36 (dd, 1H), 1.23 (s, 3H), 1.04 (d, 3H), 0.85 (d, 3H).

### Example 144A

### N-Benzyl-2-chloro-N-(1,4-dihydroxybutan-2-yl)propanamide [diastereomer mixture, 4 isomers]

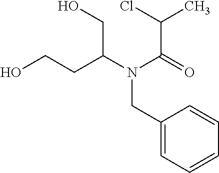 [SMILES: CC(Cl)C(=O)N(Cc1ccccc1)C(CO)CCO]

20.6 g (106 mmol) of 2-(benzylamino)butane-1,4-diol [racemate] [lit.: B. L. Feringa, B. de Lange, Heterocycles 1988, 27, 1197-1205] were initially charged in isopropanol (500 ml), the mixture was cooled to 0° C. and 21.4 g (29.4 ml, 211 mmol) of triethylamine were added. 16.1 g (12.6 ml, 127 mmol) of 2-chloropropionyl chloride [racemate] were then added dropwise. After 30 min of stirring, a further 10.4 g (8.37 ml, 84.4 mmol) of 2-chloropropionyl chloride [racemate] were added dropwise, and the reaction solution was allowed to warm to RT. The reaction solution was concentrated under reduced pressure and the residue was taken up in ethyl acetate (500 ml) and washed with 0.5 N aqueous hydrogen chloride solution (400 ml). The aqueous phase was extracted repeatedly with ethyl acetate. The organic phases were dried over sodium sulphate, filtered and concentrated under reduced pressure. The crude product was used without further purification in the next step. Yield: 37.5 g (78% of theory, purity: 63%, diastereomer ratio about 2:1).

LC-MS (Method 1A): Rₜ=0.71 min (diastereomer 1, 2 isomers), Rₜ=0.72 min (diastereomer 2, 2 isomers);

MS (ESIpos): m/z=286 [M+H]⁺.

### Example 145A

### 4-Benzyl-5-(2-hydroxyethyl)-2-methylmorpholin-3-one [diastereomer mixture, 4 isomers]

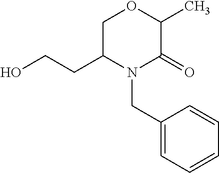 [SMILES: CC1OCC(CCO)N(Cc2ccccc2)C1=O]

37.5 g (82.5 mmol, purity: 63%) of N-benzyl-2-chloro-N-(1,4-dihydroxybutan-2-yl)propanamide [diastereomer mixture, 4 isomers] were initially charged in isopropanol (500 ml), the mixture was cooled to 0° C. and 73.5 g (655 mmol) of potassium tert-butoxide were then added in one portion. The mixture was stirred at 0° C. for 1 h and most of the isopropanol was then removed under reduced pressure. The residue was taken up in ethyl acetate and washed with 1 N aqueous hydrogen chloride solution (400 ml). The organic phase was dried over sodium sulphate, filtered and concentrated under reduced pressure. The crude product was used without further purification in the next step. Yield: 28.8 g (quant., purity: 82%, diastereomer ratio about 2.5:1).

LC-MS (Method 7A): Rₜ=1.42 min (diastereomer 1, 2 isomers), Rₜ=1.46 min (diastereomer 2, 2 isomers);

MS (ESIpos): m/z=250 [M+H]⁺.

### Example 146A

### 2-(4-Benzyl-6-methylmorpholin-3-yl)ethanol [diastereomer mixture, 4 isomers]

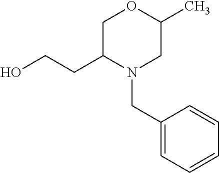 [SMILES: CC1CN(Cc2ccccc2)C(CCO)CO1]

28.8 g (94.7 mmol, purity: 82%) of 4-benzyl-5-(2-hydroxyethyl)-2-methylmorpholin-3-one [diastereomer mixture, 4 isomers] were initially charged in tetrahydrofuran (800 ml), 231 ml (462 mmol) of 2 M borane/dimethyl sulphide complex solution in tetrahydrofuran were added under argon and the mixture was stirred under reflux for 2 h. The mixture was subsequently cooled to 0° C., methanol (220 ml) was added carefully and the mixture was stirred under reflux for 30 min. This was followed by complete concentration under reduced pressure. Yield: 19.2 g (crude product).

LC-MS (Method 1A): Rₜ=0.26 min (diastereomer 1, 2 isomers), Rₜ=0.28 min (diastereomer 2, 2 isomers).

MS (ESIpos): m/z=236 [M+H]⁺.

### Example 147A

### (4-Benzyl-6-methylmorpholin-3-yl)acetaldehyde [diastereomer mixture, 4 isomers]

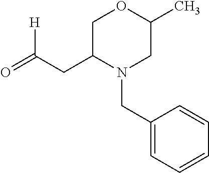 [SMILES: CC1CN(Cc2ccccc2)C(CC=O)CO1]

64.7 g (44.5 ml, 510 mmol) of oxalyl chloride were initially charged in dichloromethane (340 ml), and 79.7 g (72.4 ml, 1.02 mol) of dimethyl sulphoxide in dichloromethane (60 ml) were then added slowly at −78° C. 12.0 g (about 51.0 mmol, crude product) of 2-(4-benzyl-6-methylmorpholin-3-yl)ethanol [diastereomer mixture, 4 isomers] in dichloromethane (60 ml) were then added and the reaction mixture was stirred at −78° C. for 1 h. Over 20 min, 155 g (213 ml, 1.53 mol) of triethylamine were slowly added to the cold reaction mixture, and the reaction mixture was allowed to warm to RT. The reaction mixture was poured into water, and after separation of the phases the organic phase was washed with water. The organic phase was then dried over sodium sulphate, filtered and concentrated under reduced pressure. The crude product was then purified by flash chromatography on silica gel (cyclohexane/ethyl acetate 10:1-1:1). Yield: 7.14 g (48% of theory, purity: 80%).

LC-MS (method 4A:): Rₜ=2.57 min; MS (ESIpos): m/z=234 [M+H]⁺.

### Example 148A

### 1-(4-Benzyl-6-methylmorpholin-3-yl)propan-2-ol [diastereomer mixture, 8 isomers]

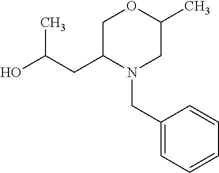 [SMILES: CC(O)CC1COC(C)CN1Cc1ccccc1]

2.30 g (9.86 mmol) of 4-benzyl-6-methylmorpholin-3-yl)acetaldehyde [diastereomer mixture, 4 isomers] were initially charged in tetrahydrofuran (42.2 ml), and 11.8 ml (11.8 mmol) of a 1 M solution of methylmagnesium bromide in tetrahydrofuran were added at −78° C. The mixture was stirred at −78° C. for 15 min and then allowed to warm to RT. Saturated aqueous ammonium chloride solution (70 ml) was added carefully to the reaction solution, most of the tetrahydrofuran was removed under reduced pressure and the residue was taken up in dichloromethane. After separation of the phases, the organic phase was washed with water, dried over sodium sulphate, filtered and concentrated under reduced pressure. The crude product was used without further purification in the next step. Yield: 1.94 g of crude product.

LC-MS (Method 4A): Rₜ=2.34 min (diastereomer 1, 2 isomers), Rₜ=2.40 min (diastereomer 2, 2 isomers), Rₜ=2.47 min (diastereomer 3, 2 isomers); one diastereomer obscured; MS (ESIpos): m/z=250 [M+H]⁺.

### Example 149A

### 1-(4-Benzyl-6-methylmorpholin-3-yl)acetone [diastereomer mixture, 4 isomers]

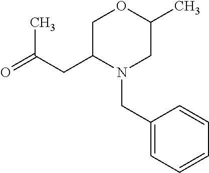 [SMILES: CC(=O)CC1COC(C)CN1Cc1ccccc1]

At −78° C., 11.9 g (10.8 ml, 152 mmol) of dimethyl sulphoxide in dichloromethane (9.0 ml) were added slowly to 9.67 g (6.65 ml, 76.2 mmol) of oxalyl chloride in dichloromethane (50 ml). 1.90 g (about 7.62 mmol, crude product) of 1-(4-benzyl-6-methylmorpholin-3-yl)propan-2-ol [diastereomer mixture, 8 isomers] in dichloromethane (9.0 ml) were then added and the reaction mixture was stirred at −78° C. for 1 h. Over 20 min, 23.1 g (31.9 ml, 229 mmol) of triethylamine were slowly added to the cold reaction mixture, and the reaction mixture was then allowed to warm to RT. The reaction mixture was poured into water, and after separation of the phases the organic phase was washed with water. The organic phase was dried over sodium sulphate, filtered and concentrated under reduced pressure. The residue was purified by preparative RP-HPLC (acetonitrile/water). Yield: 544 mg (28% of theory, diastereomer ratio: about 1:1).

LC-MS (Method 1A): Rₜ=0.45 min (diastereomer 1, 2 isomers), Rₜ=0.47 min (diastereomer 2, 2 isomers)

MS (ESIpos): m/z=248 [M+H]⁺.

### Example 150A

### 1-(4-Benzyl-6-methylmorpholin-3-yl)-2-methylpropan-2-ol [diastereomer 1, 2 isomers+diastereomer 2, 2 isomers]

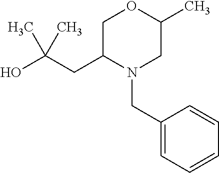 [SMILES: CC1CN(Cc2ccccc2)C(CC(C)(C)O)CO1]

544 mg (2.20 mmol) of 1-(4-benzyl-6-methylmorpholin-3-yl)acetone [diastereomer mixture, 4 isomers] were initially charged in tetrahydrofuran (9.42 ml), and 2.86 ml (2.86 mmol) of a 1 M solution of methylmagnesium bromide in tetrahydrofuran were added at −78° C. The mixture was stirred at −78° C. for 15 min and then allowed to warm to RT. Saturated aqueous ammonium chloride solution (70 ml) was then added carefully to the reaction solution, most of the tetrahydrofuran was removed under reduced pressure and the residue was taken up in dichloromethane. After separation of the phases, the organic phase was washed with water, dried over sodium sulphate, filtered and concentrated under reduced pressure. The residue was purified by preparative RP-HPLC (acetonitrile/water, isocratic) and separated into the two diastereomers in the process. Yield: 109 mg (19% of theory, diastereomer 1, 2 isomers), 109 mg (19% of theory, diastereomer 2, 2 isomers).

LC-MS (Method 1A): Rₜ=0.49 min (diastereomer 1, 2 isomers), Rₜ=0.54 min (diastereomer 2, 2 isomers); MS (ESIpos): m/z=264 [M+H]⁺.

### Example 151A

### 2-Methyl-1-(6-methylmorpholin-3-yl)propan-2-ol [diastereomer 2, 2 isomers]

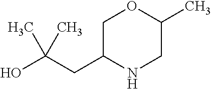

110 mg (0.416 mmol) of 1-(4-benzyl-6-methylmorpholin-3-yl)-2-methylpropan-2-ol [diastereomer 2, 2 isomers, Example 150A] were initially charged in ethanol (4.2 ml), 10.4 mg of palladium on carbon (10%) and 5.2 mg of palladium hydroxide on carbon (20%) were added under argon, and the mixture was then stirred under an atmosphere of hydrogen at standard pressure overnight. The reaction solution was filtered through kieselguhr and the filter residue was washed with ethanol. The filtrate was concentrated under reduced pressure and the product was dried under high vacuum. Yield: 74.4 mg (quant.).

MS (method 1C): m/z=174 [M+H]⁺.

### Example 152A

### [4-Benzyl-6,6-dimethylmorpholin-3-yl]acetaldehyde [enantiomer mixture, 2 isomers]

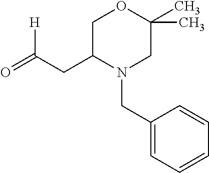 [SMILES: CC1(C)CN(Cc2ccccc2)C(CC=O)CO1]

At −78° C., 861 mg (782 μl, 11.0 mmol) of dimethyl sulphoxide in dichloromethane (5.0 ml) were added slowly to 800 mg (550 μl, 6.30 mmol) of oxalyl chloride in dichloromethane (20 ml). 947 mg (3.80 mmol) of 2-[(3R)-4-benzyl-6,6-dimethylmorpholin-3-yl]ethanol [enantiomer mixture, 2 isomers] in dichloromethane (6.0 ml) were added and the reaction mixture was stirred at −78° C. for 1 h. Over 20 min, 2.11 g (2.91 ml, 20.9 mmol) of triethylamine were slowly added to the cold reaction mixture, and the reaction mixture was then allowed to warm to RT. The reaction mixture was poured into water, and after separation of the phases the organic phase was washed with water. The organic phase was dried over sodium sulphate, filtered and concentrated under reduced pressure. The crude product was then purified by flash chromatography on silica gel (cyclohexane/ethyl acetate 10:1). Yield: 860 mg (91% of theory), proportional racemization at this stage.

LC-MS (method 1A:): Rₜ=0.49 min; MS (ESIpos): m/z=249 [M+H]⁺;

¹H-NMR (400 MHz, DMSO-d₆): δ [ppm]=9.78 (t, 1H), 7.37-7.16 (m, 5H), 3.81 (d, 1H), 3.67 (dd, 1H), 3.52 (dd, 1H), 3.16 (d, 1H), 2.89-2.80 (m, 1H), 2.72-2.64 (m, 2H), 2.32 (d, 1H), 1.92 (d, 1H), 1.13 (s, 3H), 1.08 (s, 3H).

### Example 153A

### 1-[4-Benzyl-6,6-dimethylmorpholin-3-yl]propan-2-ol [diastereomer mixture, 4 isomers]

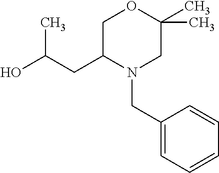 [SMILES: CC(O)CC1COC(C)(C)CN1Cc1ccccc1]

860 mg (3.48 mmol) of [4-benzyl-6,6-dimethylmorpholin-3-yl]acetaldehyde [enantiomer mixture, 2 isomers] were initially charged in tetrahydrofuran (14.9 ml), and 4.17 ml (4.17 mmol) of a 1 M solution of methylmagnesium bromide in tetrahydrofuran were added at −78° C. The mixture was stirred at −78° C. for 15 min and then allowed to warm to RT. Saturated aqueous ammonium chloride solution (50 ml) was then added carefully to the reaction solution, most of the tetrahydrofuran was removed under reduced pressure and the residue was taken up in dichloromethane. After separation of the phases, the organic phase was washed with water, dried over sodium sulphate, filtered and concentrated under reduced pressure. The residue was directly purified by preparative RP-HPLC (acetonitrile/water, isocratic). Yield: 571 mg (62% of theory, diastereomer ratio: about 1:1).

LC-MS (Method 1A): Rₜ=0.47 min (diastereomer 1, 2 isomers), Rₜ=0.50 min (diastereomer 2, 2 isomers).

MS (ESIpos): m/z=264 [M+H]⁺.

### Example 154A

### 1-[6,6-Dimethylmorpholin-3-yl]propan-2-ol [diastereomer mixture, 4 isomers]

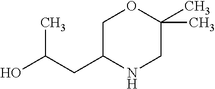

565 mg (2.15 mmol) of 1-[4-benzyl-6,6-dimethylmorpholin-3-yl]propan-2-ol [diastereomer mixture, 4 isomers] were initially charged in ethanol (21.6 ml), 53.9 mg of palladium on carbon (10%) and 27.0 mg of palladium hydroxide on carbon (20%) were added under argon, and the mixture was then stirred under an atmosphere of hydrogen at standard pressure overnight. The reaction solution was filtered through kieselguhr and the filter residue was washed with ethanol. The filtrate was concentrated under reduced pressure and the product was dried under high vacuum. Yield: 366 mg (98% of theory).

MS (method 2C): m/z=174 [M+H]⁺.

### Example 155A

### 4-Benzyl-5-({[tert-butyl(dimethyl)silyl]oxy}methyl)-2-methylmorpholin-3-one [diastereomer mixture, 4 isomers]

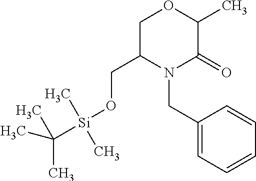 [SMILES: CC1OCC(CO[Si](C)(C)C(C)(C)C)N(Cc2ccccc2)C1=O]

21.5 g (91.4 mmol) of 4-benzyl-5-(hydroxymethyl)-2-methylmorpholin-3-one [diastereomer mixture, 4 isomers] were initially charged in N,N-dimethylformamide (126 ml), and 12.4 g (183 mmol) of imidazole and then 14.5 g (96.0 mmol) of tert-butyldimethylsilyl chloride were added at RT. The mixture was stirred for 2 h, and most of the solvent was then removed under reduced pressure. The residue was taken up in ethyl acetate/water and the organic phase was washed with water, 0.4 N aqueous hydrogen chloride solution, saturated aqueous sodium bicarbonate solution and water. The organic phase was dried over sodium sulphate, filtered and concentrated under reduced pressure. The crude product was used without further purification in the next step. Yield: 31.2 g (97% of theory, diastereomer ratio about 7:3).

LC-MS (method 1A): Rₜ=1.41 min; MS (ESIpos): m/z=350 [M+H]⁺;

¹H-NMR (400 MHz, DMSO-d₆): δ [ppm]=7.38-7.18 (m, 5H), 5.00 (d, 0.3H), 4.95 (d, 0.7H), 4.32-4.19 (m, 2H), 3.92-3.85 (m, 1H), 3.75-3.62 (m, 3H), 3.32-3.26 (m, 0.3H), 3.19-3.13 (m., 0.7H), 1.35 (d, 0.9H), 1.32 (d, 2.1H), 0.84-0.80 (m, 9H), 0.04-0.03 (m, 6H).

### Example 156A

### 2-Allyl-4-benzyl-5-({[tert-butyl(dimethyl)silyl]oxy}methyl)-2-methylmorpholin-3-one [diastereomer mixture, 4 isomers]

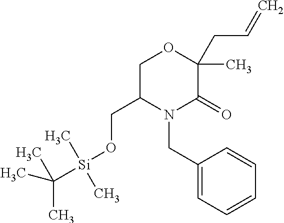 [SMILES: C=CCC1(C)OCC(CO[Si](C)(C)C(C)(C)C)N(Cc2ccccc2)C1=O]

30.6 g (87.5 mmol) of 4-benzyl-5-({[tert-butyl(dimethyl)silyl]oxy}methyl)-2-methylmorpholin-3-one [diastereomer mixture, 4 isomers] were initially charged in tetrahydrofuran (1.13 l), 123 ml (123 mmol) of 1 M lithium hexamethyldisilazide solution in tetrahydrofuran were added under argon and at −78° C. and the mixture was then stirred for 15 min. Subsequently, 17.6 g (9.61 ml, 105 mmol) of allyl iodide were added, and the reaction mixture was allowed to warm to RT and stirred overnight. The reaction was terminated by addition of saturated aqueous ammonium chloride solution and the mixture was then extracted with ethyl acetate. The organic phase was washed with saturated aqueous sodium chloride solution, dried over sodium sulphate, filtered and concentrated under reduced pressure. The crude product was used without further purification in the next step. Yield: 36.6 g (100% of theory).

LC-MS (method 1A): Rₜ=1.53 min; MS (ESIpos): m/z=390 [M+H]⁺.

### Example 157A

### [4-Benzyl-5-({[tert-butyl(dimethyl)silyl]oxy}methyl)-2-methyl-3-oxomorpholin-2-yl]acetaldehyde [diastereomer mixture, 4 isomers]

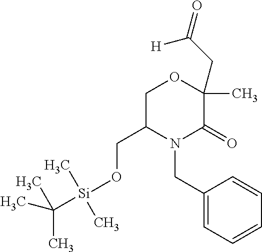 [SMILES: CC1(CC=O)OCC(CO[Si](C)(C)C(C)(C)C)N(Cc2ccccc2)C1=O]

At 0° C., 1.16 ml (0.427 mmol) of a 2.5% solution of osmium tetroxide in tert-butanol and 2.96 g (13.9 mmol) of sodium periodate were added to 1.80 g (4.62 mmol) of 2-allyl-4-benzyl-5-({[tert-butyl(dimethyl)silyl]oxy}methyl)-2-methylmorpholin-3-one [diastereomer mixture, 4 isomers] in tetrahydrofuran (100 ml) and water (60 ml). The mixture was then allowed to warm to RT and stirred for 20 h. The reaction solution was filtered through kieselguhr and the filter residue was washed with ethyl acetate. After separation of the phases, the organic phase was dried over sodium sulphate, filtered and concentrated under reduced pressure. The crude product was used without further purification in the next step. Yield: 2.02 g (87% of theory, purity: 78%).

LC-MS (method 2A): Rₜ=1.42 min; MS (ESIpos): m/z=392 [M+H]⁺.

### Example 158A

### 4-Benzyl-5-({[tert-butyl(dimethyl)silyl]oxy}methyl)-2-(2-hydroxyethyl)-2-methylmorpholin-3-one [diastereomer mixture, 4 isomers]

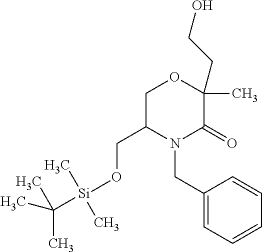 [SMILES: CC1(CCO)OCC(CO[Si](C)(C)C(C)(C)C)N(Cc2ccccc2)C1=O]

22.2 g (32.5 mmol, purity: 57%) of [4-benzyl-5-({[tert-butyl(dimethyl)silyl]oxy}-methyl)-2-methyl-3-oxomorpholin-2-yl]acetaldehyde [diastereomer mixture, 4 isomers] were initially charged in methanol (242 ml), and 1.84 g (48.7 mmol) of sodium borohydride were added at 0° C. The mixture was then allowed to warm to RT and stirred for 30 min. Water was then added to the reaction solution, most of the methanol was removed under reduced pressure and the residue was extracted with ethyl acetate. The organic phase was dried over sodium sulphate, filtered and concentrated under reduced pressure. The crude product was used without further purification in the next step. Yield: 22.8 g (quant., purity: 62%).

LC-MS (method 1A): Rₜ=1.28 min; MS (ESIpos): m/z=394 [M+H]⁺.

### Example 159A

### 4-Benzyl-5-({[tert-butyl(dimethyl)silyl]oxy}methyl)-2-(2-{[tert-butyl(diphenyl)silyl]oxy}ethyl)-2-methylmorpholin-3-one [diastereomer mixture, 4 isomers]

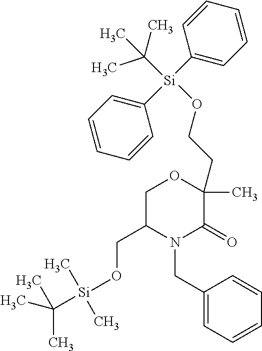 [SMILES: CC1(CCO[Si](c2ccccc2)(c2ccccc2)C(C)(C)C)OCC(CO[Si](C)(C)C(C)(C)C)N(Cc2ccccc2)C1=O]

15.0 g (38.1 mmol) of 4-benzyl-5-({[tert-butyl(dimethyl)silyl]oxy}methyl)-2-(2-hydroxyethyl)-2-methylmorpholin-3-one [diastereomer mixture, 4 isomers], 7.73 g (114 mmol) of imidazole and 4-(dimethylamino)pyridine (about 6 mg) were initially charged in N,N-dimethylformamide (52.8 ml), and 15.7 g (57.2 mmol) of tert-butyldiphenylsilyl chloride were added at 0° C. The mixture was stirred for 60 h and allowed to warm to RT during this time. Subsequently, most of the solvent was removed under reduced pressure, the residue was taken up in ethyl acetate/water and the organic phase was washed with water and saturated aqueous sodium chloride solution. The organic phase was dried over magnesium sulphate, filtered and concentrated under reduced pressure. The crude product obtained was purified by silica gel chromatography (cyclohexane/ethyl acetate 9:1). Yield: 15.6 g (49% of theory, purity: 50%).

LC-MS (method 7A): Rₜ=6.96 min; MS (ESIpos): m/z=633 [M+H]⁺.

### Example 160A

### 4-Benzyl-2-(2-{[tert-butyl(diphenyl)silyl]oxy}ethyl)-5-(hydroxymethyl)-2-methylmorpholin-3-one [diastereomer mixture, 4 isomers]

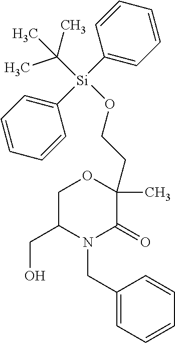 [SMILES: CC1(CCO[Si](c2ccccc2)(c2ccccc2)C(C)(C)C)OCC(CO)N(Cc2ccccc2)C1=O]

15.6 g (about 12.3 mmol, crude product) of 4-benzyl-5-({[tert-butyl(dimethyl)silyl]oxy}methyl)-2-(2-{[tert-butyl(diphenyl)silyl]oxy}ethyl)-2-methylmorpholin-3-one [diastereomer mixture, 4 isomers] were dissolved in concentrated acetic acid, tetrahydrofuran and water (250 ml, 3:1:1), and the mixture was stirred at RT overnight. The reaction solution was then diluted with ethyl acetate and washed three times with water, once with saturated aqueous sodium bicarbonate solution and then with saturated aqueous sodium chloride solution. The organic phase was dried over sodium sulphate, filtered and concentrated under reduced pressure. The crude product obtained was purified by silica gel chromatography (cyclohexane/ethyl acetate 7:3-1:1). Yield: 2.55 g (19% of theory).

LC-MS (method 1A): Rₜ=1.43 min; MS (ESIpos): m/z=518 [M+H]⁺.

### Example 161A

### 4-Benzyl-5-(fluoromethyl)-2-(2-hydroxyethyl)-2-methylmorpholin-3-one [diastereomer mixture, 4 isomers]

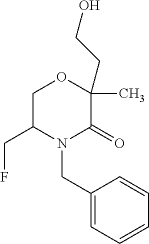 [SMILES: CC1(CCO)OCC(CF)N(Cc2ccccc2)C1=O]

2.00 g (3.86 mmol) of 4-benzyl-2-(2-{[tert-butyl(diphenyl)silyl]oxy}ethyl)-5-(hydroxymethyl)-2-methylmorpholin-3-one [diastereomer mixture, 4 isomers] were initially charged in tetrahydrofuran (40.0 ml), and 20.0 ml (46.5 mmol) of bis(2-methoxyethyl)aminosulphur trifluoride (Deoxofluor, 50% strength solution in tetrahydrofuran) were added slowly at RT. 1 drop of methanol was then added and the mixture was stirred at RT for 2 h and then under reflux for 2 h. The reaction solution was carefully added dropwise to saturated aqueous sodium bicarbonate solution, the phases were separated and the aqueous phase was extracted with dichloromethane. The combined organic phases were dried over sodium sulphate, filtered and concentrated under reduced pressure. The crude product was taken up in tetrahydrofuran (40.0 ml), and 15.5 ml (15.5 mmol) of tetra-n-butylammonium fluoride solution (1.0 M in tetrahydrofuran) were added. The reaction solution was stirred at RT overnight and then concentrated under reduced pressure. The residue was taken up in dichloromethane and washed with water and the organic phase was dried over sodium sulphate, filtered and concentrated under reduced pressure. The crude product was used without further purification in the next step. Yield: 2.74 g (74% of theory, purity: 29%).

LC-MS (method 1A): Rₜ=0.76 min; MS (ESIpos): m/z=282 [M+H]⁺.

### Example 162A

### 2-[4-Benzyl-5-(fluoromethyl)-2-methylmorpholin-2-yl]ethanol [diastereomer mixture, 4 isomers]

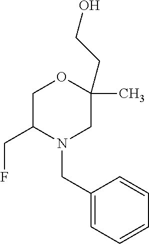 [SMILES: CC1(CCO)CN(Cc2ccccc2)C(CF)CO1]

2.74 g (2.87 mmol, purity: 29%) of 4-benzyl-5-(fluoromethyl)-2-(2-hydroxyethyl)-2-methylmorpholin-3-one [diastereomer mixture, 4 isomers] were initially charged in tetrahydrofuran (29.1 ml), 5.74 ml (11.5 mmol) of 2 M borane/dimethyl sulphide complex solution in tetrahydrofuran were added under argon and the mixture was stirred under reflux for 3 h. The mixture was subsequently cooled to 0° C., methanol (7.5 ml) was added carefully and the mixture was stirred under reflux for 30 min. The mixture was then concentrated completely under reduced pressure and the residue was purified by preparative RP-HPLC (acetonitrile/water, isocratic). Yield: 769 mg (97% of theory, diastereomer ratio: about 3:2).

LC-MS (Method 1A): Rₜ=0.63 min (diastereomer 1, 2 isomers), Rₜ=0.65 min (diastereomer 2, 2 isomers).

MS (ESIpos): m/z=268 [M+H]⁺.

### Example 163A

### 2-[5-(Fluoromethyl)-2-methylmorpholin-2-yl]ethanol [diastereomer mixture, 4 isomers]

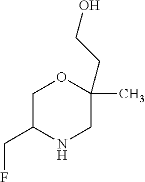

769 mg (2.80 mmol) of 4-benzyl-5-(fluoromethyl)-2-(2-hydroxyethyl)-2-methylmorpholin-3-one [diastereomer mixture, 4 isomers] were initially charged in ethanol (41.8 ml), 154 mg of palladium on carbon (10%) and 77.0 mg of palladium hydroxide on carbon (20%) were added under argon, and the mixture was then stirred under an atmosphere of hydrogen at standard pressure for 4 h. The reaction solution was filtered through kieselguhr and the filter residue was washed with ethanol. The filtrate was concentrated under reduced pressure and repeatedly co-evaporated with dichloromethane, and the product was dried under high vacuum. Yield: 521 mg (99% of theory).

MS (method 1C): m/z=178 [M+H]⁺.

### Example 164A

### 4-Benzyl-2-[2-(benzyloxy)ethyl]-5-({[tert-butyl(dimethyl)silyl]oxy}methyl)-2-methylmorpholin-3-one [diastereomer mixture, 4 isomers]

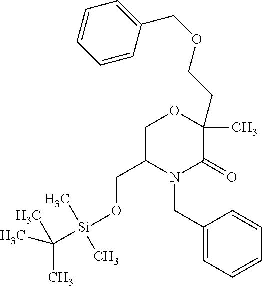 [SMILES: CC1(CCOCc2ccccc2)OCC(CO[Si](C)(C)C(C)(C)C)N(Cc2ccccc2)C1=O]

20.5 g (31.3 mmol, purity: 60%) of 4-benzyl-5-({[tert-butyl(dimethyl)silyl]oxy}methyl)-2-(2-hydroxyethyl)-2-methylmorpholin-3-one [diastereomer mixture, 4 isomers] were initially charged in N,N-dimethylformamide (205 ml) under argon, and 1.12 g (46.9 mmol, 60% suspension in paraffin oil) of sodium hydride were added at 0° C. 4.09 ml (5.88 g, 34.4 mmol) of benzyl bromide were then added dropwise, and the mixture was stirred at RT overnight. A further 560 mg (23.4 mmol, 60% suspension in paraffin oil) of sodium hydride, 2.04 ml (2.96 g, 17.2 mmol) of benzyl bromide and catalytic amounts of tetra-n-butylammonium iodide (about 50 mg) were added and the mixture was stirred at RT for 2 h. The mixture was then stirred at 50° C. for 1 h, another 560 mg (23.4 mmol, 60% suspension in paraffin oil) of sodium hydride were added and the mixture was stirred at 50° C. for 1 h. The reaction mixture was concentrated under reduced pressure and the residue was taken up in ethyl acetate and washed with water. The organic phase was dried over sodium sulphate, filtered and concentrated under reduced pressure. The crude product was purified by preparative RP-HPLC (acetonitrile/water). Yield: 7.19 g (44% of theory).

LC-MS (method 1A): Rₜ=1.58 min; MS (ESIpos): m/z=484 [M+H]⁺.

### Example 165A

### 4-Benzyl-2-[2-(benzyloxy)ethyl]-5-(hydroxymethyl)-2-methylmorpholin-3-one [diastereomer mixture, 4 isomers]

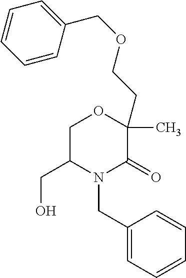 [SMILES: CC1(CCOCc2ccccc2)OCC(CO)N(Cc2ccccc2)C1=O]

7.19 g (13.7 mmol) of 4-benzyl-2-[2-(benzyloxy)ethyl]-5-({[tert-butyl(dimethyl)silyl]oxy}methyl)-2-methylmorpholin-3-one [diastereomer mixture, 4 isomers] were initially charged in tetrahydrofuran (100 ml), and 34.3 ml (34.3 mmol) of tetra-n-butylammonium fluoride solution (1.0 M in tetrahydrofuran) were added at RT. The reaction solution was stirred at RT for 4 h and the reaction solution was then concentrated under reduced pressure. The residue was taken up in ethyl acetate and washed with water and the organic phase was dried over sodium sulphate, filtered and concentrated under reduced pressure. The crude product was then purified by preparative RP-HPLC (acetonitrile/water). Yield: 4.83 g (93% of theory).

LC-MS (method 1A): Rₜ=0.99 min; MS (ESIpos): m/z=370 [M+H]⁺.

### Example 166A

### 4-Benzyl-6-[2-(benzyloxy)ethyl]-6-methyl-5-oxomorpholine-3-carbaldehyde [diastereomer mixture, 4 isomers]

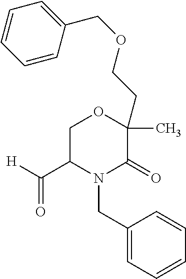 [SMILES: CC1(CCOCc2ccccc2)OCC(C=O)N(Cc2ccccc2)C1=O]

At −78° C., 4.40 g (4.00 ml, 56.3 mmol) of dimethyl sulphoxide in dichloromethane (4.0 ml) were added slowly to 4.09 g (2.81 ml, 32.2 mmol) of oxalyl chloride in dichloromethane (8.0 ml). 3.66 g (9.71 mmol) of 4-benzyl-2-[2-(benzyloxy)ethyl]-5-(hydroxymethyl)-2-methylmorpholin-3-one [diastereomer mixture, 4 isomers] in dichloromethane (8.0 ml) were then added. 10.8 g (14.9 ml, 107 mmol) of triethylamine were slowly added to the cold reaction mixture, and the reaction mixture was allowed to warm to RT and stirred overnight. The reaction mixture was poured into water, and after separation of the phases the organic phase was washed with water, dried over sodium sulphate, filtered and concentrated under reduced pressure. The crude product obtained was purified by silica gel chromatography (cyclohexane/ethyl acetate 2:1). Yield: 3.54 g (86% of theory, purity: 86%).

LC-MS (Method 1A): Rₜ=0.94 min (hydrate), Rₜ=1.11 min (aldehyde)

MS (ESIpos): m/z=368 [M+H]⁺.

### Example 167A

### 4-Benzyl-2-[2-(benzyloxy)ethyl]-5-(difluormethyl)-2-methylmorpholin-3-one [diastereomer mixture, 4 isomers]

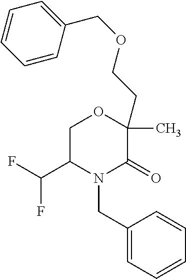 [SMILES: CC1(CCOCc2ccccc2)OCC(C(F)F)N(Cc2ccccc2)C1=O]

3.18 g (8.65 mmol) of 4-benzyl-6-[2-(benzyloxy)ethyl]-6-methyl-5-oxomorpholin-3-carbaldehyde [diastereomer mixture, 4 isomers] were initially charged in dichloromethane (127 ml), and 8.0 ml (18.6 mmol) of bis(2-methoxyethyl)aminosulphur trifluoride (Deoxofluor, 50% strength solution in tetrahydrofuran) were added slowly at RT. 1 drop of methanol was then added and the mixture was subsequently stirred at RT overnight. A further 8.0 ml (18.6 mmol) of bis(2-methoxyethyl)aminosulphur trifluoride (Deoxofluor, 50% strength solution in tetrahydrofuran) were added and the mixture was stirred at RT for 48 h. The reaction solution was carefully added dropwise to saturated aqueous sodium bicarbonate solution and the aqueous phase was extracted with dichloromethane. The combined organic phases were dried over sodium sulphate, filtered and concentrated under reduced pressure. The crude product was used without further purification in the next step. Yield: 4.29 g (91% of theory, purity: 71%, diastereomer ratio about 2:1).

LC-MS (Method 1A): Rₜ=1.22 min (diastereomer 1, 2 isomers), Rₜ=1.24 min (diastereomer 2, 2 isomers).

MS (ESIpos): m/z=390 [M+H]⁺.

### Example 168A

### 4-Benzyl-2-[2-(benzyloxy)ethyl]-5-(difluoromethyl)-2-methylmorpholine [diastereomer 1, 2 isomers+diastereomer 2, 2 isomers]

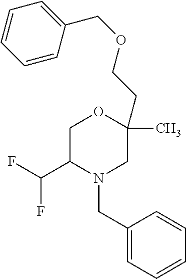 [SMILES: CC1(CCOCc2ccccc2)CN(Cc2ccccc2)C(C(F)F)CO1]

4.29 g (7.90 mmol, purity: 71%) of 4-benzyl-2-[2-(benzyloxy)ethyl]-5-(difluoromethyl)-2-methylmorpholin-3-one [diastereomer mixture, 4 isomers] were initially charged in tetrahydrofuran (80.0 ml), 15.8 ml (31.6 mmol) of 2 M borane/dimethyl sulphide complex solution in tetrahydrofuran were added under argon and the mixture was stirred under reflux for 3 h. A further 6.0 ml (12.0 mmol) of 2 M borane/dimethyl sulphide complex solution in tetrahydrofuran were added and the mixture was stirred under reflux for 1 h. Methanol (40 ml) was then added carefully and the mixture was stirred under reflux for 30 min. The mixture was concentrated completely under reduced pressure and the residue was purified on an achiral phase according to Method 2G and separated into the two diastereomers. Yield: 514 mg (17% of theory, diastereomer 1, 2 isomers, minor isomer); 935 mg (31% of theory, diastereomer 2, 2 isomers, main isomer).

diastereomer 1, 2 isomers, minor isomer:

LC-MS (Method 1A): Rₜ=1.43 min (diastereomer 1, 2 isomers); MS (ESIpos): m/z=376 [M+H]⁺.

¹H-NMR (400 MHz, DMSO-d₆): δ [ppm]=7.38-7.20 (m, 10H), 6.65 (dt, 1H), 4.37 (s, 2H), 3.83 (br. t., 2H), 3.70-3.59 (m, 2H), 3.39 (t, 2H), 2.82 (m_{c}, 1H), 2.19 (d, 1H), 2.03 (dt, 1H), 1.66 (dt, 1H), 1.09 (s, 3H), 1 proton obscured).

diastereomer 2, 2 isomers, main isomer:

LC-MS (Method 1A): Rₜ=1.43 min (diastereomer 2, 2 isomers), MS (ESIpos): m/z=376 [M+H]⁺.

¹H-NMR (400 MHz, DMSO-d₆): δ [ppm]=7.40-7.16 (m, 10H), 6.43 (dt, 1H), 4.37 (s, 2H), 3.90-3.80 (m, 2H), 3.73-3.59 (m, 2H), 3.42 (t, 2H), 2.82 (m_{c}, 1H), 2.60 (d, 1H), 2.11 (d, 1H), 1.89 (dt, 1H), 1.69 (dt, 1H), 1.13 (s, 3H)).

### Example 169A

### 2-[5-(Difluoromethyl)-2-methylmorpholin-2-yl]ethanol [diastereomer 1, 2 isomers]

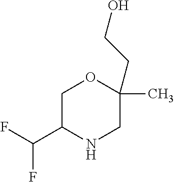

510 g (1.36 mmol) of 4-benzyl-2-[2-(benzyloxy)ethyl]-5-(difluoromethyl)-2-methylmorpholine [diastereomer 1, 2 isomers, Example 168A] were initially charged in ethanol (63.8 ml), 130 mg of palladium on carbon (10%) and 65.0 mg of palladium hydroxide on carbon (20%) were added under argon, and the mixture was then stirred under an atmosphere of hydrogen at standard pressure overnight. The reaction solution was filtered through kieselguhr and the filter residue was washed with ethanol. The filtrate was concentrated under reduced pressure and the product was then dried under high vacuum. Yield: 281 mg (99% of theory).

MS (method 1C): m/z=196 [M+H]⁺.

¹H-NMR (400 MHz, DMSO-d₆): δ [ppm]=5.91 (dt, 1H), 4.28 (t, 1H), 3.61-3.40 (m, 4H), 2.87 (br. s., 1H), 2.67-2.58 (m, 1H), 1.71-1.61 (m, 1H), 1.60-1.50 (m, 1H), 1.18 (s, 3H), 2 protons obscured

### Example 170A

### 4-Benzyl-5-({[tert-butyl(dimethyl)silyl]oxy}methyl)-2-(2-hydroxypropyl)-2-methylmorpholin-3-one [diastereomer mixture, 8 isomers]

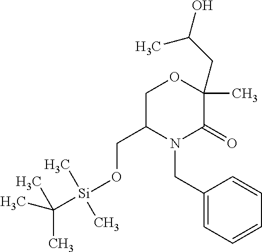 [SMILES: CC(O)CC1(C)OCC(CO[Si](C)(C)C(C)(C)C)N(Cc2ccccc2)C1=O]

10.0 g (17.9 mmol, purity: 70%) of [4-benzyl-5-({[tert-butyl(dimethyl)silyl]oxy}-methyl)-2-methyl-3-oxomorpholin-2-yl]acetaldehyde [diastereomer mixture, 4 isomers] were initially charged in tetrahydrofuran (75.4 ml), and 26.8 ml (26.8 mmol) of a 1 M solution of methylmagnesium bromide in tetrahydrofuran were added at −78° C. The mixture was stirred at −78° C. for 15 min and then allowed to warm to RT. Saturated aqueous ammonium chloride solution was then added carefully to the reaction solution, most of the tetrahydrofuran was removed under reduced pressure and the residue was taken up in dichloromethane. After separation of the phases, the organic phase was washed with water, dried over sodium sulphate, filtered and concentrated under reduced pressure. The crude product was used without further purification in the next step. Yield: 9.65 g of crude product.

LC-MS (Method 1A): Rₜ=1.25 min (diastereomer 1), Rₜ=1.27 min (diastereomer 2), 1.39 (diastereomer 3), one diastereomer obscured.

MS (ESIpos): m/z=408 [M+H]⁺.

### Example 171A

### 4-Benzyl-5-({[tert-butyl(dimethyl)silyl]oxy}methyl)-2-(2-{[tert-butyl(diphenyl)silyl]oxy}propyl)-2-methylmorpholin-3-one [diastereomer mixture, 8 isomers]

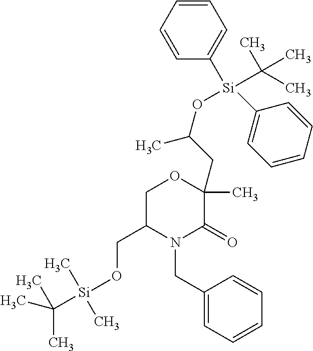 [SMILES: CC(CC1(C)OCC(CO[Si](C)(C)C(C)(C)C)N(Cc2ccccc2)C1=O)O[Si](c1ccccc1)(c1ccccc1)C(C)(C)C]

9.65 g (22.0 mmol) of 4-benzyl-5-({[tert-butyl(dimethyl)silyl]oxy}methyl)-2-(2-hydroxypropyl)-2-methylmorpholin-3-one [diastereomer mixture, 8 isomers], 3.00 g (44.0 mmol) of imidazole and 134 mg (1.10 mmol) of 4-(dimethylamino)pyridine were initially charged in dichloromethane (500 ml), and 6.66 g (6.30 ml, 24.2 mmol) of tert-butyldiphenylsilyl chloride were added at 0° C. The mixture was stirred for 48 h and allowed to warm to RT during this time. The mixture was then stirred at 40° C. for 24 h. After addition of a further 1.06 g (1.00 ml, 4.06 mmol), the mixture was stirred at 40° C. until the reaction had gone to completion. Subsequently, the reaction solution was diluted with dichloromethane and the organic phase was washed with water. The organic phase was dried over magnesium sulphate, filtered and concentrated under reduced pressure. The crude product was used without further purification in the next step. Yield: 17.2 g of crude product.

LC-MS (Method 1A): Rₜ=1.82 min (diastereomer 1+2, 4 isomers), Rₜ=1.87 min (diastereomer 3+4, 4 isomers)

MS (ESIpos): m/z=647 [M+H]⁺.

### Example 172A

### 4-Benzyl-2-(2-{[tert-butyl(diphenyl)silyl]oxy}propyl)-5-(hydroxymethyl)-2-methylmorpholin-3-one [diastereomer mixture, 8 isomers]

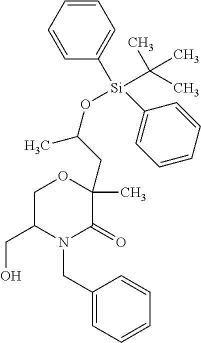 [SMILES: CC(CC1(C)OCC(CO)N(Cc2ccccc2)C1=O)O[Si](c1ccccc1)(c1ccccc1)C(C)(C)C]

17.2 g (about 6.12 mmol, crude product) of 4-benzyl-5-({[tert-butyl(dimethyl)silyl]oxy}methyl)-2-(2-{[tert-butyl(diphenyl)silyl]oxy}propyl)-2-methylmorpholin-3-one [diastereomer mixture, 8 isomers] were dissolved in concentrated acetic acid (111 ml), tetrahydrofuran (36.0 ml) and water (36.0 ml), and the mixture was stirred at RT for 5 d. The reaction solution was then diluted with ethyl acetate and washed once with water, three times with saturated aqueous sodium bicarbonate solution and once with saturated aqueous sodium chloride solution. The organic phase was dried over sodium sulphate, filtered and concentrated under reduced pressure. The crude product obtained was purified by silica gel chromatography (cyclohexane/ethyl acetate 20:1-1:1). Yield: 2.22 g (68% of theory).

LC-MS (method 1A): Rₜ=1.50 min; MS (ESIpos): m/z=532 [M+H]⁺.

### Example 173A

### 4-Benzyl-2-(2-{[tert-butyl(diphenyl)silyl]oxy}propyl)-5-(fluoromethyl)-2-methylmorpholin-3-one [diastereomer mixture, 8 isomers]

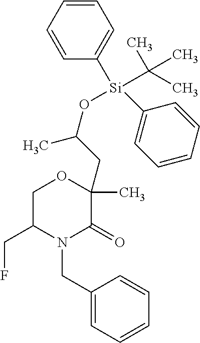 [SMILES: CC(CC1(C)OCC(CF)N(Cc2ccccc2)C1=O)O[Si](c1ccccc1)(c1ccccc1)C(C)(C)C]

2.10 g (3.95 mmol) of 4-benzyl-2-(2-{[tert-butyl(diphenyl)silyl]oxy}propyl)-5-(hydroxymethyl)-2-methylmorpholin-3-one [diastereomer mixture, 8 isomers] were initially charged in tetrahydrofuran (105 ml), and 11.7 ml (27.1 mmol) of bis(2-methoxyethyl)aminosulphur trifluoride (Deoxofluor, 50% strength solution in tetrahydrofuran) were added slowly at RT. 2 drops of ethanol were then added and the mixture was stirred under reflux for 5 h. The reaction solution was carefully added dropwise to saturated aqueous sodium bicarbonate solution and the aqueous phase was extracted with dichloromethane. The combined organic phases were dried over sodium sulphate, filtered and concentrated under reduced pressure. The crude product was used without further purification in the next step. Yield: 3.73 g (quant., purity: 82%).

LC-MS (method 1A): Rₜ=1.63 min; MS (ESIpos): m/z=534 [M+H]⁺.

### Example 174A

### 4-Benzyl-5-(fluoromethyl)-2-(2-hydroxypropyl)-2-methylmorpholin-3-one [diastereomer mixture, 8 isomers]

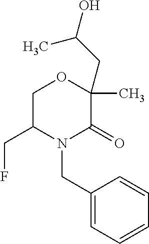 [SMILES: CC(O)CC1(C)OCC(CF)N(Cc2ccccc2)C1=O]

3.70 g (5.68 mmol, purity: 82%) of 4-benzyl-2-(2-{[tert-butyl(diphenyl)silyl]oxy}propyl)-5-(fluoromethyl)-2-methylmorpholin-3-one [diastereomer mixture, 8 isomers] were initially charged in tetrahydrofuran (124 ml), and 19.7 ml (19.7 mmol) of tetra-n-butylammonium fluoride solution (1.0 M in tetrahydrofuran) were added at RT. The reaction solution was stirred at RT overnight and then concentrated under reduced pressure. The residue was taken up in dichloromethane and washed with water and the organic phase was dried over sodium sulphate, filtered and concentrated under reduced pressure. The crude product was purified by preparative RP-HPLC (acetonitrile/water). Yield: 728 mg (43% of theory).

LC-MS (Method 1A): Rₜ=0.85 min (diastereomer 1+2, 4 isomers), Rₜ=0.86 min (diastereomer 3+4, 4 isomers)

MS (ESIpos): m/z=296 [M+H]⁺.

### Example 175A

### 1-[4-Benzyl-5-(fluoromethyl)-2-methylmorpholin-2-yl]propan-2-ol trifluoroacetate [diastereomer 1, 2 isomers+diastereomer 2, 2 isomers+diastereomer 3, 2 isomers+diastereomer 4, 2 isomers]

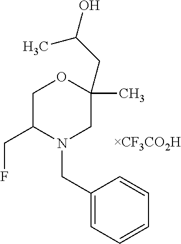 [SMILES: CC(O)CC1(C)CN(Cc2ccccc2)C(CF)CO1]

728 mg (2.46 mmol) of 4-benzyl-5-(fluoromethyl)-2-(2-hydroxypropyl)-2-methylmorpholin-3-one [diastereomer mixture, 8 isomers] were initially charged in tetrahydrofuran (24.2 ml), 4.93 ml (9.86 mmol) of 2 M borane/dimethyl sulphide complex solution in tetrahydrofuran were added under argon and the mixture was then stirred under reflux for 2 h. Methanol (10 ml) was then added carefully at 0° C. and the mixture was stirred under reflux for 30 min. The mixture was concentrated completely under reduced pressure and the residue was purified by preparative RP-HPLC (acetonitrile/water). The isomer mixture (262 mg) was then separated on an achiral phase according to Method 3G into the four diastereomers. Yield: 15.2 mg (2% of theory, diastereomer 1, 2 isomers); 166 mg (23% of theory, diastereomer 2, 2 isomers), 44.7 mg (6% of theory, diastereomer 3, 2 isomers), 92.5 mg (13% of theory, diastereomer 4, 2 isomers).

LC-MS (Method 1A): Rₜ=0.68 min (diastereomer 1, 2 isomers), Rₜ=0.69 min (diastereomer 2, 2 isomers), 0.73 min (diastereomer 3, 2 isomers), Rₜ=0.68 min (diastereomer 4, 2 isomers).

MS (ESIpos): m/z=282 [M+H-TFA]⁺.

### Example 176A

### 1-[5-(Fluoromethyl)-2-methylmorpholin-2-yl]propan-2-ol trifluoroacetate [diastereomer 2, 2 isomers]

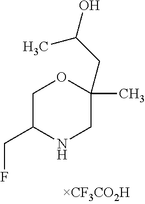

166 mg (0.421 mmol) of 1-[4-benzyl-5-(fluoromethyl)-2-methylmorpholin-2-yl]propan-2-ol trifluoroacetate [diastereomer 2, 2 isomers, Example 175A] were initially charged in ethanol (4.23 ml), 17.0 mg of palladium on carbon (10%) and 8.0 mg of palladium hydroxide on carbon (20%) were added under argon, and the mixture was then stirred under an atmosphere of hydrogen at standard pressure overnight. The reaction solution was filtered through kieselguhr and the filter residue was washed with ethanol. The filtrate was concentrated under reduced pressure and the product was then dried under high vacuum. Yield: 126 mg (98% of theory).

GC-MS (method 2B): Rₜ=3.97 min

### Example 177A

### 1-Benzyl 2-methyl (4R)-4-(2,2-difluoroethoxy)pyrrolidine-1,2-dicarboxylate [diastereomer mixture, 2 isomers]

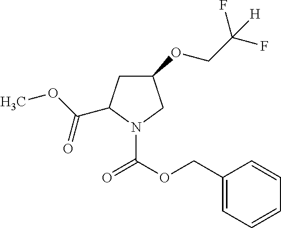 [SMILES: COC(=O)C1C[C@@H](OCC(F)F)CN1C(=O)OCc1ccccc1]

8.20 g (24.7 mmol, purity: 84%) of 1-benzyl 2-methyl (4R)-4-hydroxypyrrolidine-1,2-dicarboxylate and 5.81 g (27.1 mmol) of 2,2-difluoroethyl trifluoromethanesulphonate were initially charged in N,N-dimethylformamide (200 ml) under argon, and 1.28 g (32.1 mmol, 60% suspension in paraffin oil) of sodium hydride were added at 0° C. The mixture was allowed to warm to RT and stirred for 1 h. The reaction mixture was concentrated under reduced pressure, and the residue was taken up in dichloromethane and then washed with water and saturated aqueous sodium chloride solution. The organic phase was dried over sodium sulphate, filtered and concentrated under reduced pressure. The crude product was purified by preparative RP-HPLC (acetonitrile/water). Yield: 6.55 g (69% of theory, purity: 90%, diastereomer ratio: about 5:4).

LC-MS (Method 2A): Rₜ=0.98 min (enantiomerically pure isomer 1), Rₜ=0.99 min (enantiomerically pure isomer 2);

MS (ESIpos): m/z=344 [M+H]⁺.

### Example 178A

### Benzyl (4R)-4-(2,2-difluoroethoxy)-2-(hydroxymethyl)pyrrolidine-1-carboxylate [diastereomer mixture, 2 isomers]

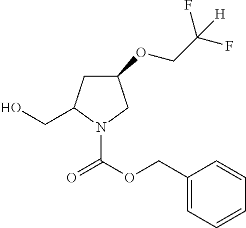 [SMILES: O=C(OCc1ccccc1)N1C[C@H](OCC(F)F)CC1CO]

6.50 g (17.0 mmol, purity: 90%) of 1-benzyl 2-methyl (4R)-4-(2,2-difluoroethoxy)pyrrolidine-1,2-dicarboxylate [diastereomer mixture, 2 isomers] were initially charged in tetrahydrofuran (200 ml), and 1.11 g (51.1 mmol) of lithium borohydride were added at 0° C. The reaction mixture was warmed to RT and then stirred overnight. Water (50 ml) was added carefully to the reaction mixture and the tetrahydrofuran was removed under reduced pressure. The residue was taken up in dichloromethane and washed with 1 M sodium carbonate solution. The organic phase was dried over sodium sulphate, filtered and concentrated under reduced pressure. The crude product was purified by preparative RP-HPLC (acetonitrile/water). Yield: 4.25 g (79% of theory, diastereomer ratio: about 1:1).

LC-MS (Method 2A): Rₜ=0.87 min (enantiomerically pure isomer 1), Rₜ=0.88 min (enantiomerically pure isomer 2);

MS (ESIpos): m/z=316 [M+H]⁺.

### Example 179A

### Benzyl (4R)-4-(2,2-difluoroethoxy)-2-(hydroxymethyl)pyrrolidine-1-carboxylate [enantiomerically pure isomer 1]

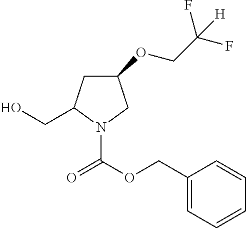 [SMILES: O=C(OCc1ccccc1)N1C[C@H](OCC(F)F)CC1CO]

Diastereomer separation on a chiral phase of 1.06 g of the compound from Example 178A according to Method 27D gave 447 mg of Example 179A (enantiomerically pure isomer 1) and 510 mg of Example 180A (enantiomerically pure isomer 2).

HPLC (Method 25E): Rₜ=1.90 min, >99.0% de; LC-MS (method 2A): Rₜ=0.88 min; MS (ESIpos): m/z=316 [M+H]⁺.

### Example 180A

Benzyl (4R)-4-(2,2-difluoroethoxy)-2-(hydroxymethyl)pyrrolidine-1-carboxylate [enantiomerically pure isomer 2]

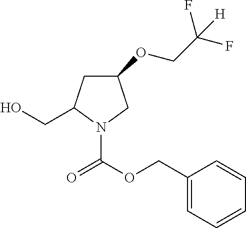 [SMILES: O=C(OCc1ccccc1)N1C[C@H](OCC(F)F)CC1CO]

Diastereomer separation on a chiral phase of 1.06 g of the compound from Example 178A according to Method 27D gave 447 mg of Example 179A (enantiomerically pure isomer 1) and 510 mg of Example 180A (enantiomerically pure isomer 2).

HPLC (Method 25E): Rₜ=2.97 min, >99.0% de; LC-MS (method 2A): Rₜ=0.87 min; MS (ESIpos): m/z=316 [M+H]⁺.

### Example 181A

[(4R)-4-(2,2-Difluoroethoxy)pyrrolidin-2-yl]methanol [enantiomerically pure isomer 2]

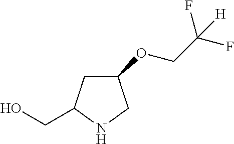 [SMILES: OCC1C[C@@H](OCC(F)F)CN1]

510 mg (1.62 mmol) of benzyl (4R)-4-(2,2-difluoroethoxy)-2-(hydroxymethyl)pyrrolidine-1-carboxylate [enantiomerically pure isomer 2, Example 180A] were initially charged in methanol (10.4 ml), 52.0 mg of palladium on carbon (10%) were added under argon and the mixture was stirred under a hydrogen atmosphere at standard pressure until the hydrogen uptake had ended. The reaction solution was filtered through kieselguhr, the filter cake was washed with methanol and the filtrate was concentrated under reduced pressure. Yield: 299 mg (quant.).

MS (method 2C): m/z=182 [M+H]⁺.

### Example 182A

### 1-Benzyl 2-methyl (4R)-4-[(1,1-²H₂)ethyloxy]pyrrolidine-1,2-dicarboxylate [diastereomer mixture, 2 isomers]

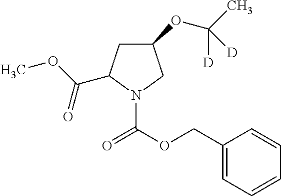 [SMILES: [2H]C([2H])(C)O[C@@H]1CC(C(=O)OC)N(C(=O)OCc2ccccc2)C1]

Under argon, 1.35 g (33.8 mmol, 60% suspension in paraffin oil) of sodium hydride were added to 6.29 g (22.5 mmol) of 1-benzyl 2-methyl (4R)-4-hydroxypyrrolidine-1,2-dicarboxylate in N,N-dimethylformamide (100 ml) at 0° C. The mixture was stirred for 30 min, 5.00 g (9.37 ml, 45.1 mmol) of 1-bromo(2,2-²H₂)propane and 832 mg (2.25 mmol) of tetra-n-butylammonium iodide were added and the mixture was warmed to RT and stirred for 3 h. Water was added carefully, and the reaction mixture was then concentrated under reduced pressure. The residue was taken up in ethyl acetate and washed with water and saturated aqueous sodium chloride solution. The organic phase was dried over sodium sulphate, filtered and concentrated under reduced pressure. The crude product was used without further purification in the next step. Yield: 5.07 g (60% of theory, purity: 83%, diastereomer ratio: about 4:5).

LC-MS (Method 1A): Rₜ=0.98 min (enantiomerically pure isomer 1), Rₜ=0.99 min (enantiomerically pure isomer 2);

MS (ESIpos): m/z=310 [M+H]⁺.

### Example 183A

### Benzyl (4R)-4-[(1,1-²H₂)ethyloxy]-2-(hydroxymethyl)pyrrolidine-1-carboxylate [diastereomer mixture, 2 isomers]

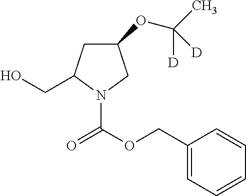 [SMILES: [2H]C([2H])(C)O[C@@H]1CC(CO)N(C(=O)OCc2ccccc2)C1]

4.00 g (10.8 mmol, purity: 83%) of 1-benzyl 2-methyl (4R)-4-[(1,1-²H₂)ethyloxy]pyrrolidine-1,2-dicarboxylate [diastereomer mixture, 2 isomers] were initially charged in tetrahydrofuran (26.0 ml), and 270 mg (12.4 mmol) of lithium borohydride were added at 0° C. The reaction mixture was warmed to RT and then stirred overnight. Water was then added carefully and the mixture was subsequently acidified with 2 N aqueous hydrogen chloride solution. The aqueous phase was extracted repeatedly with ethyl acetate and the combined organic phases were washed with saturated aqueous sodium chloride solution, dried over sodium sulphate, filtered and concentrated under reduced pressure. The crude product was used without further purification in the next step. Yield: 1.01 g (29% of theory, purity: 88%, diastereomer ratio: about 1:1).

LC-MS (Method 1A): Rₜ=0.81 min (enantiomerically pure isomer 1), Rₜ=0.84 min (enantiomerically pure isomer 2);

MS (ESIpos): m/z=282 [M+H]⁺.

### Example 184A

{(4R)-4-[(1,1-²H₂)Ethyloxy]pyrrolidin-2-yl}methanol [diastereomer mixture, 2 isomers]

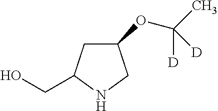

1.00 g (3.55 mmol) of benzyl (4R)-4-[(1,1-²H₂)ethyloxy]-2-(hydroxymethyl)pyrrolidine-1-carboxylate [diastereomer mixture, 2 isomers] were initially charged in methanol (23.0 ml), 110 mg of palladium on carbon (10%) and 55.0 mg of platinum(IV) oxide were added under argon and the mixture was then stirred under a hydrogen atmosphere at standard pressure until the hydrogen uptake had ended. The reaction solution was filtered through kieselguhr, the filter cake was washed with methanol and the filtrate was concentrated under reduced pressure. Yield: 576 mg (quant.).

MS (method 2C): m/z=148 [M+H]⁺.

### Example 185A

### 4-Benzyl-5-(methoxymethyl)-2-methylmorpholin-3-one [diastereomer mixture, 4 isomers]

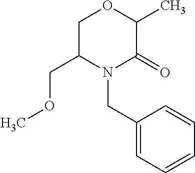 [SMILES: COCC1COC(C)C(=O)N1Cc1ccccc1]

15.0 g (63.8 mmol) of 4-benzyl-5-(hydroxymethyl)-2-methylmorpholin-3-one [diastereomer mixture, 4 isomers] were initially charged in N,N-dimethylformamide (600 ml), and 5.10 g (128 mmol, 60% suspension in paraffin oil) of sodium hydride and 22.6 g (9.92 ml, 159 mmol) of iodomethane were added. The mixture was stirred for 2 h and the reaction was then terminated by slowly adding water (30 ml). The mixture was concentrated under reduced pressure, and the residue was taken up in water and extracted repeatedly with ethyl acetate. The organic phase was dried over sodium sulphate, filtered and concentrated under reduced pressure. The residue was taken up in toluene and washed with water and saturated aqueous sodium chloride solution. The organic phase was dried over sodium sulphate, filtered and concentrated under reduced pressure. The crude product was purified by preparative RP-HPLC (acetonitrile/water). Yield: 16.7 g (96% of theory).

LC-MS (method 1A): Rₜ=0.82 min; MS (ESIpos): m/z=250 [M+H]⁺.

### Example 186A

### 2-Allyl-4-benzyl-5-(methoxymethyl)-2-methylmorpholin-3-one [diastereomer mixture, 4 isomers]

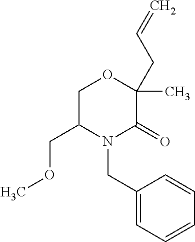 [SMILES: C=CCC1(C)OCC(COC)N(Cc2ccccc2)C1=O]

4.00 g (15.5 mmol) of 4-benzyl-5-(methoxymethyl)-2-methylmorpholin-3-one [diastereomer mixture, 4 isomers] were initially charged in tetrahydrofuran (200 ml), 21.7 ml (21.7 mmol) of 1 M lithium hexamethyldisilazide solution in tetrahydrofuran were added under argon and at −78° C. and the reaction mixture was then stirred for 15 min. Subsequently, at −78° C., 3.12 g (1.70 ml, 18.6 mmol) of allyl iodide were added, and the reaction mixture was warmed to RT and stirred overnight. The reaction was terminated by addition of saturated aqueous ammonium chloride solution and the mixture was extracted with ethyl acetate. The organic phase was washed with saturated aqueous sodium chloride solution, dried over magnesium sulphate, filtered and concentrated under reduced pressure. The crude product was used without further purification in the next step. Yield: 5.26 g (99% of theory, purity: 84%).

LC-MS (method 1A): Rₜ=1.04 min; MS (ESIpos): m/z=290 [M+H]⁺.

### Example 187A

### [4-Benzyl-5-(methoxymethyl)-2-methyl-3-oxomorpholin-2-yl]acetaldehyde [diastereomer mixture, 4 isomers]

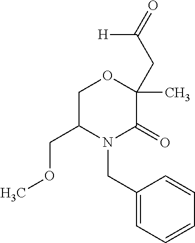 [SMILES: COCC1COC(C)(CC=O)C(=O)N1Cc1ccccc1]

5.26 g (15.4 mmol, purity: 84%) of 2-allyl-4-benzyl-5-(methoxymethyl)-2-methylmorpholin-3-one [diastereomer mixture, 4 isomers] were initially charged in tetrahydrofuran (333 ml) and water (199 ml), and 3.87 ml (1.42 mmol) of a 2.5% solution of osmium tetroxide in tert-butanol and 9.88 g (46.2 mmol) of sodium periodate were added at 0° C. The mixture was then warmed to RT and stirred for 20 h. The reaction solution was filtered through kieselguhr and the tetrahydrofuran was removed under reduced pressure. The aqueous phase was extracted with ethyl acetate and the organic phase was washed with saturated aqueous sodium chloride solution. The organic phase was dried over sodium sulphate, filtered and concentrated under reduced pressure. The crude product was used without further purification in the next step. Yield: 5.06 g (91% of theory, purity: 81%).

LC-MS (Method 1A): Rₜ=0.85 min (diastereomer 1, 2 isomers), Rₜ=0.88 min (diastereomer 2, 2 isomers);

MS (ESIpos): m/z=292 [M+H]⁺.

### Example 188A

### 4-Benzyl-2-(2-hydroxyethyl)-5-(methoxymethyl)-2-methylmorpholin-3-one [diastereomer mixture, 4 isomers]

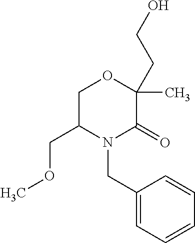 [SMILES: COCC1COC(C)(CCO)C(=O)N1Cc1ccccc1]

5.00 g (13.7 mmol, purity: 80%) of [4-benzyl-5-(methoxymethyl)-2-methyl-3-oxomorpholin-2-yl]acetaldehyde [diastereomer mixture, 4 isomers] were initially charged in methanol (102 ml), and 1.56 g (41.2 mmol) of sodium borohydride were added at 0° C. The mixture was then warmed to RT and stirred for 30 min. Water was added, and the reaction solution was extracted with ethyl acetate. The organic phase was dried over sodium sulphate, filtered and concentrated under reduced pressure. The crude product was used without further purification in the next step. Yield: 4.84 g (65% of theory, purity: 54%).

LC-MS (Method 2A): Rₜ=0.74 min (diastereomer 1, 2 isomers), Rₜ=0.75 min (diastereomer 2, 2 isomers);

MS (ESIpos): m/z=294 [M+H]⁺.

### Example 189A

### 2-[4-Benzyl-5-(methoxymethyl)-2-methylmorpholin-2-yl]ethanol [diastereomer mixture, 4 isomers]

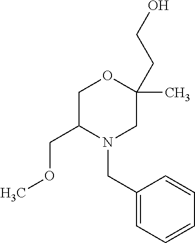 [SMILES: COCC1COC(C)(CCO)CN1Cc1ccccc1]

4.84 g (18.9 mmol, purity: 54%) of 4-benzyl-2-(2-hydroxyethyl)-5-(methoxymethyl)-2-methylmorpholin-3-one [diastereomer mixture, 4 isomers] were initially charged in tetrahydrofuran (88 ml), 44.6 ml (89.2 mmol) of 2 M borane/dimethyl sulphide complex solution in tetrahydrofuran were added under argon and the mixture was stirred under reflux for 1 h. The mixture was subsequently cooled to RT, ethanol (40 ml) was added carefully and the mixture was stirred under reflux for 1 h. The mixture was then concentrated completely under reduced pressure, and the residue was taken up in acetonitrile and purified by preparative RP-HPLC (acetonitrile/water, isocratic). Yield: 2.65 g (quant., diastereomer ratio about 3:2).

LC-MS (Method 1A): Rₜ=0.49 min (diastereomer 1, 2 isomers), Rₜ=0.53 min (diastereomer 2, 2 isomers);

MS (ESIpos): m/z=280 [M+H]⁺.

### Example 190A

### 2-[5-(Methoxymethyl)-2-methylmorpholin-2-yl]ethanol [diastereomer mixture, 4 isomers]

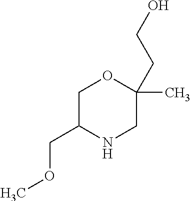

2.65 g (9.49 mmol) of 2-[4-benzyl-5-(methoxymethyl)-2-methylmorpholin-2-yl)ethanol [diastereomer mixture, 4 isomers] were initially charged in ethanol (37.9 ml), 250 mg of palladium on carbon (10%) and 125 mg of palladium hydroxide on carbon (20%) were added under argon, and the mixture was stirred under an atmosphere of hydrogen at standard pressure for 20 h. The reaction solution was filtered through kieselguhr and the filter residue was washed with methanol. The filtrate was concentrated under reduced pressure and the product was dried under high vacuum. Yield: 1.75 g (92% of theory).

MS (method 2C): m/z=190 [M+H]⁺.

### Example 191A

### N-Benzyl-2-chloro-N-[(2R)-1-hydroxybutan-2-yl]propanamide [diastereomer mixture, 2 isomers]

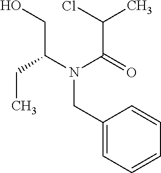 [SMILES: CC[C@H](CO)N(Cc1ccccc1)C(=O)C(C)Cl]

39.24 g (219 mmol) of (2R)-2-(benzylamino)butan-1-ol [lit.: P. Deniz et al., Tetrahedron 2011, 67, 6227-6232] were initially charged in isopropanol (500 ml), and 46.5 g (64.0 ml, 459 mmol) of triethylamine were added. 30.5 g (23.4 ml, 109 mmol) of 2-chloropropionyl chloride [racemate] were then added dropwise and the reaction solution was stirred at RT for 4 h. The mixture was then concentrated under reduced pressure, water was added to the residue and the mixture was extracted with dichloromethane. The organic phases were dried over magnesium sulphate, filtered and concentrated under reduced pressure. The crude product was used without further purification in the next step. Yield: 49.0 g (83% of theory, diastereomer ratio about 1:1).

LC-MS (Method 1A): Rₜ=0.86 min (enantiomerically pure isomer 1), Rₜ=0.88 min (enantiomerically pure isomer 2);

MS (ESIpos): m/z=270 [M+H]⁺.

### Example 192A

### (5R)-4-Benzyl-5-ethyl-2-methylmorpholin-3-one [diastereomer mixture, 2 isomers]

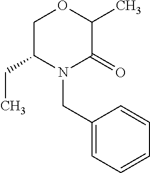 [SMILES: CC[C@@H]1COC(C)C(=O)N1Cc1ccccc1]

25.0 g (92.7 mmol) of N-benzyl-2-chloro-N-[(2R)-1-hydroxybutan-2-yl]propanamide [diastereomer mixture, 2 isomers] were initially charged in isopropanol (400 ml), the mixture was cooled to 0° C. and 34.3 g (306 mmol) of potassium tert-butoxide were then added in one portion. The mixture was gradually warmed to RT and stirred overnight. Most of the isopropanol was removed under reduced pressure and the residue was taken up in ethyl acetate. The organic phase was washed twice with water, once with 1 N aqueous hydrogen chloride solution and once with saturated aqueous sodium chloride solution. The organic phase was dried over magnesium sulphate, filtered and concentrated under reduced pressure. The crude product was used without further purification in the next step. Yield: 19.5 g (90% of theory).

LC-MS (method 1A): Rₜ=0.93 min; MS (ESIpos): m/z=234 [M+H]⁺.

### Example 193A

### (5R)-2-Allyl-4-benzyl-5-ethyl-2-methylmorpholin-3-one [diastereomer mixture, 2 isomers]

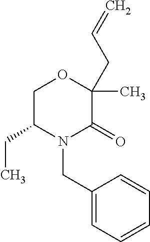 [SMILES: C=CCC1(C)OC[C@@H](CC)N(Cc2ccccc2)C1=O]

17.5 g (75.0 mmol) of (5R)-4-benzyl-5-ethyl-2-methylmorpholin-3-one [diastereomer mixture, 2 isomers] were initially charged in tetrahydrofuran (320 ml), 82.5 ml (82.5 mmol) of 1 M lithium hexamethyldisilazide solution in tetrahydrofuran were added under argon and at −78° C. and the mixture was stirred for 30 min. Subsequently, at −78° C., 10.9 g (7.79 ml, 90.0 mmol) of allyl bromide in tetrahydrofuran (20 ml) were added dropwise. The reaction mixture was allowed to warm to RT and stirred overnight. The reaction was terminated by addition of water and the mixture was then extracted with ethyl acetate. The organic phase was washed with saturated aqueous sodium chloride solution, dried over magnesium sulphate, filtered and concentrated under reduced pressure. The crude product was purified by chromatography on silica gel (cyclohexane/ethyl acetate gradient). Yield: 13.5 g (65% of theory).

LC-MS (method 1A): Rₜ=1.11 min; MS (ESIpos): m/z=274 [M+H]⁺.

### Example 194A

### [(5R)-4-Benzyl-5-ethyl-2-methyl-3-oxomorpholin-2-yl]acetaldehyde [diastereomer mixture, 2 isomers]

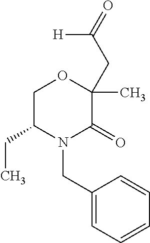 [SMILES: CC[C@@H]1COC(C)(CC=O)C(=O)N1Cc1ccccc1]

10.5 g (38.4 mmol) of (5R)-2-allyl-4-benzyl-5-ethyl-2-methylmorpholin-3-one [diastereomer mixture, 2 isomers] were initially charged in methanol (300 ml), and ozone-containing oxygen was then introduced into the solution at −78° C. for 30 min (ozone generator LAB2B from Triogen/Degrémont Technologies Ltd., ozone concentration: about 30-50 mg/l). To remove excess ozone, pure oxygen was then introduced into the reaction solution for a few minutes. At −78° C., 23.9 g (28.2 ml, 384 mmol) of dimethyl sulphide were added and the reaction solution was stirred overnight while being allowed to warm to RT. The reaction solution was concentrated under reduced pressure and the residue was taken up in ethyl acetate and washed with water, 1 N aqueous hydrogen chloride solution and saturated aqueous sodium chloride solution. The organic phase was dried over magnesium sulphate, filtered and concentrated under reduced pressure. The crude product was used without further purification in the next step. Yield: 10.1 g of crude product.

LC-MS (method 1A): Rₜ=0.90 min; MS (ESIpos): m/z=276 [M+H]⁺.

### Example 195A

### (5R)-4-Benzyl-5-ethyl-2-(2-hydroxyethyl)-2-methylmorpholin-3-one [diastereomer mixture, 2 isomers]

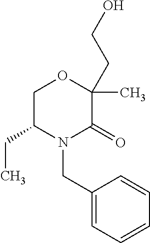 [SMILES: CC[C@@H]1COC(C)(CCO)C(=O)N1Cc1ccccc1]

9.80 g (about 35.6 mmol, crude product) of [(5R)-4-benzyl-5-ethyl-2-methyl-3-oxomorpholin-2-yl]acetaldehyde [diastereomer mixture, 2 isomers] were initially charged in methanol (100 ml), and 1.41 g (37.4 mmol) of sodium borohydride were added at 0° C. The mixture was then allowed to warm to RT and stirred for 30 min. Water was added and the reaction solution was extracted with ethyl acetate. The organic phase was dried over magnesium sulphate, filtered and concentrated under reduced pressure. The crude product was purified by chromatography on silica gel (cyclohexane/ethyl acetate gradient). Yield: 9.79 g (99% of theory).

LC-MS (method 1A): Rₜ=0.79 min; MS (ESIpos): m/z=278 [M+H]⁺.

### Example 196A

### 2-[(5R)-4-Benzyl-5-ethyl-2-methylmorpholin-2-yl]ethanol [diastereomer mixture, 2 isomers]

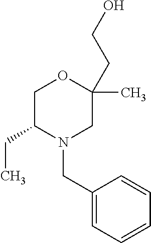 [SMILES: CC[C@@H]1COC(C)(CCO)CN1Cc1ccccc1]

9.79 g (35.3 mmol) of (5R)-4-benzyl-5-ethyl-2-(2-hydroxyethyl)-2-methylmorpholin-3-one [diastereomer mixture, 2 isomers] were initially charged in tetrahydrofuran (180 l), 106 ml (212 mmol) of 2 M borane/dimethyl sulphide complex solution in tetrahydrofuran were added under argon and the mixture was stirred at RT overnight. Ethanol was added until the evolution of gas had ended and the mixture was stirred under reflux for 30 min. The mixture was then concentrated completely under reduced pressure, and the residue was taken up in acetonitrile and purified directly by flash chromatography on silica gel (cyclohexane/ethyl acetate gradient). Yield: 8.60 g (92% of theory, diastereomer ratio about 2:1).

LC-MS (Method 2A): Rₜ=0.46 min (enantiomerically pure isomer 1), Rₜ=0.49 min (enantiomerically pure isomer 2);

MS (ESIpos): m/z=274 [M+H]⁺.

### Example 197A

### 2-[(5R)-5-Ethyl-2-methylmorpholin-2-yl]ethanol [diastereomer mixture, 2 isomers]

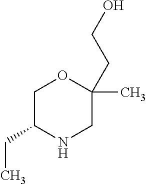

4.00 g (15.2 mmol) of 2-[(5R)-4-benzyl-5-ethyl-2-methylmorpholin-2-yl]ethanol [diastereomer mixture, 2 isomers] were initially charged in ethanol (150 ml), 400 mg of palladium on carbon (10%) and 200 mg of palladium hydroxide on carbon (20%) were added under argon, and the mixture was stirred under an atmosphere of hydrogen at standard pressure for 40 h. The reaction solution was filtered through kieselguhr and the filter residue was washed with ethanol. The filtrate was concentrated under reduced pressure and the product was dried under high vacuum. Yield: 2.55 g (90% of theory).

MS (method 1C): m/z=174 [M+H]⁺.

### Example 198A

### tert-Butyl 2-{[(1-methoxy-1-oxobutan-2-yl)amino]methyl}azetidine-1-carboxylate [diastereomer mixture, 4 isomers]

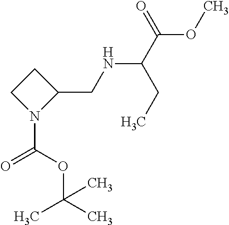 [SMILES: CCC(NCC1CCN1C(=O)OC(C)(C)C)C(=O)OC]

1.80 g (9.66 mmol) of tert-butyl 2-(aminomethyl)azetidine-1-carboxylate [racemate] were dissolved in 25 ml of dichloromethane, 1.62 ml (1.17 g, 11.6 mmol) of triethylamine and 1.11 ml (1.75 g, 9.66 mmol) of methyl 2-bromobutanoate [racemate] were added and the mixture was stirred under reflux overnight. 1.35 ml (0.98 g, 9.66 mmol) of triethylamine and 0.89 ml (1.40 g, 7.73 mmol) of methyl 2-bromobutanoate [racemate] were added, and the mixture was stirred under reflux overnight. After cooling to room temperature, water was added and the phases were separated. The aqueous phase was extracted twice with dichloromethane and the combined organic phases were washed with saturated aqueous sodium chloride solution, dried over sodium sulphate, filtered and then freed of the solvent under reduced pressure. This gave 2.64 g (83% of theory, purity: 87%) of the desired product.

LC-MS (Method 6A): Rₜ=2.16 min (diastereomer 1, 2 isomers), Rₜ=2.22 min (diastereomer 2, 2 isomers);

MS (ESIpos): m/z=287 [M+H]⁺

### Example 199A

### tert-Butyl 2-({[(benzyloxy)carbonyl](1-methoxy-1-oxobutan-2-yl)amino}methyl)azetidine-1-carboxylate [diastereomer mixture, 4 isomers]

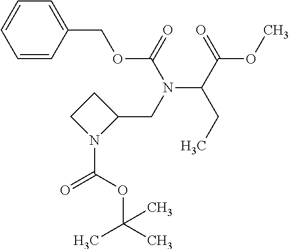 [SMILES: CCC(C(=O)OC)N(CC1CCN1C(=O)OC(C)(C)C)C(=O)OCc1ccccc1]

At 0° C., a solution of 1.88 ml (2.25 g, 13.2 mmol) of benzyl chloroformate in 7 ml of toluene was slowly added dropwise to 3.75 g (8.77 mmol) of tert-butyl 2-{[(1-methoxy-1-oxobutan-2-yl)amino]methyl}azetidine-1-carboxylate [diastereomer mixture, 4 isomers] in 100 ml of THF. A solution of 2.20 ml (1.59 g, 15.8 mmol) of triethylamine in 10 ml of THF was slowly added dropwise and the mixture was stirred at room temperature overnight. The mixture was concentrated under reduced pressure, and water and ethyl acetate were added to the residue. After phase separation, the aqueous phase was extracted twice with ethyl acetate, and the combined organic phases were washed with saturated aqueous sodium chloride solution. The mixture was dried over sodium sulphate and filtered, the filtrate was concentrated under reduced pressure and the residue was dried under high vacuum. Cyclohexane and ethyl acetate were then added to the residue and the product was purified by silica gel chromatography (cyclohexane/ethyl acetate 10:3). This gave 2.31 g (38% of theory, purity: 62%) of the desired product.

### Example 200A

### Methyl 2-{(azetidin-2-ylmethyl)[(benzyloxy)carbonyl]amino}butanoate trifluoroacetate [diastereomer mixture, 4 isomers]

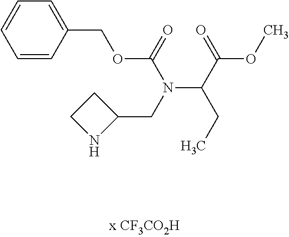 [SMILES: CCC(C(=O)OC)N(CC1CCN1)C(=O)OCc1ccccc1]

2.62 ml (3.88 g, 34.1 mmol) of trifluoroacetic acid were added to 2.31 g (3.41 mmol, purity: 62%) of tert-butyl 2-({[(benzyloxy)carbonyl](1-methoxy-1-oxobutan-2-yl)amino}methyl)azetidine-1-carboxylate [diastereomer mixture, 4 isomers] in 45 ml of dichloromethane, and the mixture was stirred overnight. The mixture was then concentrated under reduced pressure, dichloromethane and water were added to the residue and the phases were separated. The aqueous phase was extracted twice with dichloromethane and the combined organic phases were washed with saturated aqueous sodium chloride solution. After drying over sodium sulphate, the mixture was concentrated under reduced pressure and the residue was dried under high vacuum. This gave 1.41 g (28% of theory, purity: 29%) of the desired product.

LC-MS (method 1A): Rₜ=0.72 min; MS (ESIpos): m/z=320 [M+H-TFA]⁺

### Example 201A

### Benzyl 3-ethyl-2-oxo-1,4-diazabicyclo[4.2.0]octane-4-carboxylate [diastereomer mixture, 4 isomers]

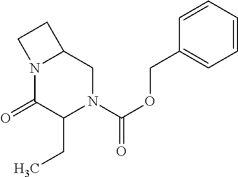 [SMILES: CCC1C(=O)N2CCC2CN1C(=O)OCc1ccccc1]

0.65 g (4.71 mmol) of potassium carbonate were added to 1.41 g (0.94 mmol, purity: 29%) of methyl 2-{(azetidin-2-ylmethyl) [(benzyloxy)carbonyl]amino}butanoate trifluoroacetate [diastereomer mixture, 4 isomers] in 30 ml of methanol, and the mixture was stirred at room temperature overnight. The mixture was then concentrated under reduced pressure, water and ethyl acetate were added to the residue and the phases were separated. The aqueous phase was extracted twice with ethyl acetate and the combined organic phases were washed with saturated aqueous sodium chloride solution. After drying over sodium sulphate, the mixture was concentrated under reduced pressure and the residue was dried under high vacuum. The residue was dissolved in methanol and water and purified by preparative HPLC (RP18 column, mobile phase: acetonitrile/water gradient). This gave 383 mg (quant.) of the desired product.

LC-MS (method 1A): Rₜ=0.89 min; MS (ESIpos): m/z=289 [M+H]⁺

### Example 202A

### 3-Ethyl-1,4-diazabicyclo[4.2.0]octan-2-one [diastereomer mixture, 4 isomers]

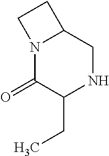

Under argon, 424 mg (0.39 mmol) of 10% palladium on activated carbon were added to 383 mg (0.78 mmol, purity: 60%) of benzyl 3-ethyl-2-oxo-1,4-diazabicyclo[4.2.0]-octane-4-carboxylate [diastereomer mixture, 4 isomers, Example 201A] in 30 ml of methanol, and the mixture was hydrogenated at RT and standard pressure for 4 h. The mixture was then filtered, the filtrate was concentrated under reduced pressure and the residue was dried under high vacuum. This gave 74.8 mg (61% of theory) of the desired product.

MS (method 1C): m/z=155 [M+H]⁺

### Example 203A

### tert-Butyl 2-{[(1,3-dimethoxy-1-oxopropan-2-yl)amino]methyl}azetidine-1-carboxylate [diastereomer mixture, 4 isomers]

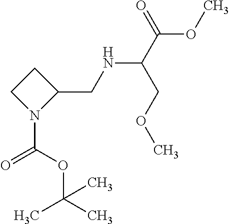 [SMILES: COCC(NCC1CCN1C(=O)OC(C)(C)C)C(=O)OC]

3.00 g (16.1 mmol) of tert-butyl 2-(aminomethyl)azetidine-1-carboxylate [racemate] were dissolved in 40 ml of dichloromethane, 2.69 ml (1.96 g, 19.3 mmol) of triethylamine and 3.17 g (16.1 mmol) of methyl 2-bromo-3-methoxypropanoate [racemate] were added and the mixture was stirred at room temperature overnight. 1.12 ml (0.82 g, 8.05 mmol) of triethylamine and 0.90 g (4.59 mmol) of methyl 2-bromo-3-methoxypropanoate [racemate] were added and the mixture was stirred under reflux overnight. 2.69 ml (1.96 g, 19.3 mmol) of triethylamine and 3.17 g (16.1 mmol) of methyl 2-bromo-3-methoxypropanoate [racemate] were then added and the mixture was stirred under reflux overnight. After cooling, the precipitate was filtered off, water was added to the filtrate and the phases were separated. The aqueous phase was extracted twice with dichloromethane and the combined organic phases were washed with saturated aqueous sodium chloride solution. After drying over sodium sulphate, the mixture was freed of the solvent under reduced pressure and the residue was dried under high vacuum. This gave 6.89 g (94% of theory, purity: 67%) of the desired product.

LC-MS (Method 6A): 1.91 min (diastereomer 1, 2 isomers), Rₜ=1.96 min (diastereomer 2, 2 isomers);

MS (ESIpos): m/z=303 [M+H]⁺

### Example 204A

### Methyl N-(azetidin-2-ylmethyl)-N-[(benzyloxy)carbonyl]-O-methylserinate trifluoroacetate [diastereomer mixture, 4 isomers]

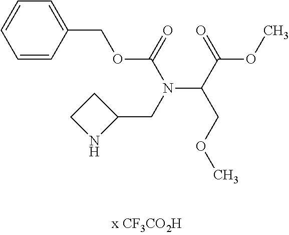 [SMILES: COCC(C(=O)OC)N(CC1CCN1)C(=O)OCc1ccccc1]

17.6 ml (25.9 g, 227 mmol) of trifluoroacetic acid were added to 6.89 g (15.26 mmol, purity: 67%) of tert-butyl 2-{[(1,3-dimethoxy-1-oxopropan-2-yl)amino]methy}azetidine-1-carboxylate [diastereomer mixture, 4 isomers] in 150 ml of dichloromethane, and the mixture was stirred overnight. After the addition of 8.8 ml (12.9 g, 113 mmol) of trifluoroacetic acid, the mixture was stirred overnight at room temperature and then concentrated under reduced pressure and the residue was dissolved in dichloromethane. The solution was concentrated under reduced pressure and the residue obtained was redissolved in dichloromethane and then freed of the solvent under reduced pressure. After drying under high vacuum, the crude product obtained was, without purification, used further in Example 205A.

### Example 205A

### 3-(Methoxymethyl)-1,4-diazabicyclo[4.2.0]octan-2-one [diastereomer mixture, 4 isomers]

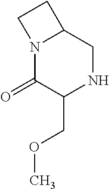

11.4 g (82.7 mmol) of potassium carbonate were added to 10.9 g (20.6 mmol, purity: 60%) of methyl N-(azetidin-2-ylmethyl)-N-[(benzyloxy)carbonyl]-O-methyl serinate trifluoroacetate [diastereomer mixture, 4 isomers] in 150 ml of methanol, and the mixture was stirred at room temperature overnight. The mixture was then concentrated under reduced pressure and the residue was dried under high vacuum. This gave 22.5 g of crude product which was used in Example 206A without further purification.

MS (method 1C): m/z=171 [M+H]⁺

### Example 206A

### Benzyl 3-(methoxymethyl)-2-oxo-1,4-diazabicyclo[4.2.0]octane-4-carboxylate [diastereomer mixture, 4 isomers]

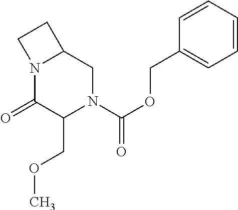 [SMILES: COCC1C(=O)N2CCC2CN1C(=O)OCc1ccccc1]

At 0° C., a solution of 2.82 ml (3.38 g, 19.8 mmol) of benzyl chloroformate in 6.5 ml of toluene was added dropwise to 22.48 g (19.8 mmol, purity: 19%) of 3-(methoxymethyl)-1,4-diazabicyclo[4.2.0]octan-2-one [diastereomer mixture, 4 isomers] in 200 ml of THF. A solution of 3.13 ml (2.41 g, 23.8 mmol) of triethylamine in 10 ml of THF was slowly added dropwise and the mixture was stirred at room temperature overnight. The mixture was then cooled to 0° C. and first a solution of 1.69 ml (2.03 g, 11.9 mmol) of benzyl chloroformate in 4 ml toluene and then, slowly, a solution of 1.93 ml (1.40 g, 13.9 mmol) of triethylamine in 10 ml of THF were added dropwise and the mixture was stirred at room temperature overnight. After filtration, the filtrate was concentrated under reduced pressure, and water and ethyl acetate were then added. After phase separation, the aqueous phase was extracted twice with ethyl acetate, and the combined organic phases were washed with saturated aqueous sodium chloride solution. The mixture was dried over sodium sulphate and filtered, the filtrate was concentrated under reduced pressure and the residue was dried under high vacuum. The residue was dissolved in acetonitrile and water and purified by preparative HPLC (RP18 column, mobile phase: acetonitrile/water gradient). This gave 1.08 g (18% of theory) of the desired product.

LC-MS (method 1A): Rₜ=0.83 min; MS (ESIpos): m/z=305 [M+H]⁺

### Example 207A

### Benzyl 3-(methoxymethyl)-2-oxo-1,4-diazabicyclo[4.2.0]octane-4-carboxylate [enantiomerically pure isomer 3]

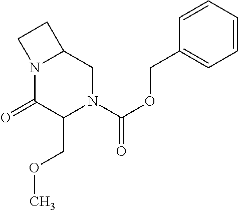 [SMILES: COCC1C(=O)N2CCC2CN1C(=O)OCc1ccccc1]

1.08 g of benzyl 3-(methoxymethyl)-2-oxo-1,4-diazabicyclo[4.2.0]octane-4-carboxylate [diastereomer mixture, 4 isomers] (Example 206A) were separated into the isomers on a chiral phase [Method 28D].

Yield: enantiomerically pure isomer 3: 266.5 mg (99.8% ee) enantiomerically pure isomer 3: Rₜ=9.74 min [Method 26E].

LC-MS (method 1A): Rₜ=0.79 min; MS (ESIpos): m/z=305 [M+H]⁺

### Example 208A

### 3-(Methoxymethyl)-1,4-diazabicyclo[4.2.0]octan-2-one [enantiomerically pure isomer 3]

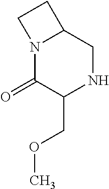

Under argon, 465 mg (0.44 mmol) of 10% palladium on activated carbon were added to 266.5 mg of benzyl 3-(methoxymethyl)-2-oxo-1,4-diazabicyclo[4.2.0]octane-4-carboxylate [enantiomerically pure isomer 3, Example 207A] in 25 ml of methanol, and the mixture was hydrogenated at RT and standard pressure overnight. The mixture was then filtered, the filtrate was concentrated under reduced pressure and the residue was dried under high vacuum. This gave 138 mg (92% of theory) of the desired product.

MS (method 1C): m/z=171 [M+H]⁺

### Example 209A

### tert-Butyl 4-benzyl-5-oxo-4,7-diazaspiro[2.5]octane-7-carboxylate

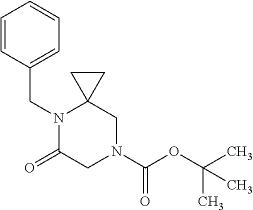 [SMILES: CC(C)(C)OC(=O)N1CC(=O)N(Cc2ccccc2)C2(CC2)C1]

Under argon and at 0° C., 2.47 g (61.9 mmol) of sodium hydride were added a little at a time to 2.50 g (8.84 mmol) of tert-butyl 5-oxo-4,7-diazaspiro[2.5]octane-7-carboxylate in 80 ml of THF, and the mixture was stirred at 0° C. for 30 min 1.26 ml (1.81 g, 10.6 mmol) of benzyl bromide were then added dropwise, and the mixture was stirred at room temperature overnight. The mixture was then cooled to 0° C., 1.24 g (30.9 mmol) of sodium hydride were added and the mixture was stirred at 0° C. for 30 min 0.63 ml (0.91 g, 5.3 mmol) of benzyl bromide was added dropwise, and the mixture was stirred at room temperature overnight. Subsequently, at 0° C., first ethanol and then water and ethyl acetate were added. After phase separation, the aqueous phase was extracted twice with ethyl acetate and the combined organic phases were dried over sodium sulphate. After filtration, the filtrate was concentrated under reduced pressure and the residue was dried under high vacuum and purified by silica gel chromatography (cyclohexane/ethyl acetate 10:1) and then by preparative HPLC (RP18 column, mobile phase: acetonitrile/water gradient). This gave 1.98 g (71% of theory) of the desired product.

LC-MS (method 1A): Rₜ=1.09 min; MS (ESIpos): m/z=317 [M+H]⁺

¹H-NMR (400 MHz, DMSO-d₆): δ [ppm]=7.38-7.14 (m, 5H), 4.41 (s, 2H), 4.16 (br. s., 2H), 1.40 (br. s., 9H), 0.98-0.89 (m, 2H), 0.79-0.72 (m, 2H).

### Example 210A

### tert-Butyl 4-benzyl-6-methyl-5-oxo-4,7-diazaspiro[2.5]octane-7-carboxylate [racemate]

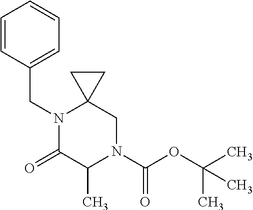 [SMILES: CC1C(=O)N(Cc2ccccc2)C2(CC2)CN1C(=O)OC(C)(C)C]

At −78° C. and under argon, 11.38 ml (11.38 mmol) of a 1 molar solution of lithium hexamethyldisilazide in THF were added dropwise to 1.20 g (3.79 mmol) of tert-butyl 4-benzyl-5-oxo-4,7-diazaspiro[2.5]octane-7-carboxylate in 48 ml of THF, and the mixture was stirred at −78° C. for 30 min 0.47 ml (7.59 mmol) of methyl iodide was then added dropwise, and the mixture was stirred for 1.5 h. At 0° C., first saturated aqueous ammonium chloride solution and then ethyl acetate were added. After phase separation, the aqueous phase was extracted twice with ethyl acetate, and the combined organic phases were washed with saturated aqueous sodium chloride solution and then dried over sodium sulphate. After filtration, the filtrate was concentrated under reduced pressure and the residue was dried under high vacuum, then dissolved in acetonitrile and water and purified by preparative HPLC (RP18 column, mobile phase: acetonitrile/water gradient). This gave 0.54 g (41% of theory) of the desired product.

### Example 211A

### tert-Butyl 6-methyl-5-oxo-4,7-diazaspiro[2.5]octane-7-carboxylate [racemate]

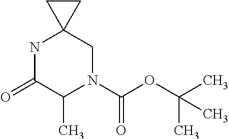 [SMILES: CC1C(=O)NC2(CC2)CN1C(=O)OC(C)(C)C]

At −78° C., 107 mg (15.5 mmol) of lithium were added to about 10 ml of ammonia, and the mixture was stirred for a few minutes. 540 mg (1.55 mmol) of tert-butyl 4-benzyl-6-methyl-5-oxo-4,7-diazaspiro[2.5]octane-7-carboxylate [racemate] in 5 ml of THF were then added dropwise, and the mixture was slowly warmed to room temperature and then stirred at room temperature overnight. At 0° C., first saturated aqueous ammonium chloride solution and then ethyl acetate were added. After phase separation, the aqueous phase was extracted twice with ethyl acetate, and the combined organic phases were washed with saturated aqueous sodium chloride solution and then dried over sodium sulphate. After filtration, the filtrate was concentrated under reduced pressure and the residue was dried under high vacuum. This gave 353 mg of the crude product which was used without further purification.

MS (method 1C): m/z=241 [M+H]⁺

### Example 212A

### 6-Methyl-4,7-diazaspiro[2.5]octan-5-one [racemate]

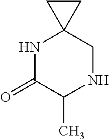

1.06 ml (1.57 g, 13.8 mmol) of trifluoroacetic acid were added to 331 mg (1.38 mmol) of tert-butyl 6-methyl-5-oxo-4,7-diazaspiro[2.5]octane-7-carboxylate [racemate] in 10 ml of dichloromethane, and the mixture was stirred at room temperature for 2 h. The mixture was then concentrated under reduced pressure and the residue was dissolved in dichloromethane. The solution was concentrated under reduced pressure and the residue obtained was re-dissolved in dichloromethane, freed of the solvent under reduced pressure and dried under high vacuum. The crude product obtained (605 mg) was used further without purification.

MS (method 1C): m/z=141 [M+H]⁺

### Example 213A

### [(tert-Butoxycarbonyl) {2-[methoxy(methyl)amino]-2-oxoethyl}amino]acetic acid

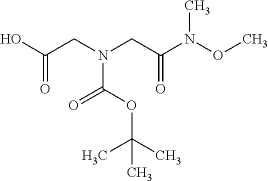 [SMILES: CON(C)C(=O)CN(CC(=O)O)C(=O)OC(C)(C)C]

At 0° C., 33.9 g (177 mmol) of N-ethyl-N′-(3-dimethylaminopropyl)carbodiimide hydrochloride were added a little at a time to 35 g (150 mmol) of 2,2′[(tert-butoxycarbonyl)imino]diacetic acid in N,N-dimethylformamide (250 ml), and the mixture was stirred at room temperature for 1 h. The mixture was cooled to 0° C., and a solution of 17.3 g (177 mmol) of N,O-dimethylhydroxylamine hydrochloride and 22.8 g (30.8 ml, 177 mmol) of N,N-diisopropylethylamine in 150 ml of dimethylformamide was added dropwise at from 0° C. to 5° C. The mixture was stirred at room temperature overnight. The mixture was then added to a mixture of ice and aqueous 1 M hydrogen chloride solution, and ethyl acetate was then added. After phase separation, the aqueous phase was extracted twice with ethyl acetate and the combined organic phases were washed twice with aqueous 1 M hydrogen chloride solution and once with saturated aqueous sodium chloride solution and dried over sodium sulphate. After filtration, the filtrate was concentrated under reduced pressure and the residue was dried under high vacuum. Diethyl ether was added to the residue and the mixture was treated in an ultrasonic bath for 20 min. The solid formed was filtered off and dried under high vacuum. This gave 27.8 g (64% of theory) of the desired product.

LC-MS (method 1A): Rₜ=0.64 min; MS (ESIpos): m/z=277 [M+H]⁺

¹H-NMR (400 MHz, DMSO-d₆): δ [ppm]=12.62 (br. s., 1H), 4.19-4.11 (m, 2H), 3.88 (d, 2H), 3.68 (d, 3H), 3.10 (d, 3H), 1.35 (d, 9H).

### Example 214A

### N-(tert-Butoxycarbonyl)-N-(2-oxopropyl)glycine

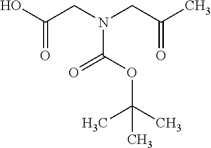 [SMILES: CC(=O)CN(CC(=O)O)C(=O)OC(C)(C)C]

At 0° C., 75.2 ml (26.9 g, 225 mmol) of a 3-molar solution of methylmagnesium bromide in diethyl ether were slowly added dropwise to 13 g (45 mmol) of [(tert-butoxycarbonyl){2-[methoxy(methyl)amino]-2-oxoethyl}amino]acetic acid in 260 ml of THF. The mixture was stirred at room temperature for 2 h. At 0° C., 78 ml of saturated aqueous ammonium chloride solution and 78 ml of water were then added dropwise, and the mixture was slowly allowed to warm to room temperature. Diethyl ether was added, and after phase separation the organic phase was washed with 1 N aqueous sodium hydroxide solution. The aqueous phase was cooled to 0° C., acidified with concentrated hydrogen chloride solution and diluted with diethyl ether. The phases were separated and the aqueous phase was washed twice with ethyl acetate. The combined organic phases were washed with saturated aqueous sodium chloride solution and dried over sodium sulphate. After filtration, the filtrate was concentrated under reduced pressure and the residue was dried under high vacuum. This gave 8.85 g (64% of theory, purity: 75%) of the crude product which was used further without purification.

### Example 215A

### tert-Butyl 4-benzyl-3-methyl-5-oxopiperazine-1-carboxylate [racemate]

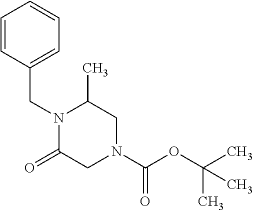 [SMILES: CC1CN(C(=O)OC(C)(C)C)CC(=O)N1Cc1ccccc1]

### i) N-[2-(Benzylamino)propyl]-N-(tert-butoxycarbonyl)glycine

1.76 g (1.81 ml, 16.5 mmol) of benzylamine, 1.53 g (1.46 ml, 25 mmol) of concentrated acetic acid and 7.02 g (16.6 mmol) of sodium triacetoxyborohydride were added to 8.8 g (25 mmol, purity: 67%) of N-(tert-butoxycarbonyl)-N-(2-oxopropyl)glycine in 160 ml of 1,2-dichloroethane, and the mixture was stirred at room temperature overnight. The mixture was then concentrated under reduced pressure.

LC-MS (method 1A): Rₜ=0.71 min; MS (ESIpos): m/z=323 [M+H]⁺

### ii) tert-Butyl 4-benzyl-3-methyl-5-oxopiperazine-1-carboxylate

The crude product N-[2-(benzylamino)propyl]-N-(tert-butoxycarbonyl)glycine from i) was dissolved in 132 ml of DMF, and 4.88 g (25 mmol) of 1-(3-dimethylaminopropyl)-3-ethylcarbodiimide hydrochloride were added. The mixture was stirred at room temperature overnight, 2.44 g (12.7 mmol) of 1-(3-dimethylaminopropyl)-3-ethylcarbodiimide hydrochloride were added and the mixture was once more stirred overnight. The mixture was washed with saturated aqueous sodium bicarbonate solution and saturated aqueous sodium chloride solution and dried over sodium sulphate. After filtration, the filtrate was concentrated under reduced pressure, the residue was dried under high vacuum and purified by silica gel chromatography (dichloromethane, then dichloromethane/methanol 100:2). This gave 6.64 g (79% of theory) of the desired product.

LC-MS (method 1A): Rₜ=1.02 min; MS (ESIpos): m/z=305 [M+H]+

### Example 216A

### tert-Butyl 4-benzyl-2-ethyl-5-methyl-3-oxopiperazine-1-carboxylate [diastereomer mixture, 4 isomers]

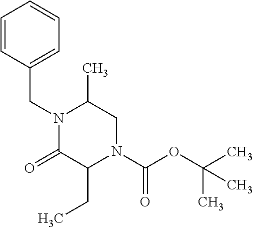 [SMILES: CCC1C(=O)N(Cc2ccccc2)C(C)CN1C(=O)OC(C)(C)C]

At −78° C. and under argon, 5.91 ml (5.91 mmol) of a 1 molar solution of lithium hexamethyldisilazide in THF were added dropwise to 600 mg (1.97 mmol) of tert-butyl 4-benzyl-3-methyl-5-oxopiperazine-1-carboxylate [racemate] in 24 ml of THF, and the mixture was stirred at −78° C. for 30 min 0.51 ml (3.94 mmol) of ethyl trifluoromethanesulphonate was then added dropwise, and the mixture was stirred for 2 h. The mixture was then added to saturated aqueous ammonium chloride solution, and ethyl acetate was added. After phase separation, the aqueous phase was extracted twice with ethyl acetate, and the combined organic phases were washed with saturated aqueous sodium chloride solution and dried over sodium sulphate. After filtration, the filtrate was concentrated under reduced pressure and the residue was dried under high vacuum. This gave 821 mg (87% of theory; purity: 70%) of the desired crude product.

LC-MS (method 1A): Rₜ=1.18 min; MS (ESIpos): m/z=333 [M+H]⁺

### Example 217A

### tert-Butyl 2-ethyl-5-methyl-3-oxopiperazine-1-carboxylate [diastereomer mixture]

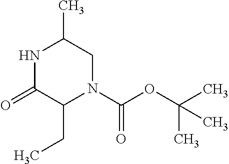 [SMILES: CCC1C(=O)NC(C)CN1C(=O)OC(C)(C)C]

At −78° C., 120 mg (17.3 mmol) of lithium were added to 18 ml of ammonia, and the mixture was stirred for a few minutes. 821 mg (1.72 mmol) of tert-butyl 4-benzyl-2-ethyl-5-methyl-3-oxopiperazine-1-carboxylate [diastereomer mixture, 4 isomers, Example 216A] in 6 ml of THF were then added dropwise, and the mixture was slowly warmed to room temperature and stirred at room temperature overnight. Subsequently, first saturated aqueous ammonium chloride solution and then ethyl acetate were added. After phase separation, the aqueous phase was extracted twice with ethyl acetate, and the combined organic phases were washed with saturated aqueous sodium chloride solution and dried over sodium sulphate. After filtration, the filtrate was concentrated under reduced pressure and the residue was dried under high vacuum. This gave 240 mg of the crude product which was used without further purification.

MS (Method 1C): m/z=243 [M+H]+

### Example 218A

### 3-Ethyl-6-methylpiperazin-2-one trifluoroacetate [diastereomer mixture, 4 isomers]

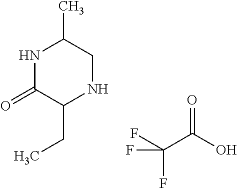

1.46 ml (2.16 g, 18.9 mmol) of trifluoroacetic acid were added to 657 mg (2.71 mmol) of tert-butyl 2-ethyl-5-methyl-3-oxopiperazine-1-carboxylate [diastereomer mixture, 4 isomers, Example 217A] in 22 ml of dichloromethane, and the mixture was stirred at room temperature overnight. The mixture was then concentrated under reduced pressure and the residue was dissolved in dichloromethane. The solution was concentrated under reduced pressure and the residue obtained was dissolved in dichloromethane and then freed of the solvent under reduced pressure and dried under high vacuum. The crude product obtained (1.00 g) was used further without purification.

MS (method 1C): m/z=142 [M+H]⁺

### Example 219A

### Benzyl 2-ethyl-5-methyl-3-oxopiperazine-1-carboxylate [diastereomer mixture, 4 isomers]

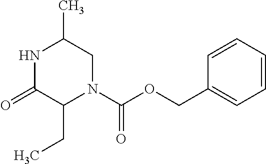 [SMILES: CCC1C(=O)NC(C)CN1C(=O)OCc1ccccc1]

At 0° C., a solution of 0.31 ml (0.37 g, 2.18 mmol) of benzyl chloroformate in 1.2 ml of toluene was added dropwise to 1.55 g (2.18 mmol, purity: 20%) of 3-ethyl-6-methylpiperazin-2-one trifluoroacetate [diastereomer mixture, 4 isomers] in 1 ml of THF. A solution of 0.36 ml (0.26 g, 2.62 mmol) of triethylamine in 0.5 ml of THF was slowly added dropwise, the mixture was stirred at room temperature overnight and water and ethyl acetate were then added. After phase separation, the aqueous phase was extracted twice with ethyl acetate, and the combined organic phases were washed with saturated aqueous sodium chloride solution. The mixture was dried over sodium sulphate and filtered, the filtrate was concentrated under reduced pressure and the residue was dried under high vacuum, dissolved in methanol and water and purified by preparative HPLC (RP18 column, mobile phase: acetonitrile/water gradient). This gave 84 mg (14% of theory) of the desired product.

LC-MS (method 1A): Rₜ=0.83 min; MS (ESIpos): m/z=277 [M+H]⁺

### Example 220A

### 3-Ethyl-6-methylpiperazin-2-one [diastereomer mixture, 4 isomers]

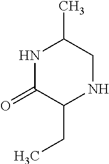

Under argon, 97 mg (0.09 mmol) of 10% palladium on activated carbon were added to 84 mg of benzyl 2-ethyl-5-methyl-3-oxopiperazine-1-carboxylate [diastereomer mixture, 4 isomers, Example 219A] in 10 ml of methanol, and the mixture was hydrogenated at RT and standard pressure for 4 h. The mixture was then filtered, the filtrate was concentrated under reduced pressure and the residue was dried under high vacuum. This gave 41 mg (95% of theory) of the desired product.

MS (method 1C): m/z=143 [M+H]⁺

### Example 221A

### Ethyl (5-methylpyridin-2-yl)acetate

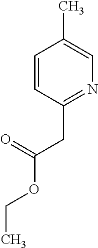

At −50° C., 179 ml (287 mmol) of a 1.6-molar solution of n-butyllithium in hexane were added dropwise to 40.2 ml (29.1 g, 287 mmol) of N,N-diisopropylethylamine and 14.0 ml (10.8 g, 92.8 mmol) of N,N,N,N-tetramethylethylenediamine in 115 ml of THF, and the mixture was stirred at −50° C. for 1 h. 15.1 ml (14.0 g, 130 mmol) of 2,5-dimethylpyridine were then added dropwise, and the mixture was stirred at 0° C. for 1 h. 12.5 ml (14.2 g, 131 mmol) of ethyl chloroformate were then added dropwise at −78° C., and the mixture was stirred at room temperature overnight. At 0° C., first 40 ml of saturated aqueous ammonium chloride solution and then 30 ml of saturated aqueous sodium chloride solution were added dropwise. At room temperature, ethyl acetate was added, the phases were separated, the aqueous phase was extracted twice with ethyl acetate and the combined organic phases washed with saturated aqueous sodium chloride solution and dried over sodium sulphate. After filtration, the filtrate was concentrated under reduced pressure and the residue was dried under high vacuum, dissolved in cyclohexane/ethyl acetate and purified by silica gel chromatography (cyclohexane/ethyl acetate 10:1-10:5). This gave 6.12 g (26% of theory) of the desired product.

LC-MS (method 8A): Rₜ=0.83 min; MS (ESIpos): m/z=180 [M+H]⁺

### Example 222A

### Ethyl (5-methylpiperidin-2-yl)acetate hydrochloride [diastereomer mixture, 4 isomers]

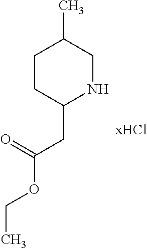

Under argon, 51 mg (0.21 mmol) of platinum(IV) oxide hydrate were added to 2.56 g (13.9 mmol) of ethyl (5-methylpyridin-2-yl)acetate (Example 221A) in 66 ml of acetic acid, and the mixture was hydrogenated at room temperature and standard pressure overnight. The mixture was then filtered through silica gel, the filtrate was concentrated under reduced pressure and 100 ml of 1 N aqueous hydrogen chloride solution were added to the residue. The mixture was concentrated under reduced pressure and the residue was dried under high vacuum. This gave 2.44 g (78% of theory) of the desired product.

MS (method 1C): m/z=223 [M+H]+

### Example 223A

### 2-(5-Methylpiperidin-2-yl)ethanol [diastereomer mixture, 4 isomers]

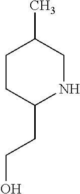

At 0° C., 2.71 ml (2.71 mmol) of a 1.0 molar solution of lithium aluminium hydride in THF were added dropwise to 400 mg (1.80 mmol) of ethyl (5-methylpiperidin-2-yl)acetate hydrochloride [diastereomer mixture, 4 isomers] in 16 ml of THF, and the mixture was stirred at room temperature overnight. 1.44 ml (1.44 mmol) of a 1.0 molar solution of lithium aluminium hydride in THF were then added dropwise at 0° C., and the mixture was stirred at room temperature overnight. At 0° C., 144 μl of water, 156 μl of 3 M aqueous sodium hydroxide solution and another 372 μl of water were added and the precipitate formed was filtered off. The filtrate was concentrated under reduced pressure and the residue was dried under high vacuum. This gave 275 mg (quant.) of the desired product.

MS (method 1C): m/z=144 [M+H]⁺

### Example 224A

### 5-Methylpiperidine-2-carboxamide acetate [diastereomer mixture]

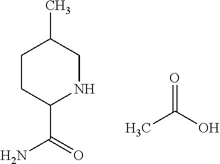

Under argon, 26 mg (0.11 mmol) of platinum(IV) oxide hydrate and 115 mg (0.11 mmol) of 10% palladium on activated carbon were added to 1.00 g (7.19 mmol) of 5-methylpyridine-2-carboxamide in 50 ml of acetic acid, and the mixture was hydrogenated at RT and standard pressure overnight. 26 mg (0.11 mmol) of platinum(IV) oxide hydrate and 115 mg (0.11 mmol) of 10% palladium on activated carbon were added and the mixture was hydrogenated at RT and standard pressure for 3 d. 26 mg (0.11 mmol) of platinum(IV) oxide hydrate and 115 mg (0.11 mmol) of 10% palladium on activated carbon were then added and the mixture was hydrogenated at RT and standard pressure overnight. The mixture was filtered through silica gel, the filtrate was concentrated under reduced pressure and the residue was dried under high vacuum. This gave 2.09 g of the desired crude product.

MS (method 1C): m/z=143 [M+H]⁺

### Example 225A

### 4-Benzyl-6,6-dimethyl-5-oxomorpholine-3-carboxylic acid [racemate]

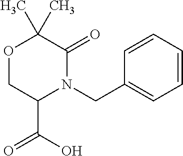 [SMILES: CC1(C)OCC(C(=O)O)N(Cc2ccccc2)C1=O]

At room temperature, 37.65 g (165 mmol) of periodic acid were added to 19.5 g (43.8 mmol) of 4-benzyl-5-(hydroxymethyl)-2,2-dimethylmorpholin-3-one [racemate] in 1200 ml of acetonitrile, and the mixture was stirred for 15 min. At 0° C., 647 mg (3.00 mmol) of pyridinium chlorochromate in 45 ml acetonitrile were then added and the mixture was stirred at 0° C. for 2 h. The mixture was concentrated under reduced pressure, water was then added to the residue and the mixture was washed with ethyl acetate. After separation of the phases, the organic phase was dried over sodium sulphate. After filtration, the filtrate was concentrated under reduced pressure and the residue was dried under high vacuum. This gave 18.4 g (56% of theory, purity: 60%) of the desired crude product.

LC-MS (method 1A): Rₜ=0.69 min; MS (ESIpos): m/z=264 [M+H]⁺

### Example 226A

### Methyl 4-benzyl-6,6-dimethyl-5-oxomorpholine-3-carboxylate [racemate]

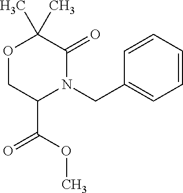 [SMILES: COC(=O)C1COC(C)(C)C(=O)N1Cc1ccccc1]

At 0° C., 12.2 ml (16.7 g, 141 mmol) of thionyl chloride were slowly added dropwise to 18.5 g (70.4 mmol, purity: 60%) of 4-benzyl-6,6-dimethyl-5-oxomorpholine-3-carboxylic acid [racemate] in 232 ml of methanol. With stirring, the mixture was then heated at reflux for 2 h. The mixture was concentrated under reduced pressure and the residue was dried under high vacuum. This gave 18.4 g (80% of theory, purity: 85%) of the desired crude product.

LC-MS (method 1A): Rₜ=0.87 min; MS (ESIpos): m/z=278 [M+H]⁺

### Example 227A

### 4-Benzyl-5-(2-hydroxypropan-2-yl)-2,2-dimethylmorpholin-3-one [racemate]

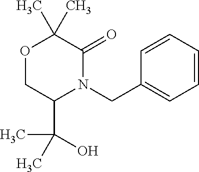 [SMILES: CC1(C)OCC(C(C)(C)O)N(Cc2ccccc2)C1=O]

At 0° C., 32.5 ml (97.6 mmol) of a 3 M solution of methylmagnesium bromide in diethyl ether were slowly added dropwise to 9.1 g (27.9 mmol, purity: 85%) of methyl 4-benzyl-6,6-dimethyl-5-oxomorpholine-3-carboxylate [racemate] in 507 ml of THF. Subsequently, the mixture was stirred at 0° C. for 1 h and then at room temperature overnight. At 0° C., 16.7 ml (50.2 mmol) of a 3 M solution of methylmagnesium bromide in diethyl ether were added and the mixture was stirred at room temperature overnight. At 0° C., saturated aqueous ammonium chloride solution was then added and the mixture was freed from THF under reduced pressure. Dichloromethane and water were added to the residue, the phases were separated and the organic phase was washed twice with water. The aqueous phase was then washed with dichloromethane. The combined organic phases were washed with saturated aqueous sodium chloride solution and then dried over sodium sulphate. After filtration, the filtrate was concentrated under reduced pressure and the residue was dried under high vacuum and purified by preparative HPLC (RP18 column, mobile phase: acetonitrile/water gradient). This gave 3.36 g (43% of theory) of the desired product.

LC-MS (method 1A): Rₜ=0.85 min; MS (ESIpos): m/z=278 [M+H]⁺

¹H-NMR (400 MHz, DMSO-d₆): δ [ppm]=7.41-6.99 (m, 5H), 5.35-5.06 (m, 1H), 4.76 (s, 1H), 4.52 (d, 1H), 4.25-3.95 (m, 1H), 3.89-3.57 (m, 1H), 3.12-2.97 (m, 1H), 1.38-1.33 (m, 6H), 1.28-1.23 (m, 3H), 1.20 (s, 3H).

### Example 228A

### 2-(4-Benzyl-6,6-dimethylmorpholin-3-yl)propan-2-ol [racemate]

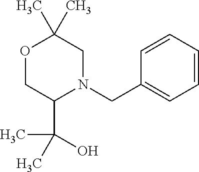 [SMILES: CC1(C)CN(Cc2ccccc2)C(C(C)(C)O)CO1]

At room temperature, 59.3 ml (118 mmol) of a 2 M solution of dimethyl sulphide/borane complex in THF were slowly added dropwise to 3.36 g (11.8 mmol) of 4-benzyl-5-(2-hydroxypropan-2-yl)-2,2-dimethylmorpholin-3-one [racemate] in 421 ml of methanol. The mixture was stirred at room temperature overnight and then under reflux for 7 h. Subsequently, 300 ml of methanol were added slowly at room temperature and the mixture was, with stirring, heated at reflux for 4 h. Subsequently, the mixture was concentrated and the residue was dried under high vacuum and purified by preparative HPLC (RP18 column, mobile phase: acetonitrile/water gradient). This gave 1.51 g (48% of theory) of the desired product.

LC-MS (method 1A): Rₜ=0.58 min; MS (ESIpos): m/z=264 [M+H]⁺

¹H-NMR (400 MHz, DMSO-d₆): δ [ppm]=7.40-7.15 (m, 5H), 5.04 (d, 1H), 4.59 (s, 1H), 3.56 (dd, 1H), 3.41 (t, 1H), 3.02-2.89 (m, 1H), 2.41-2.26 (m, 2H), 1.83 (d, 1H), 1.24 (s, 3H), 1.15 (s, 3H), 1.09 (s, 3H), 0.96 (s, 3H).

### Example 229A

### 2-(4-Benzyl-6,6-dimethylmorpholin-3-yl)propan-2-ol [enantiomerically pure isomer 1]

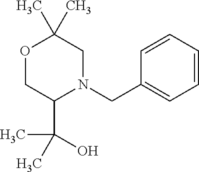 [SMILES: CC1(C)CN(Cc2ccccc2)C(C(C)(C)O)CO1]

1.51 g of 2-(4-benzyl-6,6-dimethylmorpholin-3-yl)propan-2-ol [racemate, Example 228A] were separated into the enantiomers on a chiral phase [Method 29D].

Yield: enantiomerically pure isomer 1: 448 mg (100% ee)

enantiomerically pure isomer 1: Rₜ=5.40 min [Method 27E].

LC-MS (method 1A): Rₜ=0.66 min; MS (ESIpos): m/z=264 [M+H]⁺

¹H-NMR (400 MHz, DMSO-d₆): δ [ppm]=7.39-7.11 (m, 5H), 5.04 (d, 1H), 4.59 (s, 1H), 3.56 (dd, 1H), 3.46-3.35 (m, 1H), 3.30 (s, 1H), 2.96 (d, 1H), 2.40-2.26 (m, 2H), 1.24 (s, 3H), 1.15 (s, 3H), 1.09 (s, 3H), 0.96 (s, 3H).

### Example 230A

### 2-(6,6-Dimethylmorpholin-3-yl)propan-2-ol [enantiomerically pure isomer 1]

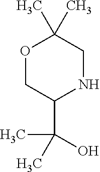

Under argon, 60 mg (0.56 mmol) of 10% palladium on activated carbon and 30 mg (0.21 mmol) of palladium(II) hydroxide were added to 477 mg (1.81 mmol) of 2-(4-benzyl-6,6-dimethylmorpholin-3-yl)propan-2-ol] [enantiomerically pure isomer 1] in 20 ml of ethanol, and the mixture was hydrogenated at RT and standard pressure overnight. The mixture was then filtered through silica gel, the filtrate was concentrated under reduced pressure and the residue was dried under high vacuum. This gave 307 mg (98% of theory) of the desired product.

MS (method 1C): m/z=174 [M+H]+

¹H-NMR (400 MHz, DMSO-d₆): δ [ppm]=4.41-4.23 (m, 1H), 3.50-3.26 (m, 3H), 2.72-2.60 (m, 1H), 2.37 (dd, 1H), 1.18 (s, 3H), 1.10-0.97 (m, 9H).

### Example 231A

### 4-Benzyl-5-(1-hydroxycyclopropyl)-2,2-dimethylmorpholin-3-one [racemate]

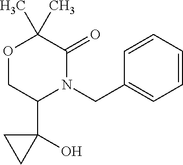 [SMILES: CC1(C)OCC(C2(O)CC2)N(Cc2ccccc2)C1=O]

At room temperature, 0.83 ml (0.79 g, 2.79 mmol) of titanium(IV) tetraprop-2-oxide and then 19.7 ml (59.1 mmol) of a 3 M solution of ethylmagnesium bromide in diethyl ether were slowly added dropwise to 9.1 g (27.9 mmol, purity: 85%) of methyl 4-benzyl-6,6-dimethyl-5-oxomorpholine-3-carboxylate [racemate] in 350 ml of diethyl ether. The mixture was stirred at room temperature overnight, and 0.83 ml (0.79 g, 2.79 mmol) of titanium(IV) tetraprop-2-oxide and 19.7 ml (59.1 mmol) of a 3 M solution of ethylmagnesium bromide in diethyl ether were then slowly added dropwise, and the mixture was stirred overnight. 0.21 ml (0.19 g, 0.69 mmol) of titanium(IV) tetraprop-2-oxide and 4.92 ml (14.7 mmol) of a 3 M solution of ethylmagnesium bromide in diethyl ether were then slowly added dropwise, and the mixture was stirred at room temperature for 4 h. The mixture was added to cooled 10% strength aqueous sulphuric acid and diluted with diethyl ether. The phases were separated, the aqueous phase was washed twice with diethyl ether and the combined organic phases were washed with saturated aqueous sodium chloride solution and then dried over sodium sulphate. After filtration, the filtrate was concentrated under reduced pressure and the residue was dried under high vacuum and purified by preparative HPLC (RP18 column, mobile phase: acetonitrile/water gradient). This gave 1.67 g (20% of theory) of the desired product.

LC-MS (method 1A): Rₜ=0.81 min; MS (ESIpos): m/z=276 [M+H]⁺

¹H-NMR (400 MHz, DMSO-d₆): δ [ppm]=7.42-7.07 (m, 5H), 5.48 (s, 1H), 5.38 (d, 1H), 4.29-4.13 (m, 1H), 4.08-3.94 (m, 1H), 3.83 (dd, 1H), 2.79 (dd, 1H), 1.47-1.25 (m, 6H), 0.80-0.66 (m, 1H), 0.62-0.45 (m, 1H), 0.42-0.21 (m, 2H).

### Example 232A

### 1-(4-Benzyl-6,6-dimethylmorpholin-3-yl)cyclopropanol [racemate]

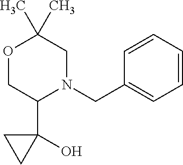 [SMILES: CC1(C)CN(Cc2ccccc2)C(C2(O)CC2)CO1]

At room temperature, 26.8 ml (53.6 mmol) of a 2 M solution of dimethyl sulphide/borane complex in THF were slowly added dropwise to 1.57 g (5.36 mmol) of 4-benzyl-5-(1-hydroxycyclopropyl)-2,2-dimethylmorpholin-3-one [racemate] in 300 ml of methanol. The mixture was stirred at room temperature overnight and then under reflux for 2 h. Subsequently, 300 ml of methanol were added slowly at room temperature and the mixture was heated at reflux for 4 h. The mixture was then concentrated and the residue was purified by preparative HPLC (RP18 column, mobile phase: acetonitrile/water gradient). This gave 1.08 g (77% of theory) of the desired product.

LC-MS (method 1A): Rₜ=0.49 min; MS (ESIpos): m/z=262 [M+H]⁺

¹H-NMR (400 MHz, DMSO-d₆): δ [ppm]=7.47-7.15 (m, 5H), 4.96 (s, 1H), 4.68 (d, 1H), 3.91 (t, 1H), 3.53 (dd, 1H), 2.80 (d, 1H), 2.32 (d, 1H), 1.70 (d, 1H), 1.53 (dd, 1H), 1.26-1.14 (m, 3H), 0.95 (s, 3H), 0.82-0.70 (m, 1H), 0.58-0.45 (m, 2H), 0.40-0.30 (m, 1H)

### Example 233A

### 1-(4-Benzyl-6,6-dimethylmorpholin-3-yl)cyclopropanol [enantiomerically pure isomer 1]

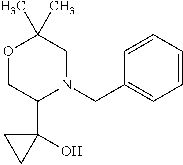 [SMILES: CC1(C)CN(Cc2ccccc2)C(C2(O)CC2)CO1]

1.08 g of 1-(4-benzyl-6,6-dimethylmorpholin-3-yl)cyclopropanol [racemate, Example 232A] were separated into the enantiomers on a chiral phase [Method 30D].

Yield: enantiomerically pure isomer 1: 340 mg (100% ee)

enantiomerically pure isomer 1: Rₜ=4.53 min [Method 27E].

LC-MS (method 1A): Rₜ=0.55 min; MS (ESIpos): m/z=262 [M+H]⁺

¹H-NMR (400 MHz, DMSO-d₆): δ [ppm]=7.47-7.15 (m, 5H), 4.96 (s, 1H), 4.68 (d, 1H), 3.91 (dd, 1H), 3.53 (dd, 1H), 2.80 (d, 1H), 2.32 (d, 1H), 1.70 (d, 1H), 1.53 (dd, 1H), 1.26-1.14 (m, 3H), 0.95 (s, 3H), 0.82-0.70 (m, 1H), 0.58-0.45 (m, 2H), 0.40-0.30 (m, 1H).

### Example 234A

### 1-(6,6-Dimethylmorpholin-3-yl)cyclopropanol [enantiomerically pure isomer 1]

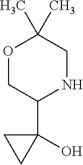

Under argon, 43 mg (0.40 mmol) of 10% palladium on activated carbon and 21 mg (0.15 mmol) of palladium(II) hydroxide were added to 339 mg (1.29 mmol) of 1-(4-benzyl-6,6-dimethylmorpholin-3-yl)cyclopropanol] [enantiomerically pure isomer 1] in 15 ml of ethanol, and the mixture was hydrogenated at RT and standard pressure overnight. The mixture was then filtered through silica gel, the filtrate was concentrated under reduced pressure and the residue was dried under high vacuum. This gave 217 mg (98% of theory) of the desired product.

MS (method 1C): m/z=172 [M+H]⁺

### Example 235A

### 4-Benzyl-6,6-dimethyl-5-oxomorpholine-3-carbaldehyde [racemate]

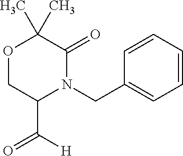 [SMILES: CC1(C)OCC(C=O)N(Cc2ccccc2)C1=O]

At −50° C., a solution of 2.42 ml (2.67 g, 34.2 mmol) of DMSO in 35 ml of dichloromethane was slowly added dropwise to 1.67 ml (2.43 g, 19 mmol) of ethanedioyl dichloride in 195 ml of dichloromethane, and the mixture was stirred at −50° C. for 10 min. A solution of 3.48 g (13.7 mmol) of 4-benzyl-5-(hydroxymethyl)-2,2-dimethylmorpholin-3-one [racemate] in 45 ml of dichloromethane was then slowly added dropwise, and the mixture was stirred at −50° C. for 10 min. At −78° C., a solution of 9.53 ml (6.92 g, 68.3 mmol) of triethylamine in 25 ml of dichloromethane was added dropwise. The mixture was stirred at −78° C. for 2 h and then allowed to slowly warm to room temperature. Saturated aqueous sodium bicarbonate solution and dichloromethane were added and the phases were separated. The aqueous phase was washed twice with dichloromethane. The combined organic phases were washed with saturated aqueous sodium chloride solution and then dried over sodium sulphate. After filtration, the filtrate was concentrated under reduced pressure and the residue was dried under high vacuum. This gave 3.86 g (purity: 47%) of the desired crude product.

LC-MS (method 1A): Rₜ=0.84 min; MS (ESIpos): m/z=248 [M+H]⁺

### Example 236A

### 4-Benzyl-5-(1-hydroxyethyl)-2,2-dimethylmorpholin-3-one [diastereomer mixture, 4 isomers]

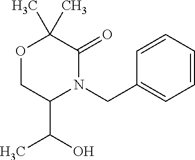 [SMILES: CC(O)C1COC(C)(C)C(=O)N1Cc1ccccc1]

At 0° C., 15.6 ml (46.8 mmol) of a 3 M solution of methylmagnesium bromide in diethyl ether were slowly added dropwise to 3.86 g (15.6 mmol) of 4-benzyl-6,6-dimethyl-5-oxomorpholine-3-carbaldehyde [racemate] in 50 ml of THF, and the mixture was stirred at room temperature for 1.5 h. Saturated aqueous ammonium chloride solution was added and the mixture was freed from THF under reduced pressure. Dichloromethane and water were added to the residue and the phases were separated. The organic phase was washed with water and dried over sodium sulphate. After filtration, the filtrate was concentrated under reduced pressure and the residue was dried under high vacuum. This gave 3.65 g (82% of theory, purity: 92%) of the desired product.

LC-MS (method 1A): Rₜ=0.80 min; MS (ESIpos): m/z=264 [M+H]⁺

¹H-NMR (400 MHz, DMSO-d₆): δ [ppm]=7.40-7.12 (m, 5H), 5.17-4.91 (m, 2H), 4.31-4.20 (m, 1H), 4.10-3.95 (m, 1H), 3.89-3.66 (m, 2H), 3.09-2.93 (m, 1H), 1.41-1.27 (m, 6H), 1.11 (d, 3H).

### Example 237A

### 1-(4-Benzyl-6,6-dimethylmorpholin-3-yl)ethanol [diastereomer mixture, 4 isomers]

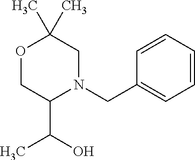 [SMILES: CC(O)C1COC(C)(C)CN1Cc1ccccc1]

At room temperature, 64.5 ml (129 mmol) of a 2 M solution of dimethyl sulphide/borane complex in THF were slowly added dropwise to 3.65 g (12.9 mmol, purity: 92%) of 4-benzyl-5-(1-hydroxyethyl)-2,2-dimethylmorpholin-3-one [diastereomer mixture, 4 isomers] in 400 ml of methanol. The mixture was stirred at room temperature overnight and then under reflux for 2 h. Subsequently, 400 ml of methanol were added slowly at room temperature and the mixture was then heated at reflux for 4 h. After concentration, the residue was purified by silica gel chromatography (dichloromethane/methanol 100:2-100:3). This gave 2.11 g (65% of theory) of the desired product.

LC-MS (method 1A): Rₜ=0.50 min; MS (ESIpos): m/z=250 [M+H]+

### Example 238A

### 1-(4-Benzyl-6,6-dimethylmorpholin-3-yl)ethanol [enantiomerically pure isomer 2]

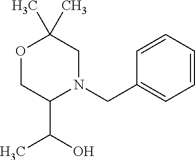 [SMILES: CC(O)C1COC(C)(C)CN1Cc1ccccc1]

2.11 g of 1-(4-benzyl-6,6-dimethylmorpholin-3-yl)ethanol [diastereomer mixture, 4 isomers, Example 237A] were separated into the enantiomers on a chiral phase [Method 31D].

Yield: enantiomerically pure isomer 2: 577 mg (100% ee)

enantiomerically pure isomer 2: Rₜ=6.55 min [Method 28E].

LC-MS (method 1A): Rₜ=0.52 min; MS (ESIpos): m/z=250 [M+H]⁺

¹H-NMR (400 MHz, DMSO-d₆): δ [ppm]=7.40-7.16 (m, 5H), 4.66 (d, 1H), 4.31-4.19 (m, 1H), 4.05 (d, 1H), 3.72-3.48 (m, 2H), 3.00 (d, 1H), 2.40-2.23 (m, 2H), 1.79 (d, 1H), 1.20-1.06 (m, 6H), 0.98 (s, 3H).

### Example 239A

### 1-(6,6-Dimethylmorpholin-3-yl)ethanol [enantiomerically pure isomer 2]

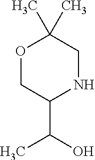

Under argon, 76 mg (0.71 mmol) of 10% palladium on activated carbon and 38 mg (0.27 mmol) of palladium(II) hydroxide were added to 575 mg (2.31 mmol) of 1-(4-benzyl-6,6-dimethylmorpholin-3-yl)ethanol] [enantiomerically pure isomer 2] in 26 ml of ethanol, and the mixture was hydrogenated at RT and standard pressure overnight. The mixture was then filtered through silica gel, the filtrate was concentrated under reduced pressure and the residue was dried under high vacuum. This gave 217 mg (98% of theory) of the desired product.

MS (method 1C): m/z=160 [M+H]⁺

¹H-NMR (400 MHz, DMSO-d₆): δ [ppm]=6.93-6.58 (m, 1H), 4.70-4.48 (m, 2H), 4.43-4.31 (m, 1H), 2.64 (d, 2H), 2.33 (ddd, 2H), 1.36 (s, 3H), 1.25-1.12 (m, 6H).

### Example 240A

### tert-Butyl 2-[(benzylamino)methyl]azetidine-1-carboxylate [racemate]

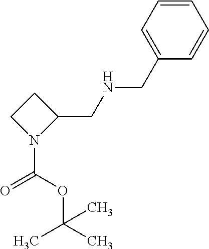 [SMILES: CC(C)(C)OC(=O)N1CCC1CNCc1ccccc1]

10.0 g (53.7 mmol) of tert-butyl 2-(aminomethyl)azetidine-1-carboxylate and 2.03 g (37.8 mmol) of benzaldehyde in 100 ml of methanol were heated under reflux for 2.5 h. The mixture was then cooled to 0° C., and sodium borohydride was added slowly at this temperature over a period of 15 min. The mixture was stirred at RT overnight. The mixture was then concentrated under reduced pressure, dichloromethane and water were added to the residue, the phases were separated and the aqueous phase was extracted twice with dichloromethane. The combined organic phases were washed with saturated aqueous sodium chloride solution, dried over sodium sulphate and filtered, and the filtrate was concentrated under reduced pressure. Dichloromethane was added to the residue obtained, and the product was purified by silica gel chromatography (dichloromethane, then dichloromethane/methanol=100:4). Yield: 7.43 g (50% of theory).

LC-MS (method 6A): Rₜ=2.41 min; MS (ESIpos): m/z=277 [M+H]⁺.

### Example 241A

### tert-Butyl 2-{[benzyl(1-methoxy-1-oxopropan-2-yl)amino]methyl}azetidine-1-carboxylate [diastereomer mixture, 4 isomers]

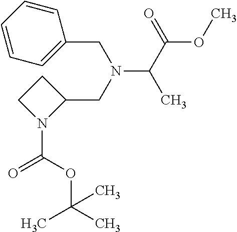 [SMILES: COC(=O)C(C)N(Cc1ccccc1)CC1CCN1C(=O)OC(C)(C)C]

2.50 g (9.05 mmol) of tert-butyl 2-[(benzylamino)methyl]azetidine-1-carboxylate [racemate] were dissolved in dichloromethane (150 ml), 5.55 ml (4.03 g, 39.8 mmol) of triethylamine and 3.04 ml (4.53 g, 27.1 mmol) of methyl 2-bromopropanoate [racemate] were added and the mixture was stirred at RT overnight. 5.55 ml (4.03 g, 39.8 mmol) of triethylamine and 3.04 ml (4.53 g, 27.1 mmol) of methyl 2-bromopropanoate [racemate] were added, and the mixture was stirred at 40° C. overnight. A further 5.55 ml (4.03 g, 39.8 mmol) of triethylamine and 3.04 ml (4.53 g, 27.1 mmol) of methyl 2-bromopropanoate [racemate] were then added, and the mixture was stirred at 40° C. overnight. After cooling to room temperature, the mixture was diluted with water and dichloromethane, and the phases were separated. The aqueous phase was extracted twice with dichloromethane and the combined organic phases were washed with saturated aqueous sodium chloride solution, dried over sodium sulphate, filtered and then freed of the solvent under reduced pressure. The crude product obtained was purified by silica gel chromatography (dichloromethane, then dichloromethane/methanol=100:1). Yield: 3.22 g (94% of theory).

LC-MS (Method 1A): Rₜ=1.00 min (diastereomer 1), Rₜ=1.13 min (diastereomer 2);

MS (ESIpos): m/z=363 [M+H]⁺;

¹H-NMR (400 MHz, DMSO-d₆): δ [ppm]=7.35-7.28 (m, 4H), 7.27-7.20 (m, 1H), 4.18-3.98 (m, 1H), 3.85-3.73 (m, 1H), 3.71-3.51 (m, 6H), 3.51-3.38 (m, 1H), 3.04-2.88 (m, 1H), 2.85-2.69 (m, 1H), 2.15-1.96 (m, 1H), 1.93-1.65 (m, 1H), 1.34 (d, 9H), 1.26-1.15 (m, 3H).

### Example 242A

### Methyl N-(azetidin-2-ylmethyl)-N-benzylalaninate hydrochloride [diastereomer mixture, 4 isomers]

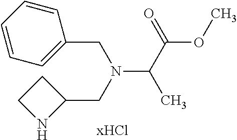 [SMILES: COC(=O)C(C)N(Cc1ccccc1)CC1CCN1]

14.9 ml (59.7 mmol) of a 4 N solution of hydrogen chloride in 1,4-dioxane were added to 3.2 g (8.5 mmol) of tert-butyl 2-{[benzyl(1-methoxy-1-oxopropan-2-yl)amino]methyl}-azetidine-1-carboxylate [diastereomer mixture, 4 isomers] in dioxane (74 ml), and the mixture was stirred at room temperature overnight. A further 14 ml (59.7 mmol) of a 4 N solution of hydrogen chloride in 1,4-dioxane were then added, and the mixture was stirred at RT overnight. The mixture was then concentrated under reduced pressure and the product was dried under high vacuum. Yield: 3.13 g (98% of theory, purity: 80%).

LC-MS (Method 1A): Rₜ=0.68 min (diastereomer 1, 2 isomers), Rₜ=0.70 min (diastereomer 2, 2 isomers);

MS (ESIpos): m/z=263 [M+H-HCl]⁺.

### Example 243A

### 4-Benzyl-3-methyl-1,4-diazabicyclo[4.2.0]octan-2-one [enantiomerically pure isomer 3]

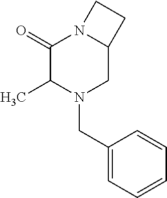 [SMILES: CC1C(=O)N2CCC2CN1Cc1ccccc1]

21.8 g (51.0 mmol, purity: 70%) of methyl N-(azetidin-2-ylmethyl)-N-benzylalaninate [diastereomer mixture, 4 isomers] were initially charged in methanol (562 ml), 28.2 g (204 mmol) of potassium carbonate were added and the mixture was then stirred at RT for 2.5 d. The reaction solution was filtered and most of the solvent was removed at 20° C. under reduced pressure. The residue was taken up in water and extracted repeatedly with dichloromethane and chloroform/isopropanol (7:3). The collected organic phases were dried over sodium sulphate, filtered and concentrated under reduced pressure. Using Method 7D, the crude product (12.1 g) was separated into the corresponding isomers. Here, the target compound eluted as third component. Yield: 2.47 g (21% of theory).

HPLC (Method 6E): Rₜ=7.49 min, 99.0% ee;

LC-MS (method 1A): Rₜ=0.50 min; MS (ESIpos): m/z=231 [M+H]⁺.

### Example 244A

### 3-Methyl-1,4-diazabicyclo[4.2.0]octan-2-one [enantiomerically pure isomer 3]

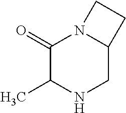

2.40 g (10.4 mmol) of 4-benzyl-3-methyl-1,4-diazabicyclo[4.2.0]octan-2-one [enantiomerically pure isomer 3] were initially charged in ethanol (85 ml), 250 mg of palladium on carbon (10%) and 130 mg of palladium hydroxide on carbon (20%) were added under argon and the mixture was then stirred under an atmosphere of hydrogen at standard pressure overnight. The reaction solution was filtered through kieselguhr and the filter residue was washed with hot ethanol (100 ml). The filtrate was concentrated under reduced pressure and the product was dried under high vacuum. Yield: 1.56 g (quant.).

GC-MS (Method 2B): Rₜ=4.50 min; MS (EIpos): m/z=140 [M]⁺;

MS (method 1C): m/z=141 [M+H]⁺;

¹H-NMR (400 MHz, DMSO-d₆): δ [ppm]=4.59 (m_{c}, 1H), 4.09-3.89 (m, 2H), 3.27 (q, 1H), 2.95 (dd, 1H), 2.58-2.53 (m, 2H), 2.33-2.04 (m, 2H), 1.12 (d, 3H).

### WORKING EXAMPLES

### Example 1

### 4-[(2-{[(1R)-1-(3-Chlorophenyl)ethyl]amino}-8-methoxy[1,2,4]triazolo[1,5-a]pyridin-6-yl)carbonyl]-3-methyl-1,4-diazabicyclo[4.2.0]octan-2-one [enantiomerically pure isomer]

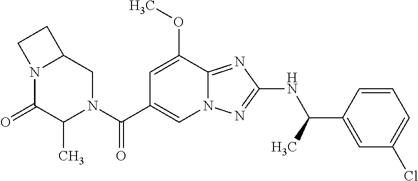 [SMILES: COc1cc(C(=O)N2CC3CCN3C(=O)C2C)cn2nc(N[C@H](C)c3cccc(Cl)c3)nc12]

46.1 mg (0.133 mmol) of 2-{[(1R)-1-(3-chlorophenyl)ethyl]amino}-8-methoxy[1,2,4]triazolo[1,5-a]pyridine-6-carboxylic acid [enantiomerically pure isomer] and 28.0 mg (0.199 mmol) of 3-methyl-1,4-diazabicyclo[4.2.0]octan-2-one [enantiomerically pure isomer 3, Example 23A] were initially charged in N,N-dimethylformamide (1.50 ml), and 68.7 mg (93 μl, 0.532 mmol) of N,N-diisopropylethylamine were added. Subsequently, 60.7 mg (0.160 mmol) of HATU were added at RT and the mixture was stirred overnight. Without further work-up, the reaction solution was purified by preparative RP-HPLC (acetonitrile/water). Yield: 40.6 mg (65% of theory).

LC-MS (method 1A): Rₜ=0.90 min; MS (ESIpos): m/z=469 [M+H]⁺;

¹H-NMR (400 MHz, DMSO-d₆): δ [ppm]=8.38 (s, 1H), 7.47 (s, 1H), 7.40-7.19 (m, 4H), 6.94 (s, 1H), 4.92-4.81 (m, 1H), 4.64-4.56 (m, 1H), 4.24 (q, 1H), 4.10-4.01 (m, 1H), 3.98-3.76 (m, 6H), 3.55-3.41 (m, 1H), 1.43 (d, 3H), 1.39 (d, 3H), one proton obscured.

### Example 2

### {2-[(3-Chlorobenzyl)amino]-8-methoxy[1,2,4]triazolo[1,5-a]pyridin-6-yl}[5-(3-hydroxycyclobutyl)-2-methylmorpholin-4-yl]methanone [diastereomer mixture, 4 isomers]

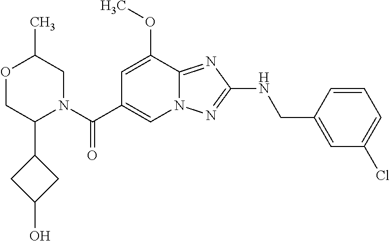 [SMILES: COc1cc(C(=O)N2CC(C)OCC2C2CC(O)C2)cn2nc(NCc3cccc(Cl)c3)nc12]

100 mg (0.301 mmol) of 2-[(3-chlorobenzyl)amino]-8-methoxy[1,2,4]triazolo[1,5-a]pyridine-6-carboxylic acid and 61.8 mg (0.361 mmol) of 3-(6-methylmorpholin-3-yl)cyclobutanol [4 isomers] were initially charged in N,N-dimethylformamide (1.38 ml), and 136 mg (183 μl, 1.05 mmol) of N,N-diisopropylethylamine were added. Subsequently, 137 mg (0.361 mmol) of HATU were added at RT and the mixture was stirred overnight. Without further work-up, the reaction solution was then purified by preparative RP-HPLC (acetonitrile/water). Yield: 97.6 mg (67% of theory).

LC-MS (method 1A): Rₜ=0.87 min; MS (ESIpos): m/z=486 [M+H]⁺;

### Example 3

### {2-[(3-Chlorobenzyl)amino]-8-methoxy[1,2,4]triazolo[1,5-a]pyridin-6-yl}[5-(3-hydroxycyclobutyl)-2-methylmorpholin-4-yl]methanone [diastereomer 1+diastereomer 2]

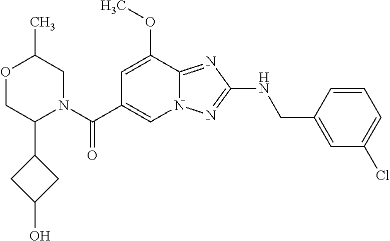 [SMILES: COc1cc(C(=O)N2CC(C)OCC2C2CC(O)C2)cn2nc(NCc3cccc(Cl)c3)nc12]

Diastereomer separation on a chiral phase of 93.0 mg of the compound from Example 2 according to Method 12D gave 30.2 mg of Example 3 (diastereomer 1+diastereomer 2) and 34.8 mg of Example 4 (diastereomer 3+diastereomer 4).

HPLC (method 12E): Rₜ=11.5 min;

LC-MS (Method 1A): Rₜ=0.87 min (diastereomer 1), Rₜ=0.88 min (diastereomer 2);

MS (ESIpos): m/z=486 [M+H]⁺.

### Example 4

### {2-[(3-Chlorobenzyl)amino]-8-methoxy[1,2,4]triazolo[1,5-a]pyridin-6-yl}[5-(3-hydroxycyclobutyl)-2-methylmorpholin-4-yl]methanone [diastereomer 3+diastereomer 4]

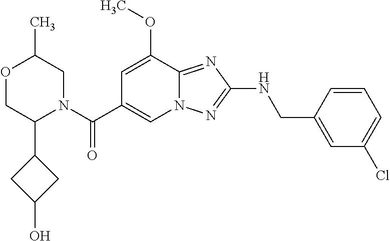 [SMILES: COc1cc(C(=O)N2CC(C)OCC2C2CC(O)C2)cn2nc(NCc3cccc(Cl)c3)nc12]

Diastereomer separation on a chiral phase of 93.0 mg of the compound from Example 2 according to Method 12D gave 30.2 mg of Example 3 (diastereomer 1+diastereomer 2) and 34.8 mg of Example 4 (diastereomer 3+diastereomer 4).

HPLC (method 12E): Rₜ=25 0 min;

LC-MS (Method 1A): Rₜ=0.87 min (diastereomer 3), Rₜ=0.88 min (diastereomer 4);

MS (ESIpos): m/z=486 [M+H]⁺.

### Example 5

### (2-{[1-(3-Chlorophenyl)-2-fluoroethyl]amino}-8-methoxy[1,2,4]triazolo[1,5-a]pyridin-6-yl) [(5R)-2,5-dimethylmorpholin-4-yl]methanone [enantiomerically pure isomer]

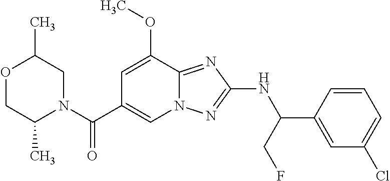 [SMILES: COc1cc(C(=O)N2CC(C)OC[C@H]2C)cn2nc(NC(CF)c3cccc(Cl)c3)nc12]

190 mg (0.513 mmol) of (2-bromo-8-methoxy[1,2,4]triazolo[1,5-a]pyridin-6-yl)[(5R)-2,5-dimethylmorpholin-4-yl]methanone [enantiomerically pure isomer], 129 mg (0.616 mmol) of 1-(3-chlorophenyl)-2-fluoroethanamine hydrochloride [enantiomerically pure isomer 2], 157 mg (873 μl, 1.75 mmol, 2 M solution in tetrahydrofuran) of sodium tert-butoxide, 44.3 mg (0.077 mmol) of bis(dibenzylideneacetone)palladium(0) and 44.5 mg (0.077 mmol) of 4,5-bis(diphenylphosphino)-9,9-dimethylxanthene (XantPhos) were initially charged in degassed 1,4-dioxane (7.58 ml) in a microwave tube. The tube was sealed and the reaction mixture was subsequently stirred at 160° C. in the microwave oven (Biotage Synthesizer) for 1 h. The reaction solution was poured onto water and extracted with dichloromethane, and the organic phase was dried over sodium sulphate, filtered and concentrated under reduced pressure. The residue was purified initially by preparative RP-HPLC (acetonitrile/water) and then re-purified by Method 2F. Yield: 11.2 mg (5% of theory).

LC-MS (method 1A): Rₜ=0.98 min; MS (ESIpos): m/z=461 [M+H]⁺;

¹H-NMR (400 MHz, DMSO-d₆): δ [ppm]=8.33 (s, 1H), 7.64-7.53 (m, 2H), 7.49-7.28 (m, 3H), 6.89 (br. s., 1H), 5.18 (m_{c}, 1H), 4.75-3.86 (m, 6H), 3.49-3.39 (m, 3H), 3.19-2.64 (s, 1H), 1.37-0.89 (m, 6H), one proton obscured

### Example 6

### {2-[(3-Chlorobenzyl)amino]-8-methoxy[1,2,4]triazolo[1,5-a]pyridin-6-yl}[(5R)-2-(2-hydroxyethyl)-2,5-dimethylmorpholin-4-yl]methanone [enantiomerically pure isomer]

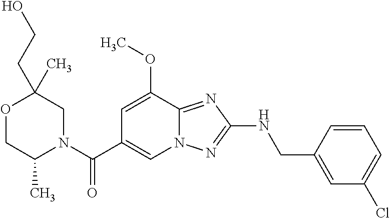 [SMILES: COc1cc(C(=O)N2CC(C)(CCO)OC[C@H]2C)cn2nc(NCc3cccc(Cl)c3)nc12]

100 mg (0.301 mmol) of 2-[(3-chlorobenzyl)amino]-8-methoxy[1,2,4]triazolo[1,5-a]pyridine-6-carboxylic acid and 57.4 mg (0.361 mmol) of 2-[(5R)-2,5-dimethylmorpholin-2-yl]ethanol [enantiomerically pure isomer 2] were initially charged in N,N-dimethylformamide (1.38 ml), and 136 mg (183 μl, 1.05 mmol) of N,N-diisopropylethylamine were added. Subsequently, 137 mg (0.361 mmol) of HATU were added at RT and the mixture was stirred overnight. Without further work-up, the reaction solution was then purified by preparative RP-HPLC (acetonitrile/water). Yield: 97.6 mg (67% of theory).

LC-MS (method 1A): Rₜ=0.88 min; MS (ESIpos): m/z=474 [M+H]⁺;

¹H-NMR (400 MHz, DMSO-d₆): δ [ppm]=8.30 (s, 1H), 7.40 (s, 1H), 7.37-7.20 (m, 4H), 6.86 (d, 1H), 4.46 (d, 2H), 4.28 (t, 1H), 4.19 (br.s., 1H), 3.93 (s, 3H), 3.78 (dd, 1H), 3.68 (br.s., 1H), 3.48-3.32 (m, 3H), 3.10-2.98 (m, 1H), 2.00 (m_{c}, 1H), 1.49 (m_{c}, 1H), 1.25 (d, 3H), 1.09 (s, 3H).

### Example 7

### {2-[(3-Chlorobenzyl)amino]-8-methoxy[1,2,4]triazolo[1,5-a]pyridin-6-yl}[(5R)-5-(2-hydroxyethyl)-2,2-dimethylmorpholin-4-yl]methanone [enantiomer mixture, 2 isomers]

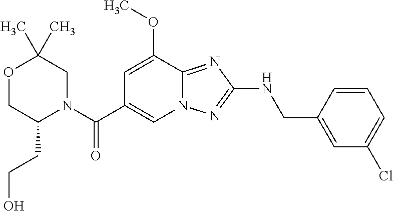 [SMILES: COc1cc(C(=O)N2CC(C)(C)OC[C@H]2CCO)cn2nc(NCc3cccc(Cl)c3)nc12]

70.0 mg (0.210 mmol) of 2-[(3-chlorobenzyl)amino]-8-methoxy[1,2,4]triazolo[1,5-a]pyridine-6-carboxylic acid and 40.2 mg (0.252 mmol) of 2-[(3R)-6,6-dimethylmorpholin-3-yl]ethanol [enantiomer mixture, 2 isomers] were initially charged in N,N-dimethylformamide (1.00 ml), and 95.1 mg (128 μl, 0.736 mmol) of N,N-diisopropylethylamine were added. 96.0 mg (0.252 mmol) of HATU were then added at RT, and the mixture was stirred for 1 h. Without further work-up, the reaction solution was then purified by preparative RP-HPLC (acetonitrile/water). Yield: 86.2 mg (85% of theory, enantiomer ratio about 85:15).

LC-MS (method 1A): Rₜ=0.89 min; MS (ESIpos): m/z=474 [M+H]⁺;

¹H-NMR (400 MHz, DMSO-d₆): δ [ppm]=8.32 (br. s., 1H), 7.39 (s, 1H), 7.36-7.21 (m, 4H), 6.87 (s, 1H), 4.46 (d, 2H), 4.42 (t, 1H), 3.93 (s, 3H), 3.83 (br. d., 1H), 3.50-3.37 (m, 3H), 2.00-1.78 (m, 2H), 1.20-1.02 (m, 6H), three protons obscured

### Example 8

### {2-[(3-Chlorobenzyl)amino]-8-methoxy[1,2,4]triazolo[1,5-a]pyridin-6-yl}[5-(hydroxymethyl)-2,2,5-trimethylmorpholin-4-yl]methanone [racemate]

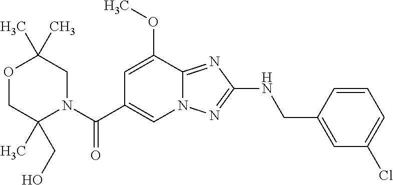 [SMILES: COc1cc(C(=O)N2CC(C)(C)OCC2(C)CO)cn2nc(NCc3cccc(Cl)c3)nc12]

120 mg (0.361 mmol) of 2-[(3-chlorobenzyl)amino]-8-methoxy[1,2,4]triazolo[1,5-a]pyridine-6-carboxylic acid and 68.9 mg (0.433 mmol) of (3,6,6-trimethylmorpholin-3-yl)methanol [racemate] were initially charged in N,N-dimethylformamide (1.11 ml), and 163 mg (220 μl, 1.26 mmol) of N,N-diisopropylethylamine were added. Subsequently, 165 mg (0.433 mmol) of HATU were added at RT and the mixture was stirred overnight. Without further work-up, the reaction solution was then purified by preparative RP-HPLC (acetonitrile/water). Yield: 61.1 mg (36% of theory).

LC-MS (method 1A): Rₜ=0.93 min; MS (ESIpos): m/z=474 [M+H]⁺;

¹H-NMR (400 MHz, DMSO-d₆): δ [ppm]=8.23 (d, 1H), 7.40 (s, 1H), 7.37-7.21 (m, 4H), 6.83 (d, 1H), 4.89 (t, 1H), 4.45 (d, 2H), 4.01-3.83 (m, 5H), 3.65 (dd, 1H), 3.30-3.21 (m, 3H), 1.41 (s, 3H), 1.12 (s, 3H), 1.07 (s, 3H).

### Example 9

### {2-[(3-Chlorobenzyl)amino]-8-methoxy[1,2,4]triazolo[1,5-a]pyridin-6-yl}[5-(hydroxymethyl)-2,2,5-trimethylmorpholin-4-yl]methanone [enantiomerically pure isomer 1]

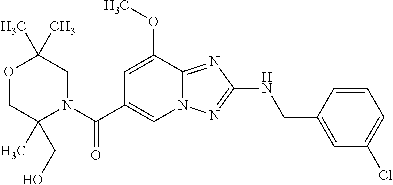 [SMILES: COc1cc(C(=O)N2CC(C)(C)OCC2(C)CO)cn2nc(NCc3cccc(Cl)c3)nc12]

Enantiomer separation on a chiral phase of 143 mg of the compound from Example 8 according to Method 13D gave 47.8 mg of Example 9 (enantiomerically pure isomer 1) and 58.0 mg of Example 10 (enantiomerically pure isomer 2).

HPLC (Method 4E): Rₜ=12.1 min, >99.0% ee;

LC-MS (method 1A): Rₜ=0.95 min; MS (ESIpos): m/z=474 [M+H]⁺;

¹H-NMR (400 MHz, DMSO-d₆): δ [ppm]=8.23 (d, 1H), 7.40 (s, 1H), 7.37-7.24 (m, 4H), 6.83 (d, 1H), 4.89 (t, 1H), 4.45 (d, 2H), 3.98-3.84 (m, 5H), 3.65 (dd, 1H), 3.30-3.22 (m, 3H), 1.41 (s, 3H), 1.12 (s, 3H), 1.07 (s, 3H).

### Example 10

### {2-[(3-Chlorobenzyl)amino]-8-methoxy[1,2,4]triazolo[1,5-a]pyridin-6-yl}[5-(hydroxymethyl)-2,2,5-trimethylmorpholin-4-yl]methanone [enantiomerically pure isomer 2]

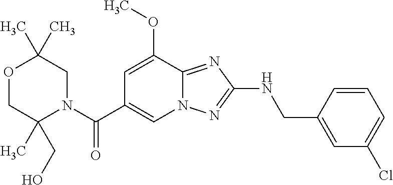 [SMILES: COc1cc(C(=O)N2CC(C)(C)OCC2(C)CO)cn2nc(NCc3cccc(Cl)c3)nc12]

Enantiomer separation on a chiral phase of 143 mg of the compound from Example 8 according to Method 13D gave 47.8 mg of Example 9 (enantiomerically pure isomer 1) and 58.0 mg of Example 10 (enantiomerically pure isomer 2).

HPLC (Method 4E): Rₜ=18.6 min, 93.0% ee;

LC-MS (method 1A): Rₜ=0.95 min; MS (ESIpos): m/z=474 [M+H]⁺;

¹H-NMR (400 MHz, DMSO-d₆): δ [ppm]=8.23 (d, 1H), 7.40 (s, 1H), 7.37-7.20 (m, 4H), 6.83 (d, 1H), 4.89 (t, 1H), 4.45 (d, 2H), 4.00-3.83 (m, 5H), 3.65 (dd, 1H), 3.30-3.22 (m, 3H), 1.41 (s, 3H), 1.12 (s, 3H), 1.07 (s, 3H).

### Example 11

### {2-[(3-Chlorobenzyl)amino]-8-methoxy[1,2,4]triazolo[1,5-a]pyridin-6-yl}[5-(methoxymethyl)-2,2-dimethylmorpholin-4-yl]methanone [racemate]

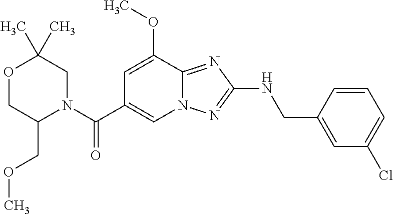 [SMILES: COCC1COC(C)(C)CN1C(=O)c1cc(OC)c2nc(NCc3cccc(Cl)c3)nn2c1]

120 mg (0.361 mmol) of 2-[(3-chlorobenzyl)amino]-8-methoxy[1,2,4]triazolo[1,5-a]pyridine-6-carboxylic acid and 68.9 mg (0.433 mmol) of 5-(methoxymethyl)-2,2-dimethylmorpholine [racemate] were initially charged in N,N-dimethylformamide (1.66 ml), and 163 mg (220 μl, 1.26 mmol) of N,N-diisopropylethylamine were added. Subsequently, 165 mg (0.433 mmol) of HATU were added at RT and the mixture was stirred overnight. Without further work-up, the reaction solution was purified by preparative RP-HPLC (acetonitrile/water). Yield: 160 mg (93% of theory).

LC-MS (method 1A): Rₜ=0.98 min; MS (ESIpos): m/z=474 [M+H]⁺;

¹H-NMR (400 MHz, DMSO-d₆): δ [ppm]=8.35 (br. s., 1H), 7.39 (s, 1H), 7.37-7.22 (m, 4H), 6.91 (br. s., 1H), 4.46 (d, 2H), 3.92 (s, 3H), 3.87-3.40 (m, 4H), 3.28 (s, 3H), 3.16-2.76 (m, 1H), 1.21-1.09 (d, 6H), two protons obscured

### Example 12

### {2-[(3-Chlorobenzyl)amino]-8-methoxy[1,2,4]triazolo[1,5-a]pyridin-6-yl}[5-(fluoromethyl)-2,2-dimethylmorpholin-4-yl]methanone [racemate]

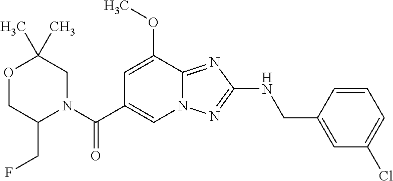 [SMILES: COc1cc(C(=O)N2CC(C)(C)OCC2CF)cn2nc(NCc3cccc(Cl)c3)nc12]

80.0 mg (0.240 mmol) of 2-[(3-chlorobenzyl)amino]-8-methoxy[1,2,4]triazolo[1,5-a]pyridine-6-carboxylic acid and 42.5 mg (0.289 mmol) of 5-(fluoromethyl)-2,2-dimethylmorpholine [racemate] were initially charged in N,N-dimethylformamide (1.11 ml), and 109 mg (147 μl, 0.841 mmol) of N,N-diisopropylethylamine were added. Subsequently, 110 mg (0.289 mmol) of HATU were added at RT and the mixture was stirred overnight. Without further work-up, the reaction solution was purified by preparative RP-HPLC (acetonitrile/water). Yield: 64.8 mg (54% of theory).

LC-MS (method 3A): Rₜ=2.08 min; MS (ESIpos): m/z=462 [M+H]⁺;

¹H-NMR (400 MHz, DMSO-d₆): δ [ppm]=8.34 (s, 1H), 7.39 (s, 1H), 7.37-7.20 (m, 4H), 6.85 (d, 1H), 4.89-4.51 (m, 2H), 4.46 (d, 2H), 3.92 (s, 4H), 3.55 (br. s., 1H), 3.08 (br. s., 1H), 1.29-1.07 (m, 6H), two protons obscured

### Example 13

### {2-[(3-Chlorobenzyl)amino]-8-methoxy[1,2,4]triazolo[1,5-a]pyridin-6-yl}[5-(fluoromethyl)-2,2-dimethylmorpholin-4-yl]methanone [enantiomerically pure isomer 1]

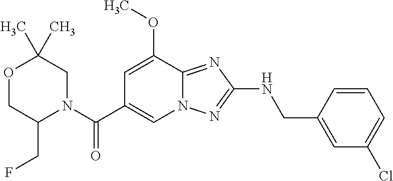 [SMILES: COc1cc(C(=O)N2CC(C)(C)OCC2CF)cn2nc(NCc3cccc(Cl)c3)nc12]

Enantiomer separation on a chiral phase of 60.0 mg of the compound from Example 12 according to Method 14D gave 24.8 mg of Example 13 (enantiomerically pure isomer 1) and 23.4 mg of Example 14 (enantiomerically pure isomer 2).

HPLC (Method 13E): Rₜ=8.77 min, >99.0% ee;

LC-MS (method 1A): Rₜ=1.00 min; MS (ESIpos): m/z=462 [M+H]⁺;

¹H-NMR (400 MHz, DMSO-d₆): δ [ppm]=8.34 (s, 1H), 7.39 (s, 1H), 7.37-7.21 (m, 4H), 6.85 (s, 1H), 4.90-4.51 (m, 2H), 4.46 (d, 2H), 3.98-3.82 (m, 4H), 3.54 (br. s., 1H), 3.08 (br. s., 1H), 1.29-1.10 (m, 6H), two protons obscured

### Example 14

### {2-[(3-Chlorobenzyl)amino]-8-methoxy[1,2,4]triazolo[1,5-a]pyridin-6-yl}[5-(fluoromethyl)-2,2-dimethylmorpholin-4-yl]methanone [enantiomerically pure isomer 2]

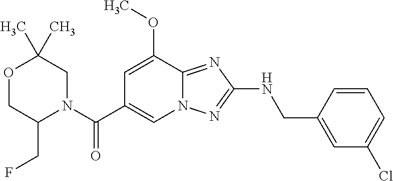 [SMILES: COc1cc(C(=O)N2CC(C)(C)OCC2CF)cn2nc(NCc3cccc(Cl)c3)nc12]

Enantiomer separation on a chiral phase of 60.0 mg of the compound from Example 12 according to Method 14D gave 24.8 mg of Example 13 (enantiomerically pure isomer 1) and 23.4 mg of Example 14 (enantiomerically pure isomer 2).

HPLC (Method 13E): Rₜ=14.9 min, >99.0% ee;

LC-MS (method 1A): Rₜ=1.00 min; MS (ESIpos): m/z=462 [M+H]⁺;

¹H-NMR (400 MHz, DMSO-d₆): δ [ppm]=8.34 (s, 1H), 7.39 (s, 1H), 7.37-7.24 (m, 4H), 6.85 (d, 1H), 4.88-4.50 (m, 2H), 4.46 (d, 2H), 3.97-3.84 (m, 4H), 3.54 (br. s., 1H), 3.08 (br. s., 1H), 1.29-1.09 (m, 6H), two protons obscured

### Example 15

### {2-[(3-Chlorobenzyl)amino]-8-methoxy[1,2,4]triazolo[1,5-a]pyridin-6-yl}[5-(2-hydroxyethyl)-2-methylmorpholin-4-yl]methanone [racemate]

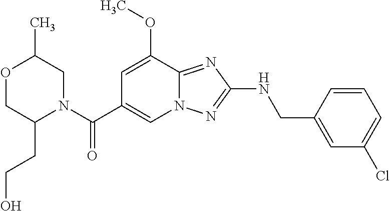 [SMILES: COc1cc(C(=O)N2CC(C)OCC2CCO)cn2nc(NCc3cccc(Cl)c3)nc12]

120 mg (0.361 mmol) of 2-[(3-chlorobenzyl)amino]-8-methoxy[1,2,4]triazolo[1,5-a]pyridine-6-carboxylic acid and 62.8 mg (0.433 mmol) of 2-(6-methylmorpholin-3-yl)ethanol [racemate] were initially charged in N,N-dimethylformamide (1.66 ml), and 163 mg (220 μl, 1.26 mmol) of N,N-diisopropylethylamine were added. Subsequently, 165 mg (0.433 mmol) of HATU were added at RT and the mixture was stirred overnight. Without further work-up, the reaction solution was purified by preparative RP-HPLC (acetonitrile/water). Yield: 143 mg (86% of theory).

LC-MS (method 1A): Rₜ=0.88 min; MS (ESIpos): m/z=460 [M+H]⁺;

¹H-NMR (400 MHz, DMSO-d₆): δ [ppm]=8.33 (br. s., 1H), 7.39 (s, 1H), 7.37-7.24 (m, 4H), 6.89 (br. s., 1H), 4.59-4.06 (m, 4H), 3.93 (s, 3H), 3.86-3.39 (m, 5H), 2.01-1.76 (m, 2H), 1.24-0.94 (m, 3H), two protons obscured

### Example 16

### {2-[(3-Chlorobenzyl)amino]-8-methoxy[1,2,4]triazolo[1,5-a]pyridin-6-yl}[5-(2-hydroxyethyl)-2-methylmorpholin-4-yl]methanone [enantiomerically pure isomer 1]

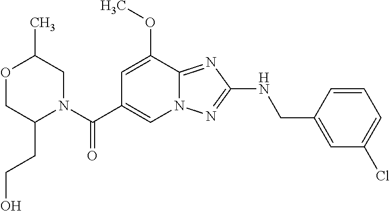 [SMILES: COc1cc(C(=O)N2CC(C)OCC2CCO)cn2nc(NCc3cccc(Cl)c3)nc12]

Enantiomer separation on a chiral phase of 138 mg of the compound from Example 15 according to Method 15D gave 35.2 mg of Example 16 (enantiomerically pure isomer 1) and 35.9 mg of Example 17 (enantiomerically pure isomer 2).

HPLC (Method 14E): Rₜ=5.43 min, >99.0% ee;

LC-MS (method 1A): Rₜ=0.87 min; MS (ESIpos): m/z=460 [M+H]⁺;

¹H-NMR (400 MHz, DMSO-d₆): δ [ppm]=8.32 (br. s., 1H), 7.39 (s, 1H), 7.37-7.23 (m, 4H), 6.89 (br. s., 1H), 4.61-4.08 (m, 4H), 3.93 (s, 3H), 3.86-3.37 (m, 5H), 2.01-1.76 (m, 2H), 1.22-0.91 (m, 3H), two protons obscured

### Example 17

### {2-[(3-Chlorobenzyl)amino]-8-methoxy[1,2,4]triazolo[1,5-a]pyridin-6-yl}[5-(2-hydroxyethyl)-2-methylmorpholin-4-yl]methanone [enantiomerically pure isomer 2]

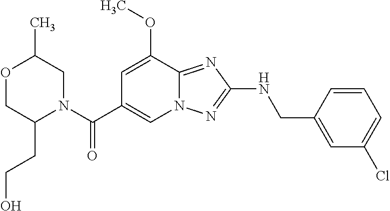 [SMILES: COc1cc(C(=O)N2CC(C)OCC2CCO)cn2nc(NCc3cccc(Cl)c3)nc12]

Enantiomer separation on a chiral phase of 138 mg of the compound from Example 15 according to Method 15D gave 35.2 mg of Example 16 (enantiomerically pure isomer 1) and 35.9 mg of Example 17 (enantiomerically pure isomer 2).

HPLC (Method 14E): Rₜ=9.08 min, >99.0% ee;

LC-MS (method 1A): Rₜ=0.87 min; MS (ESIpos): m/z=460 [M+H]⁺;

¹H-NMR (400 MHz, DMSO-d₆): δ [ppm]=8.32 (br. s., 1H), 7.39 (s, 1H), 7.37-7.22 (m, 4H), 6.89 (br. s., 1H), 4.62-4.09 (m, 4H), 3.93 (s, 3H), 3.84-3.38 (m, 5H), 2.00-1.72 (m, 2H), 1.19-0.93 (m, 3H), two protons obscured

### Example 18

### (2-{[1-(3-Chlorophenyl)-2-fluoroethyl]amino}-8-methoxy[1,2,4]triazolo[1,5-a]pyridin-6-yl) [5-(2-hydroxyethyl)-2-methylmorpholin-4-yl]methanone [enantiomerically pure isomer 1]

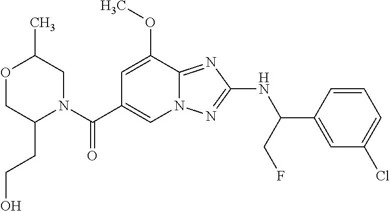 [SMILES: COc1cc(C(=O)N2CC(C)OCC2CCO)cn2nc(NC(CF)c3cccc(Cl)c3)nc12]

Enantiomer separation on a chiral phase of 24.1 mg of the compound from Example 76 according to Method 16D gave 7.90 mg of Example 18 (enantiomerically pure isomer 1) and 9.00 mg of Example 19 (enantiomerically pure isomer 2).

HPLC (Method 15E): Rₜ=8.93 min, >99.0% ee;

¹H-NMR (400 MHz, DMSO-d₆): δ [ppm]=8.32 (br. s., 1H), 7.65-7.54 (m, 2H), 7.50-7.42 (m, 1H), 7.41-7.28 (m, 2H), 6.89 (br. s., 1H), 5.17 (m_{c}, 1H), 4.75-4.10 (m, 4H), 3.92 (s, 3H), 3.83-3.38 (m, 5H), 3.16-2.61 (m, 1H), 2.00-1.74 (m, 2H), 1.20-0.92 (m, 3H), one proton obscured

### Example 19

### (2-{[1-(3-Chlorophenyl)-2-fluoroethyl]amino}-8-methoxy[1,2,4]triazolo[1,5-a]pyridin-6-yl) [5-(2-hydroxyethyl)-2-methylmorpholin-4-yl]methanone [enantiomerically pure isomer 2]

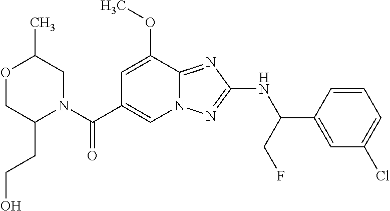 [SMILES: COc1cc(C(=O)N2CC(C)OCC2CCO)cn2nc(NC(CF)c3cccc(Cl)c3)nc12]

Enantiomer separation on a chiral phase of 24.1 mg of the compound from Example 76 according to Method 16D gave 7.90 mg of Example 18 (enantiomerically pure isomer 1) and 9.00 mg of Example 19 (enantiomerically pure isomer 2).

HPLC (Method 15E): Rₜ=12.2 min, >99.0% ee;

¹H-NMR (400 MHz, DMSO-d₆): δ [ppm]=8.32 (br. s., 1H), 7.64-7.54 (m, 2H), 7.49-7.43 (m, 1H), 7.41-7.27 (m, 2H), 6.89 (br. s., 1H), 5.17 (m_{c}, 1H), 4.73-4.11 (m, 4H), 3.92 (s, 3H), 3.85-3.36 (m, 5H), 3.18-2.62 (m, 1H), 1.99-1.73 (m, 2H), 1.20-0.93 (m, 3H), one proton obscured

### Example 20

### {2-[(3-Chlorobenzyl)amino]-8-methoxy[1,2,4]triazolo[1,5-a]pyridin-6-yl}[(cis)-2-hydroxy-7-methyl-8-oxa-5-azaspiro[3.5]non-5-yl]methanone [enantiomerically pure isomer 1]

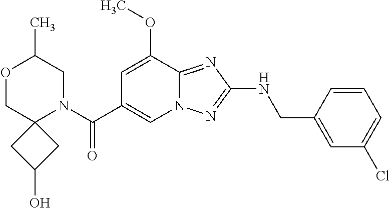 [SMILES: COc1cc(C(=O)N2CC(C)OCC23CC(O)C3)cn2nc(NCc3cccc(Cl)c3)nc12]

60.0 mg (0.180 mmol) of 2-[(3-chlorobenzyl)amino]-8-methoxy[1,2,4]triazolo[1,5-a]pyridine-6-carboxylic acid and 41.9 mg (0.216 mmol) of cis-7-methyl-8-oxa-5-azaspiro[3.5]nonan-2-ol hydrochloride [enantiomerically pure isomer 1, Example 101A] were initially charged in N,N-dimethylformamide (0.83 ml), and 81.6 mg (110 μl, 0.631 mmol) of N,N-diisopropylethylamine were added. Subsequently, 82.2 mg (0.216 mmol) of HATU were added at RT and the mixture was stirred overnight. Without further work-up, the reaction solution was purified by preparative RP-HPLC (acetonitrile/water). Yield: 49.9 mg (58% of theory).

LC-MS (method 1A): Rₜ=0.88 min; MS (ESIpos): m/z=472 [M+H]⁺;

¹H-NMR (400 MHz, DMSO-d₆): δ [ppm]=8.38 (d, 1H), 7.44-7.23 (m, 5H), 6.93 (d, 1H), 5.11 (d, 1H), 4.46 (d, 2H), 3.94 (s, 3H), 3.89 (q, 1H), 3.69-3.55 (m, 2H), 3.49 (d, 1H), 2.98 (dd, 1H), 2.75-2.62 (m, 1H), 2.43-2.30 (m, 1H), 2.17 (dd, 1H), 1.93 (t, 1H), 0.93 (d, 3H), one proton obscured

### Example 21

### {2-[(3-Chlorobenzyl)amino]-8-methoxy[1,2,4]triazolo[1,5-a]pyridin-6-yl}[(trans)-2-hydroxy-7-methyl-8-oxa-5-azaspiro[3.5]non-5-yl]methanone [enantiomerically pure isomer 1]

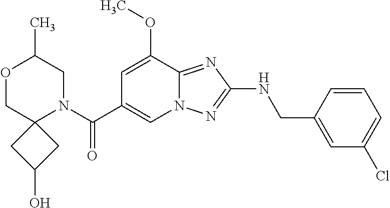 [SMILES: COc1cc(C(=O)N2CC(C)OCC23CC(O)C3)cn2nc(NCc3cccc(Cl)c3)nc12]

60.0 mg (0.180 mmol) of 2-[(3-chlorobenzyl)amino]-8-methoxy[1,2,4]triazolo[1,5-a]pyridine-6-carboxylic acid and 41.9 mg (0.216 mmol) of trans-7-methyl-8-oxa-5-azaspiro[3.5]nonan-2-ol hydrochloride [enantiomerically pure isomer 1, Example 103A] were initially charged in N,N-dimethylformamide (0.83 ml), and 81.6 mg (110 μl, 0.631 mmol) of N,N-diisopropylethylamine were added. Subsequently, 82.2 mg (0.216 mmol) of HATU were added at RT and the mixture was stirred overnight. Without further work-up, the reaction solution was purified by preparative RP-HPLC (acetonitrile/water). Yield: 51.2 mg (60% of theory).

LC-MS (method 1A): Rₜ=0.88 min; MS (ESIpos): m/z=472 [M+H]⁺;

¹H-NMR (400 MHz, DMSO-d₆): δ [ppm]=8.38 (d, 1H), 7.44-7.21 (m, 5H), 6.93 (d, 1H), 5.01 (d, 1H), 4.46 (d, 2H), 4.26 (m_{c}, 1H), 3.93 (s, 3H), 3.71 (d, 1H), 3.54 (dd, 1H), 2.96 (dd, 1H), 2.78-2.67 (m, 1H), 2.48-2.40 (m, 1H), 2.10 (d, 1H), 1.84 (d, 1H), 0.92 (d, 3H), two protons obscured

### Example 22

### (2-{[1-(3-Chlorophenyl)-2-fluoroethyl]amino}-8-methoxy[1,2,4]triazolo[1,5-a]pyridin-6-yl)[(cis)-2-hydroxy-7-methyl-8-oxa-5-azaspiro[3.5]non-5-yl]methanone [enantiomerically pure isomer 1]

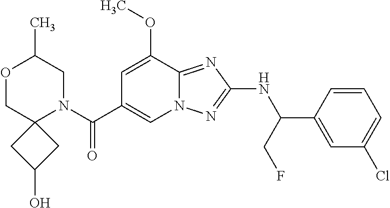 [SMILES: COc1cc(C(=O)N2CC(C)OCC23CC(O)C3)cn2nc(NC(CF)c3cccc(Cl)c3)nc12]

150 mg (0.411 mmol) of 2-{[1-(3-chlorophenyl)-2-fluoroethyl]amino}-8-methoxy[1,2,4]triazolo[1,5-a]pyridine-6-carboxylic acid [enantiomerically pure isomer] and 159 mg (0.822 mmol) of cis-7-methyl-8-oxa-5-azaspiro[3.5]nonan-2-ol hydrochloride [enantiomerically pure isomer 1, Example 101A] were initially charged in N,N-dimethylformamide (2.74 ml), and 372 mg (500 μl, 2.88 mmol) of N,N-diisopropylethylamine were added. 188 mg (0.493 mmol) of HATU were then added at RT, and the mixture was stirred for 2 h. Without further work-up, the reaction solution was purified by preparative RP-HPLC (acetonitrile/water). Yield: 113 mg (54% of theory).

LC-MS (method 1A): Rₜ=0.89 min; MS (ESIpos): m/z=504 [M+H]⁺;

optical rotation: [α]_{D}^{19.7}=43.33° (c=0.51, methanol);

¹H-NMR (400 MHz, DMSO-d₆): δ [ppm]=8.37 (d, 1H), 7.64 (d, 1H), 7.58 (br. s., 1H), 7.49-7.43 (m, 1H), 7.42-7.29 (m, 2H), 6.92 (d, 1H), 5.27-5.06 (m, 2H), 4.74-4.61 (m, 1H), 4.59-4.46 (m, 1H), 3.94 (s, 3H), 3.92-3.81 (m, 1H), 3.68-3.45 (m, 3H), 2.97 (dd, 1H), 2.75-2.60 (m, 1H), 2.39 (m_{c}, 1H), 2.19 (dd, 1H), 1.93 (t, 1H), 0.91 (d, 3H), one proton obscured

### Example 23

### (2-{[1-(3-Chlorophenyl)-2-fluoroethyl]amino}-8-methoxy[1,2,4]triazolo[1,5-a]pyridin-6-yl)[(cis)-2-hydroxy-7-methyl-8-oxa-5-azaspiro[3.5]non-5-yl]methanone [enantiomerically pure isomer 2]

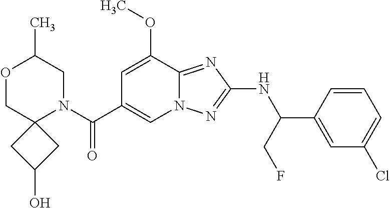 [SMILES: COc1cc(C(=O)N2CC(C)OCC23CC(O)C3)cn2nc(NC(CF)c3cccc(Cl)c3)nc12]

150 mg (0.411 mmol) of 2-{[1-(3-chlorophenyl)-2-fluoroethyl]amino}-8-methoxy[1,2,4]triazolo[1,5-a]pyridine-6-carboxylic acid [enantiomerically pure isomer] and 159 mg (0.822 mmol) of cis-7-methyl-8-oxa-5-azaspiro[3.5]nonan-2-ol hydrochloride [enantiomerically pure isomer 2, Example 102A] were initially charged in N,N-dimethylformamide (2.74 ml), and 372 mg (500 μl, 2.88 mmol) of N,N-diisopropylethylamine were added. 188 mg (0.493 mmol) of HATU were then added at RT, and the mixture was stirred for 60 h. Without further work-up, the reaction solution was purified by preparative RP-HPLC (acetonitrile/water). Yield: 154 mg (74% of theory).

LC-MS (method 1A): Rₜ=0.87 min; MS (ESIpos): m/z=504 [M+H]⁺;

optical rotation: [α]_{D}^{19.7}=153.3° (c=0.525, methanol);

¹H-NMR (400 MHz, DMSO-d₆): δ [ppm]=8.37 (d, 1H), 7.64 (d, 1H), 7.57 (s, 1H), 7.49-7.42 (m, 1H), 7.41-7.28 (m, 2H), 6.93 (s, 1H), 5.28-5.03 (m, 2H), 4.72-4.62 (m, 1H), 4.60-4.49 (m, 1H), 3.94 (s, 3H), 3.91-3.82 (m, 1H), 3.71-3.45 (m, 3H), 2.97 (dd, 1H), 2.73-2.62 (m, 1H), 2.38 (m_{c}, 1H), 2.17 (dd, 1H), 1.93 (t, 1H), 0.92 (d, 3H), one proton obscured

### Example 24

### (2-{[1-(3-Chlorophenyl)-2-fluoroethyl]amino}-8-methoxy[1,2,4]triazolo[1,5-a]pyridin-6-yl) [(trans)-2-hydroxy-7-methyl-8-oxa-5-azaspiro[3.5]non-5-yl]methanone [enantiomerically pure isomer 1]

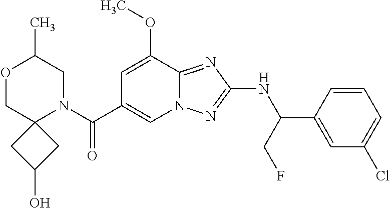 [SMILES: COc1cc(C(=O)N2CC(C)OCC23CC(O)C3)cn2nc(NC(CF)c3cccc(Cl)c3)nc12]

150 mg (0.411 mmol) of 2-{[1-(3-chlorophenyl)-2-fluoroethyl]amino}-8-methoxy[1,2,4]triazolo[1,5-a]pyridine-6-carboxylic acid [enantiomerically pure isomer] and 159 mg (0.822 mmol) of trans-7-methyl-8-oxa-5-azaspiro[3.5]nonan-2-ol hydrochloride [enantiomerically pure isomer 1, Example 103A] were initially charged in N,N-dimethylformamide (2.74 ml), and 372 mg (500 μl, 2.88 mmol) of N,N-diisopropylethylamine were added. 188 mg (0.493 mmol) of HATU were then added at RT, and the mixture was stirred for 60 h. Without further work-up, the reaction solution was purified by preparative RP-HPLC (acetonitrile/water). Yield: 89.9 mg (43% of theory).

LC-MS (method 1A): Rₜ=0.89 min; MS (ESIpos): m/z=504 [M+H]⁺;

optical rotation: [α]_{D}^{19.6}=137.5° (c=0.555, methanol);

¹H-NMR (400 MHz, DMSO-d₆): δ [ppm]=8.37 (d, 1H), 7.64 (d, 1H), 7.57 (s, 1H), 7.49-7.42 (m, 1H), 7.41-7.28 (m, 2H), 6.93 (d, 1H), 5.16 (m_{c}, 1H), 4.73-4.61 (m, 1H), 4.58-4.49 (m, 1H), 4.25 (m_{c}, 1H), 3.93 (s, 3H), 3.71 (d, 1H), 3.53 (d, 1H), 3.35 (m_{c}, 1H), 2.95 (dd, 1H), 2.71 (dd, 1H), 2.48-2.40 (m, 1H), 2.10 (br. d., 1H), 1.84 (br. d., 1H), 0.91 (d, 3H), two protons obscured

### Example 25

### (2-{[1-(3-Chlorophenyl)-2-fluoroethyl]amino}-8-methoxy[1,2,4]triazolo[1,5-a]pyridin-6-yl) [(trans)-2-hydroxy-7-methyl-8-oxa-5-azaspiro[3.5]non-5-yl]methanone [enantiomerically pure isomer 2]

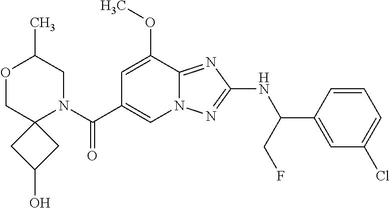 [SMILES: COc1cc(C(=O)N2CC(C)OCC23CC(O)C3)cn2nc(NC(CF)c3cccc(Cl)c3)nc12]

150 mg (0.411 mmol) of 2-{[1-(3-chlorophenyl)-2-fluoroethyl]amino}-8-methoxy[1,2,4]triazolo[1,5-a]pyridine-6-carboxylic acid [enantiomerically pure isomer] and 159 mg (0.822 mmol) of trans-7-methyl-8-oxa-5-azaspiro[3.5]nonan-2-ol hydrochloride [enantiomerically pure isomer 2, Example 104A] were initially charged in N,N-dimethylformamide (2.74 ml), and 372 mg (500 μl, 2.88 mmol) of N,N-diisopropylethylamine were added. 188 mg (0.493 mmol) of HATU were then added at RT, and the mixture was stirred for 60 h. Without further work-up, the reaction solution was then purified directly by preparative RP-HPLC (acetonitrile/water). Yield: 131 mg (63% of theory).

LC-MS (method 1A): Rₜ=0.89 min; MS (ESIpos): m/z=504 [M+H]⁺;

optical rotation: [α]_{D}^{19.4}=53.91° (c=0.52, methanol);

¹H-NMR (400 MHz, DMSO-d₆): δ [ppm]=8.38 (d, 1H), 7.64 (d, 1H), 7.57 (s, 1H), 7.50-7.43 (m, 1H), 7.41-7.28 (m, 2H), 6.93 (d, 1H), 5.16 (m_{c}, 1H), 5.01 (d, 1H), 4.72-4.61 (m, 1H), 4.59-4.48 (m, 1H), 4.26 (m_{c}, 1H), 3.93 (s, 3H), 3.71 (d, 1H), 3.52 (dd, 1H), 2.95 (dd, 1H), 2.73 (dd, 1H), 2.47-2.40 (m, 1H), 2.09 (br. d., 1H), 1.84 (br. d., 1H), 0.91 (d, 3H), two protons obscured

### Example 26

### 2-[(3-Chlorobenzyl)amino]-8-methoxy[1,2,4]triazolo[1,5-a]pyridin-6-yl](2,5,5-trimethylmorpholin-4-yl)methanone [racemate]

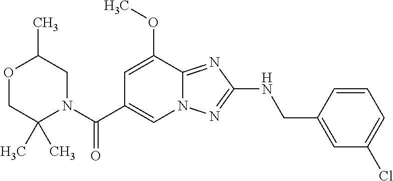 [SMILES: COc1cc(C(=O)N2CC(C)OCC2(C)C)cn2nc(NCc3cccc(Cl)c3)nc12]

120 mg (0.361 mmol) of 2-[(3-chlorobenzyl)amino]-8-methoxy[1,2,4]triazolo[1,5-a]pyridine-6-carboxylic acid and 55.9 mg (0.433 mmol) of 2,5,5-trimethylmorpholine [racemate] were initially charged in N,N-dimethylformamide (1.66 ml), and 163 mg (220 μl, 1.26 mmol) of N,N-diisopropylethylamine were added. Subsequently, 165 mg (0.433 mmol) of HATU were added at RT and the mixture was stirred overnight. Without further work-up, the reaction solution was purified by preparative RP-HPLC (acetonitrile/water). Yield: 160 mg (96% of theory).

LC-MS (method 1A): Rₜ=1.03 min; MS (ESIpos): m/z=444 [M+H]⁺;

¹H-NMR (400 MHz, DMSO-d₆): δ [ppm]=8.33 (d, 1H), 7.39 (s, 1H), 7.37-7.21 (m, 4H), 6.90 (d, 1H), 4.47 (s, 2H), 3.93 (s, 3H), 3.81-3.69 (m, 1H), 3.54-3.36 (m, 3H), 2.87 (dd, 1H), 1.40 (s, 3H), 1.37 (s, 3H), 1.02 (d, 3H).

### Example 27

### 2-[(3-Chlorobenzyl)amino]-8-methoxy[1,2,4]triazolo[1,5-a]pyridin-6-yl](2,5,5-trimethylmorpholin-4-yl)methanone [enantiomerically pure isomer 1]

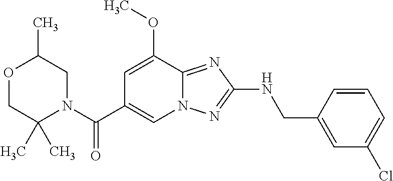 [SMILES: COc1cc(C(=O)N2CC(C)OCC2(C)C)cn2nc(NCc3cccc(Cl)c3)nc12]

Enantiomer separation on a chiral phase of 151 mg of the compound from Example 26 according to Method 17D gave 35.4 mg of Example 27 (enantiomerically pure isomer 1) and 42.5 mg of Example 28 (enantiomerically pure isomer 2).

HPLC (Method 17E): Rₜ=8.27 min, >99.9% ee;

LC-MS (method 1A): Rₜ=1.03 min; MS (ESIpos): m/z=444 [M+H]⁺;

¹H-NMR (400 MHz, DMSO-d₆): δ [ppm]=8.33 (d, 1H), 7.39 (s, 1H), 7.37-7.21 (m, 4H), 6.90 (d, 1H), 4.47 (s, 2H), 3.93 (s, 3H), 3.81-3.69 (m, 1H), 3.54-3.36 (m, 3H), 2.87 (dd, 1H), 1.40 (s, 3H), 1.37 (s, 3H), 1.02 (d, 3H).

### Example 28

### 2-[(3-Chlorobenzyl)amino]-8-methoxy[1,2,4]triazolo[1,5-a]pyridin-6-yl](2,5,5-trimethylmorpholin-4-yl)methanone [enantiomerically pure isomer 2]

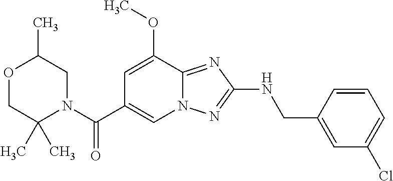 [SMILES: COc1cc(C(=O)N2CC(C)OCC2(C)C)cn2nc(NCc3cccc(Cl)c3)nc12]

Enantiomer separation on a chiral phase of 151 mg of the compound from Example 26 according to Method 17D gave 35.4 mg of Example 27 (enantiomerically pure isomer 1) and 42.5 mg of Example 28 (enantiomerically pure isomer 2).

HPLC (Method 17E): Rₜ=9.85 min, 99.4% ee;

LC-MS (method 1A): Rₜ=1.03 min; MS (ESIpos): m/z=444 [M+H]⁺;

¹H-NMR (400 MHz, DMSO-d₆): δ [ppm]=8.33 (d, 1H), 7.39 (s, 1H), 7.37-7.21 (m, 4H), 6.90 (d, 1H), 4.47 (s, 2H), 3.93 (s, 3H), 3.81-3.69 (m, 1H), 3.54-3.36 (m, 3H), 2.87 (dd, 1H), 1.40 (s, 3H), 1.37 (s, 3H), 1.02 (d, 3H).

### Example 29

### {2-[(3-Chlorobenzyl)amino]-8-methoxy[1,2,4]triazolo[1,5-a]pyridin-6-yl}[5-(hydroxymethyl)-2,5-dimethylmorpholin-4-yl]methanone [enantiomerically pure isomer 1]

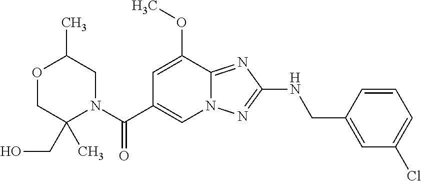 [SMILES: COc1cc(C(=O)N2CC(C)OCC2(C)CO)cn2nc(NCc3cccc(Cl)c3)nc12]

60.0 mg (0.180 mmol) of 2-[(3-chlorobenzyl)amino]-8-methoxy[1,2,4]triazolo[1,5-a]pyridine-6-carboxylic acid and 31.4 mg (0.216 mmol) of (3,6-dimethylmorpholin-3-yl)methanol [enantiomerically pure isomer 1, Example 116A] were initially charged in N,N-dimethylformamide (0.83 ml), and 81.6 mg (110 μl, 0.631 mmol) of N,N-diisopropylethylamine were added. Subsequently, 82.3 mg (0.216 mmol) of HATU were added at RT and the mixture was stirred overnight. Without further work-up, the reaction solution was purified by preparative RP-HPLC (acetonitrile/water). Yield: 43.2 mg (52% of theory).

LC-MS (method 1A): Rₜ=0.90 min; MS (ESIpos): m/z=460 [M+H]⁺;

¹H-NMR (400 MHz, DMSO-d₆): δ [ppm]=8.31 (s, 1H), 7.39 (s, 1H), 7.36-7.22 (m, 4H), 6.87 (s, 1H), 4.89 (t, 1H), 4.46 (d, 2H), 3.92 (s, 3H), 3.88 (d, 1H), 3.80-3.62 (m, 3H), 3.52 (dd, 1H), 3.25 (d, 1H), 2.94 (dd, 1H), 1.34 (s, 3H), 1.00 (d, 3H).

### Example 30

### {2-[(3-Chlorobenzyl)amino]-8-methoxy[1,2,4]triazolo[1,5-a]pyridin-6-yl}[5-(hydroxymethyl)-2,5-dimethylmorpholin-4-yl]methanone [enantiomerically pure isomer 4]

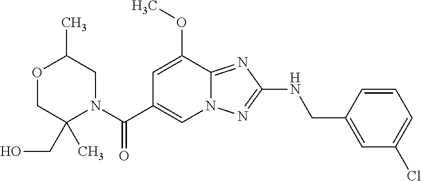 [SMILES: COc1cc(C(=O)N2CC(C)OCC2(C)CO)cn2nc(NCc3cccc(Cl)c3)nc12]

60.0 mg (0.180 mmol) of 2-[(3-chlorobenzyl)amino]-8-methoxy[1,2,4]triazolo[1,5-a]pyridine-6-carboxylic acid and 31.4 mg (0.216 mmol) of (3,6-dimethylmorpholin-3-yl)methanol [enantiomerically pure isomer 4, Example 117A] were initially charged in N,N-dimethylformamide (0.83 ml), and 81.6 mg (110 μl, 0.631 mmol) of N,N-diisopropylethylamine were added. Subsequently, 82.3 mg (0.216 mmol) of HATU were added at RT and the mixture was stirred overnight. Without further work-up, the reaction solution was purified by preparative RP-HPLC (acetonitrile/water). Yield: 46.2 mg (55% of theory).

LC-MS (method 1A): Rₜ=0.94 min; MS (ESIpos): m/z=460 [M+H]⁺;

¹H-NMR (400 MHz, DMSO-d₆): δ [ppm]=8.30 (d, 1H), 7.39 (s, 1H), 7.36-7.21 (m, 4H), 6.87 (d, 1H), 4.75 (t, 1H), 4.46 (d, 2H), 3.92 (s, 3H), 3.87-3.62 (m, 4H), 3.57-3.44 (m, 2H), 2.99 (dd, 1H), 1.32 (s, 3H), 1.01 (d, 3H).

### Example 31

### {2-[(3-Chlorobenzyl)amino]-8-methoxy[1,2,4]triazolo[1,5-a]pyridin-6-yl}[2-(2-hydroxyethyl)-2,5,5-trimethylmorpholin-4-yl]methanone [racemate]

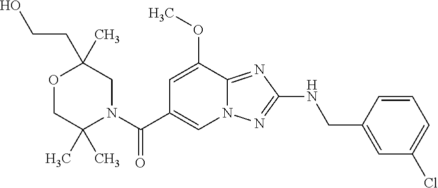 [SMILES: COc1cc(C(=O)N2CC(C)(CCO)OCC2(C)C)cn2nc(NCc3cccc(Cl)c3)nc12]

120 mg (0.361 mmol) of 2-[(3-chlorobenzyl)amino]-8-methoxy[1,2,4]triazolo[1,5-a]pyridine-6-carboxylic acid and 75.0 mg (0.433 mmol) of 2-(2,5,5-trimethylmorpholin-2-yl)ethanol [racemate] were initially charged in N,N-dimethylformamide (1.66 ml), and 163 mg (220 μl, 1.26 mmol) of N,N-diisopropylethylamine were added. Subsequently, 165 mg (0.433 mmol) of HATU were added at RT and the mixture was stirred overnight. Without further work-up, the reaction solution was purified by preparative RP-HPLC (acetonitrile/water). Yield: 74.3 mg (42% of theory).

LC-MS (method 1A): Rₜ=0.94 min; MS (ESIpos): m/z=488 [M+H]⁺;

¹H-NMR (400 MHz, DMSO-d₆): δ [ppm]=8.22 (d, 1H), 7.40 (s, 1H), 7.36-7.20 (m, 4H), 6.81 (d, 1H), 4.46 (d, 2H), 4.27 (t, 1H), 3.92 (s, 3H), 3.53-3.33 (m, 5H), 3.27-3.18 (m, 1H), 1.81-1.67 (m, 1H), 1.63-1.51 (m, 1H), 1.45 (d, 6H), 1.12 (s, 3H).

### Example 32

### {2-[(3-Chlorobenzyl)amino]-8-methoxy[1,2,4]triazolo[1,5-a]pyridin-6-yl}[2-(2-hydroxyethyl)-2,5,5-trimethylmorpholin-4-yl]methanone [enantiomerically pure isomer 1]

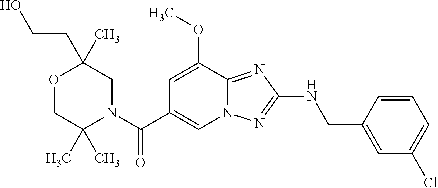 [SMILES: COc1cc(C(=O)N2CC(C)(CCO)OCC2(C)C)cn2nc(NCc3cccc(Cl)c3)nc12]

Enantiomer separation on a chiral phase of 68 mg of the compound from Example 31 according to Method 19D gave 33.0 mg of Example 32 (enantiomerically pure isomer 1) and 34.0 mg of Example 33 (enantiomerically pure isomer 2).

HPLC (Method 19E): Rₜ=4.97 min, >99.9% ee;

LC-MS (method 1A): Rₜ=0.92 min; MS (ESIpos): m/z=488 [M+H]⁺;

¹H-NMR (400 MHz, DMSO-d₆): δ [ppm]=8.22 (d, 1H), 7.40 (s, 1H), 7.37-7.23 (m, 4H), 6.81 (d, 1H), 4.46 (d, 2H), 4.29 (t, 1H), 3.92 (s, 3H), 3.48-3.37 (m, 4H), 3.27-3.20 (m, 1H), 1.81-1.68 (m, 1H), 1.64-1.51 (m, 1H), 1.45 (d, 6H), 1.12 (s, 3H).

### Example 33

### {2-[(3-Chlorobenzyl)amino]-8-methoxy[1,2,4]triazolo[1,5-a]pyridin-6-yl}[2-(2-hydroxyethyl)-2,5,5-trimethylmorpholin-4-yl]methanone [enantiomerically pure isomer 2]

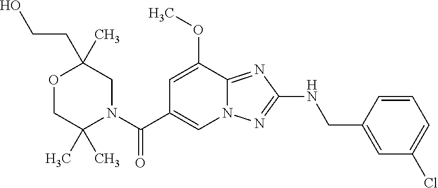 [SMILES: COc1cc(C(=O)N2CC(C)(CCO)OCC2(C)C)cn2nc(NCc3cccc(Cl)c3)nc12]

Enantiomer separation on a chiral phase of 68 mg of the compound from Example 31 according to Method 19D gave 33.0 mg of Example 32 (enantiomerically pure isomer 1) and 34.0 mg of Example 33 (enantiomerically pure isomer 2).

HPLC (Method 19E): Rₜ=6.86 min, >99.9% ee;

LC-MS (method 1A): Rₜ=0.92 min; MS (ESIpos): m/z=488 [M+H]⁺;

¹H-NMR (400 MHz, DMSO-d₆): δ [ppm]=8.22 (d, 1H), 7.40 (s, 1H), 7.37-7.23 (m, 4H), 6.81 (s, 1H), 4.46 (d, 2H), 4.29 (t, 1H), 3.92 (s, 3H), 3.49-3.36 (m, 4H), 3.27-3.19 (m, 1H), 1.81-1.69 (m, 1H), 1.64-1.51 (m, 1H), 1.45 (d, 6H), 1.12 (s, 3H).

### Example 34

### {2-[(3-Chlorobenzyl)amino]-8-methoxy[1,2,4]triazolo[1,5-a]pyridin-6-yl}[2-(2-hydroxypropyl)-2,5,5-trimethylmorpholin-4-yl]methanone [diastereomer 1, 2 isomers]

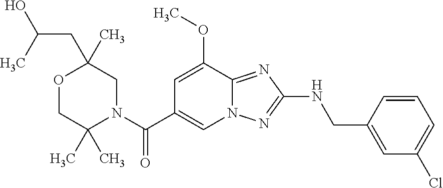 [SMILES: COc1cc(C(=O)N2CC(C)(CC(C)O)OCC2(C)C)cn2nc(NCc3cccc(Cl)c3)nc12]

120 mg (0.361 mmol) of 2-[(3-chlorobenzyl)amino]-8-methoxy[1,2,4]triazolo[1,5-a]pyridine-6-carboxylic acid and 75.0 mg (0.433 mmol) of 1-(2,5,5-trimethylmorpholin-2-yl)propan-2-ol [diastereomer 1, 2 isomers, Example 125A] were initially charged in N,N-dimethylformamide (1.66 ml), and 163 mg (220 μl, 1.26 mmol) of N,N-diisopropylethylamine were added. Subsequently, 165 mg (0.433 mmol) of HATU were added at RT and the mixture was stirred overnight. Without further work-up, the reaction solution was purified by preparative RP-HPLC (acetonitrile/water). Yield: 108 mg (59% of theory).

LC-MS (method 1A): Rₜ=0.97 min; MS (ESIpos): m/z=502 [M+H]⁺;

¹H-NMR (400 MHz, DMSO-d₆): δ [ppm]=8.21 (d, 1H), 7.40 (s, 1H), 7.37-7.22 (m, 4H), 6.81 (s, 1H), 4.46 (d, 2H), 4.17 (d, 1H), 3.92 (s, 3H), 3.78-3.64 (m, 1H), 3.54-3.34 (m, 4H), 1.60-1.39 (m, 8H), 1.18 (s, 3H), 1.03 (d, 3H).

### Example 35

### {2-[(3-Chlorobenzyl)amino]-8-methoxy[1,2,4]triazolo[1,5-a]pyridin-6-yl}[2-(2-hydroxypropyl)-2,5,5-trimethylmorpholin-4-yl]methanone [enantiomerically pure isomer 1]

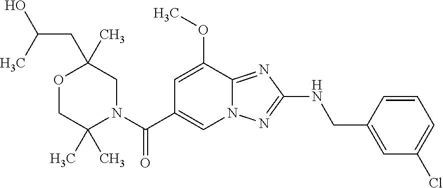 [SMILES: COc1cc(C(=O)N2CC(C)(CC(C)O)OCC2(C)C)cn2nc(NCc3cccc(Cl)c3)nc12]

Enantiomer separation on a chiral phase of 102 mg of the compound from Example 34 according to Method 20D gave 23.6 mg of Example 35 (enantiomerically pure isomer 1) and 27.7 mg of Example 36 (enantiomerically pure isomer 2).

HPLC (Method 19E): Rₜ=5.74 min, >99.9% ee;

LC-MS (method 1A): Rₜ=0.98 min; MS (ESIpos): m/z=502 [M+H]⁺;

¹H-NMR (400 MHz, DMSO-d₆): δ [ppm]=8.21 (d, 1H), 7.40 (s, 1H), 7.37-7.22 (m, 4H), 6.81 (s, 1H), 4.46 (d, 2H), 4.17 (d, 1H), 3.92 (s, 3H), 3.78-3.64 (m, 1H), 3.54-3.34 (m, 4H), 1.60-1.39 (m, 8H), 1.18 (s, 3H), 1.03 (d, 3H).

### Example 36

### {2-[(3-Chlorobenzyl)amino]-8-methoxy[1,2,4]triazolo[1,5-a]pyridin-6-yl}[2-(2-hydroxypropyl)-2,5,5-trimethylmorpholin-4-yl]methanone [enantiomerically pure isomer 2]

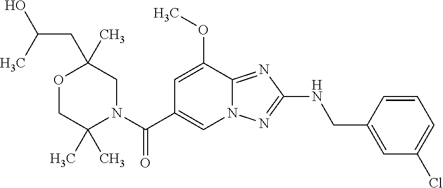 [SMILES: COc1cc(C(=O)N2CC(C)(CC(C)O)OCC2(C)C)cn2nc(NCc3cccc(Cl)c3)nc12]

Enantiomer separation on a chiral phase of 102 mg of the compound from Example 34 according to Method 20D gave 23.6 mg of Example 35 (enantiomerically pure isomer 1) and 27.7 mg of Example 36 (enantiomerically pure isomer 2).

HPLC (Method 19E): Rₜ=7.09 min, 96.4% ee;

LC-MS (method 1A): Rₜ=0.98 min; MS (ESIpos): m/z=502 [M+H]⁺;

¹H-NMR (400 MHz, DMSO-d₆): δ [ppm]=8.21 (d, 1H), 7.40 (s, 1H), 7.37-7.22 (m, 4H), 6.81 (s, 1H), 4.46 (d, 2H), 4.17 (d, 1H), 3.92 (s, 3H), 3.78-3.64 (m, 1H), 3.54-3.34 (m, 4H), 1.60-1.39 (m, 8H), 1.18 (s, 3H), 1.03 (d, 3H).

### Example 37

### {2-[(3-Chlorobenzyl)amino]-8-methoxy[1,2,4]triazolo[1,5-a]pyridin-6-yl}[2-(2-hydroxypropyl)-2,5,5-trimethylmorpholin-4-yl]methanone [diastereomer 2, 2 isomers]

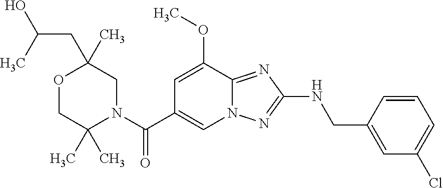 [SMILES: COc1cc(C(=O)N2CC(C)(CC(C)O)OCC2(C)C)cn2nc(NCc3cccc(Cl)c3)nc12]

100 mg (0.301 mmol) of 2-[(3-chlorobenzyl)amino]-8-methoxy[1,2,4]triazolo[1,5-a]pyridine-6-carboxylic acid and 67.5 mg (0.361 mmol) of 1-(2,5,5-trimethylmorpholin-2-yl)propan-2-ol [diastereomer 2, 2 isomers, Example 126A] were initially charged in N,N-dimethylformamide (1.38 ml), and 136 mg (183 μl, 1.05 mmol) of N,N-diisopropylethylamine were added. Subsequently, 137 mg (0.361 mmol) of HATU were added at RT and the mixture was stirred overnight. Without work-up, the reaction solution was purified directly by preparative RP-HPLC (acetonitrile/water). Yield: 101 mg (67% of theory).

LC-MS (method 1A): Rₜ=0.97 min; MS (ESIpos): m/z=502 [M+H]⁺;

¹H-NMR (400 MHz, DMSO-d₆): δ [ppm]=8.24 (d, 1H), 7.40 (s, 1H), 7.37-7.22 (m, 4H), 6.82 (d, 1H), 4.46 (d, 2H), 4.19 (d, 1H), 3.92 (s, 3H), 3.75 (m_{c}, 1H), 3.59-3.39 (m, 3H), 3.22 (d, 1H), 1.58 (dd, 1H), 1.52-1.36 (m, 7H), 1.20 (s, 3H), 1.00 (d, 3H).

### Example 38

### {2-[(3-Chlorobenzyl)amino]-8-methoxy[1,2,4]triazolo[1,5-a]pyridin-6-yl}[2-(2-hydroxypropyl)-2,5,5-trimethylmorpholin-4-yl]methanone [enantiomerically pure isomer 1]

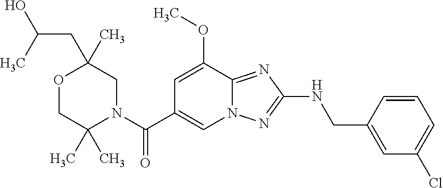 [SMILES: COc1cc(C(=O)N2CC(C)(CC(C)O)OCC2(C)C)cn2nc(NCc3cccc(Cl)c3)nc12]

Enantiomer separation on a chiral phase of 96.0 mg of the compound from Example 37 according to Method 21D gave 44.0 mg of Example 38 (enantiomerically pure isomer 1) and 42.2 mg of Example 39 (enantiomerically pure isomer 2).

HPLC (Method 21E): Rₜ=6.53 min, >99.9% ee;

LC-MS (method 1A): Rₜ=0.97 min; MS (ESIpos): m/z=502 [M+H]⁺;

¹H-NMR (400 MHz, DMSO-d₆): δ [ppm]=8.24 (d, 1H), 7.40 (s, 1H), 7.37-7.22 (m, 4H), 6.82 (d, 1H), 4.46 (d, 2H), 4.19 (d, 1H), 3.92 (s, 3H), 3.75 (m_{c}, 1H), 3.59-3.39 (m, 3H), 3.22 (d, 1H), 1.58 (dd, 1H), 1.52-1.36 (m, 7H), 1.20 (s, 3H), 1.00 (d, 3H).

### Example 39

### {2-[(3-Chlorobenzyl)amino]-8-methoxy[1,2,4]triazolo[1,5-a]pyridin-6-yl}[2-(2-hydroxypropyl)-2,5,5-trimethylmorpholin-4-yl]methanone [enantiomerically pure isomer 2]

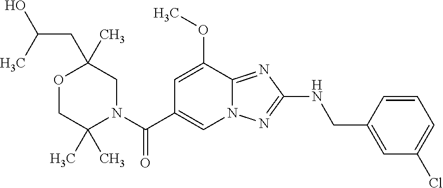 [SMILES: COc1cc(C(=O)N2CC(C)(CC(C)O)OCC2(C)C)cn2nc(NCc3cccc(Cl)c3)nc12]

Enantiomer separation on a chiral phase of 96.0 mg of the compound from Example 37 according to Method 21D gave 44.0 mg of Example 38 (enantiomerically pure isomer 1) and 42.2 mg of Example 39 (enantiomerically pure isomer 2).

HPLC (Method 21E): Rₜ=8.54 min, >99.9% ee;

LC-MS (method 1A): Rₜ=0.97 min; MS (ESIpos): m/z=502 [M+H]⁺;

¹H-NMR (400 MHz, DMSO-d₆): δ [ppm]=8.24 (d, 1H), 7.40 (s, 1H), 7.37-7.22 (m, 4H), 6.82 (d, 1H), 4.46 (d, 2H), 4.19 (d, 1H), 3.92 (s, 3H), 3.75 (m_{c}, 1H), 3.59-3.39 (m, 3H), 3.22 (d, 1H), 1.58 (dd, 1H), 1.52-1.36 (m, 7H), 1.20 (s, 3H), 1.00 (d, 3H).

### Example 40

### {2-[(3-Chlorobenzyl)amino]-8-methoxy[1,2,4]triazolo[1,5-a]pyridin-6-yl}[5-(2-hydroxypropan-2-yl)-2-methylmorpholin-4-yl]methanone [enantiomerically pure isomer 1]

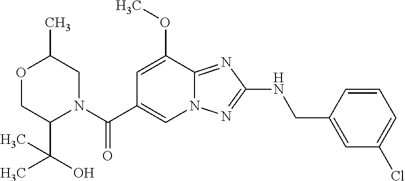 [SMILES: COc1cc(C(=O)N2CC(C)OCC2C(C)(C)O)cn2nc(NCc3cccc(Cl)c3)nc12]

81.8 mg (0.246 mmol) of 2-[(3-chlorobenzyl)amino]-8-methoxy[1,2,4]triazolo[1,5-a]pyridine-6-carboxylic acid and 47.0 mg (0.295 mmol) of 2-(6-methylmorpholin-3-yl)propan-2-ol [enantiomerically pure isomer 1, Example 135A] were initially charged in N,N-dimethylformamide (1.13 ml), and 111 mg (150 μl, 0.861 mmol) of N,N-diisopropylethylamine were added. Subsequently, 112 mg (0.295 mmol) of HATU were added at RT and the mixture was stirred overnight. Without further work-up, the reaction solution was purified by preparative RP-HPLC (acetonitrile/water). Yield: 85.8 mg (73% of theory).

LC-MS (method 1A): Rₜ=0.92 min; MS (ESIpos): m/z=474 [M+H]⁺;

¹H-NMR (400 MHz, DMSO-d₆): δ [ppm]=8.49-8.35 (m, 1H), 7.39 (s, 1H), 7.37-7.24 (m, 4H), 6.90 (s, 1H), 4.65-4.53 (m, 1H), 4.46 (d, 2H), 4.35-4.24 (m, 2H), 3.98-3.87 (m, 3H), 3.63-3.44 (m, 2H), 1.29 (s, 3H), 1.21 (s, 3H), 0.97 (d, 3H), 2 protons obscured

### Example 41

### {2-[(3-Chlorobenzyl)amino]-8-methoxy[1,2,4]triazolo[1,5-a]pyridin-6-yl}[(5R)-2-(2-hydroxypropyl)-2,5-dimethylmorpholin-4-yl]methanone [enantiomerically pure isomer]

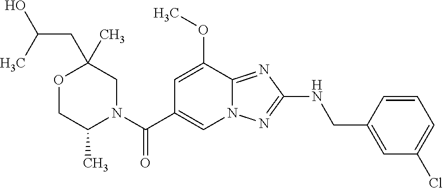 [SMILES: COc1cc(C(=O)N2CC(C)(CC(C)O)OC[C@H]2C)cn2nc(NCc3cccc(Cl)c3)nc12]

80.0 mg (0.240 mmol) of 2-[(3-chlorobenzyl)amino]-8-methoxy[1,2,4]triazolo[1,5-a]pyridine-6-carboxylic acid and 50.0 mg (0.289 mmol) of 1-[(5R)-2,5-dimethylmorpholin-2-yl]propan-2-ol [enantiomerically pure isomer 3, Example 143A] were initially charged in N,N-dimethylformamide (1.11 ml), and 109 mg (147 μl, 0.841 mmol) of N,N-diisopropylethylamine were added. Subsequently, 110 mg (0.289 mmol) of HATU were added at RT and the mixture was stirred overnight. Without further work-up, the reaction solution was purified by preparative RP-HPLC (acetonitrile/water). Yield: 104 mg (89% of theory).

LC-MS (method 1A): Rₜ=0.92 min; MS (ESIpos): m/z=488 [M+H]⁺;

¹H-NMR (400 MHz, DMSO-d₆): δ [ppm]=8.31 (s, 1H), 7.40 (s, 1H), 7.37-7.23 (m, 4H), 6.85 (d, 1H), 4.46 (d, 2H), 4.20 (d, 2H), 3.93 (s, 3H), 3.80-3.64 (m, 3H), 3.05 (d, 1H), 1.80 (d, 1H), 1.40 (dd, 1H), 1.24 (d, 3H), 1.16 (s, 3H), 1.08 (d, 3H), one proton obscured

### Example 42

### {2-[(3-Chlorobenzyl)amino]-8-methoxy[1,2,4]triazolo[1,5-a]pyridin-6-yl}[5-(2-hydroxy-2-methylpropyl)-2-methylmorpholin-4-yl]methanone [racemate]

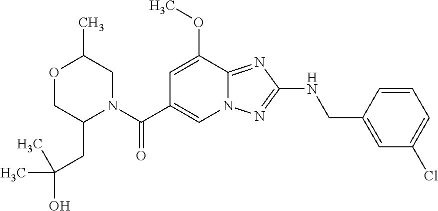 [SMILES: COc1cc(C(=O)N2CC(C)OCC2CC(C)(C)O)cn2nc(NCc3cccc(Cl)c3)nc12]

59.2 mg (0.178 mmol) of 2-[(3-chlorobenzyl)amino]-8-methoxy[1,2,4]triazolo[1,5-a]pyridine-6-carboxylic acid and 37.0 mg (0.214 mmol) of 2-methyl-1-(6-methylmorpholin-3-yl)propan-2-ol [diastereomer 2, 2 isomers, Example 151A] were initially charged in N,N-dimethylformamide (0.82 ml), and 80.5 mg (108 μl, 0.623 mmol) of N,N-diisopropylethylamine were added. Subsequently, 81.2 mg (0.214 mmol) of HATU were added at RT and the mixture was stirred overnight. Without further work-up, the reaction solution was purified by preparative RP-HPLC (acetonitrile/water). Yield: 76.7 mg (88% of theory).

LC-MS (method 1A): Rₜ=0.93 min; MS (ESIpos): m/z=488 [M+H]⁺.

### Example 43

### {2-[(3-Chlorobenzyl)amino]-8-methoxy[1,2,4]triazolo[1,5-a]pyridin-6-yl}[5-(2-hydroxy-2-methylpropyl)-2-methylmorpholin-4-yl]methanone [enantiomerically pure isomer 1]

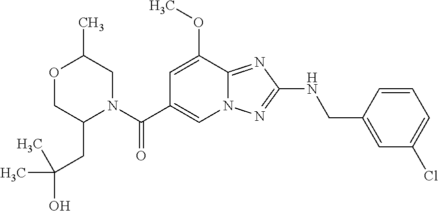 [SMILES: COc1cc(C(=O)N2CC(C)OCC2CC(C)(C)O)cn2nc(NCc3cccc(Cl)c3)nc12]

Enantiomer separation on a chiral phase of 70.0 mg of the compound from Example 42 according to Method 23D gave 31.0 mg of Example 43 (enantiomerically pure isomer 1) and 34.0 mg of Example 44 enantiomerically pure isomer 2).

HPLC (Method 4E): Rₜ=5.11 min, >99.9% ee;

LC-MS (method 1A): Rₜ=0.92 min; MS (ESIpos): m/z=488 [M+H]⁺.

### Example 44

### {2-[(3-Chlorobenzyl)amino]-8-methoxy[1,2,4]triazolo[1,5-a]pyridin-6-yl}[5-(2-hydroxy-2-methylpropyl)-2-methylmorpholin-4-yl]methanone [enantiomerically pure isomer 2]

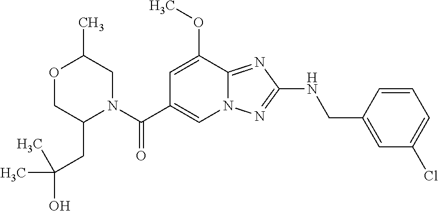 [SMILES: COc1cc(C(=O)N2CC(C)OCC2CC(C)(C)O)cn2nc(NCc3cccc(Cl)c3)nc12]

Enantiomer separation on a chiral phase of 70.0 mg of the compound from Example 42 according to Method 23D gave 31.0 mg of Example 43 (enantiomerically pure isomer 1) and 34.0 mg of Example 44 (enantiomerically pure isomer 2).

HPLC (Method 4E): Rₜ=6.51 min, >99.9% ee;

LC-MS (method 1A): Rₜ=0.91 min; MS (ESIpos): m/z=488 [M+H]⁺.

### Example 45

### {2-[(3-Chlorobenzyl)amino]-8-methoxy[1,2,4]triazolo[1,5-a]pyridin-6-yl}[5-(2-hydroxypropyl)-2,2-dimethylmorpholin-4-yl]methanone [diastereomer mixture, 4 isomers]

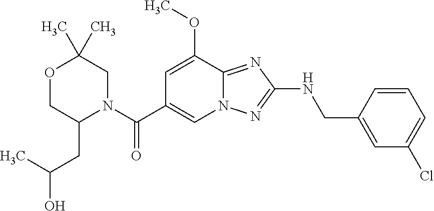 [SMILES: COc1cc(C(=O)N2CC(C)(C)OCC2CC(C)O)cn2nc(NCc3cccc(Cl)c3)nc12]

150 mg (0.451 mmol) of 2-[(3-chlorobenzyl)amino]-8-methoxy[1,2,4]triazolo[1,5-a]pyridine-6-carboxylic acid and 93.7 mg (0.541 mmol) of 1-[6,6-dimethylmorpholin-3-yl]propan-2-ol [diastereomer mixture, 4 isomers, Example 154A] were initially charged in N,N-dimethylformamide (2.15 ml), and 204 mg (275 μl, 1.58 mmol) of N,N-diisopropylethylamine were added. 206 mg (0.541 mmol) of HATU were then added at RT, and the mixture was stirred for 1 h. Without further work-up, the reaction solution was purified by preparative RP-HPLC (acetonitrile/water). Yield: 185 mg (83% of theory).

LC-MS (Method 1A): Rₜ=0.91 min (diastereomer 1, 2 isomers), Rₜ=0.92 min (diastereomer 2, 2 isomers);

MS (ESIpos): m/z=488 [M+H]⁺.

### Example 46

### {2-[(3-Chlorobenzyl)amino]-8-methoxy[1,2,4]triazolo[1,5-a]pyridin-6-yl}[5-(2-hydroxypropyl)-2,2-dimethylmorpholin-4-yl]methanone [enantiomerically pure isomer 1]

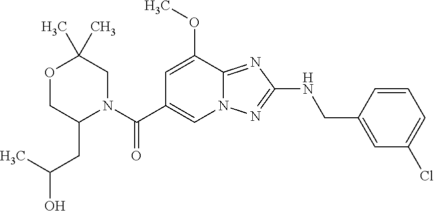 [SMILES: COc1cc(C(=O)N2CC(C)(C)OCC2CC(C)O)cn2nc(NCc3cccc(Cl)c3)nc12]

Enantiomer separation on a chiral phase of 175 mg of the compound from Example 45 according to Method 24D gave 24.5 mg of Example 46 (enantiomerically pure isomer 1), 24.0 mg of Example 47 (enantiomerically pure isomer 2), 30.0 mg of Example 48 (enantiomerically pure isomer 3) and 41.4 mg of Example 49 (enantiomerically pure isomer 4).

HPLC (Method 22E): Rₜ=4.14 min, 99.9% ee;

¹H-NMR (400 MHz, DMSO-d₆): δ [ppm]=8.29 (br. s., 1H), 7.39 (s, 1H), 7.37-7.23 (m, 4H), 6.86 (s, 1H), 4.46 (d, 3H), 3.93 (s, 3H), 3.78 (dd, 1H), 3.63 (br. s., 1H), 3.48 (d, 1H), 1.83 (br. s., 2H), 1.18-1.01 (m, 9H), 3 protons obscured

### Example 47

### {2-[(3-Chlorobenzyl)amino]-8-methoxy[1,2,4]triazolo[1,5-a]pyridin-6-yl}[5-(2-hydroxypropyl)-2,2-dimethylmorpholin-4-yl]methanone [enantiomerically pure isomer 2]

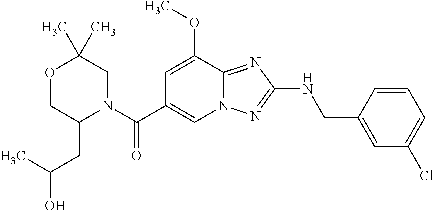 [SMILES: COc1cc(C(=O)N2CC(C)(C)OCC2CC(C)O)cn2nc(NCc3cccc(Cl)c3)nc12]

Enantiomer separation on a chiral phase of 175 mg of the compound from Example 45 according to Method 24D gave 24.5 mg of Example 46 (enantiomerically pure isomer 1), 24.0 mg of Example 47 (enantiomerically pure isomer 2), 30.0 mg of Example 48 (enantiomerically pure isomer 3) and 41.4 mg of Example 49 (enantiomerically pure isomer 4).

HPLC (Method 22E): Rₜ=4.62 min, 90% ee;

¹H-NMR (400 MHz, DMSO-d₆): δ [ppm]=8.33 (br. s., 1H), 7.39 (s, 1H), 7.36-7.21 (m, 4H), 6.88 (s, 1H), 4.46 (d, 2H), 4.36 (d, 1H), 3.93 (s, 3H), 3.87 (d, 1H), 3.73-3.38 (m, 2H), 1.99-1.86 (m, 1H), 1.73-1.51 (m, 1H), 1.21-0.95 (m, 9H), 3 protons obscured

### Example 48

### {2-[(3-Chlorobenzyl)amino]-8-methoxy[1,2,4]triazolo[1,5-a]pyridin-6-yl}[5-(2-hydroxypropyl)-2,2-dimethylmorpholin-4-yl]methanone [enantiomerically pure isomer 3]

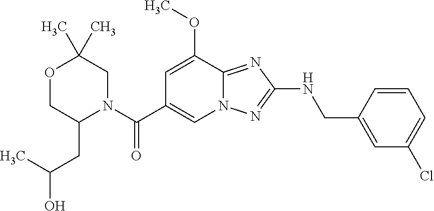 [SMILES: COc1cc(C(=O)N2CC(C)(C)OCC2CC(C)O)cn2nc(NCc3cccc(Cl)c3)nc12]

Enantiomer separation on a chiral phase of 175 mg of the compound from Example 45 according to Method 24D gave 24.5 mg of Example 46 (enantiomerically pure isomer 1), 24.0 mg of Example 47 (enantiomerically pure isomer 2), 30.0 mg of Example 48 (enantiomerically pure isomer 3) and 41.4 mg of Example 49 (enantiomerically pure isomer 4).

HPLC (Method 22E): Rₜ=5.38 min, 93% ee;

¹H-NMR (400 MHz, DMSO-d₆): δ [ppm]=8.33 (br. s., 1H), 7.39 (s, 1H), 7.37-7.22 (m, 4H), 6.88 (s, 1H), 4.46 (d, 2H), 4.36 (d, 1H), 3.93 (s, 3H), 3.87 (d, 1H), 3.70-3.39 (m, 2H), 1.99-1.87 (m, 1H), 1.67-1.52 (m, 1H), 1.22-0.95 (m, 9H), 3 protons obscured

### Example 49

### {2-[(3-Chlorobenzyl)amino]-8-methoxy[1,2,4]triazolo[1,5-a]pyridin-6-yl}[5-(2-hydroxypropyl)-2,2-dimethylmorpholin-4-yl]methanone [enantiomerically pure isomer 4]

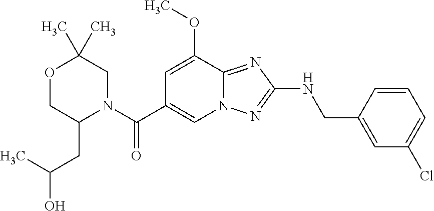 [SMILES: COc1cc(C(=O)N2CC(C)(C)OCC2CC(C)O)cn2nc(NCc3cccc(Cl)c3)nc12]

Enantiomer separation on a chiral phase of 175 mg of the compound from Example 45 according to Method 24D gave 24.5 mg of Example 46 (enantiomerically pure isomer 1), 24.0 mg of Example 47 (enantiomerically pure isomer 2), 30.0 mg of Example 48 (enantiomerically pure isomer 3) and 41.4 mg of Example 49 (enantiomerically pure isomer 4).

HPLC (Method 22E): Rₜ=5.91 min, 91% ee;

¹H-NMR (400 MHz, DMSO-d₆): δ [ppm]=8.29 (br. s., 1H), 7.39 (s, 1H), 7.35-7.22 (m, 4H), 6.86 (s, 1H), 4.46 (d, 3H), 3.93 (s, 3H), 3.78 (dd, 1H), 3.63 (br. s., 1H), 3.48 (d, 1H), 1.84 (br. s., 2H), 1.21-0.96 (m, 9H), three protons obscured

### Example 50

### {2-[(3-Chlorobenzyl)amino]-8-methoxy[1,2,4]triazolo[1,5-a]pyridin-6-yl}[5-(fluoromethyl)-2-(2-hydroxyethyl)-2-methylmorpholin-4-yl]methanone [diastereomer mixture, 4 isomers]

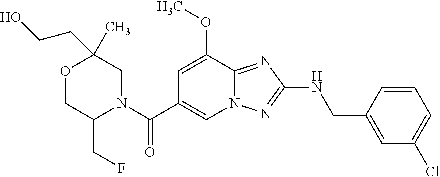 [SMILES: COc1cc(C(=O)N2CC(C)(CCO)OCC2CF)cn2nc(NCc3cccc(Cl)c3)nc12]

200 mg (0.601 mmol) of 2-[(3-chlorobenzyl)amino]-8-methoxy[1,2,4]triazolo[1,5-a]pyridine-6-carboxylic acid and 128 mg (0.721 mmol) of 2-[5-(fluoromethyl)-2-methylmorpholin-2-yl]ethanol [diastereomer mixture, 4 isomers] were initially charged in N,N-dimethylformamide (2.87 ml), and 272 mg (366 μl, 2.10 mmol) of N,N-diisopropylethylamine were added. 274 mg (0.742 mmol) of HATU were then added at RT, and the mixture was stirred for 1 h. Without further work-up, the reaction solution was purified by preparative RP-HPLC (acetonitrile/water). Yield: 221 mg (74% of theory).

LC-MS (method 1A): Rₜ=0.88 min; MS (ESIpos): m/z=488 [M+H]⁺.

### Example 51

### {2-[(3-Chlorobenzyl)amino]-8-methoxy[1,2,4]triazolo[1,5-a]pyridin-6-yl}[5-(fluoromethyl)-2-(2-hydroxyethyl)-2-methylmorpholin-4-yl]methanone [enantiomerically pure isomer 1]

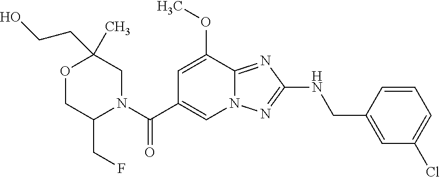 [SMILES: COc1cc(C(=O)N2CC(C)(CCO)OCC2CF)cn2nc(NCc3cccc(Cl)c3)nc12]

Enantiomer separation on a chiral phase of 215 mg of the compound from Example 50 according to Method 25D and subsequent purification on an achiral phase according to Method 1F gave 17.9 mg of Example 51 (enantiomerically pure isomer 1), 16.4 mg of Example 52 (enantiomerically pure isomer 2), 15.6 mg of Example 53 (enantiomerically pure isomer 3) and 8.1 mg of Example 54 (enantiomerically pure isomer 4).

HPLC (Method 23E): Rₜ=13.4 min, 97% ee;

LC-MS (method 1A): Rₜ=0.88 min; MS (ESIpos): m/z=492 [M+H]⁺;

¹H-NMR (400 MHz, DMSO-d₆): δ [ppm]=8.31 (s, 1H), 7.40 (s, 1H), 7.37-7.22 (m, 4H), 6.84 (s, 1H), 4.90-4.51 (m, 2H), 4.47 (d, 2H), 4.30 (br. s., 1H), 3.93 (s, 3H), 3.87-3.79 (m, 1H), 3.63-3.35 (m, 3H), 3.06 (br. s., 1H), 2.01 (br. s., 1H), 1.54 (br. s., 1H), 1.10 (br. s., 3H), 2 protons obscured

### Example 52

### {2-[(3-Chlorobenzyl)amino]-8-methoxy[1,2,4]triazolo[1,5-a]pyridin-6-yl}[5-(fluoromethyl)-2-(2-hydroxyethyl)-2-methylmorpholin-4-yl]methanone [enantiomerically pure isomer 2]

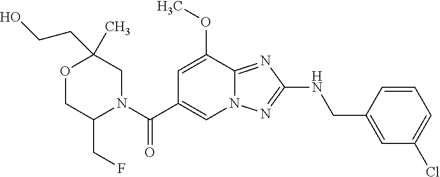 [SMILES: COc1cc(C(=O)N2CC(C)(CCO)OCC2CF)cn2nc(NCc3cccc(Cl)c3)nc12]

Enantiomer separation on a chiral phase of 215 mg of the compound from Example 50 according to Method 25D and subsequent purification on an achiral phase according to Method 1F gave 17.9 mg of Example 51 (enantiomerically pure isomer 1), 16.4 mg of Example 52 (enantiomerically pure isomer 2), 15.6 mg of Example 53 (enantiomerically pure isomer 3) and 8.1 mg of Example 54 (enantiomerically pure isomer 4).

HPLC (Method 23E): Rₜ=16.8 min, 97% ee;

LC-MS (method 1A): Rₜ=0.88 min; MS (ESIpos): m/z=492 [M+H]⁺;

¹H-NMR (400 MHz, DMSO-d₆): δ [ppm]=8.33 (s, 1H), 7.39 (s, 1H), 7.36-7.22 (m, 4H), 6.85 (s, 1H), 4.88-4.51 (m, 2H), 4.47 (d, 2H), 4.32 (br. s., 1H), 3.96-3.85 (m, 4H), 3.63-3.42 (m, 4H), 1.63 (br. s., 2H), 1.19 (br. s., 3H), 2 protons obscured

### Example 53

### {2-[(3-Chlorobenzyl)amino]-8-methoxy[1,2,4]triazolo[1,5-a]pyridin-6-yl}[5-(fluoromethyl)-2-(2-hydroxyethyl)-2-methylmorpholin-4-yl]methanone [enantiomerically pure isomer 3]

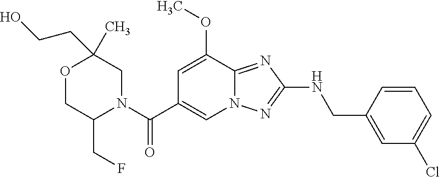 [SMILES: COc1cc(C(=O)N2CC(C)(CCO)OCC2CF)cn2nc(NCc3cccc(Cl)c3)nc12]

Enantiomer separation on a chiral phase of 215 mg of the compound from Example 50 according to Method 25D and subsequent purification on an achiral phase according to Method 1F gave 17.9 mg of Example 51 (enantiomerically pure isomer 1), 16.4 mg of Example 52 (enantiomerically pure isomer 2), 15.6 mg of Example 53 (enantiomerically pure isomer 3) and 8.1 mg of Example 54 (enantiomerically pure isomer 4).

HPLC (Method 23E): Rₜ=19.2 min, >99.9% ee;

LC-MS (method 1A): Rₜ=0.88 min; MS (ESIpos): m/z=492 [M+H]⁺;

¹H-NMR (400 MHz, DMSO-d₆): δ [ppm]=8.31 (s, 1H), 7.40 (s, 1H), 7.37-7.22 (m, 4H), 6.84 (s, 1H), 4.90-4.51 (m, 2H), 4.47 (d, 2H), 4.30 (br. s., 1H), 3.93 (s, 3H), 3.87-3.79 (m, 1H), 3.63-3.35 (m, 3H), 3.06 (br. s., 1H), 2.01 (br. s., 1H), 1.54 (br. s., 1H), 1.10 (br. s., 3H), 2 protons obscured

### Example 54

### {2-[(3-Chlorobenzyl)amino]-8-methoxy[1,2,4]triazolo[1,5-a]pyridin-6-yl}[5-(fluoromethyl)-2-(2-hydroxyethyl)-2-methylmorpholin-4-yl]methanone [enantiomerically pure isomer 4]

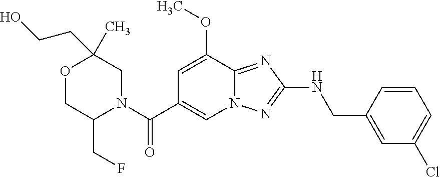 [SMILES: COc1cc(C(=O)N2CC(C)(CCO)OCC2CF)cn2nc(NCc3cccc(Cl)c3)nc12]

Enantiomer separation on a chiral phase of 215 mg of the compound from Example 50 according to Method 25D and subsequent purification on an achiral phase according to Method 1F gave 17.9 mg of Example 51 (enantiomerically pure isomer 1), 16.4 mg of Example 52 (enantiomerically pure isomer 2), 15.6 mg of Example 53 (enantiomerically pure isomer 3) and 8.1 mg of Example 54 (enantiomerically pure isomer 4).

HPLC (Method 23E): Rₜ=23.5 min, >99.9% ee;

LC-MS (method 1A): Rₜ=0.88 min; MS (ESIpos): m/z=492 [M+H]⁺;

¹H-NMR (400 MHz, DMSO-d₆): δ [ppm]=8.33 (s, 1H), 7.39 (s, 1H), 7.36-7.22 (m, 4H), 6.85 (s, 1H), 4.88-4.51 (m, 2H), 4.47 (d, 2H), 4.32 (br. s., 1H), 3.96-3.85 (m, 4H), 3.63-3.42 (m, 4H), 1.63 (br. s., 2H), 1.19 (br. s., 3H), 2 protons obscured

### Example 55

### {[(3-Chlorobenzyl)amino]-8-methoxy[1,2,4]triazolo[1,5-a]pyridin-6-yl}[5-(difluoromethyl)-2-(2-hydroxyethyl)-2-methylmorpholin-4-yl]methanone [diastereomer 1, 2 isomers]

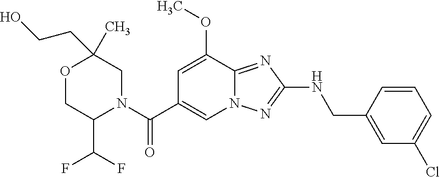 [SMILES: COc1cc(C(=O)N2CC(C)(CCO)OCC2C(F)F)cn2nc(NCc3cccc(Cl)c3)nc12]

100 mg (0.282 mmol) of 2-[(3-chlorobenzyl)amino]-8-methoxy[1,2,4]triazolo[1,5-a]pyridine-6-carboxylic acid and 70.4 mg (0.339 mmol) of 2-[5-(difluoromethyl)-2-methylmorpholin-2-yl]ethanol [diastereomer 1, 2 isomers, Example 169A] were initially charged in N,N-dimethylformamide (1.35 ml), and 128 mg (172 μl, 0.99 mmol) of N,N-diisopropylethylamine were added. Subsequently, 129 mg (0.339 mmol) of HATU were added at RT and the mixture was stirred overnight. The reaction solution was warmed to 60° C. and stirred for 3 h, then cooled to RT and stirred overnight. Without further work-up, the reaction solution was purified by preparative RP-HPLC (acetonitrile/water). Yield: 10.8 mg (7.5% of theory).

LC-MS (method 1A): Rₜ=0.94 min; MS (ESIpos): m/z=510 [M+H]⁺;

¹H-NMR (500 MHz, DMSO-d₆): δ [ppm]=8.31 (br. s., 1H), 7.46-7.23 (m, 5H), 6.82 (s, 1H), 6.45 (br. t., 1H), 4.70 (br. s., 1H), 4.47 (d, 2H), 4.35 (br. s., 1H), 4.17-3.73 (m, 6H), 3.57 (br. s., 1H), 2.16-1.83 (m, 1H), 1.71-1.43 (m, 1H), 1.21-0.94 (m, 3H), 2 protons obscured

### Example 56

### {[(3-Chlorobenzyl)amino]-8-methoxy[1,2,4]triazolo[1,5-a]pyridin-6-yl}[5-(fluoromethyl)-2-(2-hydroxypropyl)-2-methylmorpholin-4-yl]methanone [enantiomerically pure isomer 1]

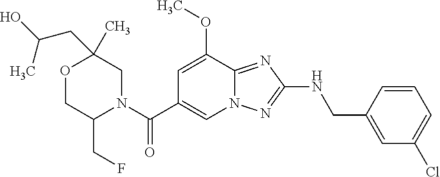 [SMILES: COc1cc(C(=O)N2CC(C)(CC(C)O)OCC2CF)cn2nc(NCc3cccc(Cl)c3)nc12]

91.8 mg (0.276 mmol) of 2-[(3-chlorobenzyl)amino]-8-methoxy[1,2,4]triazolo[1,5-a]pyridine-6-carboxylic acid and 63.3 mg (0.331 mmol) of 1-[5-(fluoromethyl)-2-methylmorpholin-2-yl]propan-2-ol [diastereomer 2, 2 isomers, Example 176A] were initially charged in N,N-dimethylformamide (1.27 ml), and 125 mg (168 μl, 0.965 mmol) of N,N-diisopropylethylamine were added. Subsequently, 126 mg (0.331 mmol) of HATU were added at RT and the mixture was stirred overnight. Without further work-up, the reaction solution was purified by preparative RP-HPLC (acetonitrile/water). Enantiomer separation on a chiral phase of 55.8 mg according to Method 26D and subsequent purification on an achiral phase gave 14.6 mg of Example 56 (enantiomerically pure isomer 1) and 15.8 mg of Example 57 (enantiomerically pure isomer 2).

HPLC (Method 24E): Rₜ=6.01 min, >99.9% ee;

LC-MS (method 1A): Rₜ=0.89 min; MS (ESIpos): m/z=506 [M+H]⁺.

### Example 57

### {2-[(3-Chlorobenzyl)amino]-8-methoxy[1,2,4]triazolo[1,5-a]pyridin-6-yl}[5-(fluoromethyl)-2-(2-hydroxypropyl)-2-methylmorpholin-4-yl]methanone [enantiomerically pure isomer 2]

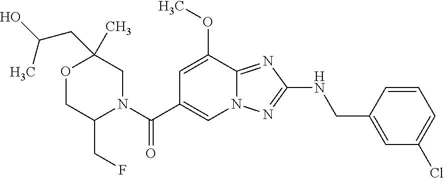 [SMILES: COc1cc(C(=O)N2CC(C)(CC(C)O)OCC2CF)cn2nc(NCc3cccc(Cl)c3)nc12]

91.8 mg (0.276 mmol) of 2-[(3-chlorobenzyl)amino]-8-methoxy[1,2,4]triazolo[1,5-a]pyridine-6-carboxylic acid and 63.3 mg (0.331 mmol) of 1-[5-(fluoromethyl)-2-methylmorpholin-2-yl]propan-2-ol [diastereomer 2, 2 isomers, Example 176A] were initially charged in N,N-dimethylformamide (1.27 ml), and 125 mg (168 μl, 0.965 mmol) of N,N-diisopropylethylamine were added. Subsequently, 126 mg (0.331 mmol) of HATU were added at RT and the mixture was stirred overnight. Without further work-up, the reaction solution was then purified directly by preparative RP-HPLC (acetonitrile/water). Enantiomer separation on a chiral phase of 55.8 mg according to Method 26D and subsequent purification on an achiral phase gave 14.6 mg of Example 56 (enantiomerically pure isomer 1) and 15.8 mg of Example 57 (enantiomerically pure isomer 2).

HPLC (Method 24E): Rₜ=8.13 min, >99.9% ee;

LC-MS (method 1A): Rₜ=0.92 min; MS (ESIpos): m/z=506 [M+H]⁺.

### Example 58

### 4-({2-[(3-Chlorobenzyl)amino]-8-methoxy[1,2,4]triazolo[1,5-a]pyridin-6-yl}carbonyl)-3-ethyl-1,4-diazabicyclo[4.2.0]octan-2-one [diastereomer mixture, 4 isomers]

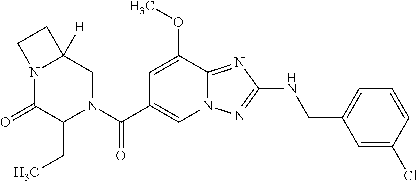 [SMILES: CCC1C(=O)N2CCC2CN1C(=O)c1cc(OC)c2nc(NCc3cccc(Cl)c3)nn2c1]

440 mg (592 μl, 3.40 mmol) of N,N-diisopropylethylamine and 443 mg (1.17 mmol) of HATU were added to 323 mg (0.973 mmol) of 2-[(3-chlorobenzyl)amino]-8-methoxy[1,2,4]triazolo[1,5-a]pyridine-6-carboxylic acid in N,N-dimethylformamide (10 ml), and the mixture was stirred at room temperature for 20 min 300 mg (1.95 mmol) of 3-ethyl-1,4-diazabicyclo[4.2.0]octan-2-one [diastereomer mixture] were added, and the mixture was stirred at room temperature for 1.5 h. The mixture was concentrated under reduced pressure, and the residue was dissolved in methanol and water and purified by preparative RP-HPLC (acetonitrile/water). This gave 313 mg (68% of theory) of the target compound.

LC-MS (method 1A): Rₜ=0.91 min; MS (ESIpos): m/z=469 [M+H]⁺

¹H-NMR (400 MHz, DMSO-d₆): δ [ppm]=8.42 (s, 1H), 7.42-7.21 (m, 5H), 6.98-6.86 (m, 1H), 4.84-4.52 (m, 1H), 4.46 (d, 2H), 4.21-4.00 (m, 2H), 4.00-3.88 (m, 4H), 3.87-3.81 (m, 1H), 3.54-3.46 (m, 1H), 2.30-1.78 (m, 2H), 1.72-1.50 (m, 1H), 1.00-0.82 (m, 3H); 2H presumably hidden under DMSO signal.

### Example 59

### 4-({2-[(3-Chlorobenzyl)amino]-8-methoxy[1,2,4]triazolo[1,5-a]pyridin-6-yl}carbonyl)-3-ethyl-1,4-diazabicyclo[4.2.0]octan-2-one [enantiomerically pure isomer 3]

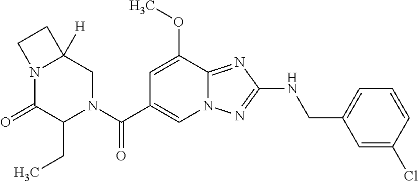 [SMILES: CCC1C(=O)N2CCC2CN1C(=O)c1cc(OC)c2nc(NCc3cccc(Cl)c3)nn2c1]

300 mg of 4-({2-[(3-chlorobenzyl)amino]-8-methoxy[1,2,4]triazolo[1,5-a]pyridin-6-yl}carbonyl)-3-ethyl-1,4-diazabicyclo[4.2.0]octan-2-one [diastereomer mixture, Example 58] were separated into the diastereomers on a chiral phase [Method 32D].

Yield: enantiomerically pure isomer 3: 23.8 mg (100% ee)

enantiomerically pure isomer 3: Rₜ=11.26 min [Method 29E].

### Example 60

### 4-({2-[(3-Chlorobenzyl)amino]-8-methoxy[1,2,4]triazolo[1,5-a]pyridin-6-yl}carbonyl)-3-(methoxymethyl)-1,4-diazabicyclo[4.2.0]octan-2-one [enantiomerically pure isomer 3]

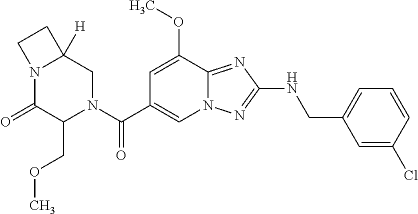 [SMILES: COCC1C(=O)N2CCC2CN1C(=O)c1cc(OC)c2nc(NCc3cccc(Cl)c3)nn2c1]

91 mg (0.12 ml, 0.71 mmol) of N,N-diisopropylethylamine and 92 mg (0.41 mmol) of HATU were added to 67 mg (0.20 mmol) of 2-[(3-chlorobenzyl)amino]-8-methoxy[1,2,4]triazolo[1,5-a]pyridine-6-carboxylic acid in N,N-dimethylformamide (2 ml), and the mixture was stirred at room temperature for 20 min 69 mg (0.41 mmol) of 3-(methoxymethyl)-1,4-diazabicyclo[4.2.0]octan-2-one [enantiomerically pure isomer 3, Example 208A] were then added, and the mixture was stirred at room temperature overnight. The mixture was concentrated under reduced pressure, and the residue was dissolved in methanol and water and purified by preparative RP-HPLC (acetonitrile/water). This gave 66 mg (67% of theory) of the target compound.

LC-MS (method 1A): Rₜ=0.85 min; MS (ESIpos): m/z=485 [M+H]⁺

¹H-NMR (400 MHz, DMSO-d₆): δ [ppm]=8.44-8.37 (m, 1H), 7.42-7.24 (m, 5H), 6.99-6.92 (m, 1H), 4.62-4.52 (m, 1H), 4.50-4.44 (m, 1H), 4.44-4.37 (m, 1H), 4.11-4.01 (m, 1H), 3.93 (s, 5H), 3.87-3.78 (m, 1H), 3.70-3.63 (m, 1H), 3.63-3.56 (m, 1H), 3.55-3.45 (m, 1H), 3.27 (s, 3H), 2.13-2.00 (m, 1H); 1H presumably hidden under DMSO signal.

### Example 61

### 4-({2-[(3-Chlorobenzyl)amino]-8-methoxy[1,2,4]triazolo[1,5-a]pyridin-6-yl}carbonyl)-3-methyl-1,4-diazabicyclo[4.2.0]octan-2-one [enantiomerically pure isomer]

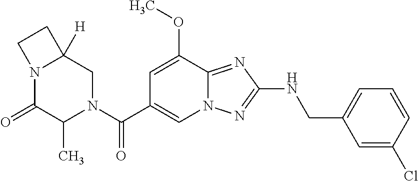 [SMILES: COc1cc(C(=O)N2CC3CCN3C(=O)C2C)cn2nc(NCc3cccc(Cl)c3)nc12]

120 mg (0.361 mmol) of 2-[(3-chlorobenzyl)amino]-8-methoxy[1,2,4]triazolo[1,5-a]pyridine-6-carboxylic acid and 75.8 mg (0.541 mmol) of methyl 1,4-diazabicyclo[4.2.0]octan-2-one [enantiomerically pure isomer 3, Example 24A] were initially charged in N,N-dimethylformamide (1.66 ml), and 186 mg (251 μl, 1.44 mmol) of N,N-diisopropylethylamine were added. Subsequently, 165 mg (0.433 mmol) of HATU were added at RT and the mixture was stirred overnight. Without further work-up, the reaction solution was purified by preparative RP-HPLC (acetonitrile/water). Yield: 160 mg (97% of theory).

LC-MS (method 1A): Rₜ=0.85 min; MS (ESIpos): m/z=455 [M+H]⁺;

¹H-NMR (400 MHz, DMSO-d₆): δ [ppm]=8.42 (d, 1H), 7.39 (s, 1H), 7.37-7.25 (m, 4H), 6.96 (d, 1H), 4.62 (m_{c}, 1H), 4.46 (d, 2H), 4.25 (q, 1H), 4.11-4.02 (m, 1H), 4.00-3.90 (m, 4H), 3.87 (dd, 1H), 3.53-3.42 (m, 1H), 2.47-2.38 (m, 1H), 2.14-2.02 (m, 1H), 1.40 (d, 3H).

### Example 62

### 7-({2-[(3-Chlorobenzyl)amino]-8-methoxy[1,2,4]triazolo[1,5-a]pyridin-6-yl}carbonyl)-6-methyl-4,7-diazaspiro[2.5]octan-5-one [enantiomerically pure isomer 1]

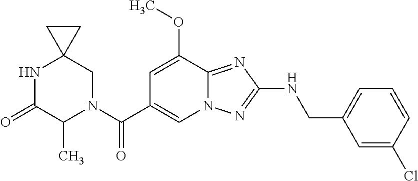 [SMILES: COc1cc(C(=O)N2CC3(CC3)NC(=O)C2C)cn2nc(NCc3cccc(Cl)c3)nc12]

203 mg (274 μl, 1.57 mmol) of N,N-diisopropylethylamine and 144 mg (0.38 mmol) of HATU were added to 104 mg (0.315 mmol) of 2-[(3-chlorobenzyl)amino]-8-methoxy[1,2,4]triazolo[1,5-a]pyridine-6-carboxylic acid in N,N-dimethylformamide (3 ml), and the mixture was stirred at room temperature for 20 min 200 mg (0.63 mmol) of 6-methyl-4,7-diazaspiro[2.5]octan-5-one [racemate, Example 212A] were added and the mixture was stirred at room temperature overnight. The mixture was concentrated under reduced pressure, and the residue was dissolved in methanol and water and purified by preparative RP-HPLC (acetonitrile/water). This gave 89 mg (63% of theory) of the target compound as a racemate.

LC-MS (method 1A): Rₜ=0.82 min; MS (ESIpos): m/z=455 [M+H]⁺

¹H-NMR (400 MHz, DMSO-d₆): δ [ppm]=8.36 (s, 1H), 8.18 (s, 1H), 7.42-7.24 (m, 5H), 6.89 (d, 1H), 4.82-4.64 (m, 1H), 4.46 (d, 2H), 3.92 (s, 3H), 3.78-3.63 (m, 1H), 1.45 (d, 3H), 0.82-0.51 (m, 4H); 1H presumably hidden under DMSO signal.

The racemate was separated into the enantiomers on a chiral phase [Method 33D].

Yield: enantiomerically pure isomer 1: 46 mg (100% ee)

enantiomerically pure isomer 1: Rₜ=5.72 min [Method 30E].

LC-MS (method 1A): Rₜ=0.82 min; MS (ESIpos): m/z=455 [M+H]⁺

¹H-NMR (400 MHz, DMSO-d₆): δ [ppm]=8.36 (s, 1H), 8.18 (s, 1H), 7.42-7.21 (m, 5H), 6.92-6.85 (m, 1H), 4.46 (d, 2H), 3.98-3.88 (m, 3H), 3.73 (d, 1H), 2.66-2.53 (m, 1H), 1.48-1.40 (m, 3H), 0.90-0.55 (m, 4H); 1H presumably hidden under DMSO signal.

### Example 63

### 4-({2-[(3-Chlorobenzyl)amino]-8-methoxy[1,2,4]triazolo[1,5-a]pyridin-6-yl}carbonyl)-3-ethyl-6-methylpiperazin-2-one [enantiomerically pure isomer 4]

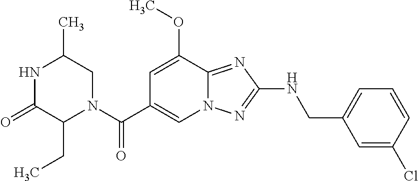 [SMILES: CCC1C(=O)NC(C)CN1C(=O)c1cc(OC)c2nc(NCc3cccc(Cl)c3)nn2c1]

72 mg (96 μl, 0.56 mmol) of N,N-diisopropylethylamine and 72 mg (0.19 mmol) of HATU were added to 52.8 mg (0.159 mmol) of 2-[(3-chlorobenzyl)amino]-8-methoxy[1,2,4]triazolo[1,5-a]pyridine-6-carboxylic acid in N,N-dimethylformamide (2 ml), and the mixture was stirred at room temperature for 20 min 45 mg (0.63 mmol) of 3-ethyl-6-methylpiperazin-2-one [diastereomer mixture, 4 isomers, Example 220A] were added, and the mixture was stirred at room temperature for 2.5 h. The mixture was concentrated under reduced pressure, and the residue was dissolved in methanol and water and purified by preparative RP-HPLC (acetonitrile/water). This gave 50 mg (69% of theory) of the target compound as a diastereomer mixture.

LC-MS (Method 1A): Rₜ=0.86 min (diastereomer 1, 2 isomers), Rₜ=0.87 min (diastereomer 2, 2 isomers);

MS (ESIpos): m/z=457 [M+H]⁺

¹H-NMR (400 MHz, DMSO-d₆): δ [ppm]=8.46-8.39 (m, 1H), 8.07-7.97 (m, 1H), 7.41-7.24 (m, 5H), 6.90 (s, 1H), 4.79-4.53 (m, 1H), 4.46 (d, 2H), 3.96-3.89 (m, 3H), 3.78-3.47 (m, 2H), 1.92-1.78 (m, 2H), 1.09-0.76 (m, 6H); 1H presumably hidden under DMSO signal.

The diastereomer mixture, 4 isomers were separated into the enantiomers on a chiral phase [Method 34D].

Yield: enantiomerically pure isomer 4: 5 mg (97% ee)

enantiomerically pure isomer 4: Rₜ=19.8 min [Method 31E].

LC-MS (method 1A): Rₜ=0.89 min; MS (ESIpos): m/z=457 [M+H]+

### Example 64

### {2-[(3-Chlorobenzyl)amino]-8-methoxy[1,2,4]triazolo[1,5-a]pyridin-6-yl}[2-(2-hydroxyethyl)-5-methylpiperidin-1-yl]methanone [diastereomer mixture, 4 isomers]

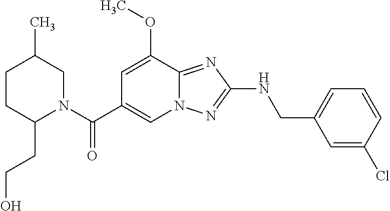 [SMILES: COc1cc(C(=O)N2CC(C)CCC2CCO)cn2nc(NCc3cccc(Cl)c3)nc12]

299 mg (403 μl, 2.31 mmol) of N,N-diisopropylethylamine and 301 mg (0.793 mmol) of HATU were added to 220 mg (0.661 mmol) of 2-[(3-chlorobenzyl)amino]-8-methoxy[1,2,4]triazolo[1,5-a]pyridine-6-carboxylic acid in N,N-dimethylformamide (7 ml), and the mixture was stirred at room temperature for 20 min. 113 mg (0.793 mmol) of 3-ethyl-6-methylpiperazin-2-one [diastereomer mixture, 4 isomers, Example 223A] were added, and the mixture was stirred at room temperature for 3 h. The mixture was concentrated under reduced pressure, and the residue was dissolved in methanol and water and purified by preparative RP-HPLC (acetonitrile/water). This gave 46 mg (14% of theory) of the target compound as a diastereomer mixture, 4 isomers.

LC-MS (method 1A): Rₜ=0.97 min; MS (ESIpos): m/z=458 [M+H]⁺

¹H-NMR (500 MHz, DMSO-d₆): δ [ppm]=8.26 (s, 1H), 7.42-7.19 (m, 5H), 6.89-6.82 (m, 1H), 4.82-4.67 (m, 1H), 4.29-4.11 (m, 1H), 4.46 (d, 2H), 3.93 (s, 3H), 1.92-1.81 (m, 1H), 1.70-1.51 (m, 4H), 1.38-1.12 (m, 2H), 0.95-0.71 (m, 3H); 4H presumably hidden under DMSO signal.

### Example 65

### 1-({2-[(3-Chlorobenzyl)amino]-8-methoxy[1,2,4]triazolo[1,5-a]pyridin-6-yl}carbonyl)-5-methylpiperidine-2-carboxamide [diastereomer mixture, 4 isomers]

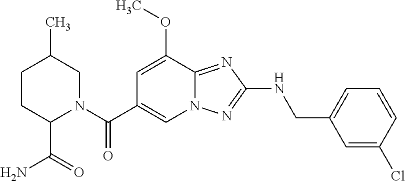 [SMILES: COc1cc(C(=O)N2CC(C)CCC2C(N)=O)cn2nc(NCc3cccc(Cl)c3)nc12]

680 mg (916 μl, 5.26 mmol) of N,N-diisopropylethylamine and 480 mg (1.26 mmol) of HATU were added to 350 mg (1.05 mmol) of 2-[(3-chlorobenzyl)amino]-8-methoxy[1,2,4]triazolo[1,5-a]pyridine-6-carboxylic acid in N,N-dimethylformamide (10.5 ml), and the mixture was stirred at room temperature for 20 min 1.27 g (6.31 mmol) of 5-methylpiperidine-2-carboxamide acetate [diastereomer mixture, 4 isomers, Example 224A] were then added, and the mixture was stirred at room temperature overnight. The mixture was concentrated under reduced pressure, and the residue was dissolved in acetonitrile and water and purified by preparative RP-HPLC (acetonitrile/water). This gave 310 mg (58% of theory) of the target compound as a diastereomer mixture, 4 isomers.

¹H-NMR (400 MHz, DMSO-d₆): δ [ppm]=8.63 (t, 1H), 7.47-7.28 (m, 4H), 7.19 (d, 1H), 6.96-6.66 (m, 2H), 5.07 (br. s., 1H), 4.54-4.48 (m, 2H), 4.41-4.22 (m, 1H), 3.94-3.82 (m, 3H), 2.27-2.03 (m, 1H), 1.70-1.46 (m, 3H), 1.09-0.93 (m, 1H), 0.92-0.64 (m, 3H); 2H presumably hidden under DMSO signal.

### Example 66

### {2-[(3-Chlorobenzyl)amino]-8-methoxy[1,2,4]triazolo[1,5-a]pyridin-6-yl}[5-(2-hydroxypropan-2-yl)-2,2-dimethylmorpholin-4-yl]methanone [enantiomerically pure isomer 1]

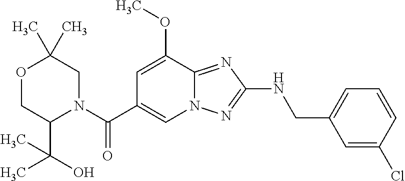 [SMILES: COc1cc(C(=O)N2CC(C)(C)OCC2C(C)(C)O)cn2nc(NCc3cccc(Cl)c3)nc12]

130 mg (176 μl, 1.01 mmol) of N,N-diisopropylethylamine and 131 mg (0.35 mmol) of HATU were added to 96 mg (0.29 mmol) of 2-[(3-chlorobenzyl)amino]-8-methoxy[1,2,4]triazolo[1,5-a]pyridine-6-carboxylic acid in N,N-dimethylformamide (1.8 ml), and the mixture was stirred at room temperature for 20 min. 100 mg (0.577 mmol) of 2-(6,6-dimethylmorpholin-3-yl)propan-2-ol [enantiomerically pure isomer 1, Example 230A] were then added, and the mixture was stirred at room temperature overnight. The mixture was concentrated under reduced pressure, the residue was dissolved in ethyl acetate and water and the phases were separated. The aqueous phase was washed twice with ethyl acetate and the combined organic phases were dried over sodium sulphate. After filtration, the filtrate was concentrated under reduced pressure and the residue was dried under high vacuum and purified by preparative RP-HPLC (acetonitrile/water). This gave 24.3 mg (17% of theory) of the target compound.

LC-MS (method 1A): Rₜ=0.94 min; MS (ESIpos): m/z=488 [M+H]⁺

¹H-NMR (400 MHz, DMSO-d₆): δ [ppm]=8.53 (d, 1H), 7.42-7.24 (m, 5H), 6.93-6.86 (m, 1H), 5.76 (s, 1H), 4.65 (s, 1H), 4.46 (d, 2H), 4.39 (t, 1H), 3.96-3.87 (m, 4H), 3.76-3.67 (m, 1H), 3.54-3.43 (m, 1H), 1.25-1.06 (m, 6H), 1.02-0.93 (m, 6H).

### Example 67

### {2-[(3-Chlorobenzyl)amino]-8-methoxy[1,2,4]triazolo[1,5-a]pyridin-6-yl}[5-(1-hydroxycyclopropyl)-2,2-dimethylmorpholin-4-yl]methanone [enantiomerically pure isomer 1]

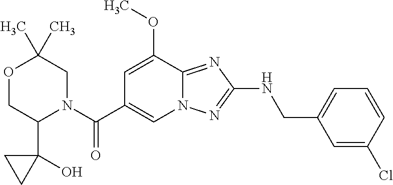 [SMILES: COc1cc(C(=O)N2CC(C)(C)OCC2C2(O)CC2)cn2nc(NCc3cccc(Cl)c3)nc12]

237 mg (320 μl, 1.84 mmol) of N,N-diisopropylethylamine and 239 mg (0.631 mmol) of HATU were added to 174 mg (0.526 mmol) of 2-[(3-chlorobenzyl)amino]-8-methoxy[1,2,4]triazolo[1,5-a]pyridine-6-carboxylic acid in N,N-dimethylformamide (3.3 ml), and the mixture was stirred at room temperature for 20 min 108 mg (0.631 mmol) of 1-(6,6-dimethylmorpholin-3-yl)cyclopropanol [enantiomerically pure isomer 1, Example 234A] were then added, and the mixture was stirred at room temperature overnight. The mixture was concentrated under reduced pressure, the residue was dissolved in ethyl acetate and water and the phases were separated. The aqueous phase was washed twice with ethyl acetate and the combined organic phases were dried over sodium sulphate. After filtration, the filtrate was concentrated under reduced pressure and the residue was dried under high vacuum and purified by preparative RP-HPLC (acetonitrile/water). This gave 99.8 mg (39% of theory) of the target compound.

LC-MS (method 1A): Rₜ=0.91 min; MS (ESIpos): m/z=486 [M+H]⁺

¹H-NMR (400 MHz, DMSO-d₆): δ [ppm]=8.50 (d, 1H), 7.42-7.24 (m, 5H), 6.96 (d, 1H), 5.76 (s, 1H), 5.60 (s, 1H), 4.46 (d, 2H), 3.92 (s, 3H), 3.77 (dd, 1H), 1.21-0.84 (m, 8H), 0.72-0.46 (m, 2H); 3H presumably hidden under DMSO signal.

### Example 68

### {2-[(3-Chlorobenzyl)amino]-8-methoxy[1,2,4]triazolo[1,5-a]pyridin-6-yl}[5-(1-hydroxyethyl)-2,2-dimethylmorpholin-4-yl]methanone [enantiomerically pure isomer 2]

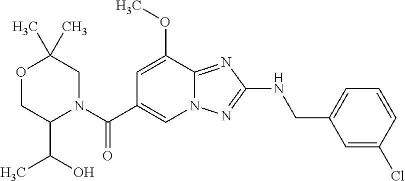 [SMILES: COc1cc(C(=O)N2CC(C)(C)OCC2C(C)O)cn2nc(NCc3cccc(Cl)c3)nc12]

142 mg (191 μl, 1.09 mmol) of N,N-diisopropylethylamine and 143 mg (0.377 mmol) of HATU were added to 104 mg (0.314 mmol) of 2-[(3-chlorobenzyl)amino]-8-methoxy[1,2,4]triazolo[1,5-a]pyridine-6-carboxylic acid in N,N-dimethylformamide (2.0 ml), and the mixture was stirred at room temperature for 20 min 100 mg (0.628 mmol) of 1-(6,6-dimethylmorpholin-3-yl)ethanol [enantiomerically pure isomer 2, Example 239A] were then added, and the mixture was stirred at room temperature overnight. The mixture was concentrated under reduced pressure, the residue was dissolved in ethyl acetate and water and the phases were separated. The aqueous phase was washed twice with ethyl acetate and the combined organic phases were dried over sodium sulphate. After filtration, the filtrate was concentrated under reduced pressure and the residue was dried under high vacuum and purified by preparative RP-HPLC (acetonitrile/water). This gave 125 mg (83% of theory) of the target compound.

LC-MS (method 1A): Rₜ=0.88 min; MS (ESIpos): m/z=474 [M+H]⁺

¹H-NMR (400 MHz, DMSO-d₆): δ [ppm]=8.48 (s, 1H), 7.46-7.16 (m, 4H), 7.08 (s, 1H), 5.25-5.20 (m, 1H), 4.46 (d, 2H), 4.20-4.03 (m, 2H), 3.92 (s, 3H), 3.81-3.72 (m, 1H), 3.51-3.37 (m, 2H), 1.32-0.93 (m, 9H); 2H presumably hidden under DMSO signal.

### Example 69

### {2-[(3-Chlorobenzyl)amino]-8-methoxy[1,2,4]triazolo[1,5-a]pyridin-6-yl}[(5R)-5-ethyl-2-(2-hydroxyethyl)-2-methylmorpholin-4-yl]methanone [diastereomer mixture, 2 isomers]

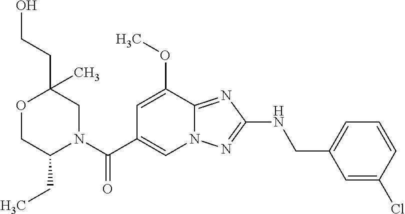 [SMILES: CC[C@@H]1COC(C)(CCO)CN1C(=O)c1cc(OC)c2nc(NCc3cccc(Cl)c3)nn2c1]

122 mg (165 μl, 0.95 mmol) of N,N-diisopropylethylamine and 123 mg (0.325 mmol) of HATU were added to 180 mg (0.270 mmol, purity: 50%) of 2-[(3-chlorobenzyl)amino]-8-methoxy[1,2,4]triazolo[1,5-a]pyridine-6-carboxylic acid and 56 mg (0.325 mmol) of 2-(5-ethyl-2-methylmorpholin-2-yl)ethanol [diastereomer mixture, 2 isomers, Example 197A] in N,N-dimethylformamide (5.0 ml), and the mixture was stirred at room temperature overnight. The mixture was concentrated under reduced pressure, and the residue was dissolved in methanol and water and purified by preparative RP-HPLC (acetonitrile/water). This gave 49.9 mg (38% of theory) of the target compound.

LC-MS (Method 8A): Rₜ=0.91 min (diastereomer 1), Rₜ=0.92 min (diastereomer 2);

MS (ESIpos): m/z=488 [M+H]⁺

¹H-NMR (400 MHz, DMSO-d₆): δ [ppm]=8.37-8.23 (m, 1H), 7.44-7.21 (m, 5H), 6.88-6.74 (m, 1H), 4.46 (br. s., 2H), 3.93 (d, 3H), 3.70-3.55 (m, 1H), 3.20-3.11 (m, 1H), 1.90-1.72 (m, 1H), 1.68-1.54 (m, 1H), 1.30-1.19 (m, 6H), 1.17-1.00 (m, 2H), 0.92-0.70 (m, 2H). 3H presumably hidden under DMSO signal.

### Example 70

### {2-[(3-Chlorobenzyl)amino]-8-methoxy[1,2,4]triazolo[1,5-a]pyridin-6-yl}[(5R)-5-ethyl-2-(2-hydroxyethyl)-2-methylmorpholin-4-yl]methanone [enantiomerically pure isomer 1]

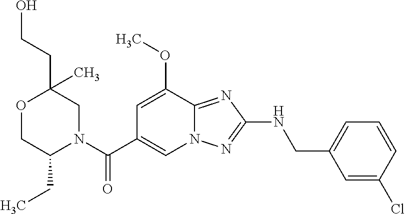 [SMILES: CC[C@@H]1COC(C)(CCO)CN1C(=O)c1cc(OC)c2nc(NCc3cccc(Cl)c3)nn2c1]

The diastereomer mixture (2 isomers) from Example 69 was separated into the enantiomers on a chiral phase [Method 35D].

Yield: enantiomerically pure isomer 1: 19 mg (100% ee)

enantiomerically pure isomer 1: Rₜ=4.45 min [Method 32E].

¹H-NMR (400 MHz, DMSO-d₆): δ [ppm]=8.30 (s, 1H), 7.41-7.22 (m, 5H), 6.82 (s, 1H), 4.46 (d, 2H), 3.93 (s, 3H), 3.88-3.72 (m, 1H), 2.99-2.86 (m, 3H), 1.89-1.73 (m, 1H), 1.68-1.57 (m, 2H), 1.20-0.99 (m, 6H), 0.92-0.71 (m, 2H); 3H presumably hidden under DMSO signal.

### Example 71

### {2-[(3-Chlorobenzyl)amino]-8-methoxy[1,2,4]triazolo[1,5-a]pyridin-6-yl}[2-(2-hydroxyethyl)-5-(methoxymethyl)-2-methylmorpholin-4-yl]methanone [diastereomer mixture, 4 isomers]

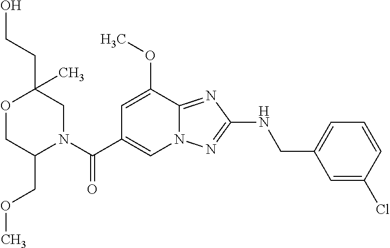 [SMILES: COCC1COC(C)(CCO)CN1C(=O)c1cc(OC)c2nc(NCc3cccc(Cl)c3)nn2c1]

298 mg (403 μl, 2.31 mmol) of N,N-diisopropylethylamine and 301 mg (0.793 mmol) of HATU were added to 219 mg (0.660 mmol) of 2-[(3-chlorobenzyl)amino]-8-methoxy[1,2,4]triazolo[1,5-a]pyridine-6-carboxylic acid in N,N-dimethylformamide (7.0 ml), and the mixture was stirred at room temperature for 20 min 150 mg (0.793 mmol) of 2-[5-(methoxymethyl)-2-methylmorpholin-2-yl]ethanol [diastereomer mixture, 4 isomers, Example 190A] were then added, and the mixture was stirred at room temperature overnight. The mixture was concentrated under reduced pressure, and the residue was dissolved in methanol and water and purified by preparative RP-HPLC (acetonitrile/water). This gave 158 mg (48% of theory) of the target compound.

LC-MS (method 8A): Rₜ=0.94 min; MS (ESIpos): m/z=504 [M+H]⁺

¹H-NMR (400 MHz, DMSO-d₆): δ [ppm]=8.38-8.30 (m, 1H), 7.51-7.16 (m, 6H), 6.91 (br. s., 1H), 4.49-4.38 (m, 2H), 4.32 (t, 1H), 3.99-3.90 (m, 4H), 3.86-3.42 (m, 4H), 3.38 (br. s., 3H), 2.76-2.64 (m, 1H), 2.40-2.30 (m, 1H), 1.64 (br. s., 1H), 1.27-0.99 (m, 3H)); 2H presumably hidden under DMSO signal.

### Example 72

### {2-[(3-Chlorobenzyl)amino]-8-methoxy[1,2,4]triazolo[1,5-a]pyridin-6-yl}[2-(2-hydroxyethyl)-5-(methoxymethyl)-2-methylmorpholin-4-yl]methanone [enantiomerically pure isomer 2]

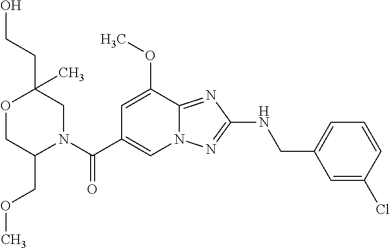 [SMILES: COCC1COC(C)(CCO)CN1C(=O)c1cc(OC)c2nc(NCc3cccc(Cl)c3)nn2c1]

The diastereomer mixture (4 isomers) from Example 71 was separated into the enantiomers on a chiral phase [Method 36D].

Yield: enantiomerically pure isomer 2: 26 mg (96% ee)

enantiomerically pure isomer 2: Rₜ=7.05 min [Method 33E].

¹H-NMR (400 MHz, DMSO-d₆): δ [ppm]=8.37-8.24 (m, 1H), 7.45-7.23 (m, 6H), 6.90 (br. s., 1H), 4.46 (d, 2H), 4.29 (t, 1H), 3.99-3.91 (m, 4H), 3.82-3.71 (m, 2H), 3.50-3.43 (m, 2H), 3.38 (br. s., 3H), 2.72-2.63 (m, 1H), 2.09-1.85 (m, 1H), 1.58-1.41 (m, 1H), 1.29-1.20 (m, 1H), 1.16-1.03 (m, 2H); 2H presumably hidden under DMSO signal.

### Example 73

### {2-[(3-Chlorobenzyl)amino]-8-methoxy[1,2,4]triazolo[1,5-a]pyridin-6-yl}[(4R)-4-[(1,1-²H₂)ethyloxy]-2-(hydroxymethyl)pyrrolidin-1-yl]methanone [diastereomer mixture, 2 isomers]

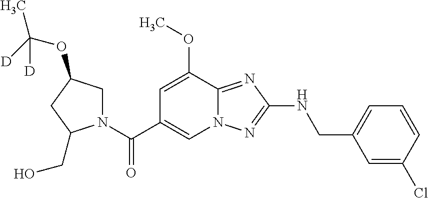 [SMILES: [2H]C([2H])(C)O[C@@H]1CC(CO)N(C(=O)c2cc(OC)c3nc(NCc4cccc(Cl)c4)nn3c2)C1]

136 mg (183 μl, 1.05 mmol) of N,N-diisopropylethylamine and 380 mg (0.361 mmol) of HATU were added to 200 mg (0.301 mmol, purity: 50%) of 2-[(3-chlorobenzyl)amino]-8-methoxy[1,2,4]triazolo[1,5-a]pyridine-6-carboxylic acid and 53 mg (0.36 mmol) of {(4R)-4-[(1,1-²H₂)ethyloxy]pyrrolidin-2-yl}methanol [diastereomer mixture, 2 isomers, Example 184A] in N,N-dimethylformamide (1.5 ml), and the mixture was stirred at room temperature overnight. The mixture was concentrated under reduced pressure, and the residue was dissolved in methanol and water and purified by preparative RP-HPLC (acetonitrile/water). This gave 69 mg (49% of theory) of the target compound.

LC-MS (Method 2A): Rₜ=0.84 min (diastereomer 1), Rₜ=0.86 min (diastereomer 2);

MS (ESIpos): m/z=488 [M+H]⁺

¹H-NMR (400 MHz, DMSO-d₆): δ [ppm]=8.45 (d, 1H), 7.41-7.23 (m, 5H), 7.04-6.94 (m, 1H), 4.76 (br. s., 1H), 4.46 (d, 2H), 4.03-3.89 (m, 4H), 3.71-3.44 (m, 2H), 2.21-1.88 (m, 2H), 1.09-0.94 (m, 3H); 2H presumably hidden under DMSO signal.

### Example 74

### {2-[(3-Chlorobenzyl)amino]-8-methoxy[1,2,4]triazolo[1,5-a]pyridin-6-yl}[(4R)-4-[(1,1-²H₂)ethyloxy]-2-(hydroxymethyl)pyrrolidin-1-yl]methanone [enantiomerically pure isomer 2]

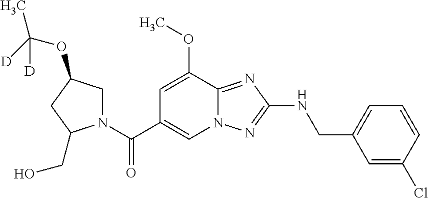 [SMILES: [2H]C([2H])(C)O[C@@H]1CC(CO)N(C(=O)c2cc(OC)c3nc(NCc4cccc(Cl)c4)nn3c2)C1]

The diastereomer mixture (2 isomers) from Example 73 was separated on a chiral phase [Method 37D].

Yield: enantiomerically pure isomer 2: 21 mg (96% ee) enantiomerically pure isomer 2: Rₜ=6.31 min [Method 34E].

¹H-NMR (400 MHz, DMSO-d₆): δ [ppm]=8.45 (d, 1H), 7.41-7.21 (m, 5H), 7.00 (s, 1H), 4.73-4.67 (m, 1H), 4.46 (d, 2H), 4.02-3.96 (m, 1H), 3.93 (s, 3H), 3.69-3.44 (m, 2H), 2.09-1.98 (m, 2H), 1.10-0.95 (m, 3H); 2H presumably hidden under DMSO signal.

### Example 75

### {2-[(3-Chlorobenzyl)amino]-8-methoxy[1,2,4]triazolo[1,5-a]pyridin-6-yl}[(4R)-4-(2,2-difluoroethoxy)-2-(hydroxymethyl)pyrrolidin-1-yl]methanone [enantiomerically pure isomer]

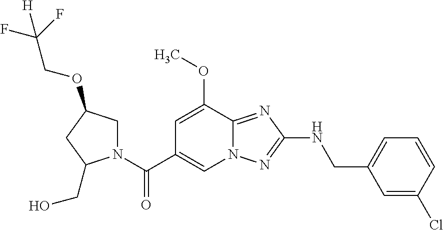 [SMILES: COc1cc(C(=O)N2C[C@H](OCC(F)F)CC2CO)cn2nc(NCc3cccc(Cl)c3)nc12]

125 mg (168 μl, 0.966 mmol) of N,N-diisopropylethylamine and 126 mg (0.331 mmol) of HATU were added to 91.8 mg (0.276 mmol) of 2-[(3-chlorobenzyl)amino]-8-methoxy[1,2,4]triazolo[1,5-a]pyridine-6-carboxylic acid in N,N-dimethylformamide (2.5 ml), and the mixture was stirred at room temperature for 20 min 60 mg (0.33 mmol) of [(4R)-4-(2,2-difluoroethoxy)pyrrolidin-2-yl]methanol [enantiomerically pure isomer 2, Example 181A] were then added, and the mixture was stirred at room temperature overnight. The mixture was concentrated under reduced pressure, and the residue was dissolved in methanol and water and purified by preparative RP-HPLC (acetonitrile/water). This gave 29.9 mg (20% of theory) of the target compound.

LC-MS (method 1A): Rₜ=0.84 min; MS (ESIpos): m/z=496 [M+H]⁺

¹H-NMR (500 MHz, DMSO-d₆): δ [ppm]=8.43 (s, 1H), 7.42-7.21 (m, 6H), 6.98 (s, 1H), 4.69 (br. s., 1H), 4.46 (d, 2H), 4.29-4.08 (m, 2H), 3.93 (s, 4H), 3.75-3.45 (m, 3H), 2.15-2.01 (m, 2H), 1.24 (s, 1H), 1.16 (d, 1H).

### Example 76

### (2-{[1-(3-Chlorophenyl)-2-fluoroethyl]amino}-8-methoxy[1,2,4]triazolo[1,5-a]pyridin-6-yl) [5-(2-hydroxyethyl)-2-methylmorpholin-4-yl]methanone [diastereomer mixture, 2 isomers]

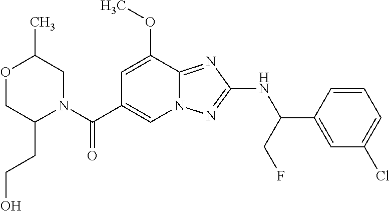 [SMILES: COc1cc(C(=O)N2CC(C)OCC2CCO)cn2nc(NC(CF)c3cccc(Cl)c3)nc12]

50.1 mg (0.077 mmol) of (2-{[1-(3-chlorophenyl)-2-fluoroethyl]amino}-8-methoxy-[1,2,4]triazolo[1,5-a]pyridin-6-yl)(2-methyl-5-{2-[(triisopropylsilyl)oxy]ethyl}morpholin-4-yl)methanone [diastereomer mixture, 2 isomers] were initially charged in tetrahydrofuran (3.44 ml), and 268 μl (0.268 mmol) of tetra-n-butylammonium fluoride solution (1.0 M in tetrahydrofuran) were added at RT. The reaction solution was stirred at RT for 2 h and then diluted with dichloromethane and washed with water. The organic phase was dried over sodium sulphate, filtered and concentrated under reduced pressure. The residue was taken up in ethyl acetate and washed with water and the organic phase was dried over sodium sulphate, filtered and concentrated under reduced pressure. The crude product was purified by preparative RP-HPLC (acetonitrile/water). Yield: 24.1 mg (63% of theory).

LC-MS (method 1A): Rₜ=0.86 min; MS (ESIpos): m/z=492 [M+H]⁺.

### Example 77

### 4-[(2-{[1-(3-Chlorophenyl)-2-fluoroethyl]amino}-8-methoxy[1,2,4]triazolo[1,5-a]pyridin-6-yl)carbonyl]-3-methyl-1,4-diazabicyclo[4.2.0]octan-2-one [enantiomerically pure isomer]

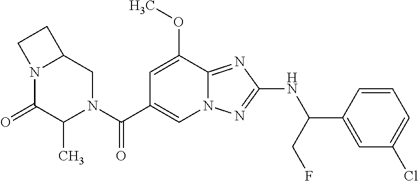 [SMILES: COc1cc(C(=O)N2CC3CCN3C(=O)C2C)cn2nc(NC(CF)c3cccc(Cl)c3)nc12]

150 mg (0.411 mmol) of 2-{[1-(3-chlorophenyl)-2-fluoroethyl]amino}-8-methoxy[1,2,4]triazolo[1,5-a]pyridine-6-carboxylic acid [enantiomerically pure isomer] and 86.5 mg (0.620 mmol) of 3-methyl-1,4-diazabicyclo[4.2.0]octan-2-one [enantiomerically pure isomer 3] were initially charged in N,N-dimethylformamide (2.74 ml), and 213 mg (287 μl, 1.65 mmol) of N,N-diisopropylethylamine were added. 188 mg (0.493 mmol) of HATU were then added at RT, and the mixture was stirred for 2 h. Without further work-up, the reaction solution was purified by preparative RP-HPLC (acetonitrile/water). Yield: 160 mg (79% of theory).

LC-MS (method 1A): Rₜ=0.88 min; MS (ESIpos): m/z=486 [M+H]⁺;

¹H-NMR (400 MHz, DMSO-d₆): δ [ppm]=8.41 (d, 1H), 7.65-7.53 (m, 2H), 7.49-7.42 (m, 1H), 7.41-7.28 (m, 2H), 6.96 (d, 1H), 5.17 (m_{c}, 1H), 4.75-4.44 (m, 3H), 4.24 (q, 1H), 4.06 (m_{c}, 1H), 4.00-3.88 (m, 4H), 3.84 (dd, 1H), 3.47 (t, 1H), 2.47-2.34 (m, 1H), 2.15-1.98 (m, 1H), 1.40 (d, 3H).

### B) ASSESSMENT OF PHYSIOLOGICAL EFFICACY

The suitability of the compounds according to the invention for treating thromboembolic disorders can be demonstrated in the following assay systems:

a) Test Descriptions (In Vitro)

### a.1) Measurement of the Thrombin Inhibition in Buffer

To determine the thrombin inhibition of the substances listed above, a biochemical test system is constructed in which the conversion of a thrombin substrate is used for determining the enzymatic activity of human thrombin. Here, thrombin cleaves aminomethylcoumarin, which is measured fluorescently, from the peptic substrate. The determinations are carried out in microtitre plates.

Substances to be tested are dissolved in various concentrations in dimethyl sulphoxide and incubated for 15 min with human thrombin (0.06 nmol/l dissolved in 50 mmol/l of Tris buffer [C,C,C-tris(hydroxymethyl)aminomethane], 100 mmol/l of sodium chloride, 0.1% BSA [bovine serum albumin], pH 7.4) at 22° C. The substrate (5 μmol/l Boc-Asp(OBzl)-Pro-Arg-AMC from Bachem) is then added. After 30 min of incubation, the sample is excited at a wavelength of 360 nm and the emission is measured at 460 nm. The measured emissions of the test mixtures with test substance are compared to the control mixtures without test substance (only dimethyl sulphoxide instead of test substance in dimethyl sulphoxide) and IC₅₀ values are calculated from the concentration/activity relationships. Representative activity data from this test are given in Table 1 below (in some cases as means of individual determinations):

TABLE 1

| Example No. | IC₅₀ [nM] | Example No. | IC₅₀ [nM] |
|---|---|---|---|
| 1 | 34 | 2 | 19 |
| 3 | 70 | 4 | 9.4 |
| 6 | 8.7 | 7 | 20 |
| 8 | 20 | 9 | 55 |
| 10 | 39 | 11 | 63 |
| 12 | 35 | 13 | 420 |
| 14 | 24 | 15 | 53 |
| 16 | 52 | 17 | 95 |
| 18 | 27 | 19 | 11 |
| 20 | 35 | 21 | 43 |
| 22 | 26 | 23 | 29 |
| 24 | 9.9 | 25 | 28 |
| 26 | 88 | 27 | 62 |
| 28 | 180 | 29 | 54 |
| 30 | 45 | 31 | 11 |
| 32 | 230 | 33 | 4.5 |
| 34 | 3.9 | 35 | 2.4 |
| 36 | 54 | 37 | 67 |
| 38 | 2800 | 39 | 30 |
| 40 | 31 | 41 | 1.3 |
| 42 | 37 | 43 | 81 |
| 44 | 29 | 45 | 33 |
| 46 | 190 | 47 | 180 |
| 48 | 39 | 49 | 19 |
| 50 | 18 | 51 | 3500 |
| 52 | 1200 | 53 | 6.9 |
| 54 | 100 | 55 | 9.6 |
| 56 | 1.3 | 57 | 1000 |
| 58 | 36 | 59 | 8.3 |
| 60 | 26 | 61 | 23 |
| 62 | 17 | 63 | 9.8 |
| 64 | 25 | 65 | 8.9 |
| 66 | 25 | 67 | 48 |
| 68 | 49 | 69 | 25 |
| 70 | 6.1 | 71 | 35 |
| 72 | 13 | 74 | 19 |
| 75 | 23 | 77 | 6.4 |

### a.2) Determination of the Selectivity

To demonstrate the selectivity of the substances with respect to thrombin inhibition, the test substances are examined for their inhibition of other human serine proteases, such as factor Xa, factor XIIa, Factor XIa, trypsin and plasmin. To determine the enzymatic activity of factor Xa (1.3 nmol/l from Kordia), factor XIIa (10 nmol/l from Kordia), factor XIa (0.4 nmol/l from Kordia), trypsin (83 mU/ml from Sigma) and plasmin (0.1 μg/ml from Kordia), these enzymes are dissolved (50 mmol/l of Tris buffer [C,C,C-tris(hydroxymethyl)aminomethane], 100 mmol/l of sodium chloride, 0.1% BSA [bovine serum albumin], 5 mmol/l of calcium chloride, pH 7.4) and incubated for 15 mM with test substance in various concentrations in dimethyl sulphoxide and also with dimethyl sulphoxide without test substance. The enzymatic reaction is then started by addition of the appropriate substrates (5 μmol/1 Boc-Ile-Glu-Gly-Arg-AMC from Bachem for FXa, 5 μmol/l H-Pro-Phe-Arg-AMC from Bachem for factor XIIa, 5 μmol/1 Boc-Ile-Glu-Gly-Arg-AMC from Bachem for trypsin, 5 μmol/1 Boc-Glu(OBzl)-Ala-Arg-AMC from Bachem for factor XIa, 50 μmol/l MeOSuc-Ala-Phe-Lys-AMC from Bachem for plasmin). After an incubation time of 30 min at 22° C., fluorescence is measured (excitation: 360 nm, emission: 460 nm). The measured emissions of the test mixtures with test substance are compared to the control mixtures without test substance (only dimethyl sulphoxide instead of test substance in dimethyl sulphoxide) and IC₅₀ values are calculated from the concentration/activity relationships.

### a.3) Determination of the Thrombin-Inhibitory Activity of the Potential Inhibitors in Plasma Samples

To determine the inhibition of thrombin in plasma samples, plasma prothrombinase is activated by ecarin. Thrombin activity and/or its inhibition by potential inhibitors is/are then measured fluorescently by addition of a substrate.

The substances to be tested are dissolved in various concentrations in dimethyl sulphoxide and diluted with water. In white 96-well flat-bottomed plates, 20 μl of substance dilution are mixed with 20 μl of ecarin solution (ecarin reagent, from Sigma E-0504, final concentration 20 mU per reaction) in Ca buffer (200 mM Hepes+560 mM sodium chloride+10 mM calcium chloride+0.4% PEG) or with 20 μl of Ca buffer (as unstimulated control). Furthermore, 20 μl of fluorogenic thrombin substrate (from Bachem 1-1120, final concentration 50 μmol/l) and 20 μl of citrate plasma (from Octapharma) are added, and the mixture is homogenized well. The plate is measured in a SpectraFluorplus Reader using a 360 nm excitation filter and a 465 nm emission filter every minute over 20 minutes. The IC₅₀ value is determined when about 70% of the maximum signal is reached (about 12 min). Representative activity data from this test are given in Table 2 below (in some cases as means of individual determinations):

TABLE 2

| Example No. | IC₅₀ [nM] | Example No. | IC₅₀ [nM] |
|---|---|---|---|
| 1 | 33 | 2 | 20 |
| 3 | 70 | 4 | 23 |
| 5 | 87 | 6 | 25 |
| 7 | 24 | 8 | 29 |
| 9 | 79 | 10 | 41 |
| 11 | 64 | 12 | 56 |
| 14 | 57 | 15 | 75 |
| 16 | 77 | 17 | 119 |
| 18 | 15 | 19 | 10 |
| 20 | 70 | 21 | 67 |
| 22 | 86 | 23 | 34 |
| 24 | 19 | 25 | 55 |
| 27 | 56 | 29 | 67 |
| 30 | 51 | 31 | 15 |
| 33 | 11 | 34 | 19 |
| 35 | 8.7 | 36 | 325 |
| 37 | 112 | 39 | 34 |
| 40 | 75 | 41 | 4.1 |
| 42 | 54 | 45 | 34 |
| 49 | 24 | 50 | 122 |
| 53 | 24 | 54 | 611 |
| 55 | 65 | 56 | 5.9 |
| 58 | 39 | 59 | 11 |
| 60 | 23 | 61 | 17 |
| 62 | 39 | 63 | 24 |
| 64 | 63 | 65 | 102 |
| 66 | 24 | 67 | 90 |
| 68 | 106 | 69 | 39 |
| 70 | 35 | 71 | 87 |
| 72 | 34 | 73 | 39 |
| 74 | 19 | 75 | 74 |
| 77 | 5.9 | | |

### a.4) Thrombin Generation Assay (Thrombogram)

The effect of the test substances on the thrombogram (thrombin generation assay according to Hemker) is determined in vitro in human plasma (Octaplas® from Octapharma). In the thrombin generation assay according to Hemker, the activity of thrombin in coagulating plasma is determined by measuring the fluorescent cleavage products of the substrate 1-1140 (Z-Gly-Gly-Arg-AMC, Bachem). To initiate the coagulation reaction, reagents from Thrombinoscope are used (PPP reagent: 30 pM recombinant tissue factor, 24 μM phospholipids in HEPES). The reaction is carried out in the presence of varying concentrations of test substance or the corresponding solvent. Moreover, a thrombin calibrator from Thrombinoscope is used whose amidolytic activity is required for calculating the thrombin activity in a plasma sample.

The test is carried out according to the manufacturer's instructions (Thrombinoscope BV): 4 μl of test substance or of the solvent, 76 μl of plasma and 20 μl of PPP reagent or thrombin calibrator are incubated at 37° C. for 5 min. After addition of 20 μl of 2.5 mM thrombin substrate in 20 mM HEPES, 60 mg/ml of BSA, 102 mM of calcium chloride, the thrombin generation is measured every 20 s over a period of 120 min. Measurement is carried out using a fluorometer (Fluoroskan Ascent) from Thermo Electron fitted with a 390/460 nm filter pair and a dispenser. Using the Thrombinoscope software, the thrombogram is calculated and represented graphically. The following parameters are calculated: lag time, time to peak, peak, ETP (endogenous thrombin potential) and start tail.

### a.5) Determination of the Anticoagulatory Activity

The anticoagulatory activity of the test substances is determined in vitro in human plasma, rabbit plasma and rat plasma. To this end, blood is drawn off in a mixing ratio of sodium citrate/blood of 1:9 using a 0.11 molar sodium citrate solution as receiver. Immediately after the blood has been drawn off, it is mixed thoroughly and centrifuged at about 4000 g for 15 minutes. The supernatant is pipetted off.

The Prothrombin time (PT, synonyms: thromboplastin time, quick test) is determined in the presence of varying concentrations of test substance or the corresponding solvent using a commercial test kit (Neoplastin® from Boehringer Mannheim or Hemoliance® RecombiPlastin from Instrumentation Laboratory). The test compounds are incubated with the plasma at 37° C. for 3 minutes. Coagulation is then started by addition of thromboplastin, and the time when coagulation occurs is determined. The concentration of test substance which effects a doubling of the prothrombin time is determined Representative activity data from this test are given in Table 3 below (in some cases as means of individual determinations):

TABLE 3

| Example No. | IC₅₀ [μM] | Example No. | IC₅₀ [μM] |
|---|---|---|---|
| 4 | 2.48 | 6 | 2.14 |
| 7 | 2.94 | 8 | 2.92 |
| 10 | 3.38 | 22 | 5.48 |
| 23 | 2.31 | 24 | 2.17 |
| 25 | 3.78 | 27 | 7.47 |
| 31 | 2.84 | 33 | 2.06 |
| 34 | 2.39 | 35 | 1.19 |
| 41 | 0.8 | 44 | 4.55 |
| 45 | 4.66 | 49 | 2.82 |
| 53 | 1.6 | 55 | 2.99 |
| 59 | 2.08 | 60 | 3.57 |
| 61 | 2.75 | 62 | 4.31 |
| 63 | 2.59 | 64 | 4.66 |
| 70 | 1.87 | 72 | 2.04 |
| 74 | 2.84 | 75 | 4.84 |

The thrombin time (TT) is determined in the presence of varying concentrations of test substance or the corresponding solvent using a commercial test kit (thrombin reagent from Roche). The test compounds are incubated with the plasma at 37° C. for 3 minutes. Coagulation is then started by addition of the thrombin reagent, and the time when coagulation occurs is determined. The concentration of test substance which effects a doubling of the thrombin time is determined.

The activated partial thromboplastin time (APTT) is determined in the presence of varying concentrations of test substance or the corresponding solvent using a commercial test kit (PTT reagent from Roche). The test compounds are incubated with the plasma and the PTT reagent (cephalin, kaolin) at 37° C. for 3 minutes. Coagulation is then started by addition of 25 mM calcium chloride, and the time when coagulation occurs is determined. The concentration of test substance which effects a doubling of the APTT is determined.

### a.6) Thromboelastography (Thromboelastogram)

The thromboelastography is carried out with the aid of the thromboelastograph ROTEM from Pentapharm and its accessories, cup and pin. The measurement is carried out in whole blood drawn off beforehand into sodium citrate monovettes from Sarstedt. The blood in the monovettes is kept in motion using a shaker and preincubated at 37° C. for 30 min

A 2-molar stock solution of calcium chloride in water is prepared. This is diluted 1:10 with an aqueous 0.9% sodium chloride solution. For the measurement, 20 μl of this 200 mM calcium chloride solution are initially charged into the cups (final concentration 12.5 mM calcium chloride). 3.2 μl of substance or solvent are added. The measurement is started by addition of 300 μl of whole blood. After the addition, using the tip of the pipette, the mixture is briefly drawn into the pipette and released again without generating air bubbles. The measurement is carried out over a period of 2.5 hours or stopped when fibrinolysis sets in. For evaluation, the following parameters are determined: CT (clotting time/[sec.]), CFT (clotting formation time/[sec.]), MCF (maximum clot firmness/[mm]) and the alpha angle [° ]. The measurement points are determined every 3 seconds and represented graphically, with the y axis for MCF [mm] and the x axis for time [sec.].

### a.7) Inhibition of the Coagulation Factor Thrombin Bound to the Thrombus

Blood clots formed either prior to the start of a therapy with anticoagulants, during therapy-free periods or in spite of therapy contain large amounts of coagulation factors which may favour progressive thrombus formation. These coagulation factors are tightly bound to the thrombus and can not be washed out. In certain clinical situations, this may result in a risk for the patient. Using the tests listed below, it is possible to demonstrate, in human thrombi, both thrombin and FXa having biological (procoagulatory) activity.

### Thrombi Formed In Vitro

Thrombi are formed in vitro from human plasma and examined for activity of the bound coagulation factors thrombin and FXa. To this end, 300 μl of plasma, 30 μl of lipid vesicles and 30 μl of an aqueous calcium chloride solution are mixed in a 48 MTP plate and incubated for 30 min. This step and the following steps are carried out at 37° C. and with constant agitation (300 rpm). The thrombi formed are transferred to a new 48 MTP plate and twice washed for 10 min in 0.9% sodium chloride solution, the thrombus being dabbed on filter paper between the washing steps. The thrombus is transferred into buffer B (Owren's Veronal buffer, 1% BSA) and incubated for 15 mM, dabbed on filter paper and incubated for 30 min in test substance in various concentrations in buffer B. The clots are then washed twice as described above. The thrombi are dabbed and transferred into buffer D: (240 μl Owren's Veronal buffer, 1% BSA and 15.6 mM calcium chloride) and incubated with or without 0.6 μM prothrombin for 45 min. The reaction is stopped with 75 μl of 1% EDTA solution. Thrombin activity is measured separately in the thrombus in buffer A (7.5 mM Na₂EDTAx2H₂O, 175 mM sodium chloride, 1% BSA, pH 8.4) or in the supernatant from the last step. To this end, the thrombin substrate used in a.1) is employed in a final concentration of 50 μM, and the resulting fluorescence is measured in a fluorescence plate reader (360/465 nm).

### a.8) Effect of the Thrombin Inhibitors on Thrombolysis in Platelet-Poor Plasma

The effect of the test substances on in vitro thrombolysis in platelet-poor plasma is tested in the presence of tissue plasminogen activator (tPA). To this end, with monitoring by turbidity measurement (UV absorption at 405 nm), initially a clot is formed in a microtitre plate in human plasma with addition of tissue factor, and the dissolution of the clot is adjusted to a certain time window by simultaneous addition of tissue plasminogen activator (tPA). Simultaneous addition of different amounts of the test substance may result in a shortening of the thrombolysis time (the time it takes from maximum turbidity to getting back to the baseline).

In a 384-well microtitre plate, 0.7 μl of an ethanol/water mixture (1:1) comprising various concentrations of the test substances, 1.7 μl of a solution of human thrombomodulin (final concentration 10 nM) and 1.7 μl of a solution of human tissue plasminogen activator (Actilyse®, final concentration 3 nM) are added to 63 μl of human plasma (German Red Cross, corresponds to 90% plasma in the test). Coagulation is initiated by addition of 3.5 μl of a tissue factor-containing solution (Recombiplastin 2G in a 1:100 dilution in 0.2 M calcium chloride solution) at 37° C. Measurement of turbidity (UV absorptions measurement at 405 nm) at one minute intervals is then started immediately. The thrombolysis time is calculated as the time it takes from maximum absorption to getting back to the baseline.

### b) Determination of Antithrombotic Activity (In Vivo)

### b.1) Arteriovenous Shunt and Haemorrhage Model (Combi-Model Rat)

Fasting male rats (strain: HSD CPB:WU) having a weight of 300-350 g are anaesthetized using Inactin (150-180 mg/kg). Thrombus formation is initiated in an arteriovenous shunt in accordance with the method described by Christopher N. Berry et al., Br. J. Pharmacol. (1994), 113, 1209 1214. To this end, the left jugular vein and the right carotid artery are exposed. The two vessels are connected by an extracorporeal shunt using a polyethylene tube (PE 60) having a length of 10 cm. In the middle, this polyethylene tube is attached to a further polyethylene tube (PE 160) having a length of 3 cm which contains a roughened nylon thread arranged to form a loop, to form a thrombogenic surface. The extracorporeal circulation is maintained for 15 minutes. The shunt is then removed and the nylon thread with the thrombus is weighed immediately. The weight of the nylon thread on its own is determined before the experiment is started.

To determine the bleeding time, immediately after opening of the shunt circulation, the tip of the tail of the rats is docked by 3 mm using a razor blade. The tail is then placed into physiological saline kept at a temperature of 37° C., and the bleeding from the cut is observed over a period of 15 minutes. What is determined is the time until bleeding ceases for at least 30 seconds (initial bleeding time), total bleeding time over a period of 15 minutes (cumulative bleeding time) and the quantitative blood loss via photometric determination of the collected haemoglobin.

Before the extracorporeal circulation is set up and the tip of the tail is docked, the test substances are administered to the animals while awake either intravenously via the contralateral jugular vein as a single bole or as a bole with subsequent continuous infusion or orally using a pharyngeal tube.

### b.2) Iron(II) Chloride Damage and Bleeding Model (Combi Model II, Rat)

Male rats (strain: HsdRCCHan:Wist) having a weight of 300 g-325 g are anaesthetized intraperitoneally with Inactin (180 mg/kg). Thrombus formation is triggered using iron(II) chloride in the carotid artery. To this end, the right carotid artery is exposed. A flow probe head is then attached, and the blood flow is recorded for 10 minutes. Artery and surroundings are then drained. Parafilm (10×8 mm) and filter paper (10×6 mm folded) are placed under the carotid artery and wetted with 20 μl iron(II) chloride solution (iron(II) chloride tetrahydrate reagent plus 99%, Sigma, 5% solution in water is prepared). A small piece of filter paper is placed on top of the carotid artery and also wetted with iron(II) chloride solution. The carotid artery prepared in this manner is covered with a moist swab and left for 5 minutes. Parafilm and filter paper are then removed and the artery is rinsed with physiological sodium chloride solution. The flow probe head is reattached and the blood flow is recorded for 30 minutes. The measurement is then stopped and the exposed section of the carotid artery is pinched off with tissue clamps and excised. The thrombus located in the vessel is removed from the vessel with the aid of a pair of tweezers and weighed immediately.

To determine the bleeding time, after injury and re-attachment of the flow probe head, the tip of the tail of the rat is docked by 3 mm using a razor blade. The tail is then placed into water kept at a temperature of 37° C., and the bleeding from the cut is observed over a period of 15 minutes. What is determined is the time until bleeding ceases for at least 30 seconds (initial bleeding time), total bleeding time over a period of 15 minutes (cumulative bleeding time) and the quantitative blood loss via photometric determination of the collected haemoglobin.

The test substances are administered either intravenously via the jugular vein as single bole directly before the start of the experiment or as a bole (prior to the start) with subsequent continuous infusion.

### b.3) Rabbit Venous Reperfusion and Bleeding Model (Combi Model Rabbit)

Male New Zealand rabbits having a weight of 2.8-3.4 kg are anaesthetized using an intramuscular ketamine/Rompun bole injection. The animal is then shaved at the places needed for the surgery. A continuous infusion of anaesthetic (ketamine/Rompun) is administered via the left auricular vein using an indwelling catheter. Left and right femoral vein and right femoral artery are catheterized with a polyethylene tube (PESO). The jugular vein is then carefully exposed such that the vessel is stressed and damaged as little as possible and no more fat is present at the vessel. Using a suitable apparatus for measuring flow (Powerlab, Transonic TS420 incl. flow probe head), the flow in the jugular vein is recorded (Lab Chart Software). Prior to the start of the experiment, twice 1.4 ml of citrated blood are removed from the rabbit via the femoral artery, and the basal bleeding time at the rim of the ear is determined. Once there has been a constant flow from the jugular vein for 10 min (complete regeneration of the vessel after preparation), a 2 cm section of the vein is pinched off using small vessel clamps. In a Petri dish, the citrated blood removed earlier (300 μl) is mixed with calcium chloride (0.25 M, 90 μl) and thrombin (25 U/ml, 60 μl). 180 μl of the blood/calcium chloride/thrombin mixture are quickly drawn into a 1 ml syringe and, via a 27G cannula, injected into the pinched-off segment of the vessel. The injection site is pinched off with a pair of tweezers for one minute so that no blood can escape. Two minutes after injection of the thrombus, the test substance is administered as bole and infusion via the left femoral vein catheter. 14 minutes after the thrombus injection, tissue plasminogen activator is administered as bole and infusion (Actilyse®, 20 μg/kg bole & 150 μg/kg/h infusion) at the right femoral vein. 15 minutes after thrombus injection, the stasis is opened and the flow probe head is attached. Blood flow in the vessel is recorded for 120 minutes, and the vessel is kept moist with warm 0.9% aqueous sodium chloride solution during this time. After 105 minutes of reperfusion, the ear bleeding time is determined again. At the end of the experiment, after 120 minutes of reperfusion, 1.4 ml of citrated blood are removed, the animal is sacrificed painlessly by a bole injection of 1.5 ml of T61 and the weight of the thrombus in the jugular vein is determined. The blood removed before and after the experiment is used to obtain plasma and to determine the ex vivo coagulation time.

The area under the blood flow/time curve (AUC) is calculated and correlated to the maximum achievable area, which is calculated from the blood flow before the experiment and the time (120 min). The area obtained with tissue plasminogen activator alone is subtracted from the area achieved using the respective substance or dosage. The resulting area is a measure of the improvement of reperfusion by the test substance.

### c) Determination of Pharmacokinetics

### c.1) Pharmacokinetics Following Intravenous Administration of the Test Substance

Male Wistar rats are anaesthetized, and a catheter is placed in the jugular vein. The next day, a defined dose of the test substance is administered as a solution by injection into the tail vein. Blood samples are collected via the catheter over a period of 7 hours (9 points in time).

A defined dose of the test substance is administered to female Beagles as a solution via the cephalic vein as a 15 min infusion. Blood samples are collected via a catheter in the cephalic vein over a period of 7 hours (12 points in time).

The blood is centrifuged in heparin tubes. To precipitate the protein, acetonitrile is added and the plasma sample is centrifuged. The test substance is quantified in the supernatant by LC/MS-MS. The test substance plasma concentrations determined are used to calculate the pharmacokinetic parameters such as AUC (area under the plasma concentration/time curve), Vₛₛ (distribution volume), Cₘₐₓ (highest concentration of the test substance in the plasma after administration), t_{1/2} (half-life) and CL (total clearance of the test substance from the plasma). To calculate the blood clearance, the blood/plasma distribution is determined by incubating the test substance in blood. After removal of the plasma by centrifugation, the concentration of the test substance in the plasma is determined by LC/MS-MS.

### c.2) Pharmacokinetics Following Oral Administration of the Test Substance

Male Wistar rats are anaesthetized, and a catheter is placed in the jugular vein. The next day, a defined dose of the test substance is administered orally. Blood samples are collected via the catheter over a period of 24 hours (9 points in time).

A defined dose of the test substance is administered orally to female Beagles. Blood samples are collected via a catheter in the cephalic vein over a period of 24 hours (9 points in time).

The blood is centrifuged in heparin tubes. To precipitate the protein, acetonitrile is added and the plasma sample is centrifuged. The test substance is quantified in the supernatant by LC/MS-MS. The test substance plasma concentrations determined are used to calculate pharmacokinetic parameters such as AUC (area under the plasma concentration/time curve), Cmax (highest concentration of the test substance in the plasma after administration), t_{1/2} (half-life) and F (bioavailability).

### c.3) Caco-2 Permeability Assay

The in vitro permeability of the test substance through a Caco-2 cell monolayer is determined using an established in vitro system for predicting the permeability through the gastrointestinal tract [1]. CaCo-2 cells (ACC No. 169, DSMZ, Deutsche Sammlung von Mikroorganismen and Zellkulturen, Brunswick, Germany) are sown in 24-well plates and cultivated for 14 to 16 days. The test substance is dissolved in DMSO and diluted to a concentration of 2 μM in transport buffer (HBSS, Hanks Buffered Salt Solution, Gibco/Invitrogen, supplemented with glucose (final concentration 19.9 mM) and HEPES (final concentration 9.8 mM)). To determine the permeability from apical to basolateral (Pₐₚₚ A-B), the test substance is added on the apical side and transport buffer is added at the basolateral side of the cell monolayer. To determine the permeability from basolateral to apical (Pₐₚₚ B-A), the test substance is added on the basolateral side and transport buffer is added at the apical side of the cell monolayer. At the start of the experiment, samples are taken from the donor compartment to determine the mass balance. After an incubation time of 2 hours at 37° C., samples were taken from the two compartments. The samples were quantified by LC-MS/MS, and the permeability coefficients were calculated. For each cell monolayer, the permeability of Lucifer Yellow was determined to ensure cell monolayer integrity. In each test run, the permeability of atenolol (marker for low permeability) and sulfasalazine (marker for active excretion) is also determined to check the quality of the cells.

Literature: Artursson, P. and Karlsson, J. (1991). Correlation between oral drug absorption in humans and apparent drug permeability coefficients in human intestinal epithelial (Caco-2) cells. Biochem. Biophys. 175 (3), 880-885.

c.4) In Vitro Clearance Determinations with Hepatocytes

Incubations with fresh primary hepatocytes are carried out at 37° C. in a total volume of 1.5 ml with a modified Janus® robot (Perkin Elmer) while shaking. The incubations typically contain 1 million living liver cells/ml, ˜1 μM substrate and 0.05 M potassium phosphate buffer (pH=7.4). The final ACN concentration in the incubation is <1%.

Aliquots of 125 μl are withdrawn from the incubations after 2, 10, 20, 30, 50, 70 and 90 min and transferred into 96-well filter plates (0.45 μm low-binding hydrophilic PTFE; Millipore: MultiScreen Solvinert). Each of these contain 250 μl of ACN to stop the reaction. After the centrifugation, the filtrates are analysed by MS/MS (typically API 3000).

The in vitro clearances are calculated from the half-lives of the substance degradation, using the following equations:

□CL′_{intrinsic}[ml/(min·kg)]=(0.693/in vitro t1/2 [min])·(liver weight [g liver/kg body weight])·(cell number[1.1·10̂8]/liver weight [g])/(cell number[1·10̂6]/incubation volume [ml])

CL_{blood} is calculated without taking into account the free fraction (“nonrestricted well stirred model”) by the following equation:

CL_{blood} well-stirred [1/(h·kg)]=(Q_{H}[1/(h·kg)]·CL′_{intrinsic}[1/(h·kg)])/(Q_{H}[1/(h·kg)]+CL′_{intrinsic}[1/(h·kg)])

The species-specific extrapolation factors used for the calculation are summarized in Table 4 below:

TABLE 4

| | male/female | | | | | |
|---|---|---|---|---|---|---|
| | Mouse | Mouse | Rat | Dog | Cyno | Man |
| | m | f | m/f | m/f | f | m/f |
| Cell number/g | 110 | 110 | 110 | 110 | 110 | 110 |
| Liver [millions | | | | | | |
| of cells] | | | | | | |
| Liver [g]/ | 50 | 43 | 32 | 39 | 30 | 21 |
| kg Body Weight | | | | | | |
| Liver Blood | 5.4 | 5.4 | 4.2 | 2.1 | 2.5 | 1.3 |
| Flow [l/(h · kg)] | | | | | | |

Fₘₐₓ values which state the maximum possible bioavailability—based on the hepatic extraction—are calculated as follows:

Fₘₐₓ well-stirred [%]=(1−(CL_{blood} well-stirred[1/(h·kg)]/Q_{H}[1/(h·kg)]))·100

### c.5) CYP Inhibition Test

Inhibitory properties of an active compound on the cytochromes P450 (CYP) of the human body may entail extensive clinical effects (drug interactions) because most prescribed medicaments are degraded (metabolized) by these enzymes. Involved in this in particular are the CYP isoenzymes of the 1A and 2C families, CYP2D6 and, with a proportion of almost 50%, CYP3A4. In order to preclude or minimize these possible drug interactions (Drug-Drug Interactions, DDI), the ability of substances to be able to inhibit CYP1A2, CYP2C8, CYP2C9, CYP2D6 and CYP3A4 in humans is investigated using human liver microsomes (pool from various individuals). This takes place by measuring CYP isoform-specific metabolites formed from standard substrates such as, for example, phenacetin, amodiaquin, diclofenac, dextromethorphan, midazolam and testosterone. The inhibitory effects are investigated at six different concentrations of the test compounds (1.5, 3.1, 6.3, 12.5, 25 and 50 μM as maximum concentration or 0.6, 1.3, 2.5, 5, 10 and 20 μM as maximum concentration), compared with the extent of the CYP isoform-specific metabolite formation of the standard substrates in the absence of the test compounds, and the corresponding IC₅₀ values are calculated. CYP isoform-specific standard inhibitors such as, for example, furafylline, montelukast, sulfaphenazole, fluoxetine and ketoconazole serve as control of the results obtained. In order to obtain indications of the possible mechanism-based inhibitors (MBI) on CYP3A4, the human liver microsomes are incubated in the presence of the inhibitor to be investigated for 30 minutes before the addition of midazolam or testosterone as standard substrates of CYP3A4. A reduction in the IC₅₀ obtained by comparison with the mixture without preincubation serves as an indicator of a mechanism-based inhibition. Mibefradil serves as positive control.

### Procedure:

The incubations of the standard substrates with human liver microsomes (14-100 μg/ml) in the presence of the test compound (as potential inhibitor) are carried out at 37° C. in 96-well plates on a workstation (Tecan, Genesis; Hamiltion, MICROLAB STARLET). The incubation times are 10-15 minutes. The test compounds are preferably dissolved in acetonitrile (1.0, 2.0 or 2.5, 5.0 mM stock solution). The 96-well plates are prepared by sequential addition of a stock solution of NADP+, EDTA, glucose 6-phosphate and glucose 6-phosphate dehydrogenase in phosphate buffer (pH 7.4), the test compound and a solution of standard substrate and human liver microsomes in phosphate buffer (pH 7.4). The total volume is 200 μl. Also located on the 96-well plate are the corresponding control incubations with and without standard inhibitor. After the respective incubation time, the incubations are stopped by addition of 100 μl of acetonitrile comprising a suitable internal standard. Precipitated proteins are removed by centrifugation (3000 rpm, 10 minutes, 10° C.). The resulting supernatants of the respective plates are combined on a plate and analysed by LC-MS/MS. From the measurement data obtained, the IC₅₀ values are generated and used to assess the inhibitory potential of the test compound.

### c.6) Cellular In Vitro Test for Determining the Induction of Drug-Degrading Cytochromal Enzymes (CYPs) in Primary Human Hepatocytes

Enzyme induction is an unwanted property of a drug which puts broad and safe use of the active compound into question. A consequence of enzyme induction is an accelerated degradation (metabolization) of drugs in the liver. Combined intake of an enzyme inducer and other medicaments such as, for example, immunosuppressives, coagulants or else contraceptives may lead to complete ineffectiveness of the drugs.

The object of the investigation is to provide substances which do not have this unwanted drug interaction. Enzyme inductors are identified with the aid of primary human hepatocytes in long-term culture. To cultivate the cells, hepatocytes are plated on a collagen I layer (density 100 000 cells/cm²), and the grown-on cells are then covered with a second collagen layer (sandwich method). (Kern A, Bader A, Pichlmayr R, and Sewing K F, Biochem Pharmacol., 54, 761-772 (1997)). To obtain the effect of the test substances on the regulation of the liver enzymes, the hepatocytes are incubated with the active compounds for several days in long-term culture.

### Assay Procedure:

After a two-day regeneration phase, the cells are treated in Williams Medium E, 10% FCS, prednisolone, insulin, glucagon and L-glutamine, penicillin and streptomycin with the test substances. To this end, stock solutions of the active compounds having a concentration of 1 mg/ml in acetonitrile or methanol are prepared and, in 8 dilution steps (1:3) in cell culture medium, pipetted to the cell cultures, which are then incubated in a cell incubator (96% atmospheric humidity, 5% v/v carbon dioxide, 37° C.) for about 5 days. The cell culture medium is changed daily. After this incubation time, the cell cultures are incubated with cytochrome P450(CYP)-specific substrates to determine the activity of the liver enzymes CYP1A2, CYP3A4, CYP2B6 and CYP2C19. The samples thus stopped are either analysed directly or stored at −20° C. until analysis.

To this end, the media of the cell cultures are chromatographed using suitable C18-reversed-phase columns and variable mixtures of acetonitrile and 10 mM ammonium formate (HPLC-MS/MS).

The mass spectrometric data serve to quantify the substrate turnover and, derived therefrom, to calculate the liver enzyme activities. Active compounds having unfavourable properties with respect to liver enzyme regulation are not pursued any further.

### C) WORKING EXAMPLES OF PHARMACEUTICAL COMPOSITIONS

The substances according to the invention can be converted to pharmaceutical preparations as follows:

### Tablet:

### Composition:

100 mg of the compound of Example 1, 50 mg of lactose (monohydrate), 50 mg of maize starch, 10 mg of polyvinylpyrrolidone (PVP 25) (from BASF, Germany) and 2 mg of magnesium stearate.

Tablet weight 212 mg. Diameter 8 mm, radius of curvature 12 mm

### Production:

The mixture of the compound of Example 1, lactose and starch is granulated with a 5% strength solution (m/m) of the PVP in water. After drying, the granules are mixed with the magnesium stearate for 5 min. This mixture is compressed in a conventional tabletting press (see above for format of the tablet).

### Oral Suspension:

### Composition:

1000 mg of the compound of Example 1, 1000 mg of ethanol (96%), 400 mg of Rhodigel (xanthan gum) (from FMC, USA) and 99 g of water.

10 ml of oral suspension correspond to a single dose of 100 mg of the compound according to the invention.

### Production:

The Rhodigel is suspended in ethanol, and the compound of Example 1 is added to the suspension. The water is added while stirring. The mixture is stirred for about 6 h until swelling of the Rhodigel is complete.

### Intravenously Administrable Solution:

### Composition:

1 mg of the compound of Example 1, 15 g of polyethylene glycol 400 and 250 g of water for injection purposes.

### Production:

The compound of Example 1 is dissolved together with polyethylene glycol 400 by stirring in the water. The solution is sterilized by filtration (pore diameter 0.22 μm) and dispensed under aseptic conditions into heat-sterilized infusion bottles. The latter are closed with infusion stoppers and crimped caps.

1. Compound of the formula 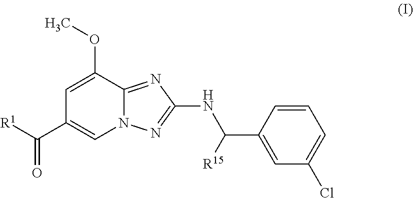
in which
R¹ represents a group of the formula 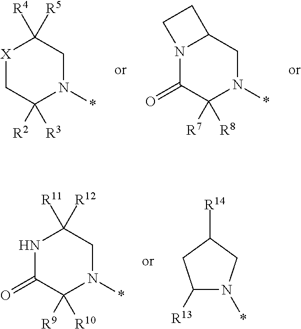
where * is the point of attachment to the carbonyl group,
X represents an oxygen atom, a sulphur atom or CH—R⁶,
where
R⁶ represents hydrogen or hydroxy,
R² represents hydrogen, aminocarbonyl, C₁-C₆-alkyl, C₃-C₆-cycloalkyl or phenyl,
where alkyl and cycloalkyl may be substituted by a substituent selected from the group consisting of hydroxy, methoxy, cyano, hydroxycarbonyl, aminocarbonyl, methylsulphonyl, difluoromethoxy and trifluoromethoxy,
or
where alkyl and cycloalkyl may be substituted by 1 to 3 fluorine substituents,
R³ represents hydrogen or C₁-C₄-alkyl,
or
R² and R³ together with the carbon atom to which they are attached form a cyclobutyl ring, cyclobutyl ring or cyclopentyl ring,
where the cyclobutyl ring and the cyclopentyl ring may be substituted by a hydroxy substituent,
R⁴ represents hydrogen or C₁-C₆-alkyl,
where alkyl may be substituted by a hydroxy substituent,
or
where alkyl may be substituted by 1 to 3 fluorine substituents,
R⁵ represents C₁-C₄-alkyl,
or
R⁴ and R⁵ together with the carbon atom to which they are attached form a cyclopropyl ring, cyclobutyl ring or cyclopentyl ring,
where the cyclobutyl ring and the cyclopentyl ring may be substituted by a hydroxy substituent,
R⁷ represents hydrogen or C₁-C₆-alkyl,
where alkyl may be substituted by one substituent selected from the group consisting of cyano, hydroxy and methoxy,
or
where alkyl may be substituted by 1 to 3 fluorine substituents,
R⁸ represents hydrogen,
R⁹ represents hydrogen or C₁-C₆-alkyl,
where alkyl may be substituted by one substituent selected from the group consisting of hydroxy and cyano,
or
where alkyl may be substituted by 1 to 3 fluorine substituents,
R¹⁰ represents hydrogen,
R¹¹ represents C₁-C₄-alkyl,
where alkyl may be substituted by a hydroxy substituent,
R¹² represents hydrogen or C₁-C₄-alkyl,
or
R¹¹ and R¹² together with the carbon atom to which they are attached form a cyclopropyl ring, cyclobutyl ring or cyclopentyl ring,
where the cyclobutyl ring and the cyclopentyl ring may be substituted by a hydroxy substituent,
R¹³ represents hydroxymethyl or hydroxyethyl,
R¹⁴ represents methoxy or ethoxy,
where methoxy and ethoxy may be substituted by 1 to 3 substituents selected from the group consisting of deuterium and fluorine,
and
R¹⁵ represents hydrogen, methyl or fluoromethyl,
or one of the salts thereof, solvates thereof or solvates of the salts thereof.

2. Compound according to claim 1, characterized in that
R¹ represents a group of the formula 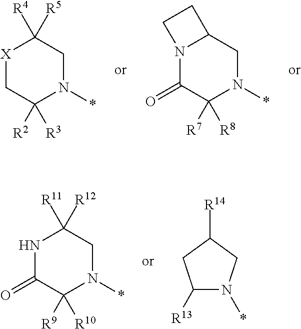
where * is the point of attachment to the carbonyl group,
X represents an oxygen atom or CH—R⁶,
where
R⁶ represents hydrogen,
R² represents aminocarbonyl, C₁-C₄-alkyl or C₃-C₆-cycloalkyl,
where alkyl and cycloalkyl may be substituted by a substituent selected from the group consisting of hydroxy, methoxy and hydroxycarbonyl,
or
where alkyl may be substituted by 1 to 3 fluorine substituents,
R³ represents hydrogen or C₁-C₄-alkyl,
or
R² and R³ together with the carbon atom to which they are attached form a cyclopbutyl ring,
where the cyclobutyl ring may be substituted by a hydroxy substituent,
R⁴ represents hydrogen or C₁-C₄-alkyl,
where alkyl may be substituted by a hydroxy substituent,
R⁵ represents C₁-C₄-alkyl,
R⁷ represents C₁-C₄-alkyl,
where alkyl may be substituted by a methoxy substituent,
R⁸ represents hydrogen,
R⁹ represents C₁-C₄-alkyl,
R¹⁰ represents hydrogen,
R¹¹ represents C₁-C₄-alkyl,
R¹² represents hydrogen,
or
R¹¹ and R¹² together with the carbon atom to which they are attached form a cyclopropyl ring,
R¹³ represents hydroxymethyl,
R¹⁴ represents ethoxy,
where ethoxy may be substituted by 1 to 3 substituents selected from the group consisting of deuterium and fluorine,
and
R¹⁵ represents hydrogen, methyl or fluoromethyl,
or one of the salts thereof, solvates thereof or solvates of the salts thereof.

3. Compound according to claim 1, characterized in that
R¹ represents a group of the formula 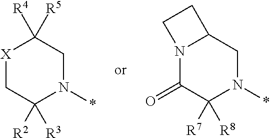
where * is the point of attachment to the carbonyl group,
X represents an oxygen atom,
R² represents C₁-C₄-alkyl or cyclobutyl,
where alkyl is substituted by a hydroxy substituent,
and
where cyclobutyl is substituted by a hydroxy substituent,
R³ represents hydrogen,
R⁴ represents hydrogen or methyl,
and
R⁵ represents methyl,
or
R² represents methyl,
where methyl may be substituted by 1 to 2 fluorine substituents,
R³ represents hydrogen or methyl,
R⁴ represents C₁-C₄-alkyl,
where alkyl is substituted by a hydroxy substituent,
and
R⁵ represents methyl,
or
R² and R³ together with the carbon atom to which they are attached form a cyclobutyl ring,
where the cyclobutyl ring is substituted by a hydroxy substituent,
R⁴ represents hydrogen,
and
R⁵ represents methyl,
R⁷ represents methyl,
R⁸ represents hydrogen,
and
R¹⁵ represents hydrogen, methyl or fluoromethyl,
or one of the salts thereof, solvates thereof or solvates of the salts thereof.

4. The compound according to claim 1, characterized in that
R¹ represents a group of the formula 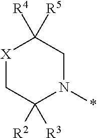
where * is the point of attachment to the carbonyl group,
X represents an oxygen atom,
R² represents C₁-C₄-alkyl or cyclobutyl,
where alkyl is substituted by a hydroxy substituent,
and
where cyclobutyl is substituted by a hydroxy substituent,
R³ represents hydrogen,
R⁴ represents hydrogen or methyl,
and
R⁵ represents methyl,
or
R² represents methyl,
where methyl may be substituted by 1 to 2 fluorine substituents,
R³ represents hydrogen or methyl,
R⁴ represents C₁-C₄-alkyl,
where alkyl is substituted by a hydroxy substituent,
and
R⁵ represents methyl,
or
R² and R³ together with the carbon atom to which they are attached form a cyclobutyl ring,
where the cyclobutyl ring is substituted by a hydroxy substituent,
R⁴ represents hydrogen,
and
R⁵ represents methyl,
and
R¹⁵ represents hydrogen, methyl or fluoromethyl,
or one of the salts thereof, solvates thereof or solvates of the salts thereof.

5. The compound according to claim 1, characterized in that
R¹ represents a group of the formula 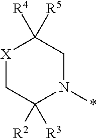
where * is the point of attachment to the carbonyl group,
X represents an oxygen atom,
R² and R³ together with the carbon atom to which they are attached form a cyclobutyl ring,
where the cyclobutyl ring is substituted by a hydroxy substituent,
R⁴ represents hydrogen,
and
R⁵ represents methyl,
and
R¹⁵ represents hydrogen, methyl or fluoromethyl,
or one of the salts thereof, solvates thereof or solvates of the salts thereof.

6. The compound according to claim 1, characterized in that the compound is
{2-[(3-chlorobenzyl)amino]-8-methoxy[1,2,4]triazolo[1,5-a]pyridin-6-yl}[5-(3-hydroxycyclobutyl)-2-methylmorpholin-4-yl]methanone [diastereomer 3+diastereomer 4]
or
{2-[(3-chlorobenzyl)amino]-8-methoxy[1,2,4]triazolo[1,5-a]pyridin-6-yl}[(5R)-2-(2-hydroxyethyl)-2,5-dimethylmorpholin-4-yl]methanone [enantiomerically pure isomer]
or
(2-{[1-(3-chlorophenyl)-2-fluoroethyl]amino}-8-methoxy[1,2,4]triazolo[1,5-a]pyridin-6-yl)[5-(2-hydroxyethyl)-2-methylmorpholin-4-yl]methanone [enantiomerically pure isomer 2]
or
(2-{[1-(3-chlorophenyl)-2-fluoroethyl]amino}-8-methoxy[1,2,4]triazolo[1,5-a]pyridin-6-yl)[(cis)-2-hydroxy-7-methyl-8-oxa-5-azaspiro[3.5]non-5-yl]methanone [enantiomerically pure isomer 1]
or
(2-{[1-(3-chlorophenyl)-2-fluoroethyl]amino}-8-methoxy[1,2,4]triazolo[1,5-a]pyridin-6-yl)[(cis)-2-hydroxy-7-methyl-8-oxa-5-azaspiro[3.5]non-5-yl]methanone [enantiomerically pure isomer 2]
or
{2-[(3-chlorobenzyl)amino]-8-methoxy[1,2,4]triazolo[1,5-a]pyridin-6-yl}[(5R)-2-(2-hydroxypropyl)-2,5-dimethylmorpholin-4-yl]methanone [enantiomerically pure isomer]
or
4-({2-[(3-chlorobenzyl)amino]-8-methoxy[1,2,4]triazolo[1,5-a]pyridin-6-yl}carbonyl)-3-methyl-1,4-diazabicyclo[4.2.0]octan-2-one [enantiomerically pure isomer]
or one of the salts, the solvates or the solvates of the salts of these compounds.

7. Process for preparing a compound of the formula (I) or one of the salts thereof, solvates thereof or solvates of the salts thereof according to claim 1, characterized in that either
[A] a compound of the formula 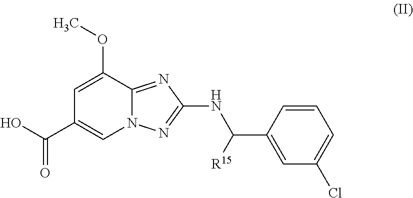
in which
R¹⁵ the meaning given in claim 1,
is reacted with a compound of the formula
R¹—H (III)
in which
R¹ the meaning given in claim 1,
in the presence of dehydrating reagents
or
[B] a compound of the formula 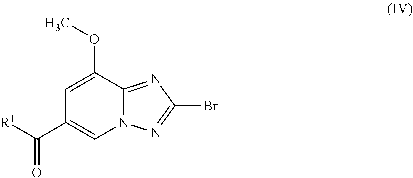
in which
R¹ has the meaning given in claim 1,
is reacted with a compound of the formula 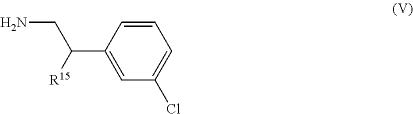
in which
R¹⁵ has the meaning given in claim 1,
in the presence of a palladium catalyst.

8. The compound according to claim 1, is for treatment and/or prophylaxis of diseases.

9. Use of the compound according to claim 1, for producing a medicament for treatment and/or prophylaxis of diseases.

10. Use of a compound according to claim 1, for producing a medicament for the treatment and/or prophylaxis of thromboembolic disorders.

11. Use of a compound according to claim 1 to 6 for producing a medicament for the treatment and/or prophylaxis of acute coronary syndrome (ACS), venous thromboembolisms, venous thromboses, in particular in deep leg veins and kidney veins, pulmonary embolisms, stroke and/or thrombosis prophylaxis in the context of surgical interventions, in particular in the context of surgical interventions in patients suffering from cancer.

12. Medicament comprising a compound according to claim 1, in combination with an inert, nontoxic, pharmaceutically suitable excipient.

13. Medicament according to claim 12 for the treatment and/or prophylaxis of thromboembolic disorders.

14. Method for the treatment of thromboembolic disorders in humans and animals by administration of a therapeutically effective amount of a compound according to claim 1, of a medicament.
